(19) United States
(12) Patent Application Publication
(10) Pub. No.: US 2026/0098062 A1
(43) Pub. Date: Apr. 9, 2026

(54) LIPID PARTICLES COMPRISING VARIANT PARAMYXOVIRUS ATTACHMENT GLYCOPROTEINS AND USES THEREOF

(71) Applicant: Sana Biotechnology, Inc., Seattle, WA (US)

(72) Inventors: Patricia CRUITE, Medford, MA (US); Katherine Elizabeth SULLIVAN, Haverhill, MA (US); Alexander Mark JUSTEN, Somerville, MA (US); Valeria BERLFEIN, Cambridge, MA (US); Shirisha AMATYA, Medford, MA (US); Albert Ruzo MATIAS, Brookline, MA (US); Kyle TRUDEAU, Boston, MA (US); Matteo STOPPATO, Stoneham, MA (US)

(73) Assignee: Sana Biotechnology, Inc., Seattle, WA (US)

(21) Appl. No.: 19/113,834

(22) PCT Filed: Sep. 21, 2023
(86) PCT No.: PCT/US2023/074804

§ 371 Date: Mar. 20, 2025

Related U.S. Application Data
(60) Provisional application No. 63/408,823, filed on Sep. 21, 2022.
(60) Provisional application No. 63/522,723, filed on Jun. 22, 2023.

(51) Int. Cl. C07K 14/005; C07K 16/28; C12N 15/86

(52) U.S. Cl. CPC C07K 14/005; C07K 16/2812; C07K 16/2815; C12N 15/86; C07K 2317/569; C07K 2317/622; C07K 2319/33; C12N 2740/15043; C12N 2740/15045; C12N 2760/18022

(57) ABSTRACT

Provided herein are lipid particles, including viral-based particles, comprising Paramyxovirus glycoproteins having reduced glycosylation, and in some aspects, comprise variant G/H proteins. Also provided are methods for producing and modifying lipid particles, including viral-based particles, wherein the particle has reduced glycosylation of the G/H protein, as well as methods for using the particles.

### CROSS-REFERENCE TO RELATED APPLICATIONS

This application claims priority from U.S. provisional application No. 63/408,823, filed Sep. 21, 2022, entitled “LIPID PARTICLES COMPRISING VARIANT PARAMYXOVIRUS ATTACHMENT GLYCOPROTEINS AND USES THEREOF,” and to U.S. provisional application No. 63/522,723, filed Jun. 22, 2023, entitled “LIPID PARTICLES COMPRISING VARIANT PARAMYXOVIRUS ATTACHMENT GLYCOPROTEINS AND USES THEREOF.” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 186152007240SeqList.xml created Sep. 21, 2023, which is 1,060,694 bytes in size. The information in the electronic format of the Sequence Listing is incorporated by reference in its entirety.

### FIELD OF THE INVENTION

The present invention relates to lipid particles and viral-based particles comprising Paramyxovirus glycoproteins, such as variant G/H proteins. The present disclosure also provides polynucleotides encoding the variant glycoproteins, as well as methods for preparing and using the lipid particles and viral-based particles.

### BACKGROUND OF THE INVENTION

Lipid particles, including viral particles and virus-like particles, are commonly used for delivery of exogenous agents to cells. Such particles can be pseudotyped using heterologous envelope proteins in order to target specific cells or cell types.

All references cited herein, including patent applications and publications, are incorporated by reference in their entirety.

### BRIEF SUMMARY OF THE INVENTION

In some aspects, provided herein is a pseudotyped viral particle or virus-like particle comprising a Paramyxovirus glycoprotein (F) and a Paramyxovirus G/H glycoprotein, wherein the G/H protein has reduced glycosylation as compared to a wild-type G/H protein.

In some aspects, provided herein is a lipid particle, comprising a lipid bilayer, a Paramyxovirus glycoprotein F, and a Paramyxovirus G/H glycoprotein, wherein the G/H protein is exposed on the outside of the lipid bilayer, and wherein the G/H protein has reduced glycosylation as compared to a wild-type G/H protein.

In some of any of the provided embodiments, the G/H protein is a variant G/H protein. In some of any of the provided embodiments, the variant G/H protein is a variant of a truncated paramyxovirus G/H glycoprotein in which is present the amino acid substitution at one or more amino acid positions. In some of any of the provided embodiments, the truncated paramyxovirus G/H glycoprotein has a truncated cytoplasmic tail as compared to the full-length G/H glycoprotein from the same Paramyxovirus, optionally wherein the full-length G/H glycoprotein is set forth in SEQ ID NO: 13. In some of any of the provided embodiments, the truncated paramyxovirus G/H glycoprotein has a truncated cytoplasmic that is set forth by any one of SEQ ID NOS: 355-557. In some of any of the provided embodiments, the truncated paramyxovirus G/H glycoprotein is a truncated Nipah G (NiV-G). In some of any of the provided embodiments, the truncated paramyxovirus G/H glycoprotein lacks up to 40, up to 30, up to 20, or up to 10 contiguous amino acids at the N-terminus. In some of any of the provided embodiments, the truncated paramyxovirus G/H glycoprotein lacks amino acid residues 2-34. In some of any of the provided embodiments, the truncated paramyxovirus G/H glycoprotein is set forth by any one of SEQ ID NOS: 1, 558-575. In some of any of the provided embodiments, the truncated G/H is set forth by a SEQ ID NO:1.

In some of any of the provided embodiments, the variant G/H protein comprises an amino acid substitution at one or more amino acid positions that reduce glycosylation of the G/H protein. In some of any of the provided embodiments, the one or more amino acid substitutions disrupts an N-linked glycosylation site. In some of any of the provided embodiments, the one or more amino acid substitutions disrupts an O-linked glycosylation site. In some of any of the provided embodiments, the one or more amino acid substitutions are at positions corresponding to positions selected from the group consisting of 39, 126, 128, 273, 345, 384, 448, and 496 of SEQ ID NO:1. In some of any of the provided embodiments, the amino acid substitution is a substitution at position 273. In some of any of the provided embodiments, the protein comprises at least three amino acid substitutions. In some of any of the provided embodiments, the amino acid substitutions are at positions 273, 384, and 496. In some of any of the provided embodiments, the amino acid substitution are at positions 273, 345, and 496. In some of any of the provided embodiments, the amino acid substitution is an asparagine substituted with a glutamine.

In some of any of the provided embodiments, the G/H protein is a NiV-G protein comprising a substitution at amino acid position 273 (N273Q) with reference to numbering of positions of SEQ ID NO: 1. In some of any of the provided embodiments, the G/H protein is a NiV-G protein comprising a substitution at amino acid position 273 (N273Q), a substitution at amino acid position 384 (N384Q), and a substitution at amino acid position 496 (N496Q), each with reference to numbering of positions of SEQ ID NO: 1. In some of any of the provided embodiments, the G/H protein is a NiV-G protein comprising a substitution at amino acid position 273 (N273Q), a substitution at amino acid position 345 (N345Q), and a substitution at amino acid position 496 (N496Q), each with reference to numbering of positions of SEQ ID NO: 1.

In some of any of the provided embodiments, the variant G/H protein comprises the sequence selected from the group consisting of SEQ ID NOs: 2-12 and 246-354. In some of any of the provided embodiments, the variant G/H protein comprises the sequence selected from the group consisting of SEQ ID NOs: 579-705. In some of any of the provided embodiments, the variant G/H protein comprises the sequence of SEQ ID NO: 5. In some of any of the provided embodiments, the variant G/H protein comprises the sequence of SEQ ID NO: 581. In some of any of the provided embodiments, the variant G/H protein comprises the sequence of SEQ ID NO: 259. In some of any of the provided embodiments, the variant G/H protein comprises the sequence of SEQ ID NO: 602. In some of any of the provided embodiments, the variant G/H protein comprises the sequence of SEQ ID NO: 263. In some of any of the provided embodiments, the variant G/H protein comprises the sequence of SEQ ID NO: 606.

In some of any of the provided embodiments, the paramyxovirus G/H protein further comprises a truncation at the N-terminus. In some of any of the provided embodiments, the truncation comprises a truncation corresponding to amino acids 1-34 of SEQ ID NO: 13. In some of any of the provided embodiments, the paramyxovirus G/H protein further comprises one or more amino acid mutations that reduce or abrogate native receptor tropism. In some of any of the provided embodiments, the amino acid mutations comprise mutations at positions corresponding to E501. W504, Q530, and E533 of SEQ ID NO: 13.

In some of any of the provided embodiments, the paramyxovirus G/H protein is fused to a targeting domain. In some of any of the provided embodiments, the targeting domain comprises a VHH or a scFv domain.

In some of any of the provided embodiments, the paramyxovirus G/H protein is a Nipah virus G protein (NiV-G).

In some of any of the provided embodiments, the pseudotyped viral particle, virus-like particle, or lipid particle further comprises a paramyxovirus F protein. In some of any of the provided embodiments, the F protein comprises a 22 amino acid truncation at the C-terminus. In some of any of the provided embodiments, the F protein is a Nipah virus F glycoprotein (NiV-F).

In some of any of the provided embodiments, the pseudotyped viral particle, virus-like particle, or lipid particle is generated from a producer cell comprising one or more mutations that disrupts protein glycosylation.

In some of any of the provided embodiments, the pseudotyped viral particle, virus-like particle, or lipid particle is generated from a producer cell contacted with an inhibitor of glycosylation.

In some of any of the provided embodiments, the pseudotyped viral particle, virus-like particle, or lipid particle is contacted with one or more glycosidases after generation.

In some of any of the provided embodiments, any one of the pseudotyped viral particles, virus-like particles, or lipid particles described herein further comprises an exogenous agent. In some of any of the provided embodiments, the exogenous agent is a protein or a nucleic acid. In some of any of the provided embodiments, the exogenous agent is a nucleic acid encoding a cargo for delivery to the target cell. In some of any of the provided embodiments, the exogenous agent is or encodes a therapeutic agent or a diagnostic agent. In some of any of the provided embodiments, the exogenous agent encodes a chimeric antigen receptor (CAR). In some of any of the provided embodiments, the exogenous agent encodes a genome modifying enzyme.

In some of any of the provided embodiments, the pseudotyped viral particle or virus-like particle or the lipid particle has reduced transduction of one or more off-target cell types. In some of any of the provided embodiments, the off-target cell type is a hepatocyte. In some of any of the provided embodiments, the off-target cell type is a hematopoietic stem cell.

In some of any of the provided embodiments, any one of the pseudotyped viral particles, virus-like particles, or lipid particles described herein does not have reduced transduction of on-target cell types. In some of any of the provided embodiments, the on target cell types comprise an immune cell.

In some aspects, provided herein is a variant paramyxovirus G/H protein comprising one or more amino acid substitutions at positions 39, 126, 128, 273, 345, 384, 448, and 496 corresponding to SEQ ID NO: 1. In some of any of the provided embodiments, the variant paramyxovirus G/H protein further comprises a truncation of amino acids 1-34 corresponding to SEQ ID NO: 13. In some of any of the provided embodiments, the variant protein is fused to a CD8 binding domain. In some of any of the provided embodiments,

In some of any of the provided embodiments, the variant protein is fused to a CD4 binding domain.

In some aspects, provided herein is a polynucleotide comprising a nucleic acid molecule encoding a variant paramyxovirus G protein comprising an amino acid substitution at one or more amino acid positions that reduce glycosylation of the variant G/H protein. In some of any of the provided embodiments, the variant G/H protein comprises an amino acid sequence selected from the group consisting of SEQ ID NO: 2-12 and 246-354. In some of any of the provided embodiments, the variant G/H protein comprises the sequence selected from the group consisting of SEQ ID NOs: 579-705. In some of any of the provided embodiments, the variant G/H protein comprises an amino acid sequence SEQ ID NO: 5. In some of any of the provided embodiments, the variant G/H protein comprises the sequence of SEQ ID NO: 581. In some of any of the provided embodiments, the variant G/H protein comprises an amino acid sequence SEQ ID NO: 259. In some of any of the provided embodiments, the variant G/H protein comprises the sequence of SEQ ID NO: 602. In some of any of the provided embodiments, the variant G/H protein comprises an amino acid sequence SEQ ID NO: 263. In some of any of the provided embodiments, the variant G/H protein comprises the sequence of SEQ ID NO: 606.

In some aspects, provided herein is a vector comprising any one of the polynucleotides described herein. In some of any of the provided embodiments, the vector is a mammalian vector, viral vector or artificial chromosome, optionally wherein the artificial chromosome is a bacterial artificial chromosome (BAC).

In some aspects, provided herein is a plasmid comprising any one of the polynucleotides described herein. In some of any of the provided embodiments, the plasmid further comprises one or more nucleic acids encoding proteins for lentivirus production

In some aspects, provided herein is a cell comprising any one of the polynucleotides, vectors, or plasmids described herein.

In some aspects, provided herein is a method of making a pseudotyped viral particle or virus-like particle comprising a variant paramyxovirus F and/or paramyxovirus G/H protein having reduced glycosylation, comprising a) providing a producer cell that comprises a viral particle or virus-like viral nucleic acid(s), and any one of the polynucleotides, vectors, or plasmids described herein; b) culturing the cell under conditions that allow for production of the viral particle or virus-like particle, and c) separating, enriching, or purifying the viral particle or virus-like particle from the cell, thereby making the pseudotyped viral particle or virus-like particle.

In some aspects, provided herein is a method of making a lipid particle comprising a variant paramyxovirus F and/or paramyxovirus G/H protein having reduced glycosylation, comprising a) providing a cell that comprises any one of the polynucleotides, vectors, or plasmids described herein; b) culturing the cell under conditions that allow for production of a lipid particle, and c) separating, enriching, or purifying the lipid particle from the cell, thereby making the lipid particle.

In some aspects, provided herein is a method of making a pseudotyped viral particle or virus-like particle comprising a paramyxovirus F and/or paramyxovirus G/H protein having reduced glycosylation, comprising separating, enriching, or purifying the pseudotyped viral particle or virus-like particle, wherein the separating, enriching, or purifying further comprises enzymatic removal of glycans attached to the paramyxovirus F protein and/or paramyxovirus G/H protein.

In some aspects, provided herein is a method method of making a lipid particle comprising a paramyxovirus F and/or paramyxovirus G/H protein having reduced glycosylation, comprising separating, enriching, or purifying the lipid particle, wherein the separating, enriching, or purifying further comprises enzymatic removal of glycans attached to the paramyxovirus F protein and/or paramyxovirus G/H protein.

In some aspects, provided herein is a method of making a pseudotyped viral particle or virus-like particle comprising a paramyxovirus F and/or paramyxovirus G/H protein having reduced glycosylation, comprising a) providing a producer cell that comprises a viral particle or virus-like viral nucleic acid(s) and paramyxovirus F and/or paramyxovirus G/H nucleic acid(s); b) culturing the cell under conditions that allow for production of the viral particle or virus-like particle, wherein the culturing comprises treating the cell with a modulator of glycosylation; and c) separating, enriching, or purifying the viral particle or virus-like particle from the cell, thereby making the pseudotyped viral particle or virus-like particle.

In some aspects, provided herein is a method of making a lipid particle comprising a paramyxovirus F and/or paramyxovirus G/H protein having reduced glycosylation, comprising a) providing a cell that comprises a paramyxovirus F and/or paramyxovirus G/H nucleic acid(s); b) culturing the cell under conditions that allow for production of a lipid particle, wherein the culturing comprises treating the cell with a modulator of glycosylation; and c) separating, enriching, or purifying the lipid particle from the cell, thereby making the lipid particle.

In some aspects, provided herein is a pseudotyped viral particle or virus-like particle made according to any one of the methods described herein.

In some aspects, provided herein is a lipid particle made according to any one of the methods described herein.

In some aspects, provided herein is a composition comprising a producer cell, cell medium, and one or more modulators of glycosylation.

In some aspects, provided herein is a method of making a pseudotyped viral particle or virus-like particle comprising a paramyxovirus F and/or paramyxovirus G/H protein having reduced glycosylation, comprising a) providing a producer cell that comprises a viral particle or virus-like viral nucleic acid(s) and paramyxovirus F and/or paramyxovirus G/H nucleic acid(s), wherein the producer cell comprises one or more genetic modifications that modulate glycosylation; b) culturing the cell under conditions that allow for production of the viral particle or virus-like particle, and c) separating, enriching, or purifying the viral particle or virus-like particle from the cell, thereby making the pseudotyped viral particle or virus-like particle.

In some aspects, provided herein is a composition comprising a producer cell, wherein the producer cell comprises one or more nucleic acids for producing a viral particle or virus-like particle comprising a paramyxovirus F and/or paramyxovirus G/H protein, and wherein the producer cell comprises one or more genetic modifications that modulate glycosylation.

In some aspects, provided herein is a method of making a lipid particle comprising a variant paramyxovirus F and/or paramyxovirus G/H protein having reduced glycosylation, comprising a) providing a cell paramyxovirus F and/or paramyxovirus G/H nucleic acid(s), wherein the cell comprises one or more genetic modifications that modulate glycosylation; b) culturing the cell under conditions that allow for production of a lipid particle, and c) separating, enriching, or purifying the lipid particle from the cell, thereby making the lipid particle.

In some aspects, provided herein is a composition comprising a producer cell, wherein the producer cell comprises one or more nucleic acids for producing a lipid particle comprising a paramyxovirus F and/or paramyxovirus G/H protein, and wherein the producer cell comprises one or more genetic modifications that modulate glycosylation.

In some aspects, provided herein is a method of transducing a cell with a with a pseudotyped viral particle, virus-like particle, or lipid particle, comprising contacting the cell with any one of the pseudotyped viral particles, virus-like particles, or lipid particles described herein.

In some aspects, provided herein is a method of delivering an exogenous agent to a cell, comprising contacting the cell with any one of the pseudotyped viral particles, virus-like particles, or lipid particles described herein, wherein the pseudotyped viral particle, virus-like particle, or lipid particle comprises the exogenous agent. In some of any of the provided embodiments, the contacting transduces the cell with the pseudotyped viral particle, virus-like particle, or lipid particle. In some of any of the provided embodiments, the contacting is in vivo in a subject.

In some aspects, provided herein is a method of reducing off-target transduction of a pseudotyped viral particle, virus-like particle, or lipid particle comprising administering any one of the pseudotyped viral particles, virus-like particles, or lipid particles described herein. In some of any of the provided embodiments, on-target transduction is not reduced.

In some aspects, provided herein is a method of treating a disease or disorder in a subject, comprising administering to the subject any one of the pseudotyped viral particles, virus-like particles, or lipid particles described herein.

### BRIEF DESCRIPTION OF THE DRAWINGS

FIG. 1A shows off-target transduction of CD8 specific fusogens, indicated by the percentage of different cell-types transduced using a CD8-specific fusogen. SupT1 are CD8 cells and PHH are liver cells.

FIG. 1B shows the transduction results of treating various fusogens with a deglycosylation mixture. The y-axis shows the percent of GFP positive cells, indicating transduction. G proteins are indicated on the x-axis, and comprised either the NivG fused to a liver specific scFv, NivG includes mutations that reduce Ephrin binding (Gm), NivG fused to a CD8-specific VHH, or VSV-G derived from Indiana vesiculovirus. The darker colored bars on the left show the results of untreated fusogens, while the lighter colored bars on the right show deglycosylation mixture treated fusogens.

FIG. 2A shows the result of transduction of humanized mice under four experimental conditions using NivG proteins with the indicated fusions/modifications. Saline was used as negative control; a fusion to a liver specific scFv was used as a positive control for liver transduction.

FIG. 2B shows transduction of different cell types using the indicated NivG construct, comprising a fusion to a CD8-specific scFv; fusion to a CD8-specific VHH; or NivG including mutations that disrupt Ephrin binding (Gm).

FIG. 3 shows a comparison of on-target vs. off-target transduction in fusogens comprising NivG fused to a CD8-specific VHH either treated with glycosidase (deglycosylation mixture) or untreated.

FIG. 4A shows transduction of CD8 cells with different NivG glycosylation mutants. Constructs are indicated along the x-axis; the y-axis shows percent GFP expressing cells.

FIG. 4B shows PHH transduction of different NivG glycosylation mutants fused to either a liver-specific scFV or a CD8-specific VHH); NB indicates NivG without fusion to a binding domain.

FIG. 5A shows PHH transduction of different NivG glycosylation mutants, all lacking a binding domain.

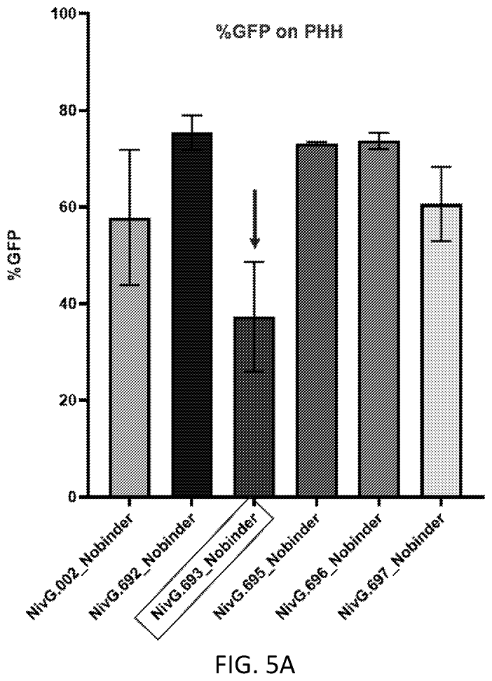

FIG. 5B shows the ratio of transduction of PHH in the indicated variants vs, a non-mutated control. The glycosylation mutant is shown along the x-axis. The y-axis depicts specific binding domains fused to each NivG mutant.

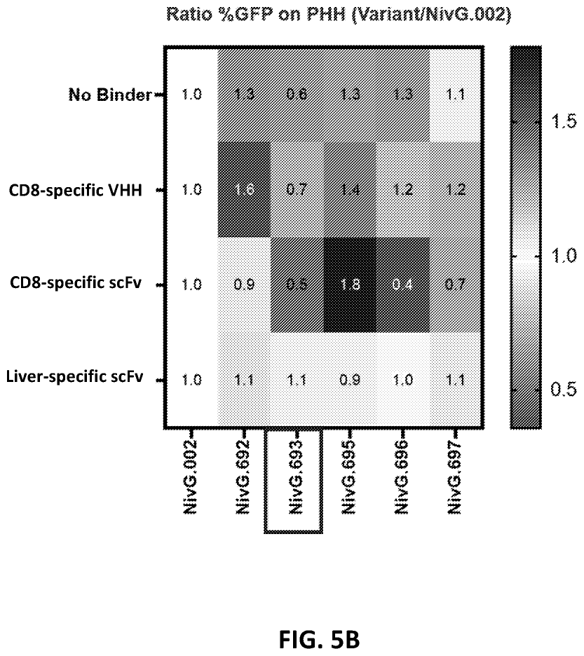

FIG. 6A shows titration curves in SupT1 cells of three NivG glycosylation mutants, fused to either a CD8-specific VHH or a CD8-specific scFv. The y-axis shows the percent of GFP expressing cells after transduction; the x-axis shows specific fusogen dilutions tested.

FIG. 6B shows titration curves in PHH of three NivG glycosylation mutants, fused to either a CD8-specific VHH or a CD8-specific scFv. The y-axis shows the percent of GFP expressing cells after transduction; the x-axis shows specific fusogen dilutions tested.

FIG. 6C shows a comparison of the titer (TU/mL) of the indicated constructs in on-target cells (SupT1, lighter colored bars) and off-target cells (PHH, darker colored bars).

FIG. 7 depicts GFP transduction in off-target cells (PHH).

### DETAILED DESCRIPTION

Provided herein are lipid particles, including viral particles and virus-like particles, having reduced glycosylation. In some embodiments, the lipid particle comprises a variant paramyxovirus G/H glycoprotein. In some embodiments, the variant paramyxovirus G/H glycoprotein comprises one or more mutations that reduces glycosylation of the protein. In some embodiments, the reduced glycosylation of the paramyxovirus G/H protein results in reduced transduction of one or more off-target cell types.

The fusion (F) and attachment (G/H) glycoproteins of Paramyxovirus family of viruses mediate cellular entry. In some embodiments, the combination of an F protein, such as the F protein from Nipah virus (NiV), and a variant G/H protein, such as a variant NiV-G protein, as provided herein is able to mediate cellular entry of a provided lipid particle (e.g. lentiviral vector). In some embodiments, the lipid particles described herein have reduced transduction of off-target cell types.

The F protein is a class I fusion protein that has structural and functional features in common with fusion proteins of many families (e.g., HIV-1 gp41 or influenza virus hemagglutinin [HA]), such as an ectodomain with a hydrophobic fusion peptide and two heptad repeat regions (White J M et al. 2008. Crit Rev Biochem Mol Biol 43:189-219). F proteins are synthesized as inactive precursors F₀ and are activated by proteolytic cleavage into the two disulfide-linked subunits F₁ and F₂ (Moll M. et al. 2004. J. Virol. 78 (18): 9705-9712).

G proteins are attachment proteins that are type II transmembrane glycoproteins containing an N-terminal cytoplasmic tail, a transmembrane domain, an extracellular stalk, and a globular head (Liu, Q. et al. 2015. Journal of Virology, 89 (3): 1838-1850). The attachment protein recognizes cellular receptors. For example, the Nipah virus attachment protein recognizes EphrinB2 and EphrinB3. Binding of the receptor to the G protein triggers a series of conformational changes that eventually lead to the triggering of the F protein, which exposes the fusion peptide of the F protein, allowing another series of conformational changes that lead to virus-cell membrane fusion (Stone J. A. et al. 2016. J Virol. 90 (23): 10762-10773). EphrinB2 was previously identified as the primary NiV receptor (Negrete et al., 2005), as well as ephrinB3 as an alternate receptor (Negrete et al., 2006). Wild-type NiV-G has a high affinity for ephrinB2 and B3, with affinity binding constants (Kd) in the picomolar range (Negrete et al., 2006) (Kd=0.06 nM and 0.58 nM for cell surface expressed ephrinB2 and B3, respectively).

In aspects of the provided embodiments, a lipid particle provided herein can be engineered to contain an F protein and G/H protein, in which both the F and G/H proteins are embedded in the lipid bilayer of the lipid particle. In some embodiments, the lipid particles can be a viral particle, a virus-like particle, or a viral vector, such as a lentiviral vector. In some embodiments, provided herein is a lentiviral vector pseudotyped with the combination of a F protein and a G/H protein.

In some aspects of the provided embodiments, the lipid particles provided herein are engineered to contain an F protein and any one of the variant G/H proteins described herein. In some embodiments, the G/H proteins comprise one or more mutations that reduce glycosylation of the protein.

In some embodiments, the variant G/H protein may be further linked to a targeting moiety, such as an antigen binding domain, to facilitate specific targeting of the lipid particle to a target molecule for fusion with a desired target cell. In some embodiments, a binding domain is any domain that binds to a cell surface molecule on a target cell. In some embodiments, the binding domain is a single domain antibody (sdAb). In some embodiments, the binding domain is a single chain variable fragment (scFv). The binding domain can be linked directly or indirectly to the variant G/H protein. In particular embodiments, the binding domain is linked to the C-terminus (C-terminal amino acid) of the variant G/H protein or the biologically active portion thereof. The linkage can be via a peptide linker, such as a flexible peptide linker.

Thus, the provided embodiments include embodiments in which the variant G/H protein may be re-targeted to any desired cell type for specific targeting of a lipid particle (e.g. lentiviral vector) and, in some cases, specific delivery to a target cell of a transgene or heterologous protein contained therein. In some embodiments, the one or more glycosylation mutations in the G/H proteins described herein reduce off-target transduction, while maintaining the same or similar transduction efficiency for on-target cells or cell types.

In some aspects of the provided embodiments, the lipid particle is produced and/or modified in such a way as to reduce glycosylation of the Paramyxovirus F and G/H proteins embedded in the lipid bilayer of the lipid particle. In some embodiments, the lipid particle is produced from a producer cell comprising one or more mutations that reduces glycosylation of proteins. In some embodiments, the lipid particle is produced from a producer cell treated with a compound that results in reduced glycosylation of proteins. In some embodiments, the lipid particle is contacted with one or more glycosidases during production and/or isolation.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Unless defined otherwise, all technical and scientific terms, acronyms, and abbreviations used herein have the same meaning as commonly understood by one of ordinary skill in the art to which the invention pertains. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, “lipid particle” refers to any biological or synthetic particle that contains a bilayer of amphipathic lipids enclosing a lumen or cavity. Typically a lipid particle does not contain a nucleus. Such lipid particles include, but are not limited to, viral particles (e.g. lentiviral particles), virus-like particles, viral vectors (e.g., lentiviral vectors) exosomes, enucleated cells, various vesicles, such as a microvesicle, a membrane vesicle, an extracellular membrane vesicle, a plasma membrane vesicle, a giant plasma membrane vesicle, an apoptotic body, a mitoparticle, a pyrenocyte, or a lysosome. In some embodiments, a lipid particle can be a fusosome. In some embodiments, the lipid particle is not a platelet. In some embodiments, the fusosome is derived from a source cell. A lipid particle also may include an exogenous agent or a nucleic acid encoding an exogenous agent, which may be present in the lumen of the lipid particle.

The terms “viral vector particle” and “viral vector” are used interchangeably herein and refer to a vector for transfer of an exogenous agent (e.g. non-viral or exogenous nucleic acid) into a recipient or target cell and that contains one or more viral structural proteins in addition to at least one non-structural viral genomic component or functional fragment thereof (i.e., a polymerase, an integrase, a protease or other non-structural component). The viral vector thus contains the exogenous agent, such as heterologous nucleic acid that includes non-viral coding sequences, to be transferred into a cell. Examples of viral vectors are retroviral vectors, such as lentiviral vectors.

The term “retroviral vector” refers to a viral vector that contains retroviral nucleic acid or is derived from a retrovirus. A retroviral vector particle includes the following components: a vector genome (retrovirus nucleic acid), a nucleocapsid encapsidating the nucleic acid, and a membrane envelope surrounding the nucleocapsid. Typically, a retroviral vector contains sufficient retroviral genetic information to allow packaging of an RNA genome, in the presence of packaging components, into a viral particle capable of infecting a target cell. Infection of the target cell may include reverse transcription and integration into the target cell genome. A retroviral vector may be a recombinant retroviral vector that is replication defective and lacks genes essential for replication, such as a functional gag-pol and/or env gene and/or other genes essential for replication. A retroviral vector also may be a self-inactivating (SIN) vector.

As used herein, a “lentiviral vector” or LV refers to a viral vector that contains lentiviral nucleic acid or is derived from a lentivirus. A lentiviral vector particle includes the following components: a vector genome (lentivirus nucleic acid), a nucleocapsid encapsidating the nucleic acid, and a membrane surrounding the nucleocapsid. Typically, a lentiviral vector contains sufficient lentiviral genetic information to allow packaging of an RNA genome, in the presence of packaging components, into a viral particle capable of infecting a target cell. Infection of the target cell may include reverse transcription and integration into the target cell genome. A lentiviral vector may be a recombinant lentiviral vector that is replication defective and lacks genes essential for replication, such as a functional gag-pol and/or env gene and/or other genes essential for replication. A lentiviral vector also may be a self-inactivating (SIN) vector.

As used herein, a “retroviral nucleic acid,” refers to a nucleic acid containing at least the minimal sequence requirements for packaging into a retroviral vector, alone or in combination with a helper cell, helper virus, or helper plasmid. In the case of “lentiviral nucleic acid” the nucleic acid refers to at least the minimal sequence requirements for packaging into a lentiviral vector, alone or in combination with a helper cell, helper virus, or helper plasmid. In some embodiments, the viral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (Y)), RRE (e.g., to bind to Rev and promote nuclear export). The viral nucleic acid can comprise RNA (e.g., when part of a virion) or DNA (e.g., when being introduced into a source cell or after reverse transcription in a recipient cell). In some embodiments, the viral nucleic acid is packaged using a helper cell, helper virus, or helper plasmid which comprises one or more of (e.g., all of) gag, pol, and env.

As used herein, “fusosome” refers to a lipid particle containing a bilayer of amphipathic lipids enclosing a lumen or cavity and a fusogen that interacts with the amphipathic lipid bilayer. In some embodiments, the fusosome is a membrane enclosed preparation. In some embodiments, the fusosome is derived from a source cell. A fusosome also may include an exogenous agent or a nucleic acid encoding an exogenous agent, which may be present in the lumen of the fusosome.

As used herein, “fusosome composition” refers to a composition comprising one or more fusosomes.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain. Examples of fusogens include paramyxovirus F and G/H proteins such as those from Nipah Virus (NiV) and biologically active portions or variants thereof including any as described.

As used herein, a “re-targeted fusogen,” such as a re-targeted G/H protein, refers to a fusogen that comprises a targeting moiety having a sequence that is not part of the naturally-occurring form of the fusogen in which the targeting moiety targets or binds a molecule on a desired cell type. In embodiments, the fusogen comprises a different targeting moiety relative to the targeting moiety in the naturally-occurring form of the fusogen. In embodiments, the naturally-occurring form of the fusogen lacks a targeting domain, and the re-targeted fusogen comprises a targeting moiety that is absent from the naturally-occurring form of the fusogen. In embodiments, the fusogen is modified to comprise a targeting moiety. In some such embodiments, the attachment of the targeting moiety to a fusogen (e.g. G protein) may be directly or indirectly via a linker, such as a peptide linker. In embodiments, the fusogen comprises one or more sequence alterations outside of the targeting moiety relative to the naturally-occurring form of the fusogen, e.g., in a transmembrane domain, fusogenically active domain, or cytoplasmic domain.

As used herein, a “target cell” refers to a cell of a type to which it is desired that a targeted lipid particle delivers an exogenous agent. In embodiments, a target cell is a cell of a specific tissue type or class, e.g., an immune effector cell, e.g., a T cell. In some embodiments, a target cell is a diseased cell, e.g., a cancer cell. In some embodiments, the fusogen, e.g., re-targeted fusogen leads to preferential delivery of the exogenous agent to a target cell compared to a non-target cell.

As used herein a “non-target cell” refers to a cell of a type to which it is not desired that a targeted lipid particle delivers an exogenous agent. In some embodiments, a non-target cell is a cell of a specific tissue type or class. In some embodiments, a non-target cell is a non-diseased cell, e.g., a non-cancerous cell. In some embodiments, the fusogen, e.g., re-targeted fusogen leads to lower delivery of the exogenous agent to a non-target cell compared to a target cell.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved binding.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe

Non-conservative substitutions will entail exchanging a member of one of these classes for another class.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “effective amount” as used herein means an amount of a pharmaceutical composition which is sufficient to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a lipid particle, such as a viral vector, refers to an agent that is neither comprised by nor encoded in the corresponding wild-type virus or fusosome made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

A “disease” or “disorder” as used herein refers to a condition where treatment is needed and/or desired.

As used herein, the terms “treat.” “treating.” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder can include obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

The terms “individual” and “subject” are used interchangeably herein to refer to an animal; for example a mammal. The term patient includes human and veterinary subjects. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder. In particular embodiments, the subject is a human, such as a human patient.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the nucleic acid can comprise sugars and phosphate groups (as may typically be found in RNA or DNA), or modified or substituted sugar or phosphate groups. Alternatively, the backbone of the nucleic acid can comprise a polymer of synthetic subunits such as phosphoramidates and thus can be an oligodeoxynucleoside phosphoramidate (P—NH₂) or a mixed phosphoramidate-phosphodiester oligomer. In addition, a double-stranded nucleic acid can be obtained from the single stranded polynucleotide product of chemical synthesis either by synthesizing the complementary strand and annealing the strands under appropriate conditions, or by synthesizing the complementary strand de novo using a DNA polymerase with an appropriate primer

### II. Variant Paramyxovirus G/H Glycoproteins

Provided herein are lipid particles, e.g. viral particles and virus-like particles, comprising variant Paramyxovirus G/H glycoproteins comprising one or more amino acid mutations that result in decreased glycosylation of the protein (hereinafter also called “deglycosylation mutations”). The variant Paramyxovirus G/H glycoproteins described herein are, or can be, incorporated into a lipid particle, such as a viral particle or a virus-like particle. In some embodiments, the lipid particle comprising the variant G/H proteins have reduced transduction of off-target cell types, e.g., hepatocytes.

The envelope attachment G proteins are type II transmembrane glycoproteins containing an N-terminal cytoplasmic tail (e.g. corresponding to amino acids 1-49 of SEQ ID NO:13), a transmembrane domain (e.g. corresponding to amino acids 50-70 of SEQ ID NO:13), and an extracellular domain containing an extracellular stalk (e.g. corresponding to amino acids 71-187 of SEQ ID NO:13), and a globular head (corresponding to amino acids 188-602 of SEQ ID NO:13). The N-terminal cytoplasmic domain is within the inner lumen of the lipid bilayer and the C-terminal portion is the extracellular domain that is exposed on the outside of the lipid bilayer. Regions of the stalk in the C-terminal region (e.g. corresponding to amino acids 71-187 of SEQ ID NO: 13) have been shown to be involved in interactions with F protein and triggering of F protein fusion (Liu et al. 2015 J of Virology 89:1838). In wild-type G protein, the globular head mediates receptor binding to henipavirus entry receptors Ephrin B2 and Ephrin B3, but is dispensable for membrane fusion (Brandel-Tretheway et al. Journal of Virology. 2019, 93 (13) e00577-19).

Binding of the G protein to a binding partner can trigger fusion mediated by a compatible paramyxovirus fusion protein (e.g., F protein) or biologically active portion thereof (such as any of the F proteins described in III.C below). G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acids identity. Studies have shown a high degree of compatibility among G proteins with F proteins of different species as demonstrated by heterotypic fusion activation (Brandel-Tretheway et al. Journal of Virology. 2019). Exemplary Henipavirus protein G sequences are provided in Table 2. In some embodiments, at least one G protein has a sequence set forth in any of SEQ ID NOS: 13, 143, 144, 145, or 146 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 13, 143, 144, 145, or 146.

Table 2. Henipavirus protein G sequence clusters. Column 1, Genbank ID includes the Genbank ID of the whole genome sequence of the virus that is the centroid sequence of the cluster. Column 2, nucleotides of CDS provides the nucleotides corresponding to the CDS of the gene in the whole genome. Column 3, Full Gene Name, provides the full name of the gene including Genbank ID, virus species, strain, and protein name. Column 4, Sequence, provides the amino acid sequence of the gene. Column 5, #Sequences/Cluster, provides the number of sequences that cluster with this centroid sequence. Column 6 provides the SEQ ID numbers for the described sequences.

TABLE 2
Exemplary Henipavirus protein G sequences

| Gen | Nucleotides | Full | | SEQ |
|---|---|---|---|---|
| bank | of | sequence | | ID |
| ID | CDS | ID | Sequence | NO |
| AF017149 | 8913-10727 | gb: | MMADSKLVSLNNNLSGKIKDQGKVIKNYYG | 143 |
| | | AF017149\| | TMDIKKINDGLLDSKILGAFNTVIALLGSI | |
| | | Organism: | IIIVMNIMIIQNYTRTTDNQALIKESLQSV | |
| | | Hendra | QQQIKALTDKIGTEIGPKVSLIDTSSTITI | |
| | | virus\| | PANIGLLGSKISQSTSSINENVNDKCKFTL | |
| | | Strain | PPLKIHECNISCPNPLPFREYRPISQGVSD | |
| | | Name: | LVGLPNQICLQKTTSTILKPRLISYTLPIN | |
| | | UNKNOWN- | TREGVCITDPLLAVDNGFFAYSHLEKIGSC | |
| | | AF017149\| | TRGIAKQRIIGVGEVLDRGDKVPSMFMTNV | |
| | | Protein | WTPPNPSTIHHCSSTYHEDFYYTLCAVSHV | |
| | | Name: | GDPILNSTSWTESLSLIRLAVRPKSDSGDY | |
| | | glycoprotein\| | NQKYIAITKVERGKYDKVMPYGPSGIKQGD | |
| | | Gene | TLYFPAVGFLPRTEFQYNDSNCPIIHCKYS | |
| | | Symbol: | KAENCRLSMGVNSKSHYILRSGLLKYNLSL | |
| | | G | GGDIILQFIEIADNRLTIGSPSKIYNSLGQ | |
| | | | PVFYQASYSWDTMIKLGDVDTVDPLRVQWR | |
| | | | NNSVISRPGQSQCPRFNVCPEVCWEGTYND | |
| | | | AFLIDRLNWVSAGVYLNSNQTAENPVFAVF | |
| | | | KDNEILYQVPLAEDDTNAQKTITDCFLLEN | |
| | | | VIWCISLVEIYDTGDSVIRPKLFAVKIPAQ | |
| | | | CSES | |
| AF212302 | 8943-10751 | gb: | MPAENKKVRFENTTSDKGKIPSKVIKSYYG | 13 |
| | | AF212302\| | TMDIKKINEGLLDSKILSAFNTVIALLGSI | |
| | | Organism: | VIIVMNIMIIQNYTRSTDNQAVIKDALQGI | |
| | | Nipah virus\| | QQQIKGLADKIGTEIGPKVSLIDTSSTITI | |
| | | Strain | PANIGLLGSKISQSTASINENVNEKCKFTL | |
| | | Name: | PPLKIHECNISCPNPLPFREYRPQTEGVSN | |
| | | UNKNOWN- | LVGLPNNICLQKTSNQILKPKLISYTLPVV | |
| | | AF212302\| | GQSGTCITDPLLAMDEGYFAYSHLERIGSC | |
| | | Protein | SRGVSKQRIIGVGEVLDRGDEVPSLFMTNV | |
| | | Name: | WTPPNPNTVYHCSAVYNNEFYYVLCAVSTV | |
| | | attachment | GDPILNSTYWSGSLMMTRLAVKPKSNGGGY | |
| | | glycoprotein\| | NQHQLALRSIEKGRYDKVMPYGPSGIKQGD | |
| | | Gene | TLYFPAVGFLVRTEFKYNDSNCPITKCQYS | |
| | | Symbol: | KPENCRLSMGIRPNSHYILRSGLLKYNLSD | |
| | | G | GENPKVVFIEISDQRLSIGSPSKIYDSLGQ | |
| | | | PVFYQASFSWDTMIKFGDVLTVNPLVVNWR | |
| | | | NNTVISRPGQSQCPRFNTCPEICWEGVYND | |
| | | | AFLIDRINWISAGVFLDSNQTAENPVFTVF | |
| | | | KDNEILYRAQLASEDTNAQKTITNCFLLKN | |
| | | | KIWCISLVEIYDTGDNVIRPKLFAVKIPEQ | |
| | | | CT | |
| JQ001776 | 8170-10275 | gb: | MLSQLQKNYLDNSNQQGDKMNNPDKKLSVN | 144 |
| | | JQ001776: | FNPLELDKGQKDLNKSYYVKNKNYNVSNLL | |
| | | 8170- | NESLHDIKFCIYCIFSLLIIITIINIITIS | |
| | | 10275\| | IVITRLKVHEENNGMESPNLQSIQDSLSSL | |
| | | Organism: | TNMINTEITPRIGILVTATSVTLSSSINY | |
| | | Cedar | VGTKTNQLVNELKDYITKSCGFKVPELKLH | |
| | | virus\| | ECNISCADPKISKSAMYSTNAYAELAGPPK | |
| | | Strain | IFCKSVSKDPDFRLKQIDYVIPVQQDRSIC | |
| | | Name: | MNNPLLDISDGFFTYIHYEGINSCKKSDSF | |
| | | CG1a\| | KVLLSHGEIVDRGDYRPSLYLLSSHYHPYS | |
| | | Protein | MQVINCVPVTCNQSSFVFCHISNNTKTLDN | |
| | | Name: | SDYSSDEYYITYFNGIDRPKTKKIPINNMT | |
| | | attachment | ADNRYIHFTFSGGGGVCLGEEFIIPVTTVI | |
| | | glycoprotein\| | NTDVFTHDYCESFNCSVQTGKSLKEICSES | |
| | | Gene | LRSPTNSSRYNLNGIMIISQNNMTDFKIQL | |
| | | Symbol: | NGITYNKLSFGSPGRLSKTLGQVLYYQSSM | |
| | | G | SWDTYLKAGFVEKWKPFTPNWMNNTVISRP | |
| | | | NQGNCPRYHKCPEICYGGTYNDIAPLDLGK | |
| | | | DMYVSVILDSDQLAENPEITVENSTTILYK | |
| | | | ERVSKDELNTRSTTTSCFLFLDEPWCISVL | |
| | | | ETNRFNGKSIRPEIYSYKIPKYC | |
| NC_ | 9117-11015 | gb: | MPQKTVEFINMNSPLERGVSTLSDKKTLNQ | 145 |
| 0252 | | NC_025256: | SKITKQGYFGLGSHSERNWKKQKNQNDHYM | |
| 56 | | 9117- | TVSTMILEILVVLGIMENLIVLTMVYYQND | |
| | | 11015\| | NINQRMAELTSNITVLNLNLNQLTNKIQRE | |
| | | Organism: | IIPRITLIDTATTITIPSAITYILATLTTR | |
| | | Bat | ISELLPSINQKCEFKTPTLVLNDCRINCT | |
| | | Paramyxovirus | PPLNPSDGVKMSSLATNLVAHGPSPCRNFS | |
| | | Eid_hel/ | SVPTIYYYRIPGLYNRTALDERCILNPRLT | |
| | | GHM74a/ | ISSTKFAYVHSEYDKNCTRGFKYYELMTFG | |
| | | GHA/2009\| | EILEGPEKEPRMFSRSFYSPTNAVNYHSCT | |
| | | Strain | PIVTVNEGYFLCLECTSSDPLYKANLSNST | |
| | | Name: | FHLVILRHNKDEKIVSMPSFNLSTDQEYV | |
| | | BatPV/ | QIIPAEGGGTAESGNLYFPCIGRLLHKRVT | |
| | | Eid_hel/ | HPLCKKSNCSRTDDESCLKSYYNQGSPQHQ | |
| | | GHM74a/ | VVNCLIRIRNAQRDNPTWDVITVDLTNTYP | |
| | | GHA/20 | GSRSRIFGSFSKPMLYQSSVSWHTLLQVAE | |
| | | 09\| | ITDLDKYQLDWLDTPYISRPGGSECPFGNY | |
| | | Protein | CPTVCWEGTYNDVYSLTPNNDLFVTVYLKS | |
| | | Name: | EQVAENPYFAIFSRDQILKEFPLDAWISSA | |
| | | glycoprotein\| | RTTTISCFMFNNEIWCIAALEITRLNDDII | |
| | | Gene | RPIYYSFWLPTDCRTPYPHTGKMTRVPLRS | |
| | | Symbol: | TYNY | |
| | | G | | |
| NC_025352 | 8716-11257 | gb: | MATNRDNTITSAEVSQEDKVKKYYGVETAE | 146 |
| | | NC_025352: | KVADSISGNKVFILMNTLLILTGAIITITL | |
| | | 8716- | NITNLTAAKSQQNMLKIIQDDVNAKLEMFV | |
| | | 11257\| | NLDQLVKGEIKPKVSLINTAVSVSIPGQIS | |
| | | Organism: | NLQTKFLQKYVYLEESITKQCTCNPLSGIF | |
| | | Mojiang | PTSGPTYPPTDKPDDDTTDDDKVDTTIKPI | |
| | | virus\| | EYPKPDGCNRTGDHFTMEPGANFYTVPNLG | |
| | | Strain | PASSNSDECYTNPSFSIGSSIYMFSQEIRK | |
| | | Name: | TDCTAGEILSIQIVLGRIVDKGQQGPQASP | |
| | | Tongguan1\| | LLVWAVPNPKIINSCAVAAGDEMGWVLCSV | |
| | | Protein | TLTAASGEPIPHMFDGFWLYKLEPDTEVVS | |
| | | Name: | YRITGYAYLLDKQYDSVFIGKGGGIQKGND | |
| | | attachment | LYFQMYGLSRNRQSFKALCEHGSCLGTGGG | |
| | | glycoprotein\| | GYQVLCDRAVMSFGSEESLITNAYLKVNDL | |
| | | Gene | ASGKPVIIGQTFPPSDSYKGSNGRMYTIGD | |
| | | Symbol: | KYGLYLAPSSWNRYLRFGITPDISVRSTTW | |
| | | G | LKSQDPIMKILSTCTNTDRDMCPEICNTRG | |
| | | | YQDIFPLSEDSEYYTYIGITPNNGGTKNFV | |
| | | | AVRDSDGHIASIDILQNYYSITSATISCFM | |
| | | | YKDEIWCIAITEGKKQKDNPQRIYAHSYKI | |
| | | | RQMCYNMKSATVTVGNAKNITIRRY | |

In some embodiments, any of the provided lipid particles (e.g., lentiviral vectors) may also contain an F protein, such as a NiV-F protein, such as a full-length NiV-F protein or a biologically active portion thereof or a variant thereof. For instance, also provided herein are viral particles or viral-like particles, such as lentiviral particles or lentiviral-like particles, that are pseudotyped with any of the provided variant NiV-G proteins and a NiV-F protein, such as a full-length NiV-F protein or a biologically active portion or a variant thereof.

In particular embodiments, the variant paramyxovirus envelope attachment protein (e.g., G protein) or functionally active variant or biologically active portion is a protein that retains fusogenic activity in conjunction with a paramyxovirus fusion protein (e.g., F protein), such as a NiV-F protein described herein. Fusogenic activity includes the activity of the paramyxovirus envelope attachment protein (e.g., G protein) in conjunction with a paramyxovirus fusion protein (e.g., F protein) to promote or facilitate fusion of two membrane lumens, such as the lumen of the targeted lipid particle having embedded in its lipid bilayer a paramyxovirus fusion protein (e.g., F protein) and paramyxovirus envelope attachment protein (e.g., G protein), and a cytoplasm of a target cell, e.g. a cell that contains a surface receptor or molecule that is recognized or bound by the targeted envelope protein. In some embodiments, the paramyxovirus fusion protein (e.g., F protein) and the paramyxovirus envelope attachment protein (e.g., G protein) are from the same paramyxovirus species (e.g. the same Henipavirus species such as NiV-G and NiV-F).

Reference to retaining fusogenic activity includes activity of a lipid particle (e.g. lentiviral vector) containing a paramyxovirus G/H protein and paramyxovirus fusion protein (e.g., F and G proteins) that is between at or about 10% and at or about 150% or more of the level or degree of binding of a reference lipid particle (e.g. lentiviral vector) that is similar, such as contains the same variant NiV-F, but that contains the corresponding wild-type G protein, such as set forth in SEQ ID NO: 13. For instance, a lipid particle (e.g. lentiviral vector) that retains fusogenic activity has at least or at least about 10% of the level or degree of fusogenic activity of the reference lipid particle that is similar (such as contains the same NiV-F) but that contains the corresponding wild-type G protein, such as at least or at least about 15% of the level or degree of fusogenic activity, at least or at least about 20% of the level or degree of fusogenic activity, at least or at least about 25% of the level or degree of fusogenic activity, at least or at least about 30% of the level or degree of fusogenic activity, at least or at least about 35% of the level or degree of fusogenic activity, at least or at least about 40% of the level or degree of fusogenic activity, at least or at least about 45% of the level or degree of fusogenic activity, at least or at least about 50% of the level or degree of fusogenic activity, at least or at least about 55% of the level or degree of fusogenic activity, at least or at least about 60% of the level or degree of fusogenic activity, at least or at least about 65% of the level or degree of fusogenic activity, at least or at least about 70% of the level or degree of fusogenic activity, at least or at least about 75% of the level or degree of fusogenic activity, at least or at least about 80% of the level or degree of fusogenic activity, at least or at least about 85% of the level or degree of fusogenic activity, at least or at least about 90% of the level or degree of fusogenic activity, at least or at least about 95% of the level or degree of fusogenic activity, at least or at least about 100% of the level or degree of fusogenic activity, or at least or at least about 120% of the level or degree of fusogenic activity.

Reference to retaining fusogenic activity includes activity of a lipid particle (e.g. lentiviral vector) containing a variant paramyxovirus G/H and paramyxovirus fusion protein (e.g., F and G proteins) that is between at or about 10% and at or about 150% or more of the level or degree of binding of a reference lipid particle (e.g. lentiviral vector) that is similar, such as contains the same NiV-F, but that contains a separate NiV-G. For instance, a lipid particle (e.g. lentiviral vector) that retains fusogenic activity has at least or at least about 10% of the level or degree of fusogenic activity of the reference lipid particle that is similar (such as contains the same NiV-F) but that contains a separate NiV-G, such as at least or at least about 15% of the level or degree of fusogenic activity, at least or at least about 20% of the level or degree of fusogenic activity, at least or at least about 25% of the level or degree of fusogenic activity, at least or at least about 30% of the level or degree of fusogenic activity, at least or at least about 35% of the level or degree of fusogenic activity, at least or at least about 40% of the level or degree of fusogenic activity, at least or at least about 45% of the level or degree of fusogenic activity, at least or at least about 50% of the level or degree of fusogenic activity, at least or at least about 55% of the level or degree of fusogenic activity, at least or at least about 60% of the level or degree of fusogenic activity, at least or at least about 65% of the level or degree of fusogenic activity, at least or at least about 70% of the level or degree of fusogenic activity, at least or at least about 75% of the level or degree of fusogenic activity, at least or at least about 80% of the level or degree of fusogenic activity, at least or at least about 85% of the level or degree of fusogenic activity, at least or at least about 90% of the level or degree of fusogenic activity, at least or at least about 95% of the level or degree of fusogenic activity, at least or at least about 100% of the level or degree of fusogenic activity, or at least or at least about 120% of the level or degree of fusogenic activity.

In some embodiments, the variant Paramyxovirus G/H glycoproteins are further modified such that the protein is re-targeted compared to the native tropism of the G/H protein. For instance, in some embodiments, the Paramyxovirus G/H glycoproteins comprises one or more mutations that reduce or abrogate binding to the cognate cellular receptor, but do not impact the association of the G/H protein with the F protein (see, e.g., Aguilar, et al. J Biol Chem. 2009; 284 (3): 1628-1635; Weise et al. J Virol. 2010; 84 (15): 7634-764; Negrete et al., J Virol. 2007; 81 (19): 10804-10814; Negrete et al. PLOS Pathog. 2006; Guillaume et al., J. Virol 2006, 80 (15) 7546-7554); Friedrich et al. Mol. Ther. 2013, 21 (4): 849-859; US20190144885). Thus, in some aspects, a variant Nipah G protein provided herein may further contain a mutation in its extracellular domain to reduce or abrogate binding to Ephrin B2 and/or Ephrin B3.

In some embodiments the variant Paramyxovirus G/H glycoproteins are further modified to comprise a modified cytoplasmic tail comprising a truncated cytoplasmic tail as compared to a G/H glycoprotein from the same Paramyxovirus species.

### A. Mutated Paramyxovirus G/H Glycoproteins Having Reduced Glycosylation

Provided herein are variant Paramyxovirus G/H glycoproteins comprising one or more amino acid mutations that result in decreased glycosylation of the protein. The one or more amino acid mutations, also called deglycosylation mutations, can be one or more amino acid substitutions (also referred to as mutations).

In some embodiments, the variant Paramyxovirus G/H glycoprotein comprises an amino acid substitution at one or more amino acid positions that reduce glycosylation of the G/H glycoprotein. In some embodiments, the one or more amino acid substitutions disrupts an N-linked glycosylation site. In some embodiments, the one or more amino acid substitutions disrupts an O-linked glycosylation site.

In some embodiments, the variant Paramyxovirus G/H glycoprotein is derived from Morbillivirus (e.g., measles virus (MeV), canine distemper virus, Cetacean morbillivirus, Peste-des-petits-ruminants virus, Phocine distemper virus, Rinderpest virus), Henipavirus (e.g., Hendra (HeV) virus, Nipah (NiV) virus, a Cedar (CedPV) virus, Mòjiāng virus, a Langya virus or bat Paramyxovirus). In some embodiments, the variant Paramyxovirus G/H glycoprotein is a variant of a Paramyxovirus G/H glycoprotein derived from Nipah virus or Measles virus. In some embodiments, the variant Paramyxovirus G/H protein is selected from the group consisting of SEQ ID NOs: 143-147, 244, and 2445, or a modified Paramyxovirus G/H glycoprotein derived from any one of 143-147, 244, and 245 containing an altered cytoplasmic tail. In some embodiments, the variant Paramyxovirus G/H protein has a sequence of amino acids that has at least 85%, at least 86%, at least 87%, at least 88%, at least 89%, at least 90%, at least 91%, at least 92%, at least 93%, at least 94% or at least 95% to any one of SEQ ID NOs: 143-147, 244, and 245 and contains the acid substitution at one or more amino acid positions that reduce glycosylation of the G/H glycoprotein as provided herein.

The location of precited glycosylation sites can be determined using the sequence of a protein. For example, N-glycosylation often occurs at sites with the sequence N-X-S/T in which “X” is any amino acid except P. Various algorithms and tools are available for prediction of both N- and O-linked glycosylation, including SprintGly (http://sparks-lab.org/server/sprint-gly/). NetNGlyc (https://services.healthtech.dtu.dk/service.php?NetNGlyc-1.0), NetOGlyc (https://services.healthtech.dtu.dk/service.php?NetOGlyc-4.0), and GlycoMine^{struct} (http://glycomine.erc.monash.edu/Lab/GlycoMine_Struct/), and methods described in Pitti et al., Sci. Reports, 9:15975 (2019) and Pakhrin et al., Molecules 26:7314 (2021). Any predicted glycosylation site may be substituted as described herein.

In some embodiments, the Paramyxovirus G/H glycoprotein to which the deglycosylation mutation is made is a NiV-G set forth in SEQ ID NO: 147 or a modified Nipah G glycoprotein (NiV-G) that has an altered cytoplasmic tail compared to native NiV-G (e.g., SEQ ID NO:147). In some embodiments, the variant Paramyxovirus G/H protein has a sequence of amino acids that has at least 85%, at least 86%, at least 87%, at least 88%, at least 89%, at least 90%, at least 91%, at least 92%, at least 93%, at least 94% or at least 95% to SEQ ID NO: 147 and contains the acid substitution at one or more amino acid positions that reduce glycosylation of the G/H glycoprotein as provided herein Exemplary modified NiV-G proteins with altered cytoplasmic tails to which the one or more amino acid substitutions for reducing glycosylation can be incorporated are described in Section II.B.

Amino acid positions for substitutions are described herein with positions “corresponding to” positions of a reference sequence. It is understood that the amino acid substitutions are not limited to being made in only the reference sequence but also can be made in similar sequences by identification of residues that align or correspond with the reference positions. For instance, positions “corresponding to” to positions of a protein in a reference sequence can be identified upon alignment of a similar sequence with the referenced sequence based on structural sequence alignment or using a standard alignment algorithm, such as the GAP algorithm. By aligning the sequences, one skilled in the art can identify corresponding residues, for example, using conserved and identical amino acid residues as guides. For instance, amino acid positions for mutations are described herein with reference to the exemplary truncated NiV-G sequence set forth in SEQ ID NO: 1; however, similar amino acid positions for mutations as described can be made in other modified NiV-G sequences, such as any as described in Section II.B, by sequence alignment and identification of the corresponding residues.

In some embodiments, the one or more amino acid mutations are at positions corresponding to positions 39, 126, 128, 273, 345, 384, 448, and 496 of SEQ ID NO:1. In some embodiments, the variant Paramyxovirus G/H glycoprotein comprises an amino acid mutation at any one of positions 39, 126, 128, 273, 345, 384, 448, and 496 of SEQ ID NO: 1. In some embodiments, the variant Paramyxovirus G/H glycoprotein comprises two or more amino acid mutations at any of positions corresponding to positions 39, 126, 128, 273, 345, 384, 448, and 496 of SEQ ID NO:1., such as mutations at 2, 3, 4, 5, 7, or 8 of the positions.

In some embodiments, the one or more amino acid mutations is at a position corresponding to position 39 of SEQ ID NO:1. In some embodiments, the one or more amino acid mutations is at a position corresponding to position 126 of SEQ ID NO:1. In some embodiments, the one or more amino acid mutations is at a position corresponding to position 128 of SEQ ID NO:1. In some embodiments, the one or more amino acid mutations is at a position corresponding to position 273 of SEQ ID NO:1. In some embodiments, the one or more amino acid mutations is at a position corresponding to position 345 of SEQ ID NO:1. In some embodiments, the one or more amino acid mutations is at a position corresponding to position 384 of SEQ ID NO:1. In some embodiments, the one or more amino acid mutations is at a position corresponding to position 448 of SEQ ID NO: 1. In some embodiments, the one or more amino acid mutations is at a position corresponding to position 496 of SEQ ID NO:1.

In some embodiments, the native amino acid at the position comprising the amino acid mutation is asparagine or serine. In some embodiments, the amino acid mutation is an amino acid substitution. In some embodiments, the mutation is an asparagine to glutamine substitution. In some embodiments, the mutation is a serine to alanine substitution.

In some embodiments, the mutation is an asparagine to glutamine substitution at a position corresponding to position 39 (N39Q) of SEQ ID NO: 1. In some embodiments, the mutation is an asparagine to glutamine substitution at a position corresponding to position 126 (N126Q) of SEQ ID NO: 1. In some embodiments, the mutation is an asparagine to glutamine substitution at a position corresponding to position 273 (N273Q) of SEQ ID NO:1. In some embodiments, the mutation is an asparagine to glutamine substitution at a position corresponding to position 345 (N345Q) of SEQ ID NO: 1. In some embodiments, the mutation is an asparagine to glutamine substitution at a position corresponding to position 384 (N384Q) of SEQ ID NO:1. In some embodiments, the mutation is an asparagine to glutamine substitution at a position corresponding to position 448 (N448Q) of SEQ ID NO: 1. In some embodiments, the mutation is an asparagine to glutamine substitution at a position corresponding to position 496 (N496Q) of SEQ ID NO:1.

In some embodiments, the mutation is a serine to alanine substitution at a position corresponding to position 128 (S128A) of SEQ ID NO:1.

In some embodiments, the G/H glycoprotein is derived from Nipah virus G protein and the one or more amino acid substitutions are at positions corresponding to positions selected from the group consisting of 39, 126, 128, 273, 345, 384, 448, and 496 of SEQ ID NO: 1. In some embodiments, the one or more amino acid substitutions are selected from N39Q, N126Q, S128A, N273Q, N345Q, N384Q, N448Q, N496Q or any combination thereof. In some embodiments, the G/H glycoprotein is a variant NiV-G containing one amino acid substitution from any one of N39Q, N126Q, S128A, N273Q, N345Q, N384Q, N448Q, N496Q. In some embodiments, the G/H glycoprotein is a variant NiV-G containing two amino acid substitutions from any two of N39Q, N126Q, S128A, N273Q, N345Q, N384Q, N448Q, N496Q. In some embodiments, the G/H glycoprotein is a variant NiV-G containing three amino acid substitutions from any three of N39Q, N126Q, S128A, N273Q, N345Q, N384Q, N448Q, N496Q. In some embodiments, the G/H glycoprotein is a variant NiV-G containing four amino acid substitutions from any one of N39Q, N126Q, S128A, N273Q, N345Q, N384Q, N448Q, N496Q. In some embodiments, the G/H glycoprotein is a variant NiV-G containing five amino acid substitutions from any one of N39Q, N126Q, S128A, N273Q, N345Q, N384Q, N448Q, N496Q. In some embodiments, the G/H glycoprotein is a variant NiV-G containing six amino acid substitutions from any one of N39Q, N126Q, S128A, N273Q, N345Q, N384Q, N448Q, N496Q. In some embodiments, the G/H glycoprotein is a variant NiV-G containing seven amino acid substitutions from any one of N39Q, N126Q, S128A, N273Q, N345Q, N384Q, N448Q, N496Q. In some embodiments, the G/H glycoprotein is a variant NiV-G containing eight amino acid substitutions from any one of N39Q, N126Q, S128A, N273Q, N345Q, N384Q, N448Q, N496Q. In some embodiments, the one or more amino acid substitutions are in the SEQ ID NO: 147 or a or a modified Nipah G glycoprotein (NiV-G) that has an altered cytoplasmic tail compared to native NiV-G (e.g., SEQ ID NO: 147). In some embodiments, the amino acid substitutions are in a modified NiV-G protein described in Section II.B. In some embodiments, the amino acid substitutions are in the NiV-G set for thin SEQ ID NO:1.

In some embodiments, the variant Nipah-G protein comprises at least three amino acid substitutions. In some embodiments, the amino acid substitutions are at positions 273, 384, and 496 of SEQ ID NO: 1. In some embodiments, the amino acid substitutions are at positions 273, 345, and 496 of SEQ ID NO: 1. In some embodiments, the amino acid substitutions are at positions 39, 126, and 128 of SEQ ID NO: 1. In some embodiments, the amino acid substitutions are at positions 39, 273, and 345 of SEQ ID NO: 1. In some embodiments, the amino acid substitutions are at positions 39, 384, and 448 of SEQ ID NO: 1. In some embodiments, the amino acid substitutions are at positions 39, 448, and 496 of SEQ ID NO: 1. In some embodiments, the amino acid substitutions are at positions 39, 128, and 273 of SEQ ID NO: 1. In some embodiments, the amino acid substitutions are at positions 39, 345, and 384 of SEQ ID NO: 1. In some embodiments, the amino acid substitutions are at positions 39, 384, and 448 of SEQ ID NO: 1.

In some embodiments, the variant Nipah-G protein comprises at least two amino acid substitutions. In some embodiments, the amino acid substitutions are at positions 273, and 496 of SEQ ID NO: 1. In some embodiments, the amino acid substitutions are at positions 345, and 496 of SEQ ID NO: 1. In some embodiments, the amino acid substitutions are at positions 39 and 128 of SEQ ID NO: 1. In some embodiments, the amino acid substitutions are at positions 39, and 345 of SEQ ID NO: 1. In some embodiments, the amino acid substitutions are at positions 39, and 448 of SEQ ID NO: 1. In some embodiments, the amino acid substitutions are at positions 39 and 496 of SEQ ID NO: 1. In some embodiments, the amino acid substitutions are at positions 39 and 273 of SEQ ID NO: 1. In some embodiments, the amino acid substitutions are at positions 39 and 384 of SEQ ID NO: 1. In some embodiments, the amino acid substitutions are at positions 384 and 448 of SEQ ID NO: 1.

In some embodiments, the amino acid substitution is at position 39 of SEQ ID NO: 1. In some embodiments, the amino acid substitution is at position 126 of SEQ ID NO: 1. In some embodiments, the amino acid substitution is at position 128 of SEQ ID NO: 1. In some embodiments, the amino acid substitution is at position 273 of SEQ ID NO: 1. In some embodiments, the amino acid substitution is at position 345 of SEQ ID NO: 1. In some embodiments, the amino acid substitution is at position 384 of SEQ ID NO: 1. In some embodiments, the amino acid substitution is at position 448 of SEQ ID NO: 1. In some embodiments, the amino acid substitution is at position 496 of SEQ ID NO: 1.

In some embodiments, the variant Nipah-G protein comprises an asparagine to glutamine substitution at position 39 of SEQ ID NO:1. In some embodiments, the variant Nipah-G protein comprises an asparagine to glutamine substitution at position 126 of SEQ ID NO: 1. In some embodiments, the variant Nipah-G protein comprises an asparagine to glutamine substitution at position 273 of SEQ ID NO:1. In some embodiments, the variant Nipah-G protein comprises an asparagine to glutamine substitution at position 345 of SEQ ID NO:1. In some embodiments, the variant Nipah-G protein comprises an asparagine to glutamine substitution at position 384 of SEQ ID NO: 1. In some embodiments, the variant Nipah-G protein comprises an asparagine to glutamine substitution at position 448 of SEQ ID NO:1. In some embodiments, the variant Nipah-G protein comprises an asparagine to glutamine substitution at position 496 of SEQ ID NO:1. In some embodiments, the variant Nipah-G protein comprises a serine to alanine substitution at position 128 of SEQ ID NO:1.

In some embodiments, the variant Nipah-G protein comprises the sequence selected from the group consisting of any one of SEQ ID NOs: 2-12 and 246-354. In some embodiments, the variant Nipah-G protein comprises the sequence of SEQ ID NO: 5. In some embodiments, the variant Nipah-G protein comprises the sequence of SEQ ID NO: 259. In some embodiments, the variant Nipah-G protein comprises the sequence of SEQ ID NO: 263.

In some embodiments, the variant Nipah-G protein comprises the sequence selected from the group consisting of any one of SEQ ID NOs: 579-705, such as any exemplary variant Nipah-G proteins set forth in Table 2A below. In some embodiments, the variant Nipah-G protein comprises the sequence of SEQ ID NO: 581. In some embodiments, the variant Nipah-G protein comprises the sequence of SEQ ID NO: 602. In some embodiments, the variant Nipah-G protein comprises the sequence of SEQ ID NO: 606.

TABLE 2A
Exemplary Mutated Paramyxovirus Nipah G Proteins

| NiV-G | SEQ | Position | Position | Position | Position | Position | Position | Position |
|---|---|---|---|---|---|---|---|---|
| (Mutated) | ID NO | 39 | 126 | 273 | 345 | 384 | 448 | 496 |
| NivG | 1 | N | N | N | N | N | N | N |
| NivG.690 | 579 | Q | N | N | N | N | N | N |
| NivG.691 | 580 | N | Q | N | N | N | N | N |
| NivG.693 | 581 | N | N | Q | N | N | N | N |
| NivG.694 | 582 | N | N | N | Q | N | N | N |
| NivG.695 | 583 | N | N | N | N | Q | N | N |
| NivG.696 | 584 | N | N | N | N | N | Q | N |
| NivG.697 | 585 | N | N | N | N | N | N | Q |
| NivG.699 | 586 | N | N | N | Q | Q | Q | N |
| NivG.700 | 587 | N | N | N | Q | N | Q | N |
| NivG.701 | 588 | N | N | N | Q | N | N | Q |
| NivG.702 | 589 | Q | Q | Q | Q | Q | Q | Q |
| NivG.740 | 590 | N | N | N | N | N | Q | Q |
| NivG.742 | 591 | N | N | N | N | Q | N | Q |
| NivG.743 | 592 | N | N | N | N | Q | Q | N |
| NivG.744 | 593 | N | N | N | N | Q | Q | Q |
| NivG.748 | 594 | N | N | N | Q | N | Q | Q |
| NivG.749 | 595 | N | N | N | Q | Q | N | N |
| NivG.750 | 596 | N | N | N | Q | Q | N | Q |
| NivG.752 | 597 | N | N | N | Q | Q | Q | Q |
| NivG.753 | 598 | N | N | Q | N | N | N | Q |
| NivG.754 | 599 | N | N | Q | N | N | Q | N |
| NivG.755 | 600 | N | N | Q | N | N | Q | Q |
| NivG.756 | 601 | N | N | Q | N | Q | N | N |
| NivG.757 | 602 | N | N | Q | N | Q | N | Q |
| NivG.758 | 603 | N | N | Q | N | Q | Q | N |
| NivG.759 | 604 | N | N | Q | N | Q | Q | Q |
| NivG.760 | 605 | N | N | Q | Q | N | N | N |
| NivG.761 | 606 | N | N | Q | Q | N | N | Q |
| NivG.762 | 607 | N | N | Q | Q | N | Q | N |
| NivG.763 | 608 | N | N | Q | Q | N | Q | Q |
| NivG.764 | 609 | N | N | Q | Q | Q | N | N |
| NivG.765 | 610 | N | N | Q | Q | Q | N | Q |
| NivG.766 | 611 | N | N | Q | Q | Q | Q | N |
| NivG.767 | 612 | N | N | Q | Q | Q | Q | Q |
| NivG.768 | 613 | N | Q | N | N | N | N | Q |
| NivG.769 | 614 | N | Q | N | N | N | Q | N |
| NivG.770 | 615 | N | Q | N | N | N | Q | Q |
| NivG.771 | 616 | N | Q | N | N | Q | N | N |
| NivG.772 | 617 | N | Q | N | N | Q | N | Q |
| NivG.773 | 618 | N | Q | N | N | Q | Q | N |
| NivG.774 | 619 | N | Q | N | N | Q | Q | Q |
| NivG.775 | 620 | N | Q | N | Q | N | N | N |
| NivG.776 | 621 | N | Q | N | Q | N | N | Q |
| NivG.777 | 622 | N | Q | N | Q | N | Q | N |
| NivG.778 | 623 | N | Q | N | Q | N | Q | Q |
| NivG.779 | 624 | N | Q | N | Q | Q | N | N |
| NivG.780 | 625 | N | Q | N | Q | Q | N | Q |
| NivG.781 | 626 | N | Q | N | Q | Q | Q | N |
| NivG.782 | 627 | N | Q | N | Q | Q | Q | Q |
| NivG.783 | 628 | N | Q | Q | N | N | N | N |
| NivG.784 | 629 | N | Q | Q | N | N | N | Q |
| NivG.785 | 630 | N | Q | Q | N | N | Q | N |
| NivG.786 | 631 | N | Q | Q | N | N | Q | Q |
| NivG.787 | 632 | N | Q | Q | N | Q | N | N |
| NivG.788 | 633 | N | Q | Q | N | Q | N | Q |
| NivG.789 | 634 | N | Q | Q | N | Q | Q | N |
| NivG.790 | 635 | N | Q | Q | N | Q | Q | Q |
| NivG.791 | 636 | N | Q | Q | Q | N | N | N |
| NivG.792 | 637 | N | Q | Q | Q | N | N | Q |
| NivG.793 | 638 | N | Q | Q | Q | N | Q | N |
| NivG.794 | 639 | N | Q | Q | Q | N | Q | Q |
| NivG.795 | 640 | N | Q | Q | Q | Q | N | N |
| NivG.796 | 641 | N | Q | Q | Q | Q | N | Q |
| NivG.797 | 642 | N | Q | Q | Q | Q | Q | N |
| NivG.798 | 643 | N | Q | Q | Q | Q | Q | Q |
| NivG.799 | 644 | Q | N | N | N | N | N | Q |
| NivG.800 | 645 | Q | N | N | N | N | Q | N |
| NivG.801 | 646 | Q | N | N | N | N | Q | Q |
| NivG.802 | 647 | Q | N | N | N | Q | N | N |
| NivG.803 | 648 | Q | N | N | N | Q | N | Q |
| NivG.804 | 649 | Q | N | N | N | Q | Q | N |
| NivG.805 | 650 | Q | N | N | N | Q | Q | Q |
| NivG.806 | 651 | Q | N | N | Q | N | N | N |
| NivG.807 | 652 | Q | N | N | Q | N | N | Q |
| NivG.808 | 653 | Q | N | N | Q | N | Q | N |
| NivG.809 | 654 | Q | N | N | Q | N | Q | Q |
| NivG.810 | 655 | Q | N | N | Q | Q | N | N |
| NivG.811 | 656 | Q | N | N | Q | Q | N | Q |
| NivG.812 | 657 | Q | N | N | Q | Q | Q | N |
| NivG.813 | 658 | Q | N | N | Q | Q | Q | Q |
| NivG.814 | 659 | Q | N | Q | N | N | N | N |
| NivG.815 | 660 | Q | N | Q | N | N | N | Q |
| NivG.816 | 661 | Q | N | Q | N | N | Q | N |
| NivG.817 | 662 | Q | N | Q | N | N | Q | Q |
| NivG.818 | 663 | Q | N | Q | N | Q | N | N |
| NivG.819 | 664 | Q | N | Q | N | Q | N | Q |
| NivG.820 | 665 | Q | N | Q | N | Q | Q | N |
| NivG.821 | 666 | Q | N | Q | N | Q | Q | Q |
| NivG.822 | 667 | Q | N | Q | Q | N | N | N |
| NivG.823 | 668 | Q | N | Q | Q | N | N | Q |
| NivG.824 | 669 | Q | N | Q | Q | N | Q | N |
| NivG.825 | 670 | Q | N | Q | Q | N | Q | Q |
| NivG.826 | 671 | Q | N | Q | Q | Q | N | N |
| NivG.827 | 672 | Q | N | Q | Q | Q | N | Q |
| NivG.828 | 673 | Q | N | Q | Q | Q | Q | N |
| NivG.829 | 674 | Q | N | Q | Q | Q | Q | Q |
| NivG.830 | 675 | Q | Q | N | N | N | N | N |
| NivG.831 | 676 | Q | Q | N | N | N | N | Q |
| NivG.832 | 677 | Q | Q | N | N | N | Q | N |
| NivG.833 | 678 | Q | Q | N | N | N | Q | Q |
| NivG.834 | 679 | Q | Q | N | N | Q | N | N |
| NivG.835 | 680 | Q | Q | N | N | Q | N | Q |
| NivG.836 | 681 | Q | Q | N | N | Q | Q | N |
| NivG.837 | 682 | Q | Q | N | N | Q | Q | Q |
| NivG.838 | 683 | Q | Q | N | Q | N | N | N |
| NivG.839 | 684 | Q | Q | N | Q | N | N | Q |
| NivG.840 | 685 | Q | Q | N | Q | N | Q | N |
| NivG.841 | 686 | Q | Q | N | Q | N | Q | Q |
| NivG.842 | 687 | Q | Q | N | Q | Q | N | N |
| NivG.843 | 688 | Q | Q | N | Q | Q | N | Q |
| NivG.844 | 689 | Q | Q | N | Q | Q | Q | N |
| NivG.845 | 690 | Q | Q | N | Q | Q | Q | Q |
| NivG.846 | 691 | Q | Q | Q | N | N | N | N |
| NivG.847 | 692 | Q | Q | Q | N | N | N | Q |
| NivG.848 | 693 | Q | Q | Q | N | N | Q | N |
| NivG.849 | 694 | Q | Q | Q | N | N | Q | Q |
| NivG.850 | 695 | Q | Q | Q | N | Q | N | N |
| NivG.851 | 696 | Q | Q | Q | N | Q | N | Q |
| NivG.852 | 697 | Q | Q | Q | N | Q | Q | N |
| NivG.853 | 698 | Q | Q | Q | N | Q | Q | Q |
| NivG.854 | 699 | Q | Q | Q | Q | N | N | N |
| NivG.855 | 700 | Q | Q | Q | Q | N | N | Q |
| NivG.856 | 701 | Q | Q | Q | Q | N | Q | N |
| NivG.857 | 702 | Q | Q | Q | Q | N | Q | Q |
| NivG.858 | 703 | Q | Q | Q | Q | Q | N | N |
| NivG.859 | 704 | Q | Q | Q | Q | Q | N | Q |
| NivG.860 | 705 | Q | Q | Q | Q | Q | Q | N |

In some embodiments, the Paramyxovirus G/H glycoprotein to which the deglycosylation mutations is made is a Measles virus H (Mev-H) protein or a modified MeV-H protein that has an altered cytoplasmic tail compared to native MeV-H (e.g., SEQ ID NO:244). In some embodiments, the variant Paramyxovirus G/H protein has a sequence of amino acids that has at least 85%, at least 86%, at least 87%, at least 88%, at least 89%, at least 90%, at least 91%, at least 92%, at least 93%, at least 94% or at least 95% to SEQ ID NO: 244 and contains the acid substitution at one or more amino acid positions that reduce glycosylation of the G/H glycoprotein as provided herein.

In some embodiments, the G/H glycoprotein is derived from Measles virus H (Mev-H) protein and the one or more amino acid substitutions are at positions corresponding to positions selected from the group consisting of 168, 187, 200, 215, 238 of SEQ ID NO: 244. In some embodiments, the variant Mev-H protein comprises at least two amino acid substitutions, such as 2, 3, 4, or 5 substitutions at positions 168, 187, 200, 215, 238 of SEQ ID NO: 244.

In some embodiments, the Paramyxovirus G/H glycoprotein to which the deglycosylation mutations is made is a Canine distemper virus H (CDV-H) protein or a modified CDV-H protein that has an altered cytoplasmic tail compared to native CDV-H (e.g., SEQ ID NO:245). In some embodiments, the variant Paramyxovirus G/H protein has a sequence of amino acids that has at least 85%, at least 86%, at least 87%, at least 88%, at least 89%, at least 90%, at least 91%, at least 92%, at least 93%, at least 94% or at least 95% to SEQ ID NO: 245 and contains the acid substitution at one or more amino acid positions that reduce glycosylation of the G/H glycoprotein as provided herein.

In some embodiments, the G/H glycoprotein is derived from Canine distemper virus H (CDV-H) protein and the one or more amino acid substitutions are at positions corresponding to positions selected from the group consisting of 19, 149, 422 of SEQ ID NO: 245. In some embodiments, the variant CDV-H protein comprises at least two amino acid substitutions, such as 2 or 3 substitutions at positions 19, 149, 422 of SEQ ID NO: 245.

In some embodiments, the variant Paramyxovirus G/H protein reduces off-target transduction of a lipid particle comprising the variant protein. In some embodiments, the off-target cells comprise a primary human hepatocyte (PHH). In some embodiments, the off-target cells comprise a hematopoietic stem cell. In some embodiments, the titer of the lipid particles in off-target cell types following transduction is reduced compared to the titer in on-target cell types. In some embodiments, the titer is reduced by about 5%, about 10%, about 20%, about 30%, about 40%, about 50%, about 60%, about 70%, about 80%, about 90%, or about 100%. In some embodiments, the titer is reduced by about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to about 100%. In some embodiments, the ratio of on-target to off-target transduction is at least about 1.1, about 1.5, about 2.0, about 2.5, about 3.0, about 3.5, about 4.0, about 4.5, about 5.0, about 6.0, about 7.0, about 8.0, about 9.0, about 10.0, about 12.0, about 14.0, about 16.0, about 18.0, or about 20.0. In some embodiments, the ratio of on-target to off-target is between about 1.1 to about 1.5, 1.5 to about 2.0, 2.0 to about 2.5, 2.5 to about 3.0, 3.0 to about 3.5, 3.5 to about 4.0, 4.0 to about 4.5, 4.5 to about 5.0, 5.0 to about 6.0, 6.0 to about 7.0, 7.0 to about 8.0, 8.0 to about 9.0, 9.0 to about 10.0, 10.0 to about 12.0, 12.0 to about 14.0, 14.0 to about 16.0, 16.0 to about 18.0, or 18.0 to about 20.0.

Any techniques for assessing or quantifying titer may be employed. Non-limiting examples of available techniques for quantifying titer include viral particle number determination and titer by plaque assay. For example, the number of viral-based particles can be determined by measuring the absorbance at A260. Similarly, titer of infectious units (i.e., viral-based particles) can also be determined by quantitative immunofluorescence of particle specific proteins using monoclonal antibodies or by plaque assay. In some embodiments, methods that calculate the titer include the plaque assay, in which titrations of the viral-based particles are grown on cell monolayers and the number of plaques is counted after several days to several weeks. In some embodiments, titer can be determined using an endpoint dilution (TCID₆₀) method, which determines the dilution of virus at which 50% of the cell cultures are infected/transduced and hence, generally, can determine the titer within a certain range, such as one log.

In some embodiments, the reduction in off-target transduction as compared to on-target transduction is determined based on the number of cells expressing a marker, e.g. a GFP marker.

### B. Modified G/H Proteins (e.g., Truncated G/H Proteins)

In some embodiments, the Paramyxovirus G/H proteins described herein comprise a modified cytoplasmic tail (e.g., truncation) at the N-terminus. In some embodiments, the Paramyxovirus G/H protein is a modified Paramyxovirus G/H protein that contains an altered cytoplasmic tail compared to native Paramyxovirus G/H protein. In some embodiments, the modified Paramyxovirus G/H protein contains an altered cytoplasmic tail compared to native Paramyxovirus G/H protein set forth in any one of SEQ ID NOS: 13, 143-147, 244 and 245. In some embodiments, the modified Paramyxovirus G/H protein contains a modified cytoplasmic tail in which the native cytoplasmic tail is truncated or is replaced by a heterologous cytoplasmic tail. In some embodiments, the truncation comprises a truncated cytoplasmic tail as compared to the full-length G/H glycoprotein from the same Paramyxovirus.

In some embodiments, modified Paramyxovirus G/H protein is a functionally active variant or biologically active portion of any one of SEQ ID NOS: 13, 143-147, 244 and 245 that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 13, 143-147, 244 and 245. In some embodiments, the Paramyxovirus G/H protein to which the amino acid substitutions can be made is a modified G protein that is a functionally active variant or biologically active portion containing one or more amino acid mutations in addition to the one or more deglycosylation mutations as described, such as one or more amino acid insertions, deletions, substitutions or truncations. In some embodiments, such mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, at least one functionally active variant or the biologically active portion thereof is a variant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein or biologically active portion thereof. In some embodiments, the wild-type G protein has the sequence set forth in any one of SEQ ID NOS: 13, 143-147, 244 and 245.

In some embodiments, modified Paramyxovirus G protein is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein. In particular embodiments, the truncation is an N-terminal truncation of all or a portion of the cytoplasmic domain. In some embodiments, truncated G protein lacks up to 49 contiguous amino acid residues at or near the N-terminus of the wild-type G protein, such as a wild-type G protein set forth in any one of SEQ ID NOS: 13, 143-147, 244 and 245. In some embodiments, the truncated G protein lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, the cytoplasmic tail is a truncated portion thereof that is at least 5 amino acids in length, from or from about 5-44, from or from about 5-40, from or from about 5-30, from or from about 5-20, from or from about 5-10, from or from about 10-44, from or from about 10-40, from or from about 10-30, from or from about 10-20, from or from about 20-44, from or from about 20-40, from or from about 20-30, from or from about 30-44, from or from about 30-40, from or from about 40-44 amino acids in length. In some embodiments, the truncated portion thereof is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 or 44 amino acids in length.

In some embodiments, the Paramyxovirus G/H has a cytoplasmic tail that is a truncated Paramyxovirus G/H cytoplasmic tail. In some embodiments, the Paramyxovirus G/H is a variant Paramyxovirus G/H. In some embodiments, the truncated paramyxovirus G/H cytoplasmic tail has a deletion of up to 40, up to 35, up to 30, up to 29, up to 28, up to 27, up to 26, up to 25, up to 24, up to 23, up to 22, up to 21, up to 20, up to 19, up to 18, up to 17, up to 16, up to 15, or up to 14 contiguous amino acid residues at or near the N-terminus of the wild-type G/H cytoplasmic tail. In some embodiments, the truncated G/H cytoplasmic tail has a deletion of at or about or up to 19 contiguous amino acid residues at or near the N-terminus of the wild-type G/H cytoplasmic tail. In some embodiments, the truncated G/H cytoplasmic tail has a deletion of at or about or up to 13 contiguous amino acid residues at or near the N-terminus of the wild-type Paramyxovirus G/H cytoplasmic tail. In some embodiments, the truncated G/H cytoplasmic tail has a deletion of at or about or up to 6 contiguous amino acid residues at or near the N-terminus of the wild-type G/H cytoplasmic tail.

In some embodiments, the variant Paramyxovirus G/H protein is derived from Nipah virus. In some embodiments, the Paramyxovirus G/H protein to which the amino acid substitutions can be introduced is a Nipah G protein (NiV-G) that contains a modified cytoplasmic tail in which at least a part of the native cytoplasmic tail (e.g. corresponding to amino acids 1-45 of SEQ ID NO: 16) is a truncated portion thereof from a glycoprotein from Nipah Virus. In some embodiments, the truncated portion thereof is directly linked to the N-terminus of the sequence set forth in SEQ ID NO: 14, and further comprises any one or more of the amino acid mutations described in Section II.A. In some embodiments, the variant NiV-G further comprises mutations in the extracellular domain that reduce or abrogate binding to an Ephrin B2 or B3 corresponding to one or more of E501A, W504A, Q530A and E533A, with numbering of residues as set forth SEQ ID NO:13. In some embodiments, the modified cytoplasmic tail with the truncated portion is directly linked to the N-terminus of the sequence set forth in SEQ ID NO: 15 and further comprises any one or more of the amino acid mutations described in Section II.A.1.

Non-limiting examples of variant NiV-G proteins, including truncated NiV-G or NiV-G with a altered or modified cytoplasmic tail, are described in WO2013148327, WO2017182585, or PCT/US2022/081872. Further exemplary variant NiV-G proteins are described in Bender et al. 2016 PLOS Pathol 12 (6): e1005641.

In some embodiments, the modified NiV-G protein to which the amino acid substitutions can be introduced comprises a modified cytoplasmic tail which comprises a truncated cytoplasmic tail from a glycoprotein from the same Nipah virus. In some embodiments, the modified NiV-G contains a modified cytoplasmic tail in which at least a part of the native cytoplasmic tail (e.g. corresponding to amino acids 1-45 of SEQ ID NO: 16) is a truncated portion thereof from a glycoprotein from Nipah Virus. In some embodiments, the cytoplasmic tail is a truncated portion thereof that is at least 5 amino acids in length, from or from about 5-44, from or from about 5-40, from or from about 5-30, from or from about 5-20, from or from about 5-10, from or from about 10-44, from or from about 10-40, from or from about 10-30, from or from about 10-20, from or from about 20-44, from or from about 20-40, from or from about 20-30, from or from about 30-44, from or from about 30-40, from or from about 40-44 amino acids in length. In some embodiments, the truncated portion thereof is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 or 44 amino acids in length. In some embodiments, the truncated NiV-G cytoplasmic tail has a deletion of up to 40, up to 35, up to 30, up to 29, up to 28, up to 27, up to 26, up to 25, up to 24, up to 23, up to 22, up to 21, up to 20, up to 19, up to 18, up to 17, up to 16, up to 15, or up to 14 contiguous amino acid residues at or near the N-terminus of the wild-type NiV-G cytoplasmic tail set forth in SEQ ID NO: 355. In some embodiments, the variant NiV-G has a deletion of between 5 and 41 contiguous amino acid residues at or near the N-terminus of the wild-type NiV-G protein cytoplasmic tail set forth in SEQ ID NO: 355. In some embodiments, the variant NiV-G has a deletion of between 26 and 40 contiguous amino acid residues at or near the N-terminus of the wild-type NiV-G protein cytoplasmic tail set forth in SEQ ID NO: 355.

In some embodiments, the modified NiV-G protein is truncated and lacks up to 5 contiguous amino acid residues at or near the N-terminus of the wild-type NiV-G protein, such as compared to wild-type NiV-G set forth in SEQ ID NO: 13. In some embodiments, the modified NiV-G protein is truncated and lacks up to 10 contiguous amino acid residues at or near the N-terminus of the wild-type NiV-G protein, such as compared to wild-type NiV-G set forth in SEQ ID NO: 13. In some embodiments, the modified NiV-G protein is truncated and lacks up to 15 contiguous amino acid residues at or near the N-terminus of the wild-type NiV-G protein, such as compared to wild-type NiV-G set forth in SEQ ID NO: 13. In some embodiments, the modified NiV-G protein is truncated and lacks up to 20 contiguous amino acid residues at or near the N-terminus of the wild-type NiV-G protein, such as compared to wild-type NiV-G set forth in SEQ ID NO: 13. In some embodiments, the modified NiV-G protein is truncated and lacks up to 25 contiguous amino acid residues at or near the N-terminus of the wild-type NiV-G protein, such as compared to wild-type NiV-G set forth in SEQ ID NO: 13. In some embodiments, the modified NiV-G protein is truncated and lacks up to 30 contiguous amino acid residues at or near the N-terminus of the wild-type NiV-G protein, such as compared to wild-type NiV-G set forth in SEQ ID NO: 13. In some embodiments, the modified NiV-G protein is truncated and lacks up to 35 contiguous amino acid residues at or near the N-terminus of the wild-type NiV-G protein, such as compared to wild-type NiV-G set forth in SEQ ID NO: 13. In some embodiments, the modified NiV-G protein contains an N-terminal methionine.

In some embodiments, the modified NiV-G protein is truncated and lacks up to amino acid 34 at or near the N-terminus of the wild-type NiV-G protein, such as compared to wild-type NiV-G set forth in SEQ ID NO: 13. In some embodiments, the modified NiV-G protein contains an N-terminal methionine. In some embodiments, the modified NiV-G protein lacks amino acids 2-34 as compared to wild-type NiV-G set forth in SEQ ID NO: 13. In some embodiments, the modified NiV-G to which the deglycosylation amino acid substitutions can be introduced is set forth in SEQ ID NO: 1.

In some embodiments, the modified NiV-G protein to which the deglycosylation amino acid substitutions can be introduced has a cytoplasmic tail deletion of amino acid residues 2-41, 2-40, 2-39, 2-38, 2-37, 2-36, 2-34, 2-35, 2-33, 2-32, 2-31, 2-30, 2-29, 2-28, 2-27, 2-26, 2-25, 2-22, 2-21, 2-16, 2-11, or 2-5 of SEQ ID NO:355. In some embodiments, the cytoplasmic tail of the modified NiV-G is the Nipah virus cytoplasmic tail set forth in any one of SEQ ID NOS: 356-378. In some embodiments, the cytoplasmic tail of the modified NiV-G is the cytoplasmic tail set forth in any one of SEQ ID NOS: 356-378 that lacks the N-terminal methionine. In some embodiments, the modified NiV-G to which the deglycosylation mutations can be introduced has a sequence in which the cytoplasmic tail, such as set forth in any one of SEQ ID NOS: 356-378, is directly linked to the N-terminus of the sequence set forth in SEQ ID NO: 14. In some embodiments, the modified NiV-G to which the deglycosylation mutations is introduced has a sequence in which the cytoplasmic tail set forth in any one of SEQ ID NOS: 356-378 is directly linked to the N-terminus of the sequence set forth in SEQ ID NO: 15.

In some embodiments, the modified NiV-G has the truncated NiV-G cytoplasmic tail set forth in SEQ ID NO: 357, 363, 369, 561, 570 or 571, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOs: 357, 363, 369, 561, 570 or 571. In some embodiments, the modified NiV-G has the truncated NiV-G cytoplasmic tail set forth in SEQ ID NO: 357, 363, 369, 561, 570 or 571.

In some embodiments, the modified NiV-G contains a modified cytoplasmic tail in which at least a part of the native cytoplasmic tail (e.g. corresponding to amino acids 1-45 of SEQ ID NO: 16) is a mutated portion thereof from a glycoprotein from Nipah Virus. In some embodiments, the cytoplasmic tail is a mutated portion of the Nipah virus cytoplasmic tail set forth in any one of SEQ ID NOS: 379-388. In some embodiments, it is understood that the truncated NiV-G cytoplasmic tail may include the sequence set forth in any one of SEQ ID NOS: 379-388 that lacks the N-terminal methionine. In some embodiments, the modified NiV-G to which the deglycosylation mutations can be introduced has a sequence in which the cytoplasmic tail set forth in any one of SEQ ID NOS: 379-388 is directly linked to the N-terminus of the sequence set forth in SEQ ID NO: 14. In some embodiments, the modified NiV-G to which the deglycosylation mutations can be introduced has a sequence in which the cytoplasmic tail set forth in any one of SEQ ID NOS: 379-388 is directly linked to the N-terminus of the sequence set forth in SEQ ID NO: 15.

In some embodiments, the modified NiV-G has a modified cytoplasmic tail which comprises a heterologous cytoplasmic tail or a truncated portion thereof from a glycoprotein from another virus. In some embodiments, the other virus is a member of the Kingdom Orthornavirae. In some embodiments, the other virus is a member of the family Paramyxoviridae, Rhabdoviridae, Arenaviridae, or Retroviridae. In some embodiments, the other virus is a member of the family Paramyxoviridae.

In some embodiments, the modified NiV-G contains a modified cytoplasmic tail in which at least a part of the native cytoplasmic tail (e.g. corresponding to amino acids 1-45 of SEQ ID NO: 16) is replaced by a heterologous cytoplasmic tail or a truncated portion thereof from a glycoprotein from another virus or viral-associated protein. In some embodiments, the replaced cytoplasmic tail is a heterologous cytoplasmic tail or a truncated portion thereof that is at least 5 amino acids in length. In some embodiments, the replaced heterologous cytoplasmic tail or a truncated portion thereof is from or from about 5-180 amino acids in length, such as from or from about 5-150, from or from about 5-100, from or from about 5-75, from or from about 5-50, from or from about 5-40, from or from about 5-30, from or from about 5-20, from or from about 5-10, from or from about 10-150, from or from about 10-100, from or from about 10-75, from or from about 10-50, from or from about 10-40, from or from about 10-30, from or from about 10-20, from or from about 20-150, from or from about 20-100, from or from about 20-75, from or from about 20-50, from or from about 20-40, from or from about 20-30, from or from about 30-150, from or from about 30-100, from or from about 30-75, from or from about 30-50, from or from about 30-40, from or from about 40-150, from or from about 40-100, from or from about 40-75, from or from about 40-50, from or from about 50-150, from or from about 50-100, from or from about 50-75, from or from about 75-150, from or from about 75-100 or from or from about 100-150 amino acids in length. In some embodiments, the replaced heterologous cytoplasmic tail or a truncated portion thereof is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids in length. In some embodiments, the modified NiV-G to which the deglycosylation mutations can be introduced has a sequence in which the heterologous cytoplasmic tail or the truncated portion thereof is directly linked to the N-terminus of the sequence set forth in SEQ ID NO: 14.

In some embodiments, the modified NiV-G contains a heterologous cytoplasmic tail that is a cytoplasmic tail or a truncated portion thereof from a glycoprotein from another virus, such as a paramyxovirus, a retrovirus, a filovirus, a rhabdovirus or an arenavirus. In some embodiments, the virus is a paramyxovirus other than a Nipah virus. For instance, the virus is a measles virus, Bat paramyxovirus, Cedar Virus, Canine Distemper Virus, Sendai virus, Hendra virus, Human Parainfluenza virus, or Newcastle Disease virus. In some embodiments, the replaced heterologous cytoplasmic tail is the native cytoplasmic tail or a truncated portion of the native cytoplasmic tail of another virus, such as a truncated portion of the cytoplasmic tail set forth in any one of SEQ ID NOS: 390-516. In some embodiments, the modified NiV-G to which the deglycosylation mutations can be introduced has a sequence in which the heterologous cytoplasmic tail or the truncated portion thereof set forth in any one of SEQ ID NOS: 390-516 is directly linked to the N-terminus of the sequence set forth in SEQ ID NO: 14. In some embodiments, the modified NiV-G contains mutations in the extracellular domain that reduce or abrogate binding to an Ephrin B2 or B3 corresponding to one or more of E501A, W504A, Q530A and E533A, with numbering of residues as set forth SEQ ID NO: 13. In some embodiments, the modified NiV-G to which the deglycosylation mutations can be introduced has a sequence in which the heterologous cytoplasmic tail or the truncated portion thereof set forth in any one of SEQ ID NOS: 390-516 is directly linked to the N-terminus of the sequence set forth in SEQ ID NO: 15. In some embodiments, it is understood that the heterologous cytoplasmic tail or the truncated portion thereof may include any sequence set forth in any one of SEQ ID NOS: 390-516 that lacks the N-terminal methionine.

In some embodiments, the virus is a retrovirus. For instance, the virus may be a baboon endogenous virus (BaEV), Gibbon Ape Leukemia virus (GaLV), murine leukemia virus, or human immunodeficiency virus 1 (HIV-1). In some embodiments, the replaced heterologous cytoplasmic tail is the native cytoplasmic tail or a truncated portion of the native cytoplasmic tail of another virus, such as set forth in any one of SEQ ID NOS: 517-518, 524-527, 529-532, or 535-549. In some embodiments, the modified NiV-G to which the deglycosylation mutations can be introduced has a sequence in which the heterologous cytoplasmic tail or the truncated portion thereof set forth in any one of SEQ ID NOS: 517-518, 524-527, 529-532, or 535-549 is directly linked to the N-terminus of the sequence set forth in SEQ ID NO: 14. In some embodiments, the modified NiV-G contains mutations in the extracellular domain that reduce or abrogate binding to an Ephrin B2 or B3 corresponding to one or more of E501A, W504A, Q530A and E533A, with numbering of residues as set forth SEQ ID NO:13. In some embodiments, the modified NiV-G to which the deglycosylation mutations can be introduced has a sequence in which the heterologous cytoplasmic tail or the truncated portion thereof set forth in any one of SEQ ID NOS: 517-518, 524-527, 529-532, or 535-549 is directly linked to the N-terminus of the sequence set forth in SEQ ID NO: 15. In some embodiments, it is understood that the heterologous cytoplasmic tail or the truncated portion thereof may include any sequence set forth in any one of SEQ ID NOS: 517-518, 524-527, 529-532, or 535-549 that lacks the N-terminal methionine.

In some embodiments, the virus is a filovirus. For instance, the virus may be an Ebola virus (EboV). In some embodiments, the replaced heterologous cytoplasmic tail is the native cytoplasmic tail or a truncated portion of the native cytoplasmic tail of another virus, such as set forth in any one of SEQ ID NOS: 522 or 523. In some embodiments, the modified NiV-G to which the deglycosylation mutations can be introduced has a sequence in which the heterologous cytoplasmic tail or the truncated portion thereof set forth in any one of SEQ ID NOS: 522 or 523 is directly linked to the N-terminus of the sequence set forth in SEQ ID NO: 14. In some embodiments, the variant NiV-G contains mutations in the extracellular domain that reduce or abrogate binding to an Ephrin B2 or B3 corresponding to one or more of E501A, W504A, Q530A and E533A, with numbering of residues as set forth SEQ ID NO:13. In some embodiments, the modified NiV-G to which the deglycosylation mutations can be introduced has a sequence in which the heterologous cytoplasmic tail or the truncated portion thereof set forth in any one of SEQ ID NOS: 522 or 523 is directly linked to the N-terminus of the sequence set forth in SEQ ID NO: 15. In some embodiments, it is understood that the heterologous cytoplasmic tail or the truncated portion thereof may include any sequence set forth in any one of SEQ ID NOS: 522 or 523 that lacks the N-terminal methionine.

In some embodiments, the virus is a rhabdovirus. For instance, the virus may be Cocal vesiculovirus (Cocal) or vesicular stomatitis virus (VSV). In some embodiments, the replaced heterologous cytoplasmic tail is the native cytoplasmic tail or a truncated portion of the native cytoplasmic tail of another virus, such as set forth in any one of SEQ ID NOS: 520, 521, 533, or 534. In some embodiments, the modified NiV-G to which the deglycosylation mutations can be introduced has a sequence in which the heterologous cytoplasmic tail or the truncated portion thereof set forth in any one of SEQ ID NOS: 520, 521, 533, or 534 is directly linked to the N-terminus of the sequence set forth in SEQ ID NO: 14. In some embodiments, the modified NiV-G contains mutations in the extracellular domain that reduce or abrogate binding to an Ephrin B2 or B3 corresponding to one or more of E501A, W504A, Q530A and E533A, with numbering of residues as set forth SEQ ID NO:13. In some embodiments, the modified NiV-G to which the deglycosylation mutations can be introduced has a sequence in which the heterologous cytoplasmic tail or the truncated portion thereof set forth in any one of SEQ ID NOS: 520, 521, 533, or 534 is directly linked to the N-terminus of the sequence set forth in SEQ ID NO: 15. In some embodiments, it is understood that the heterologous cytoplasmic tail or the truncated portion thereof may include any sequence set forth in any one of SEQ ID NOS: 520, 521, 533, or 534 that lacks the N-terminal methionine.

In some embodiments, the virus is an arenavirus. For instance, the virus may be Lymphocytic choriomeningitis virus (LCMV). In some embodiments, the replaced heterologous cytoplasmic tail is the native cytoplasmic tail or a truncated portion of the native cytoplasmic tail of another virus, such as set forth in SEQ ID NOS: 528. In some embodiments, the modified NiV-G to which the deglycosylation mutations can be introduced has a sequence in which the heterologous cytoplasmic tail or the truncated portion thereof set forth in SEQ ID NOS: 528 is directly linked to the N-terminus of the sequence set forth in SEQ ID NO: 14. In some embodiments, the modified NiV-G contains mutations in the extracellular domain that reduce or abrogate binding to an Ephrin B2 or B3 corresponding to one or more of E501A, W504A, Q530A and E533A, with numbering of residues as set forth SEQ ID NO: 13. In some embodiments, the modified NiV-G to which the deglycosylation mutations can be introduced has a sequence in which the heterologous cytoplasmic tail or the truncated portion thereof set forth in SEQ ID NOS: 528 is directly linked to the N-terminus of the sequence set forth in SEQ ID NO: 15. In some embodiments, it is understood that the heterologous cytoplasmic tail or the truncated portion thereof may include any sequence set forth in any one of SEQ ID NOS: 528 that lacks the N-terminal methionine.

In some embodiments, the modified NiV-G contains a heterologous cytoplasmic tail that is a cytoplasmic tail or a truncated portion thereof from a glycoprotein from CD63. In some embodiments, the heterologous cytoplasmic tail replaces at least a part of the native cytoplasmic tail of NiV-G (e.g. corresponding to amino acids 1-45 of SEQ ID NO: 16). In some embodiments, the heterologous tail is a contiguous sequence of 5, 6, 7, 8, 9, 10, 11, 12, 13, 14, 15, 16, 17, 18, 19, 20, 21, 22, 23, 24, or 25 N-terminal amino acids of the native cytoplasmic tail of CD63. In some embodiments, the native cytoplasmic tail of CD63 is set forth in SEQ ID NOs: 552, 551, or 552. In some embodiments, the heterologous cytoplasmic tail is a truncated portion of the CD63 cytoplasmic tail set forth in any one of SEQ ID NOS: 550-555. In some embodiments, the modified NiV-G to which the deglycosylation mutations can be introduced has a sequence in which the heterologous cytoplasmic tail set forth in any one of SEQ ID NOS: 550-555 is directly linked to the N-terminus of the sequence set forth in SEQ ID NO: 14. In some embodiments, the modified NiV-G to which the deglycosylation mutations can be introduced has a sequence in which the heterologous cytoplasmic tail set forth in any one of SEQ ID NOS: 550-555 is directly linked to the N-terminus of the sequence set forth in SEQ ID NO: 15. In some embodiments, the modified NiV-G comprises a modified cytoplasmic tail which comprises a mutated cytoplasmic tail from a glycoprotein from the same Nipah virus.

### C. Retargeted G/H Proteins

In some embodiments, the variant Paramyxovirus G/H proteins described herein may comprise further modifications to allow for specific targeting of desired cell types. In some embodiments, the modifications comprise amino acid mutations. In some embodiments, the modification comprises attachment or linkage to a binding domain that binds to a target molecule, such as a cell surface marker. For instance, provided in some aspects is a targeted lipid particle (e.g. targeted lentiviral vector) that includes a re-targeted variant Paramyxovirus G/H protein comprising any of the provided variant Paramyxovirus G/H proteins attached to a binding domain, in which the re-targeted variant Paramyxovirus G/H protein is exposed on the surface of the targeted lipid particle (e.g. targeted lentiviral vector).

In some embodiments, also provided are any of the provided variant NiV-G proteins containing one or more amino acids substitutions to reduce glycosylation that are also modified to be re-targeted compared to the native tropism of NiV-G. For instance, mutations in NiV-G that completely abrogate ephrinB2 and B3 binding, but that do not impact the association of this NiV-G with NiV-F, have been identified (Aguilar, et al. J Biol Chem. 2009; 284 (3): 1628-1635; Weise et al. J Virol. 2010; 84 (15): 7634-764; Negrete et al., J Virol. 2007; 81 (19): 10804-10814; Negrete et al. PLOS Pathog. 2006; Guillaume et al., J. Virol 2006, 80 (15) 7546-7554). Thus, in provided aspects, a variant NiV-G protein provided herein may further contain a mutation in its extracellular domain to reduce or abrogate binding to Ephrin B2 and/B3. In some embodiments, the mutations can include one or more of mutations E501A, W504A, Q530A and E533A, with reference to numbering of wild-type NiV-G set forth in SEQ ID NO: 16. In some embodiments, any of the provided variant NiV-G proteins may also be linked or fused to a binding molecule for targeted binding to a target molecule of interest. In some embodiments, the variant G protein is a fusion of a binding molecule with variant NiV-G, including a NiV-G with mutations to abrogate Ephrin B2 and/or Ephrin B3 binding. This could allow for altered G protein tropism allowing for targeting of other desired cell types that are not ephrinB2+ through the addition of the binding molecule directed against a different cell surface molecule.

In some embodiments, the variant G protein or the biologically active portion thereof is a variant of wild-type NiV-G and exhibits reduced binding to one or both of the native binding partners Ephrin B2 or Ephrin B3. In some embodiments, the variant G-protein or the biologically active portion, such as a variant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%. In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO: 13. In some embodiments, at least one G protein is a variant G protein containing one or more amino acid substitutions selected from the group consisting of E501A, W504A, Q530A and E533A with reference to numbering set forth in SEQ ID NO:13. In some embodiments, at least one G protein is a variant G protein that contains one or more amino acid substitutions elected from the group consisting of E501A, W504A, Q530A and E533A with reference to SEQ ID NO: 13 and is a biologically active portion thereof containing an N-terminal truncation. In some embodiments, the mutations can improve transduction efficiency. In some embodiments, the mutations allow for specific targeting of other desired cell types that are not Ephrin B2 or Ephrin B3. In some embodiments, the mutations result in at least the partial inability to bind at least one natural receptor, such has reduce the binding to at least one of Ephrin B2 or Ephrin B3. In some embodiments, such mutations described herein interfere with natural receptor recognition.

In some embodiments, the G/H protein contains one or more amino acid substitutions in a residue that is involved in the interaction with one or both of Ephrin B2 and Ephrin B3. In some embodiments, the amino acid substitutions correspond to one or more of positions E501, W504, Q530 and E533 with reference to SEQ ID NO: 13. In some embodiments, the one or more additional mutations comprises a substitution to alanine.

In some embodiments, any one of the variant Paramyxovirus G/H glycoproteins is further attached or linked to a binding domain that binds to a target molecule, such as a cell surface marker. For instance, provided in some aspects is a targeted lipid particle (e.g. targeted lentiviral vector) that includes a re-targeted G/H protein containing any of the provided G/H proteins attached to a binding domain, in which the re-targeted G/H protein is exposed on the surface of the targeted lipid particle (e.g. targeted lentiviral vector).

In some embodiments, the binding domain can be any agent that binds to a cell surface molecule on a target cell. In some embodiments, the binding domain can be an antibody or an antibody portion or fragment. In some embodiments, the binding domain is a single domain antibody (sdAb). In some embodiments, the binding domain is a single chain variable fragment (scFv). The binding domain can be linked directly or indirectly to the G/H protein. In particular embodiments, the binding domain is linked to the C-terminus (C-terminal amino acid) of the G/H protein or the biologically active portion thereof. The linkage can be via a peptide linker, such as a flexible peptide linker.

The binding domain may be modulated to have different binding strengths. For example, scFvs and antibodies with various binding strengths may be used to alter the fusion activity of the chimeric attachment proteins towards cells that display high or low amounts of the target antigen. For example DARPins with different affinities may be used to alter the fusion activity towards cells that display high or low amounts of the target antigen. Binding domains may also be modulated to target different regions on the target ligand, which will affect the fusion rate with cells displaying the target.

The binding domain may comprise a humanized antibody molecule, intact IgA, IgG, IgE or IgM antibody; bi- or multi-specific antibody (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BITERs; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. A targeting moiety can also include an antibody or an antigen-binding fragment thereof (e.g., Fab, Fab′, F(ab′) 2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs).

In some embodiments, the binding domain is a single chain molecule. In some embodiments, the binding domain is a single domain antibody. In some embodiments, the binding domain is a single chain variable fragment. In particular embodiments, the binding domain contains an antibody variable sequence(s) that is human or humanized.

In some embodiments, the binding domain is a single domain antibody. In some embodiments, the single domain antibody can be human or humanized. In some embodiments, the single domain antibody or portion thereof is naturally occurring. In some embodiments, the single domain antibody or portion thereof is synthetic.

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In some embodiments, the heavy chain antibody devoid of light chains is referred to as VHH. In some embodiments, the single domain antibody antibodies have a molecular weight of 12-15 kDa. In some embodiments, the single domain antibody antibodies include camelid antibodies or shark antibodies. In some embodiments, the single domain antibody molecule is derived from antibodies raised in Camelidae species, for example in camel, llama, dromedary, alpaca, vicuna and guanaco. In some embodiments, the single domain antibody is referred to as immunoglobulin new antigen receptors (IgNARs) and is derived from cartilaginous fishes. In some embodiments, the single domain antibody is generated by splitting dimeric variable domains of human or mouse IgG into monomers and camelizing critical residues.

In some embodiments, the single domain antibody can be generated from phage display libraries. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, single domain antibodies a library of human single domain antibodies is synthetically generated by introducing diversity into one or more scaffolds.

A G/H protein provided herein and a binding domain that binds to a cell surface molecule on a target cell. In some embodiments, the binding domain is a single domain antibody (sdAb). In some embodiments, the binding domain is a single chain variable fragment (scFv). The binding domain can be linked directly or indirectly to the G/H protein. In particular embodiments, the binding domain is linked to the C-terminus (C-terminal amino acid) of the G/H protein or the biologically active portion thereof. The linkage can be via a peptide linker, such as a flexible peptide linker.

In some embodiments, the C-terminus of the binding domain is attached to the C-terminus of the G/H protein or biologically active portion thereof. In some embodiments, the N-terminus of the binding domain is exposed on the exterior surface of the lipid bilayer. In some embodiments, the N-terminus of the binding domain binds to a cell surface molecule of a target cell. In some embodiments, the binding domain specifically binds to a cell surface molecule present on a target cell. In some embodiments, the cell surface molecule is a protein, glycan, lipid or low molecular weight molecule. In some embodiments, the binding domain is one of any binding domains as described above.

In some embodiments, a binding domain (e.g. sdAb or one of any binding domains as described herein) binds to a cell surface antigen of a cell. In some embodiments, a cell surface antigen is characteristic of one type of cell. In some embodiments, a cell surface antigen is characteristic of more than one type of cell.

In some embodiments, the cell surface molecule of a target cell is an antigen or portion thereof. In some embodiments, the single domain antibody or portion thereof is an antibody having a single monomeric domain antigen binding/recognition domain that is able to bind selectively to a specific antigen. In some embodiments, the single domain antibody binds an antigen present on a target cell.

Exemplary cells include polymorphonuclear cells (also known as PMN, PML, PMNL, or granulocytes), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, immune effector cells, lymphocytes, macrophages, dendritic cells, natural killer cells, T cells, cytotoxic T lymphocytes, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes.

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g. hepatocyte), or a cardiac cell (e.g. cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g. a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoietic stem cell (HSC), a liver cell or a fully differentiated cell. In some embodiments, the target cell is a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+B cell, a CD19+B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A 10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacytoid dendritic cell, a CD11c+ cell, a CD11b+ cell, a splenocyte, a B cell, a hepatocyte, a endothelial cell, or a non-cancerous cell).

In some embodiments, the binding domain (e.g. sdAb) variable domain binds a cell surface molecule or antigen. In some embodiments, the cell surface molecule is ASGR1, ASGR2, TM4SF5, CD8, CD4, CD3, or low density lipoprotein receptor (LDL-R). In some embodiments, the cell surface molecule is ASGR1. In some embodiments, the cell surface molecule is ASGR2. In some embodiments, the cell surface molecule is TM4SF5. In some embodiments, the cell surface molecule is CD8. In some embodiments, the cell surface molecule is CD4. In some embodiments, the cell surface molecule is LDL-R.

In some embodiments, the G/H protein or functionally active variant or biologically active portion thereof is linked directly to the binding domain and/or variable domain thereof. In some embodiments, the targeted envelope protein is a fusion protein that has the following structure: (N′-single domain antibody-C′)-(C′-G protein-N′).

In some embodiments, the binding domain (e.g. sdAb) variable domain binds a cell surface molecule or antigen. In some embodiments, the cell surface molecule is ASGR1, ASGR2, TM4SF5, CD3, CD8, CD4, or low density lipoprotein receptor (LDL-R). In some embodiments, the cell surface molecule is ASGR1. In some embodiments, the cell surface molecule is ASGR2. In some embodiments, the cell surface molecule is TM4SF5. In some embodiments, the cell surface molecule is CD3. In some embodiments, the cell surface molecule is CD8. In some embodiments, the cell surface molecule is CD4. In some embodiments, the cell surface molecule is LDL-R.

The viral vectors disclosed herein include one or more CD4 binding agents. For example, a CD4 binding agent may be fused to or incorporated in a protein fusogen or viral envelope protein. In another embodiment, a CD4 binding agent may be incorporated into the viral envelope via fusion with a transmembrane domain.

Exemplary CD4 binding agents include antibodies and fragments thereof (e.g., scFv. VHH) that bind to CD4. Such antibodies may be derived from any species, and may be for example, mouse, rabbit, human, humanized, or camelid antibodies. Exemplary antibodies include ibalizumab, zanolimumab, tregalizumab, priliximab, cedelizumab, clenoliximab, keliximab, and anti-CD4 antibodies disclosed in WO2002102853, WO2004083247, WO2004067554, WO2007109052, WO2008134046, WO2010074266, WO2012113348, WO2013188870, WO2017104735, WO2018035001, WO2018170096, WO2019203497, WO2019236684, WO2020228824, U.S. Pat. Nos. 5,871,732, 7,338,658, 7,722,873, 8,399,621, 8,911,728, 9,005,963, U.S. Pat. Nos. 9,587,022, 9,745,552, U.S. provisional application No. 63/326,269, U.S. provisional application No. 63/341,681; as well as antibodies B486A1, RPA-T4, CE9.1 (Novus Biologicals); GK1.5, RM4-5, RPA-T4, OKT4, 4SM95, S3.5, N1UGO (ThermoFisher); GTX50984, ST0488, 10B5, EP204 (GeneTex); GK1.3, 5A8, 10C12, W3/25, 8A5, 13B8.2, 6G5 (Absolute Antibody); VIT4, M-T466, M-T321, REA623, (Miltenyi); MEM115, MT310 (Enzo Life Sciences); H129.19, 5B4, 6A17, 18-46, A-1, C-1, OX68 (Santa Cruz); EP204, D2E6M (Cell Signaling Technology). Other exemplary binding agents include designed ankyrin repeat proteins (DARPins) (e.g., the anti-CD4 DARPin disclosed in WO2017182585) and binding agents based on fibronectin type III (Fn3) scaffolds. Each of U.S. Pat. No. 9,005,963, U.S. provisional application No. 63/326,269, and U.S. provisional application No. 63/341,681 is incorporated by reference herein in its entirety.

In some embodiments, protein fusogens or viral envelope proteins may be re-targeted by mutating amino acid residues in a fusion protein or a targeting protein (e.g. the hemagglutinin (H) protein or G protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type Niv-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi: 10.1038/nbt942, Molecular Therapy vol. 16 no. 8, 1427-1436 August 2008, doi: 10.1038/nbt1060, DOI: 10.1128/JVL76.7.3558-3563.2002, DOI: 10.1128/JVL75.17.8016-8020.2001, doi: 10.1073pnas.0604993103).

In some embodiments, protein fusogens may be re-targeted by covalently conjugating a CD4 binding agent to the fusion protein or targeting protein (e.g. the attachment or hemagglutinin protein). In some embodiments, the fusogen and CD4 binding agent are covalently conjugated by expression of a chimeric protein comprising the fusogen linked to the CD4 binding agent. In some embodiments, a single-chain variable fragment (scFv) can be conjugated to fusogens to redirect fusion activity towards cells that display the scFv binding target (doi: 10.1038/nbt1060, DOI 10.1182/blood-2012-11-468579, doi: 10.1038/nmeth.1514, doi: 10.1006/mthe.2002.0550, HUMAN GENE THERAPY 11:817-826, doi: 10.1038/nbt942, doi: 10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi: 10.1038/mt.2013.16, doi: 10.1038/mt.2010.298, doi: 10.4049/jimmunol.1500956), as well as combinations of different DARPins (doi: 10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVL76.7.3558-3563.2002). In some embodiments, a targeting protein can also include an antibody or an antigen-binding fragment thereof (e.g., Fab, Fab′, F(ab′) 2. Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD4 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVL75.17.8016-8020.2001, doi: 10.1038/nm1192). In some embodiments, altered and non-altered fusogens may be displayed on the same retroviral vector or VLP (doi: 10.1016/j.biomaterials.2014.01.051).

In some embodiments, a CD4 binding agent comprises a humanized antibody molecule, intact IgA, IgG, IgE or IgM antibody; bi- or multi-specific antibody (e.g., Zybodies®, etc); antibody fragments such as Fab fragments, Fab′ fragments, F(ab′); fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); camelid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BITE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the CD4 binding agent is a peptide. In some embodiments, the CD4 binding agent is an antibody, such as a single-chain variable fragment (scFv). In some embodiments, the CD4 binding agent is an antibody, such as a single domain antibody. In some embodiments, the antibody can be human or humanized. In some embodiments, the CD4 binding agent is a VHH. In some embodiments, the antibody or portion thereof is naturally occurring. In some embodiments, the antibody or portion thereof is synthetic.

In some embodiments, the CD4 binding agent comprises a CDR-H1, a CDR-H2, and a CDR-H3 comprising the amino acid sequence set forth in SEQ ID NO: 17, 18, and 19, respectively. In some embodiments, the CD4 binding agent comprises a CDR-L1, a CDR-L2, and a CDR-L3 comprising the amino acid sequence set forth in SEQ ID NO: 20, 21, and 22, respectively. In some embodiments, the CD4 binding agent comprises a CDR-H1, a CDR-H2, and a CDR-H3 comprising the amino acid sequence set forth in SEQ ID NO: 17, 18, and 19, respectively; and a CDR-L1, a CDR-L2, and a CDR-L3 comprising the amino acid sequence set forth in SEQ ID NO: 20, 21, and 22, respectively. In some embodiments, the CD4 binding agent comprises a CDR-H1, a CDR-H2, and a CDR-H3 comprising the amino acid sequence set forth in SEQ ID NO: 75, 76, and 77, respectively. In some embodiments, the CD4 binding agent comprises a CDR-L1, a CDR-L2, and a CDR-L3 comprising the amino acid sequence set forth in SEQ ID NO: 78, 79, and 22, respectively. In some embodiments, the CD4 binding agent comprises a CDR-H1, a CDR-H2, and a CDR-H3 comprising the amino acid sequence set forth in SEQ ID NO: 75, 76, and 77, respectively; and a CDR-L1, a CDR-L2, and a CDR-L3 comprising the amino acid sequence set forth in SEQ ID NO: 78, 79, and 22, respectively. In some embodiments, the CD4 binding agent comprises a CDR-H1, a CDR-H2, and a CDR-H3 comprising the amino acid sequence set forth in SEQ ID NO: 80, 81, and 77, respectively. In some embodiments, the CD4 binding agent comprises a CDR-L1, a CDR-L2, and a CDR-L3 comprising the amino acid sequence set forth in SEQ ID NO: 78, 79, and 22, respectively. In some embodiments, the CD4 binding agent comprises a CDR-H1, a CDR-H2, and a CDR-H3 comprising the amino acid sequence set forth in SEQ ID NO: 80, 81, and 77, respectively; and a CDR-L1, a CDR-L2, and a CDR-L3 comprising the amino acid sequence set forth in SEQ ID NO: 78, 79, and 22, respectively. In some embodiments, the CD4 binding agent comprises a heavy chain variable region (VH) comprising the amino acid sequence set forth in SEQ ID NO:23. In some embodiments, the CD4 binding agent comprises a light chain variable region (VL) comprising the amino acid sequence set forth in SEQ ID NO:24. In some embodiments, the CD4 binding agent comprises a heavy chain variable region (VH) comprising the amino acid sequence set forth in SEQ ID NO:23; and a light chain variable region (VL) comprising the amino acid sequence set forth in SEQ ID NO:24. In some embodiments, the VH and VL are joined by a linker. In some embodiments, the linker comprises the amino acid sequence set forth in SEQ ID NO:27. In some embodiments, the CD4 binding agent comprises the amino acid sequence set forth in SEQ ID NO:25.

In some embodiments, the CD4 binding agent comprises a CDR-H1, a CDR-H2, and a CDR-H3 comprising the amino acid sequence set forth in SEQ ID NO: 26, 27, and 28, respectively. In some embodiments, the CD4 binding agent comprises a CDR-L1, a CDR-L2, and a CDR-L3 comprising the amino acid sequence set forth in SEQ ID NO: 29, 30, and 31, respectively. In some embodiments, the CD4 binding agent comprises a CDR-H1, a CDR-H2, and a CDR-H3 comprising the amino acid sequence set forth in SEQ ID NO: 26, 27, and 28, respectively; and a CDR-L1, a CDR-L2, and a CDR-L3 comprising the amino acid sequence set forth in SEQ ID NO: 29, 30, and 31, respectively. In some embodiments, the CD4 binding agent comprises a CDR-H1, a CDR-H2, and a CDR-H3 comprising the amino acid sequence set forth in SEQ ID NO: 82, 83, and 84, respectively. In some embodiments, the CD4 binding agent comprises a CDR-L1, a CDR-L2, and a CDR-L3 comprising the amino acid sequence set forth in SEQ ID NO: 85, 86, and 31, respectively. In some embodiments, the CD4 binding agent comprises a CDR-H1, a CDR-H2, and a CDR-H3 comprising the amino acid sequence set forth in SEQ ID NO: 82, 83, and 84, respectively; and a CDR-L1, a CDR-L2, and a CDR-L3 comprising the amino acid sequence set forth in SEQ ID NO: 85, 86, and 31, respectively. In some embodiments, the CD4 binding agent comprises a CDR-H1, a CDR-H2, and a CDR-H3 comprising the amino acid sequence set forth in SEQ ID NO: 87, 88, and 84, respectively. In some embodiments, the CD4 binding agent comprises a CDR-L1, a CDR-L2, and a CDR-L3 comprising the amino acid sequence set forth in SEQ ID NO: 85, 86, and 31, respectively. In some embodiments, the CD4 binding agent comprises a CDR-H1, a CDR-H2, and a CDR-H3 comprising the amino acid sequence set forth in SEQ ID NO: 87, 88, and 84, respectively; and a CDR-L1, a CDR-L2, and a CDR-L3 comprising the amino acid sequence set forth in SEQ ID NO: 85, 86, and 31, respectively. In some embodiments, the CD4 binding agent comprises a heavy chain variable region (VH) comprising the amino acid sequence set forth in SEQ ID NO:32. In some embodiments, the CD4 binding agent comprises a light chain variable region (VL) comprising the amino acid sequence set forth in SEQ ID NO:33. In some embodiments, the CD4 binding agent comprises a heavy chain variable region (VH) comprising the amino acid sequence set forth in SEQ ID NO:32; and a light chain variable region (VL) comprising the amino acid sequence set forth in SEQ ID NO:33. In some embodiments, the VH and VL are joined by a linker. In some embodiments, the linker comprises the amino acid sequence set forth in SEQ ID NO:27. In some embodiments, the CD4 binding agent comprises the amino acid sequence set forth in SEQ ID NO:34.

In some embodiments, the CD4 binding agent comprises a CDR-H1, a CDR-H2, and a CDR-H3 comprising the amino acid sequence set forth in SEQ ID NO: 35, 36, and 37, respectively. In some embodiments, the CD4 binding agent comprises a CDR-L1, a CDR-L2, and a CDR-L3 comprising the amino acid sequence set forth in SEQ ID NO: 38, 39, and 40, respectively. In some embodiments, the CD4 binding agent comprises a CDR-H1, a CDR-H2, and a CDR-H3 comprising the amino acid sequence set forth in SEQ ID NO: 35, 36, and 37, respectively; and a CDR-L1, a CDR-L2, and a CDR-L3 comprising the amino acid sequence set forth in SEQ ID NO: 38, 39, and 40, respectively. In some embodiments, the CD4 binding agent comprises a CDR-H1, a CDR-H2, and a CDR-H3 comprising the amino acid sequence set forth in SEQ ID NO: 89, 90, 91, respectively. In some embodiments, the CD4 binding agent comprises a CDR-L1, a CDR-L2, and a CDR-L3 comprising the amino acid sequence set forth in SEQ ID NO: 92, 93, and 40, respectively. In some embodiments, the CD4 binding agent comprises a CDR-H1, a CDR-H2, and a CDR-H3 comprising the amino acid sequence set forth in SEQ ID NO: 89, 90, 91, respectively; and a CDR-L1, a CDR-L2, and a CDR-L3 comprising the amino acid sequence set forth in SEQ ID NO: 92, 93, and 40, respectively. In some embodiments, the CD4 binding agent comprises a CDR-H1, a CDR-H2, and a CDR-H3 comprising the amino acid sequence set forth in SEQ ID NO: 94, 95, 91, respectively. In some embodiments, the CD4 binding agent comprises a CDR-L1, a CDR-L2, and a CDR-L3 comprising the amino acid sequence set forth in SEQ ID NO: 92, 93, and 40, respectively. In some embodiments, the CD4 binding agent comprises a CDR-H1, a CDR-H2, and a CDR-H3 comprising the amino acid sequence set forth in SEQ ID NO: 94, 95, 91, respectively; and a CDR-L1, a CDR-L2, and a CDR-L3 comprising the amino acid sequence set forth in SEQ ID NO: 92, 93, and 40, respectively. In some embodiments, the CD4 binding agent comprises a heavy chain variable region (VH) comprising the amino acid sequence set forth in SEQ ID NO:41. In some embodiments, the CD4 binding agent comprises a light chain variable region (VL) comprising the amino acid sequence set forth in SEQ ID NO:42. In some embodiments, the CD4 binding agent comprises a heavy chain variable region (VH) comprising the amino acid sequence set forth in SEQ ID NO:41; and a light chain variable region (VL) comprising the amino acid sequence set forth in SEQ ID NO:42. In some embodiments, the VH and VL are joined by a linker. In some embodiments, the linker comprises the amino acid sequence set forth in SEQ ID NO:27. In some embodiments, the CD4 binding agent comprises the amino acid sequence set forth in SEQ ID NO:43.

In some embodiments, the CD4 binding agent comprises a CDR-H1, a CDR-H2, and a CDR-H3 comprising the amino acid sequence set forth in SEQ ID NO: 44, 45, and 46, respectively. In some embodiments, the CD4 binding agent comprises a CDR-L1, a CDR-L2, and a CDR-L3 comprising the amino acid sequence set forth in SEQ ID NO: 47, 48, and 49, respectively. In some embodiments, the CD4 binding agent comprises a CDR-H1, a CDR-H2, and a CDR-H3 comprising the amino acid sequence set forth in SEQ ID NO: 44, 45, and 46, respectively; and a CDR-L1, a CDR-L2, and a CDR-L3 comprising the amino acid sequence set forth in SEQ ID NO: 47, 48, and 49, respectively. In some embodiments, the CD4 binding agent comprises a CDR-H1, a CDR-H2, and a CDR-H3 comprising the amino acid sequence set forth in SEQ ID NO: 96, 97, 98, respectively. In some embodiments, the CD4 binding agent comprises a CDR-L1, a CDR-L2, and a CDR-L3 comprising the amino acid sequence set forth in SEQ ID NO: 99, 100, and 49, respectively. In some embodiments, the CD4 binding agent comprises a CDR-H1, a CDR-H2, and a CDR-H3 comprising the amino acid sequence set forth in SEQ ID NO: 96, 97, 98, respectively; and a CDR-L1, a CDR-L2, and a CDR-L3 comprising the amino acid sequence set forth in SEQ ID NO: 99, 100, and 49, respectively. In some embodiments, the CD4 binding agent comprises a CDR-H1, a CDR-H2, and a CDR-H3 comprising the amino acid sequence set forth in SEQ ID NO: 101, 102, 98 respectively. In some embodiments, the CD4 binding agent comprises a CDR-L1, a CDR-L2, and a CDR-L3 comprising the amino acid sequence set forth in SEQ ID NO: 99, 100, and 49, respectively. In some embodiments, the CD4 binding agent comprises a CDR-H1, a CDR-H2, and a CDR-H3 comprising the amino acid sequence set forth in SEQ ID NO: 101, 102, 98, respectively; and a CDR-L1, a CDR-L2, and a CDR-L3 comprising the amino acid sequence set forth in SEQ ID NO: 99, 100, and 49, respectively. In some embodiments, the CD4 binding agent comprises a heavy chain variable region (VH) comprising the amino acid sequence set forth in SEQ ID NO:50. In some embodiments, the CD4 binding agent comprises a light chain variable region (VL) comprising the amino acid sequence set forth in SEQ ID NO:51. In some embodiments, the CD4 binding agent comprises a heavy chain variable region (VH) comprising the amino acid sequence set forth in SEQ ID NO:50; and a light chain variable region (VL) comprising the amino acid sequence set forth in SEQ ID NO:51. In some embodiments, the VH and VL are joined by a linker. In some embodiments, the linker comprises the amino acid sequence set forth in SEQ ID NO:27. In some embodiments, the CD4 binding agent comprises the amino acid sequence set forth in SEQ ID NO:52.

In some embodiments, the CD4 binding agent comprises a CDR-H1, a CDR-H2, and a CDR-H3 comprising the amino acid sequence set forth in SEQ ID NO: 53, 54, and 55, respectively. In some embodiments, the CD4 binding agent comprises a CDR-L1, a CDR-L2, and a CDR-L3 comprising the amino acid sequence set forth in SEQ ID NO: 56, 39, and 57, respectively. In some embodiments, the CD4 binding agent comprises a CDR-H1, a CDR-H2, and a CDR-H3 comprising the amino acid sequence set forth in SEQ ID NO: 53, 54, and 55, respectively; and a CDR-L1, a CDR-L2, and a CDR-L3 comprising the amino acid sequence set forth in SEQ ID NO: 56, 39, and 57, respectively. In some embodiments, the CD4 binding agent comprises a CDR-H1, a CDR-H2, and a CDR-H3 comprising the amino acid sequence set forth in SEQ ID NO: 103, 104, and 105, respectively. In some embodiments, the CD4 binding agent comprises a CDR-L1, a CDR-L2, and a CDR-L3 comprising the amino acid sequence set forth in SEQ ID NO: 106, 107, and 57, respectively. In some embodiments, the CD4 binding agent comprises a CDR-H1, a CDR-H2, and a CDR-H3 comprising the amino acid sequence set forth in SEQ ID NO: 103, 104, and 105, respectively; and a CDR-L1, a CDR-L2, and a CDR-L3 comprising the amino acid sequence set forth in SEQ ID NO: 106, 107, and 57, respectively. In some embodiments, the CD4 binding agent comprises a CDR-H1, a CDR-H2, and a CDR-H3 comprising the amino acid sequence set forth in SEQ ID NO: 108, 109, and 105, respectively. In some embodiments, the CD4 binding agent comprises a CDR-L1, a CDR-L2, and a CDR-L3 comprising the amino acid sequence set forth in SEQ ID NO: 106, 107, and 57, respectively. In some embodiments, the CD4 binding agent comprises a CDR-H1, a CDR-H2, and a CDR-H3 comprising the amino acid sequence set forth in SEQ ID NO: 108, 109, and 105, respectively; and a CDR-L1, a CDR-L2, and a CDR-L3 comprising the amino acid sequence set forth in SEQ ID NO: 106, 107, and 57, respectively. In some embodiments, the CD4 binding agent comprises a heavy chain variable region (VH) comprising the amino acid sequence set forth in SEQ ID NO: 58. In some embodiments, the CD4 binding agent comprises a light chain variable region (VL) comprising the amino acid sequence set forth in SEQ ID NO:59. In some embodiments, the CD4 binding agent comprises a heavy chain variable region (VH) comprising the amino acid sequence set forth in SEQ ID NO:58; and a light chain variable region (VL) comprising the amino acid sequence set forth in SEQ ID NO:59. In some embodiments, the VH and VL are joined by a linker. In some embodiments, the linker comprises the amino acid sequence set forth in SEQ ID NO:27. In some embodiments, the CD4 binding agent comprises the amino acid sequence set forth in SEQ ID NO:60.

In some embodiments, the CD4 binding agent comprises a CDR-H1, a CDR-H2, and a CDR-H3 comprising the amino acid sequence set forth in SEQ ID NO: 61, 62, and 63, respectively. In some embodiments, the CD4 binding agent comprises a CDR-L1, a CDR-L2, and a CDR-L3 comprising the amino acid sequence set forth in SEQ ID NO: 64, 65, and 66, respectively. In some embodiments, the CD4 binding agent comprises a CDR-H1, a CDR-H2, and a CDR-H3 comprising the amino acid sequence set forth in SEQ ID NO: 61, 62, and 63, respectively; and a CDR-L1, a CDR-L2, and a CDR-L3 comprising the amino acid sequence set forth in SEQ ID NO: 64, 65, and 66, respectively. In some embodiments, the CD4 binding agent comprises a CDR-H1, a CDR-H2, and a CDR-H3 comprising the amino acid sequence set forth in SEQ ID NO: 110, 111, and 112, respectively. In some embodiments, the CD4 binding agent comprises a CDR-L1, a CDR-L2, and a CDR-L3 comprising the amino acid sequence set forth in SEQ ID NO: 113, 114, and 66, respectively. In some embodiments, the CD4 binding agent comprises a CDR-H1, a CDR-H2, and a CDR-H3 comprising the amino acid sequence set forth in SEQ ID NO: 110, 111, and 112, respectively; and a CDR-L1, a CDR-L2, and a CDR-L3 comprising the amino acid sequence set forth in SEQ ID NO: 113, 114, and 66, respectively. In some embodiments, the CD4 binding agent comprises a CDR-H1, a CDR-H2, and a CDR-H3 comprising the amino acid sequence set forth in SEQ ID NO: 115, 116, and 112, respectively. In some embodiments, the CD4 binding agent comprises a CDR-L1, a CDR-L2, and a CDR-L3 comprising the amino acid sequence set forth in SEQ ID NO: 113, 114, and 66, respectively. In some embodiments, the CD4 binding agent comprises a CDR-H1, a CDR-H2, and a CDR-H3 comprising the amino acid sequence set forth in SEQ ID NO: 115, 116, and 112, respectively; and a CDR-L1, a CDR-L2, and a CDR-L3 comprising the amino acid sequence set forth in SEQ ID NO: 113, 114, and 66, respectively. In some embodiments, the CD4 binding agent comprises a heavy chain variable region (VH) comprising the amino acid sequence set forth in SEQ ID NO: 67. In some embodiments, the CD4 binding agent comprises a light chain variable region (VL) comprising the amino acid sequence set forth in SEQ ID NO:68. In some embodiments, the CD4 binding agent comprises a heavy chain variable region (VH) comprising the amino acid sequence set forth in SEQ ID NO:67; and a light chain variable region (VL) comprising the amino acid sequence set forth in SEQ ID NO:68. In some embodiments, the VH and VL are joined by a linker. In some embodiments, the linker comprises the amino acid sequence set forth in SEQ ID NO:27. In some embodiments, the CD4 binding agent comprises the amino acid sequence set forth in SEQ ID NO:69.

In some embodiments, the CD4 binding agent is an antibody, such as a single domain antibody. In some embodiments, the antibody can be human or humanized. In some embodiments, the CD4 binding agent is a VHH. In some embodiments, the CD4 binding agent comprises a CDR-H1, a CDR-H2, and a CDR-H3 comprising the amino acid sequence set forth in SEQ ID NO: 129, 130, and 131, respectively. In some embodiments, the CD4 binding agent comprises a CDR-H1, a CDR-H2, and a CDR-H3 comprising the amino acid sequence set forth in SEQ ID NO: 70, 71, and 72, respectively. In some embodiments, the CD4 binding agent comprises a CDR-H1, a CDR-H2, and a CDR-H3 comprising the amino acid sequence set forth in SEQ ID NO: 73, 74, and 72, respectively. In some embodiments, the CD4 binding agent comprises the amino acid sequence set forth in SEQ ID NO: 132.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD4 binding agent is attached to the C-terminus of the G protein (e.g., fusogen) or biologically active portion thereof. In some embodiments, the N-terminus of the CD4 binding agent is exposed on the exterior surface of the lipid bilayer.

In some embodiments, the CD4 binding agent is the only surface displayed non-viral sequence of the viral vector. In some embodiments, the CD4 binding agent is the only membrane bound non-viral sequence of the viral vector. In some embodiments, the viral vector does not contain a molecule that engages or stimulates T cells other than the CD4 binding agent.

In some embodiments, viral vectors may display CD4 binding agents that are not conjugated to protein fusogens in order to redirect the fusion activity towards a cell that is bound by the targeting moiety, or to affect homing.

In some embodiments, a protein fusogen derived from a virus or organism that do not infect humans does not have a natural fusion targets in patients, and thus has high specificity.

The viral vectors disclosed herein include one or more CD8 binding agents. For example, a CD8 binding agent may be fused to or incorporated in a protein fusogen or viral envelope protein. In another embodiment, a CD8 binding agent may be incorporated into the viral envelope via fusion with a transmembrane domain.

The viral vectors disclosed herein include one or more CD8 binding agents. For example, a CD8 binding agent may be fused to or incorporated in a protein fusogen or viral envelope protein. In another embodiment, a CD8 binding agent may be incorporated into the viral envelope via fusion with a transmembrane domain. In some of any of the provided embodiments, the CD8 binding agent is an anti-CD8 antibody or an antigen-binding fragment. In some of any of the provided embodiments, the anti-CD8 antibody or antigen-binding fragment is mouse, rabbit, human, or humanized. In some embodiments, the antigen-binding fragment is a single chain variable fragment (scFv). In some embodiments, the anti-CD8 antibody or antigen-binding fragment is a single domain antibody. In some embodiments, the anti-CD8 antibody or antigen-binding fragment is a camelid (e.g. llama, alpaca, camel) (e.g. VHH).

In some of any of the provided embodiments, the CD8 binding agent binds to a CD8 alpha chain and/or CD8 beta chain. In some of any of the provided embodiments, the CD8 binding agent binds to a CD8 alpha chain. In some of any of the provided embodiments, the CD8 binding agent binds to a CD8 beta chain. In some of any of the provided embodiments, the CD8 binding agent binds to a CD8 alpha chain and a CD8 beta chain. Exemplary CD8 binding agents are also disclosed in 17/715,253, which is hereby incorporated in its entirety.

Further exemplary CD8 binding agents include antibodies and fragments thereof (e.g., scFv, VHH) that bind to one or more of CD8 alpha and CD8 beta. Such antibodies may be derived from any species, and may be for example, mouse, rabbit, human, humanized, or camelid antibodies. Exemplary antibodies include those disclosed in WO2014025828, WO2014164553, WO2020069433, WO2015184203, US20160176969, WO2017134306, WO2017182585, WO2019032661, WO2020257412, WO2018170096, WO2020060924, U.S. Pat. No. 10,730,944, US20200172620, and the non-human antibodies OKT8; RPA-T8, 12.C7 (Novus); 17D8, 3B5, LT8, RIV11, SP16, YTC182.20, MEM-31, MEM-87, RAVB3, C8/144B (Thermo Fisher); 2ST8.5H7, Bu88, 3C39, Hit8a, SPM548, CA-8, SK1, RPA-T8 (GeneTex); UCHT4 (Absolute Antibody); BW135/80 (Miltenyi); G42-8 (BD Biosciences); C8/1779R, mAB 104 (Enzo Life Sciences); B-Z31 (Sapphire North America); 32-M4, 5F10, MCD8, UCH-T4, 5F2 (Santa Cruz); D8A8Y, RPA-T8 (Cell Signaling Technology). Further exemplary anti-CD8 binding agents and G proteins are described in U.S. provisional application No. 63/172,518, which is incorporated by reference herein. Other exemplary binding agents include designed ankyrin repeat proteins (DARPins) and binding agents based on fibronectin type III (Fn3) scaffolds.

In some embodiments, the CD8 binding agent is an scFv that contains a VH and VL set forth from any as below, in which the VH and VL are separated by linker. In some embodiments, the CD8 binding agent is a VHH having the sequence set forth below. In some embodiments, the CD8 binding agent is linked to the C-terminus of a truncated NiV-G set forth in SEQ ID NO: 19 to provide a re-targeted NiV-G. In some embodiments, the retargeted NiV-G is pseudotyped on a lipid particle (e.g., a lentiviral vector) with the a NiV-F (e.g. set forth in SEQ ID NO:12). In some embodiments, the lipid particle (e.g., a lentiviral vector) further contains a payload gene encoding an anti-CD19 CAR. In some embodiments, the anti-CD19 CAR contains an anti-CD19 FMC63 scFv binding domain set forth in SEQ ID NO:40, a CD8 hinge set forth in SEQ ID NO:27, a CD8 transmembrane domain set forth in SEQ ID NO: 33, a 4-1bb signaling domain set forth in SEQ ID NO:36, a CD3zeta signaling domain set forth in SEQ ID NO: 38.

CD8_1
VH (SEQ ID NO.: 117):
QVQLVQSGAEVKKPGASVKVSCKASGGTFSSYAISWVRQAPGQGL
EWMGIIDPSDGNTNYAQNFQGRVTMTRDTSTSTVYMELSSLRSED
TAVYYCAKERAAAGYYYYMDVWGQGTTVTVSS
VL (SEQ ID NO.: 118:
DIQMTQSPSSLSASVGDRVTITCRASQSISSYLNWYQQKPGKAPK
LLIYAASSLQSGVPSRFSGSGSGTDFTLTISSLQPEDFATYYCQQ
SYSTPLTFGGGTKVEIKR
CD8_2
VH (SEQ ID NO.: 119):
QVQLVQSGAEVKKPGASVKVSCKASGYTFTDYYIQWVRQAPGQGL
EWMGWINPNSGGTSYAQKFQGRVTMTRDTSTSTVYMELSSLRSED
TAVYYCAKEGDYYYGMDAWGQGTMVTVSS
VL (SEQ ID NO.: 120):
DIVMTQSPLSLPVTPGEPASISCRSSQSLLHSNGYNYLDWYLQKP
GQSPQLLIYLGSNRASGVPDRFSGSGSGTDFTLKISRVEAEDVGV
YYCMQGLQTPHTFGQGTKVEIKR
CD8_3
VH (SEQ ID NO.: 121):
QVQLVQSGAEVKKPGASVKVSCKASGYTFTSYYMHWVRQAPGQGL
EWMGGFDPEDGETIYAQKFQGRVTMTRDTSTSTVYMELSSLRSED
TAVYYCARDQGWGMDVWGQGTTVTVSS
VL(SEQ ID NO.: 122):
DIQMTQSPSSLSASVGDRVTITCRASQSISSYLNWYQQKPGKAPK
LLIYAASSLQSGVPSRFSGSGSGTDFTLTISSLQPEDFATYYCQQ
TYSTPYTFGQGTKLEIKR
CD8_4
VH (SEQ ID NO.: 123):
QVQLVQSGAEVKKPGASVKVSCKASGYTFTNHYMHWVRQAPGQGL
EWMGWMNPNSGNTGYAQKFQGRVTMTRDTSTSTVYMELSSLRSED
TAVYYCASSESGSDLDYWGQGTLVTVSS
VL (SEQ ID NO.: 124):
DIQMTQSPSSLSASVGDRVTITCRASQTIGNYVNWYQQKPGKAPK
LLIYGASNLHTGVPSRFSGSGSGTDFTLTISSLQPEDFATYYCQQ
TYSAPLTFGGGTKVEIKR
In some embodiments, the CD8 binding agent
is VHH set forth as:
VHH (SEQ ID NO.: 125):
QVQLVESGGGLVQAGGSLRLSCAASGRTFSGYVMGWFRQAPGKQR
KFVAAISRGGLSTSYADSVKGRFTISRDNAKNTVFLQMNTLKPED
TAVYYCAADRSDLYEITAASNIDSWGQGTLVTVSS

In some embodiments, the G/H protein or functionally active variant or biologically active portion thereof is linked indirectly via a linker to the binding domain and/or variable domain thereof. In some embodiments, the linker is a peptide linker. In some embodiments, the linker is a chemical linker.

In some embodiments, the linker is a peptide linker and the targeted envelope protein is a fusion protein containing the G/H protein or functionally active variant or biologically active portion thereof linked via a peptide linker to the sdAb variable domain. In some embodiments, the targeted envelope protein is a fusion protein that has the following structure: (N′-single domain antibody-C′)-Linker-(C′-G protein-N′).

In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some embodiments, the peptide linker is a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

In particular embodiments, the linker is a flexible peptide linker. In some such embodiments, the linker is 1-20 amino acids, such as 1-20 amino acids predominantly composed of glycine. In some embodiments, the linker is 1-20 amino acids, such as 1-20 amino acids predominantly composed of glycine and serine. In some embodiments, the linker is a flexible peptide linker containing amino acids Glycine and Serine, referred to as GS-linkers. In some embodiments, the peptide linker includes the sequences GS, GGS, GGGGS (SEQ ID NO: 126), GGGGGS (SEQ ID NO: 127) or combinations thereof. In some embodiments, the polypeptide linker has the sequence (GGS) n, wherein n is 1 to 10. In some embodiments, the polypeptide linker has the sequence (GGGGS) n. (SEQ ID NO: 128) wherein n is 1 to 10. In some embodiments, the polypeptide linker has the sequence (GGGGGS) n (SEQ ID NO: 129), wherein n is 1 to 6.

### D. Polynucleotides Encoding the Variant Paramyxovirus Glycoproteins

Provided herein are polynucleotides comprising a nucleic acid sequence encoding a variant Paramyxovirus G/H protein described herein. The polynucleotides may include a sequence of nucleotides encoding any of the variant proteins described above. In some embodiments, the polynucleotide can be a synthetic nucleic acid. Also provided are expression vectors containing any of the provided polynucleotides.

In some of any embodiments, expression of natural or synthetic nucleic acids is typically achieved by operably linking a nucleic acid encoding the gene of interest to a promoter and incorporating the construct into an expression vector. In some embodiments, vectors can be suitable for replication and integration in eukaryotes. In some embodiments, cloning vectors contain transcription and translation terminators, initiation sequences, and promoters useful for expression of the desired nucleic acid sequence. In some of any embodiments, a plasmid comprises a promoter suitable for expression in a cell.

In some embodiments, the polynucleotides contain at least one promoter that is operatively linked to control expression of the variant Paramyxovirus G/H protein. For expression of the variant Paramyxovirus protein, at least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

In some embodiments, additional promoter elements, e.g., enhancers, regulate the frequency of transcriptional initiation. In some embodiments, additional promoter elements are located in the region 30-110 bp upstream of the start site, although a number of promoters have recently been shown to contain functional elements downstream of the start site as well. In some embodiments, spacing between promoter elements frequently is flexible, so that promoter function is preserved when elements are inverted or moved relative to one another. In some embodiments, the thymidine kinase (tk) promoter, the spacing between promoter elements can be increased to 50 bp apart before activity begins to decline. In some embodiments, depending on the promoter, individual elements can function either cooperatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring.” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202 and 5,928,906).

In some embodiments, a suitable promoter is the immediate early cytomegalovirus (CMV) promoter sequence. In some embodiments, the promoter sequence is a strong constitutive promoter sequence capable of driving high levels of expression of any polynucleotide sequence operatively linked thereto. In some embodiments, a suitable promoter is Elongation Growth Factor-la (EF-1 a). In some embodiments, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

In some embodiments, the promoter is an inducible promoter. In some embodiments, the inducible promoter provides a molecular switch capable of turning on expression of the polynucleotide sequence which it is operatively linked when such expression is desired, or turning off the expression when expression is not desired. In some embodiments, inducible promoters comprise metallothionine promoter, a glucocorticoid promoter, a progesterone promoter, and a tetracycline promoter.

In some embodiments, exogenously controlled inducible promoters can be used to regulate expression of the variant Paramyxovirus protein. For example, radiation-inducible promoters, heat-inducible promoters, and/or drug-inducible promoters can be used to selectively drive transgene expression in, for example, targeted regions. In such embodiments, the location, duration, and level of transgene expression can be regulated by the administration of the exogenous source of induction.

In some embodiments, expression of the variant Paramyxovirus G/H protein is regulated using a drug-inducible promoter. For example, in some cases, the promoter, enhancer, or transactivator comprises a Lac operator sequence, a tetracycline operator sequence, a galactose operator sequence, a doxycycline operator sequence, a rapamycin operator sequence, a tamoxifen operator sequence, or a hormone-responsive operator sequence, or an analog thereof. In some instances, the inducible promoter comprises a tetracycline response element (TRE). In some embodiments, the inducible promoter comprises an estrogen response element (ERE), which can activate gene expression in the presence of tamoxifen. In some instances, a drug-inducible element, such as a TRE, can be combined with a selected promoter to enhance transcription in the presence of drug, such as doxycycline. In some embodiments, the drug-inducible promoter is a small molecule-inducible promoter.

Any of the provided polynucleotides can be modified to remove CpG motifs and/or to optimize codons for translation in a particular species, such as human, canine, feline, equine, ovine, bovine, etc. species. In some embodiments, the polynucleotides are optimized for human codon usage (i.e., human codon-optimized). In some embodiments, the polynucleotides are modified to remove CpG motifs. In other embodiments, the provided polynucleotides are modified to remove CpG motifs and are codon-optimized, such as human codon-optimized. Methods of codon optimization and CpG motif detection and modification are well-known. Typically, polynucleotide optimization enhances transgene expression, increases transgene stability and preserves the amino acid sequence of the encoded polypeptide.

In order to assess the expression of the variant Paramyxovirus G/H proteins, the expression vector to be introduced into a cell can also contain either a selectable marker gene or a reporter gene or both to facilitate identification and selection of expressing particles, e.g. viral particles. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000, FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of the desired polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

III. Lipid Particles Comprising Paramyxovirus G/H and/or F Glycoproteins Having Reduced Glycosylation

Provided herein are lipid particles comprising a lipid bilayer, a lumen surrounded by the lipid bilayer and Paramyxovirus F and G/H glycoproteins in which the glycoproteins are embedded within the lipid bilayer. In some embodiments, the Paramyxovirus G/H glycoprotein is a variant protein, such as any of the variant proteins described herein. In some embodiments, the lipid particle is produced and/or modified to have reduced glycosylation. In some embodiments, the lipid particle may additionally comprise an exogenous agent (e.g. therapeutic agent) for delivery to a cell. In some embodiments, a lipid particle is introduced to a cell in the subject. Also provided are methods of delivering any of the provided lipid particles to a cell.

In some embodiments, the provided lipid particles exhibit fusogenic activity, which is mediated by the Paramyxovirus G/H and F proteins that facilitates merger or fusion of the two lumens of the lipid particle and the target cell membranes. Thus, among provided lipid particles are fusosomes. In some embodiments, the fusosome comprises a naturally derived bilayer of amphipathic lipids with the Paramyxovirus G/H or F glycoprotein as a fusogen. In some embodiments, the fusosome comprises (a) a lipid bilayer, (b) a lumen (e.g., comprising cytosol) surrounded by the lipid bilayer; and (c) a fusogen that is exogenous or overexpressed relative to the source cell. In some embodiments, the Paramyxovirus G/H and F proteins are disposed in the lipid bilayer. In some embodiments, the Paramyxovirus G/H protein is a variant G/H protein. In some embodiments, the fusosome comprises several different types of lipids, e.g., amphipathic lipids, such as phospholipids.

In some embodiments, the lipid particle includes a naturally derived bilayer of amphipathic lipids that encloses lumen or cavity. In some embodiments, the lipid particle comprises a lipid bilayer as the outermost surface. In some embodiments, the lipid bilayer encloses a lumen. In some embodiments, the lumen is aqueous. In some embodiments, the lumen is in contact with the hydrophilic head groups on the interior of the lipid bilayer. In some embodiments, the lumen is a cytosol. In some embodiments, the cytosol contains cellular components present in a source cell. In some embodiments, the cytosol does not contain components present in a source cell. In some embodiments, the lumen is a cavity. In some embodiments, the cavity contains an aqueous environment. In some embodiments, the cavity does not contain an aqueous environment.

In some aspects, the lipid bilayer is derived from a source cell during a process to produce a lipid-containing particle. Exemplary methods for producing lipid-containing particles are provided in Section III.D. In some embodiments, the lipid bilayer includes membrane components of the cell from which the lipid bilayer is produced, e.g., phospholipids, membrane proteins, etc. In some embodiments, the lipid bilayer includes a cytosol that includes components found in the cell from which the micro-vesicle is produced, e.g., solutes, proteins, nucleic acids, etc., but not all of the components of a cell, e.g., they lack a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In some embodiments, the lipid bilayer is a viral envelope. In some embodiments, the viral envelope is obtained from a source cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins as described herein, such as a variant Paramyxovirus G/H glycoprotein.

In other aspects, the lipid bilayer includes synthetic lipid complex. In some embodiments, the synthetic lipid complex is a liposome. In some embodiments, the lipid bilayer is a vesicular structure characterized by a phospholipid bilayer membrane and an inner aqueous medium. In some embodiments, the lipid bilayer has multiple lipid layers separated by aqueous medium. In some embodiments, the lipid bilayer forms spontaneously when phospholipids are suspended in an excess of aqueous solution. In some examples, the lipid components undergo self-rearrangement before the formation of closed structures and entrap water and dissolved solutes between the lipid bilayers.

In some embodiments, a variant Paramyxovirus G/H glycoprotein and a F protein (e.g. a Paramyxovirus F glycoprotein), such as any described including any that are exogenous or overexpressed relative to the source cell, is disposed in the lipid bilayer. In provided embodiments, the Paramyxovirus F protein and variant G/H glycoprotein (e.g. any one of the variant Paramyxovirus G/H glycoprotein proteins described herein) are exposed on the outside surface of the lipid bilayer of the lipid particle (e.g. lentiviral vector).

In some embodiments, the lipid particle comprises several different types of lipids. In some embodiments, the lipids are amphipathic lipids. In some embodiments, the amphipathic lipids are phospholipids. In some embodiments, the phospholipids comprise phosphatidylcholine, phosphatidylethanolamine, phosphatidylinositol, and phosphatidylserine. In some embodiments, the lipids comprise phospholipids such as phosphocholines and phosphoinositols. In some embodiments, the lipids comprise DMPC, DOPC, and DSPC.

In some embodiments, the bilayer may be comprised of one or more lipids of the same or different type. In some embodiments, the source cell comprises a cell selected from CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

In some embodiments, the lipid particle can be a viral particle, a virus-like particle, a nanoparticle, a vesicle, an exosome, a dendrimer, a lentivirus, a viral vector, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a lentiviral vector, a viral based particle, a virus like particle (VLP) or a cell based particle.

In particular embodiments, the lipid particle is virally derived. In some embodiments, the lipid particle can be a viral-based particle, such as a viral vector particle (e.g. lentiviral vector particle) or a virus-like particle (e.g. a lentiviral-like particle). In some embodiments, the lipid bilayer is a viral envelope. In some embodiments, the viral envelope is obtained from a host cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins.

In particular embodiments, the lipid particle is not virally derived. In some embodiments, the lipid particle can be a nanoparticle, a vesicle, an exosome, a dendrimer, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a cell derived particle.

In some embodiments, the lipid bilayer includes membrane components of the host cell from which the lipid bilayer is derived, e.g., phospholipids, membrane proteins, etc. In some embodiments, the lipid bilayer includes a cytosol that includes components found in the cell from which the vehicle is derived, e.g., solutes, proteins, nucleic acids, etc., but not all of the components of a cell, e.g., lacking a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In particular embodiments, an exogenous agent, such as a polynucleotide or polypeptide, is encapsulated within the lumen of a lipid particle. Embodiments of provided lipid particles may have various properties that facilitate delivery of a payload, such as, e.g., a desired transgene or exogenous agent, to a target cell. The exogenous agent may be a polynucleotide or a polypeptide. In some embodiments, a lipid particle provided herein is administered to a subject, e.g., a mammal, e.g., a human. In such embodiments, the subject may be at risk of, may have a symptom of, or may be diagnosed with or identified as having, a particular disease or condition. In one embodiment, the subject has cancer. In one embodiment, the subject has an infectious disease. In some embodiments, the lipid particle contains nucleic acid sequences (polynucleotide) encoding an exogenous agent or a polypeptide exogenous agent for treating the disease or condition.

The lipid particles can include spherical particles or can include particles of elongated or irregular shape.

In some embodiments, a composition of particles can be assessed for one or more features related to their size, including diameter, range of variation thereof above and below an average (mean) or median value of the diameter, coefficient of variation, polydispersity index or other measure of size of particles in a composition. Various methods for particle characterization can be used, including, but not limited to, laser diffraction, dynamic light scattering (DLS; also known as photon correlation spectroscopy) or image analysis, such as microscopy or automated image analysis.

In some embodiments, the provided lipid particle has a diameter of, or the average (mean) diameter of particles in a composition is, less than about 3 μm, less than about 2 μm, less than about 1 μm, less than about 900 nm, less than about 800 nm, less than about 700 nm, less than about 600 nm, less than about 500 m, less than about 400 nm, less than about 300, less than about 200 nm, less than about 150 nm, less than about 100 nm, less than about 50 nm, or less than about 20 nm. In some embodiments, the lipid particle has a diameter of, or the average (mean) diameter of particles in a composition is, less than about 400 nm. In another embodiment, the lipid particle has a diameter of, or the average (mean) diameter of particles in a composition is, less than about 150 nm. In some embodiments, the lipid particle has a diameter of, or the average (mean) diameter of particles in a composition is, between at or about 2 μm and at or about 1 μm, between at or about 1 μm and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments the median particle diameter in a composition of particles is between at or about 10 nm and at or about 1000 nM, between at or about 25 nm and at or about 500 nm, between at or about 40 nm and at or about 300 nm, between at or about 50 nm and at or about 250 nm, between at or about 60 nm and at or about 225 nm, between at or about 70 nm and at or about 200 nm, between at or about 80 nm and at or about 175 nm, or between at or about 90 nm and at or about 150 nm.

In some embodiments, 90% of the lipid particles in a composition fall within 50% of the median diameter of the lipid particles. In some embodiments, 90% of the lipid particles in a composition fall within 25% of the median diameter of the lipid particles. In some embodiments, 90% of the lipid particles in a composition fall within 20% of the median diameter. In some embodiments, 90% of the lipid particles in a composition fall within 15% of the median diameter of lipid particles. In some embodiments, 90% of the lipid particles in a composition fall within 10% of the median diameter of the lipid particles.

In some embodiments, 75% of the lipid particles in a composition fall within +/−2 or +/−1 St Dev standard deviations (St Dev) of the mean diameter of lipid particles. In some embodiments, 80% of the lipid particles in a composition fall within +/−2 St Dev or +/−1 St Dev of the mean diameter of lipid particles. In some embodiments, 85% of the lipid particles in a composition fall within +/−2 St Dev or +/−1 St Dev of the mean diameter of lipid particles. In some embodiments, 90% of the lipid particles in a composition fall within +/−2 St Dev or +/−1 St Dev of the mean diameter of lipid particles. In some embodiments, 95% of the lipid particles in a composition fall within +/−2 St Dev or +/−1 St Dev of the mean diameter of lipid particles.

In some embodiments, the lipid particles have an average hydrodynamic radius, e.g. as determined by dynamic light scattering (DLS), of about 100 nm to about two microns. In some embodiments, the lipid particles have an average hydrodynamic radius between at or about 2 μm and at or about 1 μm, between at or about 1 μm and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments, the lipid particles have an average geometric radius, e.g. as determined by a multi-angle light scattering, of about 100 nm to about two microns. In some embodiments, the lipid particles have an average geometric radius between at or about 2 μm and at or about 1 μm, between at or about 1 μm and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments, the coefficient of variation (COV) (i.e. standard deviation divided by the mean) of a composition of lipid particles is less than at or about 30%, less than at or about 25%, less than at or about 20%, less than at or about 15%, less than at or about 10% or less than at or about 5%.

In some embodiment, provided compositions of lipid particles are characterized by their polydispersity index, which is a measure of the size distribution of the particles wherein values between 1 (maximum dispersion) and 0 (identical size of all of the particles) are possible. In some embodiments, compositions of lipid particles provided herein have a polydispersity index of between at or about 0.05 and at or about 0.7, between at or about 0.05 and at or about 0.6, between at or about 0.05 and at or about 0.5, between at or about 0.05 and at or about 0.4, between at or about 0.05 and at or about 0.3, between at or about 0.05 and at or about 0.2, between at or about 0.05 and at or about 0.1, between at or about 0.1 and at or about 0.7, between at or about 0.1 and at or about 0.6, between at or about 0.1 and at or about 0.5, between at or about 0.1 and at or about 0.4, between at or about 0.1 and at or about 0.3, between at or about 0.1 and at or about 0.2, between at or about 0.2 and at or about 0.7, between at or about 0.2 and at or about 0.6, between at or about 0.2 and at or about 0.5, between at or about 0.2 and at or about 0.4 between at or about 0.2 and at or about 0.3, between at or about 0.3 and at or about 0.7, between at or about 0.3 and at or about 0.6, between at or about 0.3 and at or about 0.5, between at or about 0.3 and at or about 0.4, between at or about 0.4 and at or about 0.7, between at or about 0.4 and at or about 0.6, between at or about 0.4 and at or about 0.5, between at or about 0.5 and at or about 0.7, between at or about 0.5 and at or about 0.6, or between at or about 0.6 and at or about 0.7. In some embodiments, the polydispersity index is less than at or about 0.05, less than at or about 0.1, less than at or about 0.15, less than at or about 0.2, less than at or about 0.25, less than at or about 0.3, less than at or about 0.4, less than at or about 0.5, less than at or about 0.6 or less than at or about 0.7. Various lipid particles are known, any of which can be generated in accord with the provided embodiments. Non-limiting examples of lipid particles include any as described in, or contain features as described in. International published PCT Application No. WO 2017/095946; WO 2017/095944; WO 2017/095940; WO 2019/157319; WO 2018/208728; WO 2019/113512; WO 2019/161281; WO 2020/102578; WO 2019/222403; WO 2020/014209; WO 2020/102485; WO 2020/102499; WO 2020/102503; WO 2013/148327; WO 2017/182585; WO 2011/058052; or WO 2017/068077, each of which are incorporated by reference in their entirety.

### A. Viral-Based Particles

Provided herein are viral-based particles derived from a virus, including those derived from retroviruses or lentiviruses, containing a variant Paramyxovirus glycoprotein, such as any of the variant Paramyxovirus G/H proteins described in Section II. In some embodiments, the lipid particle's bilayer of amphipathic lipids is or comprises the viral envelope. In some embodiments, the lipid particle's bilayer of amphipathic lipids is or comprises lipids derived from a producer cell. In some embodiments, the viral envelope may comprise a fusogen, e.g., a fusogen that is endogenous to the virus or a pseudotyped fusogen. In some embodiments, the lipid particle's lumen or cavity comprises a viral nucleic acid, e.g., a retroviral nucleic acid, e.g., a lentiviral nucleic acid. In some embodiments, the viral nucleic acid may be a viral genome. In some embodiments, the lipid particle further comprises one or more viral non-structural proteins, e.g., in its cavity or lumen. In some embodiments, the viral-based particle is or comprises a virus-like particle (VLP). In some embodiments, the VLP does not comprise any viral genetic material. In some embodiments, the viral-based particle does not contain any virally derived nucleic acids or viral proteins, such as viral structural proteins.

Biological methods for introducing an exogenous agent to a host cell include the use of DNA and RNA vectors. DNA and RNA vectors can also be used to house and deliver polynucleotides and polypeptides. Viral vectors and virus like particles, and especially retroviral vectors, have become the most widely used method for inserting genes into mammalian, e.g., human cells. Other viral vectors and virus like particles can be derived from lentivirus, poxviruses, herpes simplex virus I, adenoviruses and adeno-associated viruses, and the like. See, for example, U.S. Pat. Nos. 5,350,674 and 5,585,362. Methods for producing cells comprising vectors and/or exogenous acids are well-known in the art. See, for example, Sambrook et al., 2001, Molecular Cloning: A Laboratory Manual, Cold Spring Harbor Laboratory, New York.

In some embodiments, the viral particles or virus-like particles bilayer of amphipathic lipids is or comprises lipids derived from an infected host cell. In some embodiments, the lipid bilayer is a viral envelope. In some embodiments, the viral particles or virus-like particles envelope is obtained from a host cell. In some embodiments, the viral particles or virus-like particles envelope is obtained by the viral capsid from the source cell plasma membrane. In some embodiments, the lipid bilayer is obtained from a membrane other than the plasma membrane of a host cell. In some embodiments, the viral particles or virus-like particles envelope lipid bilayer is embedded with viral proteins, including viral glycoproteins, including variant Paramyxovirus G/H glycoproteins.

In some embodiments, one or more transducing units of viral particles or virus-like particles, e.g. retroviral particles or retroviral-like particles, are administered to the subject. In some embodiments, at least 1, 10, 100, 1000, 10⁴, 10⁵, 10⁶, 10⁷, 10⁸, 10⁹, 10¹⁰, 10¹¹, 10¹², 10¹³, or 10¹⁴ transducing units per kg are administered to the subject. In some embodiments at least 1, 10, 100, 1000, 10⁴, 10⁵, 10⁶, 10⁷, 10⁸, 10⁹, 10¹⁰, 10¹¹, 10¹², 10¹³, or 10¹⁴ transducing units per target cell or per target cell per ml of blood are administered to the subject.

### 1. Viral Particles

In some embodiments, the lipid particle is or comprises a virus or a viral vector, e.g., a retrovirus or retroviral vector, e.g., a lentivirus or lentiviral vector. In some embodiments, the virus or viral vector is recombinant. For instance, the viral particle may be referred to as a recombinant virus and/or a recombinant viral vector, which are used interchangeably. In some embodiments, the lipid particle is a recombinant lentivirus vector particle.

In some embodiments, a lipid particle comprises a lipid bilayer comprising a retroviral vector comprising an envelope. For instance, in some embodiments, the bilayer of amphipathic lipids is or comprises the viral envelope. The viral envelope may comprise a fusogen, e.g., a variant Paramyxovirus G/H protein, such as those described in Section II and/or a Paramyxovirus F as described below in Section III.C. In some embodiments, the viral vector's lumen or cavity comprises a viral nucleic acid, e.g., a retroviral nucleic acid, e.g., a lentiviral nucleic acid. The viral nucleic acid may be a viral genome. In some embodiments, the viral vector may further comprises one or more viral non-structural proteins, e.g., in its cavity or lumen. In some embodiments, the virus based vector particles are lentivirus. In some embodiments, the lentiviral vector particle is Human Immunodeficiency Virus-1 (HIV-1).

In some aspects, the viral vector particle is limited in the number of polynucleotides that can be packaged. In some embodiments, nucleotides encoding polypeptides to be packaged can be modified such that they retain functional activity with fewer nucleotides in the coding region than that which encodes for the wild-type peptide. Such modifications can include truncations, or other deletions. In some embodiments, more than one polypeptide can be expressed from the same promoter, such that they are fusion polypeptides. In some embodiments, the insert size to be packaged (i.e., viral genome, or portions thereof; or heterologous polynucleotides as described) can be between 500-1000, 1000-2000, 2000-3000, 3000-4000, 4000-5000, 5000-6000, 6000-7000, or 7000-8000 nucleotides in length. In some embodiments, the insert can be over 8000 nucleotides, such as 9000, 10,000, or 11,000 nucleotides in length.

In some embodiments, the viral vector particle, such as retroviral vector particle, comprises one or more of gag polyprotein, polymerase (e.g., pol), integrase (e.g., a functional or non-functional variant), protease, and a fusogen. In some embodiments, the lipid particle further comprises rev. In some embodiments, one or more of the aforesaid proteins are encoded in the retroviral genome (i.e., the insert as described above), and in some embodiments, one or more of the aforesaid proteins are provided in trans, e.g., by a helper cell, helper virus, or helper plasmid. In some embodiments, the lipid particle nucleic acid (e.g., retroviral nucleic acid) comprises one or more of the following nucleic acid sequences: 5′ LTR (e.g., comprising U5 and lacking a functional U3 domain), Psi packaging element (Psi), Central polypurine tract (cPPT) Promoter operatively linked to the payload gene, payload gene (optionally comprising an intron before the open reading frame), Poly A tail sequence, WPRE, and 3′ LTR (e.g., comprising U5 and lacking a functional U3). In some embodiments, the lipid particle nucleic acid further comprises a retroviral cis-acting RNA packaging element, and a cPPT/CTS element. In some embodiments the lipid particle nucleic acid further comprises one or more insulator element. In some embodiments, the recognition sites are situated between the poly A tail sequence and the WPRE.

In some embodiments, the lipid particle comprises supramolecular complexes formed by viral proteins that self-assemble into capsids. In some embodiments, the lipid particle is a viral particle derived from viral capsids. In some embodiments, the lipid particle is a viral particle derived from viral nucleocapsids. In some embodiments, the lipid particle comprises nucleocapsid-derived that retain the property of packaging nucleic acids.

In some embodiments, the lipid particle packages nucleic acids from host cells carrying one or more viral nucleic acids (e.g. retroviral nucleic acids) during the expression process. In some embodiments, the nucleic acids do not encode any genes involved in virus replication. In particular embodiments, the lipid particle is a virus-based particle, e.g. retrovirus particle such as a lentivirus particle, that is replication defective.

In some cases, the lipid particle is a viral particle that is morphologically indistinguishable from the wild type infectious virus. In some embodiments, the viral particle presents the entire viral proteome as an antigen. In some embodiments, the viral particle presents only a portion of the proteome as an antigen.

In some embodiments, the retroviral nucleic acid comprises one or more of (e.g., all of): a 5′ promoter (e.g., to control expression of the entire packaged RNA), a 5′ LTR (e.g., that includes R (polyadenylation tail signal) and/or U5 which includes a primer activation signal), a primer binding site, a psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the retroviral nucleic acid further comprises one or more of a cPPT, a WPRE, and/or an insulator element.

A retrovirus typically replicates by reverse transcription of its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

In some embodiments the retrovirus is a Gammretrovirus. In some embodiments the retrovirus is an Epsilonretrovirus. In some embodiments the retrovirus is an Alpharetrovirus. In some embodiments the retrovirus is a Betaretrovirus. In some embodiments the retrovirus is a Deltaretrovirus. In some embodiments the retrovirus is a Lentivirus. In some embodiments the retrovirus is a Spumaretrovirus. In some embodiments the retrovirus is an endogenous retrovirus.

Illustrative lentiviruses include, but are not limited to: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV-based vector backbones (i.e., HIV cis-acting sequence elements) are used.

A viral vector can comprise a nucleic acid molecule (e.g., a transfer plasmid) that includes virus-derived nucleic acid elements that typically facilitate transfer of a nucleic acid molecule (e.g. including nucleic acid encoding an exogenous agent) or integration into the genome of a cell or to a viral particle that mediates nucleic acid transfer. Viral vector particles will typically include various viral components and sometimes also host cell components in addition to nucleic acid(s). A viral vector can comprise a virus or viral particle capable of transferring a nucleic acid into a cell (e.g. nucleic acid encoding an exogenous agent), or to the transferred nucleic acid (e.g., as naked DNA). Viral vectors and transfer plasmids can comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some vectors described herein, at least part of one or more protein coding regions that contribute to or are essential for replication may be absent compared to the corresponding wild-type virus. This makes the viral vector replication-defective. In some embodiments, the vector is capable of transducing a target non-dividing host cell and/or integrating its genome into a host genome.

The structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). The LTRs are involved in proviral integration and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are typically similar (e.g., identical) sequences that can be divided into three elements, which are called U3, R and U5. U3 is derived from the sequence unique to the 3′ end of the RNA. R is derived from a sequence repeated at both ends of the RNA and US is derived from the sequence unique to the 5′ end of the RNA. The sizes of the three elements can vary considerably among different retroviruses.

For the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some retroviruses comprise any one or more of the following genes that code for proteins that are involved in the regulation of gene expression: tot, rev, tax and rex. With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction promotes infection, e.g., by fusion of the viral membrane with the cell membrane.

In a replication-defective retroviral vector genome gag, pol and env may be absent or not functional. The R regions at both ends of the RNA are typically repeated sequences. U5 and U3 represent unique sequences at the 5′ and 3′ ends of the RNA genome respectively.

Retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. Proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11). The mechanisms of action of these two proteins are thought to be broadly similar to the analogous mechanisms in the primate viruses. In addition, an EIAV protein, Ttm, has been identified that is encoded by the first exon of tat spliced to the env coding sequence at the start of the transmembrane protein.

In addition to protease, reverse transcriptase and integrase, non-primate lentiviruses contain a fourth pol gene product which codes for a dUTPase. This may play a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. Infection of the target cell can comprise reverse transcription and integration into the target cell genome. The RLV typically carries non-viral coding sequences which are to be delivered by the vector to the target cell, such as nucleic acid encoding an exogenous agent as described herein. In embodiments, an RLV is incapable of independent replication to produce infectious retroviral particles within the target cell. Usually the RLV lacks a functional gag-pol and/or env gene and/or other genes involved in replication. The vector may be configured as a split-intron vector, e.g., as described in PCT patent application WO 99/15683, which is herein incorporated by reference in its entirety.

In some embodiments, the lipid particle (e.g., a lentiviral vector) comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

A minimal lentiviral genome may comprise, e.g., (5′) R-U5-one or more first nucleotide sequences-U3-R (3′). However, the plasmid vector used to produce the lentiviral genome within a source cell can also include transcriptional regulatory control sequences operably linked to the lentiviral genome to direct transcription of the genome in a source cell. These regulatory sequences may comprise the natural sequences associated with the transcribed retroviral sequence, e.g., the 5′ U3 region, or they may comprise a heterologous promoter such as another viral promoter, for example the CMV promoter. Some lentiviral genomes comprise additional sequences to promote efficient virus production. For example, in the case of HIV, rev and RRE sequences may be included. Alternatively or combination, codon optimization may be used, e.g., the gene encoding the exogenous agent may be codon optimized, e.g., as described in WO 01/79518, which is herein incorporated by reference in its entirety. Alternative sequences which perform a similar or the same function as the rev/RRE system may also be used. For example, a functional analogue of the rev/RRE system is found in the Mason Pfizer monkey virus. This is known as CTE and comprises an RRE-type sequence in the genome which is believed to interact with a factor in the infected cell. The cellular factor can be thought of as a rev analogue. Thus, CTE may be used as an alternative to the rev/RRE system. In addition, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non dividing cells.

The deletion of additional genes may permit vectors to be produced without the genes associated with disease in lentiviral (e.g. HIV) infections. In particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. Thus, the concentration of free dNTPs in the cytoplasm increases as Vpx degrades SAMHD1 and reverse transcription activity is increased, thus facilitating reverse transcription of the retroviral genome and integration into the target cell genome.

Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. An additional description of codon optimization is found, e.g., in WO 99/41397, which is herein incorporated by reference in its entirety.

Many viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide (nt) 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

It is within the level of a skilled artisan to empirically determine appropriate codon optimization of viral sequences. The strategy for codon optimized sequences, including gag-pol sequences, can be used in relation to any retrovirus, e.g., EIAV, FIV, BIV, CAEV, VMR, SIV, HIV-I and HIV-2. In addition this method could be used to increase expression of genes from HTLV-I. HTLV-2, HFV, HSRV and human endogenous retroviruses (HERV), MLV and other retroviruses.

In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, the lentiviral vector is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a lentivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798): 1316-1332). In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

According to certain specific embodiments, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. A variety of lentiviral vectors are described in Naldini et ah, (1996a, 1996b, and 1998); Zufferey et al., (1997); Dull et al., 1998, U.S. Pat. Nos. 6,013,516; and 5,994,136, many of which may be adapted to produce a retroviral nucleic acid.

At each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [Y] sequence) for encapsidation of the viral genome.

In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. In some aspects, provided herein is a replication incompetent (also referred to herein as replication defective) vector particle, that cannot participate in replication in the absence of the packaging cell (i.e., viral vector particles are not produced from the transduced cell). In some aspects, this is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly (A) sequence. The 3′ LTR, the 5′ LTR, or both 3′ and 5′ LTRs, may be modified LTRs. Other modifications to the viral vector, i.e., retroviral or lentiviral vector, to render said vector replication incompetent are known in the art.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

The R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

The retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et ah, 2000, Cell, 101:173, which are herein incorporated by reference in their entireties. During HIV-I reverse transcription, central initiation of the plus-strand DNA at the central polypurine tract (cPPT) and central termination at the central termination sequence (CTS) can lead to the formation of a three-stranded DNA structure: the HIV-I central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-L.

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65:1053; and Cullen et al., 1991. Cell 58:423), and the hepatitis B virus post-transcriptional regulatory element (HPRE), which are herein incorporated by reference in their entireties. Generally, the RNA export element is placed within the 3′ UTR of a gene and can be inserted as one or multiple copies.

In some embodiments, expression of heterologous sequences (e.g. nucleic acid encoding an exogenous agent) in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE.

In some embodiments, a retroviral nucleic acid described herein lacks or does not comprise a posttranscriptional regulatory element such as a WPRE or HPRE.

Elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATT AAA, AGTAAA, a bovine growth hormone polyA sequence (BGHpA), a rabbit b-globin polyA sequence (rPgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Y) packaging signal, RRE), and/or other elements that increase exogenous gene expression (e.g., poly (A) sequences), and may optionally comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a US sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

### 2. Virus-Like Particles

In some embodiments, the viral-based particles are viral-like lipid particles (VLPs) that are derived from virus. In some embodiments, the viral envelope may comprise a fusogen, e.g., a fusogen that is endogenous to the virus or a pseudotyped fusogen, e.g., a variant Paramyxovirus G/H protein as described in Section II and/or a Paramyxovirus F as described below in Section III.C. The VLPs include those derived from retroviruses or lentiviruses. While VLPs mimic native virion structure, they lack the viral genomic information necessary for independent replication within a host cell. Therefore, in some aspects, VLPs are non-infectious. In particular embodiments, a VLP does not contain a viral genome. In some embodiments, the VLP's bilayer of amphipathic lipids is or comprises the viral envelope. In some embodiments, the lipid particle's bilayer of amphipathic lipids is or comprises lipids derived from a cell. In some embodiments, a VLP contains at least one type of structural protein from a virus. In most cases this protein will form a proteinaceous capsid. In some cases, the capsid will also be enveloped in a lipid bilayer originating from the cell from which the assembled VLP has been released (e.g. VLPs comprising a human immunodeficiency virus structural protein such as GAG). In some embodiments, the VLP further comprises a targeting moiety as an envelope protein within the lipid bilayer.

In some embodiments, the vector vehicle particle comprises supramolecular complexes formed by viral proteins that self-assemble into capsids. In some embodiments, the vector vehicle particle is a virus-like particle derived from viral capsid proteins. In some embodiments, the vector vehicle particle is a virus-like particle derived from viral nucleocapsid proteins. In some embodiments, the vector vehicle particle comprises nucleocapsid-derived proteins that retain the property of packaging nucleic acids. In some embodiments, the viral-based particles, such as virus-like particles comprise only viral structural glycoproteins among proteins from the viral genome. In some embodiments, the vector vehicle particle does not contain a viral genome.

In some embodiments, the vector vehicle particle packages nucleic acids from host cells during the expression process, such as a nucleic acid encoding an exogenous agent. In some embodiments, the nucleic acids do not encode any genes involved in virus replication. In particular embodiments, the vector vehicle particle is a virus-like particle, e.g. retrovirus-like particle such as a lentivirus-like particle, that is replication defective.

In some embodiments, the vector vehicle particle is a virus-like particle which comprises a sequence that is devoid of or lacking viral RNA may be the result of removing or eliminating the viral RNA from the sequence. In some embodiments, this may be achieved by using an endogenous packaging signal binding site on gag. In some embodiments, the endogenous packaging signal binding site is on pol. In some embodiments, the RNA which is to be delivered will contain a cognate packaging signal. In some embodiments, a heterologous binding domain (which is heterologous to gag) located on the RNA to be delivered, and a cognate binding site located on gag or pol, can be used to ensure packaging of the RNA to be delivered. In some embodiments, the heterologous sequence could be non-viral or it could be viral, in which case it may be derived from a different virus. In some embodiments, the vector particles could be used to deliver therapeutic RNA, in which case functional integrase and/or reverse transcriptase is not required. In some embodiments, the vector particles could also be used to deliver a therapeutic gene of interest, in which case pol is typically included.

In some embodiments, the VLP comprises supramolecular complexes formed by viral proteins that self-assemble into capsids. In some embodiments, the VLP is derived from viral capsids. In some embodiments, the VLP is derived from viral nucleocapsids. In some embodiments, the VLP is nucleocapsid-derived and retains the property of packaging nucleic acids. In some embodiments, the VLP includes only viral structural glycoproteins. In some embodiments, the VLP does not contain a viral genome.

### B. Cell-Derived Particles

Provided herein are targeted lipid particles that comprise a naturally derived membrane containing a fusogen comprising a Paramyxovirus F and G/H glycoprotein. In some embodiments, the G/H protein is any one of the variant Paramyxovirus G/H proteins described in Section II. In some embodiments, the naturally derived membrane comprises membrane vesicles prepared from cells or tissues. In some embodiments, the targeted lipid particle comprises a vesicle that is obtainable from a cell. In some embodiments, the targeted lipid particle comprises a microvesicle, an exosome, a membrane enclosed body, an apoptotic body (from apoptotic cells), a particle (which may be derived from e.g. platelets), an ectosome (derivable from, e.g., neutrophiles and monocytes in serum), a prostatosome (obtainable from prostate cancer cells), or a cardiosome (derivable from cardiac cells).

In some embodiments, the source cell is an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial gial cell, a bone marrow stromal cell, a pancreatic progenitor cell, an endothelial progenitor cell, a blast cell), or an immortalized cell (e.g., HeEa, HEK293, HFF-1, MRC-5, WI-38, IMR 90, IMR 91, PER.C6, HT-1080, or BJ cell). In some embodiments, the source cell is other than a 293 cell, HEK cell, human endothelial cell, or a human epithelial cell, monocyte, macrophage, dendritic cell, or stem cell.

In some embodiments, the targeted lipid particle has a density of <1, 1-1.1, 1.05-1.15, 1.1-1.2, 1.15-1.25, 1.2-1.3, 1.25-1.35, or >1.35 g/ml. In some embodiments, the targeted lipid particle composition comprises less than 0.01%, 0.05%, 0.1%, 0.5%, 1%, 1.5%, 2%, 2.5%, 3%, 4%, 5%, or 10% source cells by protein mass or less than 0.01%, 0.05%, 0.1%, 0.5%, 1%, 1.5%, 2%, 2.5%, 3%, 4%, 5%, or 10% of cells having a functional nucleus.

In embodiments, the targeted lipid particle has a size, or the population of targeted lipid particles have an average size, that is less than about 0.01%, 0.05%, 0.1%, 0.5%, 1%, 2%, 3%, 4%, 5%, 10%, 20%, 30%, 40%, 50%, 60%, 70%, 80%, 90%, of that of the source cell.

In some embodiments the targeted lipid particle comprises an extracellular vesicle, e.g., a cell-derived vesicle comprising a membrane that encloses an internal space and has a smaller diameter than the cell from which it is derived. In embodiments the extracellular vesicle has a diameter from 20 nm to 1000 nm. In embodiments the targeted lipid particle comprises an apoptotic body, a fragment of a cell, a vesicle derived from a cell by direct or indirect manipulation, a vesiculated organelle, and a vesicle produced by a living cell (e.g., by direct plasma membrane budding or fusion of the late endosome with the plasma membrane). In embodiments the extracellular vesicle is derived from a living or dead organism, explanted tissues or organs, or cultured cells.

In embodiments, the targeted lipid particle comprises a nanovesicle, e.g., a cell-derived small (e.g., between 20-250 nm in diameter, or 30-150 nm in diameter) vesicle comprising a membrane that encloses an internal space, and which is generated from said cell by direct or indirect manipulation. The production of nanovesicles can, in some instances, result in the destruction of the source cell. The nanovesicle may comprise a lipid or fatty acid and polypeptide.

In embodiments, the targeted lipid particle comprises an exosome. In embodiments, the exosome is a cell-derived small (e.g., between 20-300 nm in diameter, or 40-200 nm in diameter) vesicle comprising a membrane that encloses an internal space, and which is generated from said cell by direct plasma membrane budding or by fusion of the late endosome with the plasma membrane. In embodiments, production of exosomes does not result in the destruction of the source cell. In embodiments, the exosome comprises lipid or fatty acid and polypeptide. Exemplary exosomes and other membrane-enclosed bodies are also described in WO/2017/161010, WO/2016/077639, US20160168572, US20150290343, and US20070298118, each of which is incorporated by reference herein in its entirety.

In some embodiments, the targeted lipid particle is derived from a source cell with a genetic modification which results in increased expression of an immunomodulatory agent. In some embodiments, the immunosuppressive agent is on an exterior surface of the cell. In some embodiments, the immunosuppressive agent is incorporated into the exterior surface of the targeted lipid particle. In some embodiments, the targeted lipid particle comprises an immunomodulatory agent attached to the surface of the solid particle by a covalent or non-covalent bond.

### C. Paramyxovirus F Proteins

In some embodiments, the lipid particle comprises one or more fusogens. In some embodiments, the lipid particle contains an exogenous or overexpressed fusogen. In some embodiments, the fusogen is disposed in the lipid bilayer. In some embodiments, the fusogen facilitates the fusion of the lipid particle to a membrane. In some embodiments, the membrane is a plasma cell membrane.

In some embodiments, fusogens comprise protein based, lipid based, and chemical based fusogens. In some embodiments, the lipid particle comprises a first fusogen comprising a protein fusogen and a second fusogen comprising a lipid fusogen or chemical fusogen. In some embodiments, the fusogen binds fusogen binding partner on a target cell surface.

In some embodiments, the fusogen comprises a protein with a hydrophobic fusion peptide domain. In some embodiments, the fusogen comprises a Paramyxovirus F protein molecule or biologically active portion thereof. In some embodiments, the Paramyxovirus F protein is a Henipavirus F protein. In some embodiments, the Henipavirus F protein is a Hendra (HeV) virus F protein, a Nipah (NiV) virus F-protein, a Cedar (CedPV) virus F protein, a Mòjiāng virus F protein, a Langya virus F protein, or a bat Paramyxovirus F protein or a biologically active portion thereof.

In some embodiments, the Paramyxovirus F protein exhibits fusogenic activity. In some embodiments, the F protein facilitates the fusion of the lipid particle (e.g. lentiviral vector) to a membrane. F proteins of henipaviruses, such as NiV-F, are encoded as F0 precursors containing a signal peptide (e.g. corresponding to amino acid residues 1-26 of the below). Following cleavage of the signal peptide, the mature F0 (SEQ ID NO: 131 lacking the signal peptide, i.e. set forth in SEQ ID NO: 136) is transported to the cell surface, then endocytosed and cleaved by cathepsin L (e.g. between amino acids 109-110 of NiV-F corresponding to amino acids set forth in SEQ ID NO: 131) into the mature fusogenic subunits F1 (e.g. corresponding to amino acids 110-546 of NiV-F set forth in SEQ ID NO: 131) and F2 (e.g. corresponding to amino acid residues 27-109 of NiV-F set forth in SEQ ID NO: 131). The F1 and F2 subunits are associated by a disulfide bond and recycled back to the cell surface. The F1 subunit contains the fusion peptide domain located at the N terminus of the F1 subunit (e.g. corresponding to amino acids 110-129 of the below e.g. NiV-F set forth in SEQ ID NO: 131) where it is able to insert into a cell membrane to drive fusion. In particular cases, fusion activity is blocked by association of the F protein with G protein, until G engages with a target molecule resulting in its disassociation from F and exposure of the fusion peptide to mediate membrane fusion.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019, 93 (13): e00577-19). In some aspects of the provided lipid particles, the F protein is heterologous to the G protein, i.e. the F and G protein or biologically active portions are from different henipavirus species. For example, the F protein is from Hendra virus and the G protein is from Nipah virus.

In some embodiments, the F protein or the biologically active portion thereof is a wild-type Paramyxovirus F protein, such as a Nipah virus F (NiV-F) protein or a Hendra virus F protein, or is a functionally active variant or biologically active portion thereof. For instance, in some embodiments, the F protein or the biologically active portion thereof is a wild-type NiV-F protein or a functionally active variant or a biologically active portion thereof.

In some embodiments, the F protein has the sequence of amino acids set forth in SEQ ID NO: 130, SEQ ID NO:131, SEQ ID NO: 132, SEQ ID NO:133, or SEQ ID NO:134, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 130, SEQ ID NO: 131, SEQ ID NO: 132, SEQ ID NO:133, or SEQ ID NO: 134, and retains fusogenic activity in conjunction with a Paramyxovirus G protein, such as a variant G protein as provided herein. In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:130, SEQ ID NO: 131, SEQ ID NO: 132, SEQ ID NO: 133, or SEQ ID NO:134.

In particular embodiments, the F protein has the sequence of amino acids set forth in SEQ ID NO: 135, SEQ ID NO: 136, SEQ ID NO:137, SEQ ID NO:138, or SEQ ID NO:139, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 135, SEQ ID NO:136, SEQ ID NO: 137, SEQ ID NO: 138, or SEQ ID NO: 139, and retains fusogenic activity in conjunction with a Paramyxovirus G protein, such as a variant G protein as provided herein. In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 135, SEQ ID NO: 136, SEQ ID NO: 137, SEQ ID NO: 138, or SEQ ID NO:139.

Fusogenic activity includes the activity of the F protein in conjunction with a G protein to promote or facilitate fusion of two membrane lumens, such as the lumen of the targeted lipid particle having embedded in its lipid bilayer a Paramyxovirus F and G protein, and a cytoplasm of a target cell, e.g. a cell that contains a surface receptor or molecule that is recognized or bound by the targeted envelope protein. In some embodiments, the F protein and G protein are from the same Paramyxovirus species (e.g. NiV-G and NiV-F). In some embodiments, the F protein and G protein are from different Paramyxovirus species (e.g. NiV-G and HeV-F). In particular embodiments, the F protein of the functionally active variant or biologically active portion retains the cleavage site cleaved by cathepsin L (e.g. corresponding to the cleavage site between amino acids 109-110 of SEQ ID NO:131).

Reference to retaining fusogenic activity includes activity (in conjunction with a G protein, such as a variant G protein provided herein) that is between at or about 10% and at or about 150% or more of the level or degree of binding of the corresponding wild-type F protein, such as set forth in SEQ ID NO:130, SEQ ID NO: 131, SEQ ID NO: 132, SEQ ID NO: 133, or SEQ ID NO: 134, SEQ ID NO: 135, SEQ ID NO: 136, SEQ ID NO: 137, SEQ ID NO: 138, or SEQ ID NO:139 or a cathepsin L cleaved from thereof containing an F1 and F2 subunit. In some embodiments, the fusogenic activity is at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type F protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the F protein is a mutant F protein that is a functionally active fragment or a biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (Hev) virus F protein, a Nipah (NiV) virus F-protein, a Cedar (CedPV) virus F protein, a Mojiang virus F protein, a Langya virus F protein or a bat Paramyxovirus F protein. In some embodiments, the wild-type F protein is encoded by a sequence of nucleotides that encodes any one of SEQ ID NO: 130, SEQ ID NO: 131, SEQ ID NO: 132, SEQ ID NO: 133, or SEQ ID NO:134, SEQ ID NO: 135, SEQ ID NO: 136, SEQ ID NO:137, SEQ ID NO:138, or SEQ ID NO:139 or a cathepsin L cleaved from thereof containing an F1 and F2 subunit.

In some embodiments, the mutant F protein is a biologically active portion that is truncated and lacks up to 22 contiguous amino acid residues at or near the C-terminus of the wild-type F protein, such as a wild-type F protein set forth in any one of SEQ ID NO: 130, SEQ ID NO:131, SEQ ID NO: 132, SEQ ID NO:133, or SEQ ID NO:134, SEQ ID NO: 135, SEQ ID NO:136, SEQ ID NO: 137, SEQ ID NO:138, or SEQ ID NO:139. In some embodiments, the mutant F protein is truncated and lacks up to 22 contiguous amino acids, such as up to 21, 20, 19, 18, 17, 16, 15, 14, 13, 12, 11, 10, 9, 8, 7, 6, 5, 4, 3, 2, or 1 contiguous amino acids at the C-terminus of the wild-type F protein.

In some embodiments, the Paramyxovirus F protein, such as a mutant or truncated F protein, of a provided lipid particle includes the F0 precursor or a proteolytically cleaved form thereof containing the F1 and F2 subunits, such as resulting following proteolytic cleavage at the cleavage site (e.g. between amino acids corresponding to amino acids between amino acids 109-110 of SEQ ID NO: 131) to produce two chains that can be linked by disulfide bond. In some embodiments, the F protein, such as wild-type F or a truncated or mutated F protein, is produced or encoded as an F0 precursor which then is able to be proteolytically cleaved to result in an F protein containing the F1 and F2 subunit linked by a disulfide bond. Hence, it is understood that reference to a particular sequence (SEQ ID NO) of a Paramyxovirus F protein herein is typically with reference to the F0 precursor sequence but also is understood to include the proteolytically cleaved form or sequence thereof containing the two cleaved chains, F1 and F2. For instance, NiV-F, such as a mutant or truncated NiV-F, contains an F1 subunit corresponding to amino acids 110-546 of NiV-F set forth in SEQ ID NO:131 or truncated or mutant sequence thereof, and an F2 corresponding to amino acid residues 27-109 of NiV-F set forth in SEQ ID NO:131.

In some embodiments, the mutant Paramyxovirus F protein is a biologically active portion that is truncated and lacks up to 22 contiguous amino acid residues at or near the C-terminus corresponding to the wild-type NiV-F protein, such as a wild-type NiV-F protein set forth in SEQ ID NO: 131 or SEQ ID NO:136. In some embodiments, the mutant F protein is truncated and lacks up to 22 contiguous amino acids, such as up to 21, 20, 19, 18, 17, 16, 15, 14, 13, 12, 11, 10, 9, 8, 7, 6, 5, 4, 3, 2, or 1 contiguous amino acids at the C-terminus of the wild-type NiV-F protein, such as a wild-type NiV-F protein set forth in SEQ ID NO: 131 or SEQ ID NO: 136. In some embodiments, the mutant F protein contains an F1 subunit and an F2 subunit in which (1) the F1 subunit is truncated and lacks up to 22 contiguous amino acids at or near the C-terminus of the wild-type F1 subunit, such as lacks up to 22 contiguous amino acids at or near the C-terminus of the wild-type F1 subunit corresponding to amino acids 110-546 of NiV-F set forth in SEQ ID NO:131, and (2) the F2 subunit has the sequence corresponding to amino acid residues 27-109 of NiV-F set forth in SEQ ID NO:131.

In some embodiments, the F protein is a mutant NiV-F protein that is a biologically active portion thereof that comprises a 22 amino acid truncation at or near the C-terminus of the wild-type NiV-F protein (SEQ ID NO: 131 or SEQ ID NO: 136). In some embodiments, the NiV-F protein is encoded by a nucleotide sequence that encodes the sequence set forth in SEQ ID NO: 140. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 140. In particular embodiments, the variant F protein is a mutant Niv-F protein that has the sequence of amino acids set forth in SEQ ID NO:141. In some embodiments, the NiV-F proteins is encoded by a sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 141. In some embodiments, the F protein molecule or biologically active portion thereof comprises the sequence set forth in SEQ ID NO: 141.

In some embodiments, the mutant F protein contains an F1 subunit and an F2 subunit in which (1) the F1 subunit is set forth as amino acids 110-524 of SEQ ID NO: 140, and (2) the F2 subunit is set forth as amino acids 27-109 of SEQ ID NO:140.

In some embodiments, the mutant F protein contains an F1 subunit and an F2 subunit in which (1) the F1 subunit is set forth as amino acids 84-498 of SEQ ID NO: 141, and (2) the F2 subunit is set forth as amino acids 1-83 of SEQ ID NO:141.

D. Methods of Generating Lipid particles

### 1. Methods of Generating Viral-Based Particles

Large scale viral particle production is often useful to achieve a desired viral titer. Viral particles can be produced by transfecting a transfer vector into a packaging cell line that comprises viral structural and/or accessory genes, e.g., gag, pol, env, tat, rev, vif, vpr, vpu, vpx, or nef genes or other retroviral genes.

In some embodiments, viral vector particles may be produced in multiple cell culture systems including bacteria, mammalian cell lines, insect cell lines, yeast and plant cells. Exemplary methods for producing viral vector particles are described.

In some embodiments, elements for the production of a viral vector, i.e., a recombinant viral vector such as a replication incompetent lentiviral vector, are included in a packaging cell line or are present on a packaging vector. In some embodiments, viral vectors can include packaging elements, rev, gag, and pol, delivered to the packaging cells line via one or more packaging vectors.

In embodiments, the packaging vector is an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. A retroviral, e.g., lentiviral, transfer vector can be introduced into a packaging cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides. In some embodiments, the packaging vector is a packaging plasmid.

Producer cell lines (also called packaging cell lines) include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211 A cells. In embodiments, the packaging cells are 293 cells, 293T cells, or A549 cells.

In some embodiments, a producer cell (i.e., a source cell line) includes a cell line which is capable of producing recombinant retroviral particles, comprising a packaging cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the packaging cells, e.g., by cell lysis, or collection of the supernatant of the cell culture. Optionally, the collected virus particles may be enriched or purified.

In some embodiments, the source cell comprises one or more plasmids coding for viral structural proteins and replication enzymes (e.g., gag, pol and env) which can package viral particles (i.e., a packaging plasmid). In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the exogenous agent (e.g., a retroviral nucleic acid encoding the exogenous agent) is also integrated into the source cell genome. In some embodiments a nucleic acid encoding the exogenous agent is maintained episomally. In some embodiments a nucleic acid encoding the exogenous agent is transfected into the source cell that has stably integrated Rev, Gag/Pol, and an envelope protein in the genome. See, e.g., Milani et al. EMBO Molecular Medicine, 2017, which is herein incorporated by reference in its entirety.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid.

Typically, modern retroviral vector systems include viral genomes bearing cis-acting vector sequences for transcription, reverse-transcription, integration, translation and packaging of viral RNA into the viral particles, and (2) producer cells lines which express the trans-acting retroviral gene sequences (e.g., gag, pol and env) needed for production of virus particles. By separating the cis- and trans-acting vector sequences completely, the virus is unable to maintain replication for more than one cycle of infection. Generation of live virus can be avoided by a number of strategies, e.g., by minimizing the overlap between the cis- and trans-acting sequences to avoid recombination.

A virus-like particle (VLP) which comprises a sequence that is devoid of or lacking viral RNA, such as described in Section III.2, may be the result of removing or eliminating the viral RNA from the sequence. Similar to the viral vector particles, such as described in Section III.1, VLPs contain a viral outer envelope made from the host cell (i.e., producer cell or source cell) lipid-bilayer as well as at least one viral structural protein. In some embodiments, a viral structural protein refers to any viral protein or fragment thereof which contributes to the structure of the viral core or capsid.

Generally, for lipid particles (e.g., lentiviral vector particles), expression of the gag precursor protein alone mediates vector assembly and release. In some aspects, gag proteins or fragments thereof have been demonstrated to assemble into structures analogous to viral cores. In one embodiment this may be achieved by using an endogenous packaging signal binding site on gag. Alternatively, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. The heterologous sequence could be non-viral or it could be viral, in which case it may be derived from a different virus. The VLP could be used to deliver therapeutic RNA, in which case functional integrase and/or reverse transcriptase is not required. These VLPs could also be used to deliver a therapeutic gene of interest, in which case pol is typically included.

In an embodiment, gag-pol are altered, and the packaging signal is replaced with a corresponding packaging signal. In this embodiment, the particle can package the RNA with the new packaging signal. The advantage of this approach is that it is possible to package an RNA sequence which is devoid of viral sequence for example, RNAi.

An alternative approach is to rely on over-expression of the RNA to be packaged. In one embodiment the RNA to be packaged is over-expressed in the absence of any RNA containing a packaging signal. This may result in a significant level of therapeutic RNA being packaged, and that this amount is sufficient to transduce a cell and have a biological effect.

In some embodiments, a polynucleotide comprises a nucleotide sequence encoding a viral gag protein or retroviral gag and pol proteins, wherein the gag protein or pol protein comprises a heterologous RNA binding domain capable of recognizing a corresponding sequence in an RNA sequence to facilitate packaging of the RNA sequence into a viral vector particle. In some embodiments, the heterologous RNA binding domain comprises an RNA binding domain derived from a bacteriophage coat protein, a Rev protein, a protein of the U 1 small nuclear ribonucleoprotein particle, a Nova protein, a TF111 A protein, a TIS 11 protein, a trp RNA-binding attenuation protein (TRAP) or a pseudouridine synthase.

In some embodiments, the gag protein is a polyprotein. In some embodiments, the gag protein is a polyprotein comprising a MA polypeptide, a CA polypeptide, and an NC polypeptide; ii) one or more exogenous polypeptides (such as is described in Section III.C.2); and iii) one or more heterologous protease cleavage sites, wherein at least one of the one or more heterologous protease cleavage sites is between the gag polyprotein and the one or more exogenous polypeptides.

In some embodiments, the MA, CA, and NC portions of the gag polyprotein can be of any retrovirus known in the art. For example, in some embodiments, the gag polyprotein is a gag polyprotein of an alpha retrovirus, a beta retrovirus, a gamma retrovirus, a delta retrovirus, an epsilon retrovirus, or a spumavirus. In some embodiments, the gag polyprotein is a gag polyprotein of a human immunodeficiency virus or murine leukemia virus.

In some embodiments, the gag polyprotein is a human immunodeficiency virus (HIV) gag polyprotein comprising a MA polypeptide, a CA polypeptide, a p2 polypeptide, an NC polypeptide, a pi polypeptide, and a p6 polypeptide. In some embodiments, the gag polyprotein comprises one or more heterologous protease cleavage sites between one or more of: i) the MA polypeptide and the CA polypeptide; ii) the CA polypeptide and the p2 polypeptide; iii) the p2 polypeptide and the NC polypeptide; iv) the NC polypeptide and the pi polypeptide; and v) the pi polypeptide and the p6 polypeptide. In some embodiments, a gag polyprotein can comprise: MA-heterologous protease cleavage site-CA-heterologous protease cleavage site-p2-heterologous protease cleavage site-NC-p1-p6. In some embodiments, the heterologous protease cleavage site is a TEV protease cleavage site: ENLYFQS (SEQ ID NO: 142), where cleavage occurs between the Gln and the Ser.

In some embodiments, the assembly of a viral based vector particle (i.e., a VLP) is initiated by binding of the core protein to a unique encapsidation sequence within the viral genome (e.g. UTR with stem-loop structure). In some embodiments, the interaction of the core with the encapsidation sequence facilitates oligomerization.

In some embodiments, the source cell for VLP production comprises one or more plasmids coding for viral structural proteins (e.g., gag, pol) which can package viral particles (i.e., a packaging plasmid). In some embodiments, the sequences coding for at least two of the gag and pol precursors are on the same plasmid. In some embodiments, the sequences coding for the gag and pol precursors are on different plasmids. In some embodiments, the sequences coding for the gag and pol precursors have the same expression signal, e.g., promoter. In some embodiments, the sequences coding for the gag and pol precursors have a different expression signal, e.g., different promoters. In some embodiments, expression of the gag and pol precursors is inducible.

In some embodiments, formation of VLPs or any viral-based particle, such as described above, can be detected by any suitable technique known in the art. Examples of such techniques include, e.g., electron microscopy, dynamic light scattering, selective chromatographic separation and/or density gradient centrifugation.

In some embodiments, following generation of the viral-based particle, the particles are treated with a glycosidase mixture.

### 2. Generation of Cell-Derived Particles

In some embodiments, targeted lipid particles are generated by inducing budding of an exosome, microvesicle, membrane vesicle, extracellular membrane vesicle, plasma membrane vesicle, giant plasma membrane vesicle, apoptotic body, mitoparticle, pyrenocyte, lysosome, or other membrane enclosed vesicle.

In some embodiments, targeted lipid particles are generated by inducing cell enucleation. Enucleation may be performed using assays such as genetic, chemical (e.g., using Actinomycin D, see Bayona-Bafaluy et al., “A chemical enucleation method for the transfer of mitochondrial DNA to ρ° cells” Nucleic Acids Res. 2003 Aug. 15; 31 (16): e98), mechanical methods (e.g., squeezing or aspiration, see Lee et al., “A comparative study on the efficiency of two enucleation methods in pig somatic cell nuclear transfer: effects of the squeezing and the aspiration methods.” Anim Biotechnol. 2008; 19 (2): 71-9), or combinations thereof.

In some embodiments, the targeted lipid particles are generated by inducing cell fragmentation. In some embodiments, cell fragmentation can be performed using the following methods, including, but not limited to: chemical methods, mechanical methods (e.g., centrifugation (e.g., ultracentrifugation, or density centrifugation), freeze-thaw, or sonication), or combinations thereof.

In some embodiments, the targeted lipid particle is a microvesicle. In some embodiments the microvesicle has a diameter of about 100 nm to about 2000 nm. In some embodiments, a targeted lipid particle comprises a cell ghost. In some embodiments, a vesicle is a plasma membrane vesicle, e.g. a giant plasma membrane vesicle.

In some embodiments, the source cell used to make the targeted lipid particle will not be available for testing after the targeted lipid particle is made.

In some embodiments, a characteristic of a targeted lipid particle is described by comparison to a reference cell. In embodiments, the reference cell is the source cell. In embodiments, the reference cell is a HeLa, HEK293, HFF-1, MRC-5, WI-38, IMR 90, IMR 91, PER.C6, HT-1080, or BJ cell. In some embodiments, a characteristic of a population of targeted lipid particle is described by comparison to a population of reference cells, e.g., a population of source cells, or a population of HeLa, HEK293, HFF-1, MRC-5, WI-38, IMR 90, IMR 91, PER.C6, HT-1080, or BJ cells.

### E. Lipid Particles Having Reduced Glycosylation

Provided herein are methods of producing or modifying lipid particles to have reduced or modified glycosylation.

### 1. Enzymatic Modification of Lipid Particles

In some embodiments, following the making of the lipid particle, the method further comprises enzymatic removal of glycans attached to proteins on the membrane of the lipid particle (e.g., including the F and G/H proteins). In some embodiments, the enzymatic removal comprises treating the lipid particle with one or more glycosidases (also referred to as glycoside hydrolases or glycosyl hydrolases).

Glycosidases catalyze the hydrolysis of a glycosidic linkage to release a smaller sugar unit. Glycosidases can be characterized as a retaining or inverting enzyme, depending on the stereochemical outcome of the hydrolysis reaction. Glycosidases can also be characterized as exo-acting or endo-acting, depending on whether the enzyme acts at the end of an oligosaccharide chain or at an internal residue of the oligosaccharide chain.

In some embodiments, treatment with one or more glycosidases results in complete removal of glycans from the F and G/H proteins on the lipid particle. In some embodiments, treatment with one or more glycosidases results in partial removal of glycans from on the F and G/H proteins on the lipid particle, such as removal of about 10%, about 20%, about 30%, about 40%, about 50%, about 60%, about 70%, about 80%, or about 90% of glycans from surface proteins on the lipid particle. In some embodiments, treatment with one or more glycosidases results in removal of about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, or about 90% to about 99% of glycans on the F and G/H proteins on the lipid particle.

In some embodiments, treatment with one or more glycosidases results in a simplified glycosylation pattern on the F and G/H proteins on the lipid particle, such as a monoglycosylation, diglycosylation, or triglycosylation.

In some embodiments, the lipid particle is treated with one or more endoglycosidases, such as O-glycosidase, endoglycosidase D, endoglycosidase F, endoglycosidase F1, endoglycosidase F2, endoglycosidase H, and/or endoglycosidase S.

In some embodiments, the lipid particle is treated with one or more exoglycosidases, such as one or more of α1-2 Fucosidase, α1-2,3 Mannosidase, α1-2,3,4,6 Fucosidase, α1-2,3,6 Mannosidase, α1-2,4,6 Fucosidase O, α1-3,4 Fucosidase, α1-3,4,6 Galactosidase, α1-3,6 Galactosidase, α1-6 Mannosidase, α2-3 Neuraminidase S, α2-3,6,8 Neuraminidase, α2-3,6,8,9 Neuraminidase A, α-N-Acetylgalactosaminidase, B1-3 Galactosidase, β1-3,4 Galactosidase, β1-4 Galactosidase S, β-N-Acetylglucosaminidase S, and/or β-N-Acetylhexosaminidase_{f}.

In some embodiments, the lipid particle is treated with a mixture of both endo- and exo-glycosidases.

### 2. Producer Cells

In some embodiments, the lipid particles described herein are produced from cells modified in such a way as to modulate glycosylation of the F and G/H proteins comprised in the lipid particles. In some embodiments, the modulation comprises one or more genetic modifications. In some embodiments, the modulation comprises a chemical treatment of the producer cell, such as with a glycosylation inhibitor.

a. Genetic Modifications

In some embodiments, the lipid particles described herein are produced from cells according to the methods described above in Section III.D, wherein the producer cell further comprises one or more genetic modifications that modulate glycosylation of proteins. In some embodiments, the modulation results in production of lipid particles comprising F and G/H proteins having reduced or eliminated of glycosylation of the F and G/H proteins of the lipid particles as compared to lipid particles produced from cells without the genetic modification. In some embodiments, the modulation results in production of lipid particles comprising F and G/H proteins having a modified glycosylation pattern as compared to the F and G/H proteins comprised in lipid particles produced from cells without the one or more genetic modifications.

In some embodiments, the genetic modifications are selected from knockout of one or more of FUT8, MGAT1, MGAT2, MGAT3, MGAT4A, MGAT4B, MGAT4C, MGAT5A, MGAT5B, B3GNT1, B3GNT2, B3GNT8, B4GALT1, B4GALT2, B4GALT3, B4GALT4, B4GALT5, B4GALT6, ST3GAL1, ST3GAL2, ST3GAL3, ST3GAL4, ST3GAL5, ST3GAL6, ST6GAL1, ST6GAL2, and C1GALT1, The genetic modifications can also include knock-in of one or more exogenous genes, such as endo-N-acetylglucosaminidase (EndoT).

In some embodiments, the producer cell comprises one or more genetic modifications that results in a reduction of glycans from F and G/H proteins comprised in the lipid particle, such as a reduction of about 10%, about 20%, about 30%, about 40%, about 50%, about 60%, about 70%, about 80%, or about 90% as compared to F and G/H proteins comprised in lipid particles produced from cells without the one or more genetic modifications. In some embodiments, the one or more genetic modifications results in a reduction of glycans from F and G/H proteins comprised in the lipid particle of about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, or about 90% to about 99%, as compared to F and G/H proteins comprised in lipid particles produced from cells without the one or more genetic modifications. In some embodiments, the one or more genetic modifications results in the complete elimination of detectable glycosylation of the F and G/H proteins comprised in the lipid particles.

In some embodiments, the producer cell comprises one or more genetic modification that results in lipid particles comprising F and G/H proteins having a different glycosylation profile as compared to F and G/H proteins comprised in lipid particles produced from cells without the one or more genetic modifications.

In some embodiments, the producer cell comprises one or more genetic modifications that results in reduced or defective N-linked glycosylation. In some embodiments, the producer cell comprises one or more genetic modifications that results in reduced or defective for O-linked glycosylation.

In some embodiments, the producer cell line comprises a mutation in the glycosyl transferase pathway. In some embodiments, the producer cell line comprises a mutation in GnTI (N-acetylglucosaminyl-transferase I).

In some embodiments, the producer cell line expresses a heterologous glycosidase that results in production of proteins with reduced glycosylation. In some embodiments, the heterologous glycosidase is targeted to the golgi apparatus. In some embodiments, the heterologous glycosidase is an exoglycosidase. In some embodiments, the glycosidase is an endoglycosidase. In some embodiments, some embodiments, the endoglycosidase is endoT.

In some embodiments, the producer cell line comprises both a mutation in in the glycosyl transferase pathway and expresses a heterologous glycosidase.

b. Chemical Treatment

In some embodiments, the producer cell is treated with a glycosylation inhibitor during the making of the lipid particle.

In some embodiments, treatment with one or more glycosylation inhibitors results in complete removal of glycans from F and G/H proteins on the lipid particle produced by the treated producer cell. In some embodiments, treatment with one or more glycosylation inhibitors results in partial removal of glycans from F and G/H proteins on the lipid particle, such as removal of about 10%, about 20%, about 30%, about 40%, about 50%, about 60%, about 70%, about 80%, or about 90% of glycans from the F and G/H proteins on the lipid particle. In some embodiments, treatment with one or more glycosylation inhibitors results in removal of about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, or about 90% to about 99% of glycans from F and G/H proteins on the lipid particle.

In some embodiments, treatment with one or more glycosylation inhibitors results in a simplified glycosylation pattern on the F and G/H proteins on the lipid particle, such as a monoglycosylation, diglycosylation, or triglycosylation.

In some embodiments, the glycosylation inhibitor is one or more of kinefusine, australine, castanosperimine, Deoxynojirimycin, Deoxymannojirimycin, Swainsoninne, and/or Mannostatin A.

### F. Exogenous Agents

In some embodiments, the lipid particle as described herein or pharmaceutical composition comprising same described contains an exogenous agent. In some embodiments, the lipid particle or pharmaceutical composition comprising same described herein contains a nucleic acid that encodes an exogenous agent. In some embodiments, the lipid particle contains the exogenous agent. In some embodiments, the lipid particle contains a nucleic acid that encodes an exogenous agent. Reference to the coding sequence of the nucleic acid encoding the exogenous agent also is referred to herein as a payload gene. In some embodiments, the exogenous agent or the nucleic acid encoding the exogenous agent are present in the lumen of the lipid particle.

In some embodiments, the exogenous agent is a protein or a nucleic acid (e.g., a DNA, a chromosome (e.g. a human artificial chromosome), an RNA, e.g., an mRNA or miRNA). In some embodiments, the exogenous agent comprises or encodes a membrane protein. In some embodiments, the exogenous agent comprises or encodes a therapeutic agent. In some embodiments, the therapeutic agent is chosen from one or more of a protein, e.g., an enzyme, a transmembrane protein, a receptor, or an antibody; a nucleic acid, e.g., DNA, a chromosome (e.g. a human artificial chromosome), RNA, mRNA, siRNA, or miRNA; or a small molecule.

In some embodiments, the lipid particle or pharmaceutical composition delivers to a target cell at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the lipid particle. In some embodiments, the lipid particle, e.g., fusosome, that contacts, e.g., fuses, with the target cell(s) delivers to the target cell an average of at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the lipid particles, e.g., fusosomes, that contact, e.g., fuse, with the target cell(s). In some embodiments, the lipid particle composition delivers to a target tissue at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the lipid particle compositions.

In some embodiments, the exogenous agent is not expressed naturally in the cell from which the lipid particle is derived. In some embodiments, the exogenous agent is expressed naturally in the cell from which the lipid particle is derived. In some embodiments, the exogenous agent is loaded into the lipid particle via expression in the cell from which the lipid particle is derived (e.g. expression from DNA or mRNA introduced via transfection, transduction, or electroporation). In some embodiments, the exogenous is expressed from DNA integrated into the genome or maintained episomally. In some embodiments, expression of the exogenous agent is constitutive. In some embodiments, expression of the exogenous agent is induced. In some embodiments, expression of the exogenous agent is induced immediately prior to generating the lipid particle. In some embodiments, expression of the exogenous agent is induced at the same time as expression of the fusogen.

In some embodiments, the exogenous agent is loaded into the lipid particle via electroporation into the lipid particle itself or into the cell from which the lipid particle is derived. In some embodiments, the exogenous agent is loaded into the lipid particle via transfection (e.g., of a DNA or mRNA encoding the exogenous agent) into the lipid particle itself or into the cell from which the lipid particle is derived.

In some embodiments, the exogenous agent may include one or more nucleic acid sequences, one or more polypeptides, a combination of nucleic acid sequences and/or polypeptides, one or more organelles, and any combination thereof. In some embodiments, the exogenous agent may include one or more cellular components. In some embodiments, the exogenous agent includes one or more cytosolic and/or nuclear components.

In some embodiments, the lipid particle contains an exogenous agent that is a nucleic acid or contains a nucleic acid encoding the exogenous agent. In some embodiments, the nucleic acid is operatively linked to a “positive target cell-specific regulatory element” (or positive TCSRE). In some embodiments, the positive TCSRE is a functional nucleic acid sequence. In some embodiments, the positive TCSRE comprises a promoter or enhancer. In some embodiments, the TCSRE is a nucleic acid sequence that increases the level of an exogenous agent in a target cell. In some embodiments, the positive target cell-specific regulatory element comprises a T cell-specific promoter, a T cell-specific enhancer, a T cell-specific splice site, a T cell-specific site extending half-life of an RNA or protein, a T cell-specific mRNA nuclear export promoting site, a T cell-specific translational enhancing site, or a T cell-specific post-translational modification site. In some embodiments, the T cell-specific promoter is a promoter described in Immgen consortium, herein incorporated by reference in its entirety, e.g., the T cell-specific promoter is an IL2RA (CD25), LRRC32, FOXP3, or IKZF2 promoter. In some embodiments, the T cell-specific promoter or enhancer is a promoter or enhancer described in Schmidt et a, Blood. 2014 Apr. 24; 123 (17): e68-78, herein incorporated by reference in its entirety. In some embodiments, the T cell-specific promoter is a transcriptionally active fragment of any of the foregoing. In some embodiments, the T-cell specific promoter is a variant having at least 70%, 75%, 80%, 85%, 90%, 95%, 96%, 97%, 98%, or 99% identity to any of the foregoing.

In some embodiments, the lipid particle contains an exogenous agent that is a nucleic acid or contains a nucleic acid encoding the exogenous agent. In some embodiments, the nucleic acid is operatively linked to a “negative target cell-specific regulatory element” (or negative TCSRE). In some embodiments, the negative TCSRE is a functional nucleic acid sequence. In some embodiments, the negative TCSRE is a miRNA recognition site that causes degradation of inhibition of the lipid particle in a non-target cell. In some embodiments, the exogenous agent is operatively linked to a “non-target cell-specific regulatory element” (or NTCSRE). In some embodiments, the NTCSRE comprises a nucleic acid sequence that decreases the level of an exogenous agent in a non-target cell compared to in a target cell. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a tissue-specific miRNA recognition sequence, tissue-specific protease recognition site, tissue-specific ubiquitin ligase site, tissue-specific transcriptional repression site, or tissue-specific epigenetic repression site. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a non-target cell-specific miRNA recognition sequence and the miRNA recognition sequence is able to be bound by one or more of miR3 1, miR363, or miR29c. In some embodiments, the NTCSRE is situated or encoded within a transcribed region encoding the exogenous agent, optionally wherein an RNA produced by the transcribed region comprises the miRNA recognition sequence within a UTR or coding region.

### 1. Nucleic Acids

In some embodiments, the exogenous agent may include a nucleic acid. For example, the exogenous agent may comprise RNA to enhance expression of an endogenous protein, or a siRNA or miRNA that inhibits protein expression of an endogenous protein. For example, the endogenous protein may modulate structure or function in the target cells. In some embodiments, the exogenous agent may include a nucleic acid encoding an engineered protein that modulates structure or function in the target cells. In some embodiments, the exogenous agent is a nucleic acid that targets a transcriptional activator that modulate structure or function in the target cells.

In some embodiments, a lipid particle described herein comprises a nucleic acid, e.g., RNA or DNA. In some embodiments, the nucleic acid is, comprises, or consists of one or more natural nucleic acid residues. In some embodiments, the nucleic acid is, comprises, or consists of one or more nucleic acid analogs. In some embodiments, the nucleic acid has a nucleotide sequence that encodes a functional gene product such as an RNA or protein. In some embodiments, the nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, the nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, the nucleic acid is partly or wholly single stranded; in some embodiments, the nucleic acid is partly or wholly double stranded. In some embodiments the nucleic acid has a nucleotide sequence comprising at least one element that encodes, or is the complement of a sequence that encodes, a polypeptide. The nucleic acid may include variants, e.g., having an overall sequence identity with a reference nucleic acid of at least 85%, 86%, 87%, 88%, 89%, 90%, 91%, 92%, 93%, 94%, 95%, 96%, 97%, or 99%. In some embodiments, a variant nucleic acid does not share at least one characteristic sequence element with a reference nucleic acid. In some embodiments, a variant nucleic acid shares one or more of the biological activities of the reference nucleic acid. In some embodiments, a nucleic acid variant has a nucleic acid sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant nucleic acid comprises about 10, about 9, about 8, about 7, about 6, about 5, about 4, about 3, about 2, or about 1 substituted residue as compared to a reference. In some embodiments, a variant nucleic acid comprises a very small number (e.g., fewer than about 5, about 4, about 3, about 2, or about 1) number of substituted, inserted, or deleted, functional residues that participate in a particular biological activity relative to the reference. In some embodiments, a variant nucleic acid comprises not more than about 15, about 12, about 9, about 3, or about 1 addition or deletion, and, in some embodiments, comprises no additions or deletions, as compared to the reference. In some embodiments, a variant nucleic acid comprises fewer than about 27, about 24, about 21, about 18, about 15, about 12, about 9, about 6, about 3, or fewer than about 9, about 6, about 3, or about 2 additions or deletions as compared to the reference.

In some embodiments, the exogenous agent includes a nucleic acid, e.g., DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), ERNA (enhancer RNA), satellite RNA, pcRNA (protein coding RNA), dsRNA (double stranded RNA), RNAi (interfering RNA), circRNA (circular RNA), reprograming RNAs, aptamers, and any combination thereof. In some embodiments, the nucleic acid is a wild-type nucleic acid. In some embodiments, the protein is a mutant nucleic acid. In some embodiments the nucleic acid is a fusion or chimera of multiple nucleic acid sequences.

In embodiments, the nucleic acid encodes one or more (e.g. two or more) inhibitory RNA molecules directed against one or more RNA targets. An inhibitory RNA molecule can be, e.g., a miRNA or an shRNA. In some embodiments, the inhibitory molecule can be a precursor of a miRNA, such as for example, a Pri-miRNA or a Pre-miRNA, or a precursor of an shRNA. In some embodiments, the inhibitory molecule can be an artificially derived miRNA or shRNA. In other embodiments, the inhibitory RNA molecule can be a dsRNA (either transcribed or artificially introduced) that is processed into an siRNA or the siRNA itself. In some embodiments, the inhibitory RNA molecule can be a miRNA or shRNA that has a sequence that is not found in nature, or has at least one functional segment that is not found in nature, or has a combination of functional segments that are not found in nature. In illustrative embodiments, at least one or all of the inhibitory RNA molecules are miR-155. In some embodiments, a retroviral vector described herein encodes two or more inhibitory RNA molecules directed against one or more RNA targets. Two or more inhibitory RNA molecules, in some embodiments, can be directed against different targets. In other embodiments, the two or more inhibitory RNA molecules are directed against the same target. In some embodiments, the exogenous agent comprises a shRNA. A shRNA (short hairpin RNA) can comprise a double-stranded structure that is formed by a single self complementary RNA strand. shRNA constructs can comprise a nucleotide sequence identical to a portion, of either coding or non-coding sequence, of a target gene. RNA sequences with insertions, deletions, and single point mutations relative to the target sequence can also be used. Greater than 90% sequence identity, or even 100% sequence identity, between the inhibitory RNA and the portion of the target gene can be used. In certain embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size. In embodiments, a retroviral vector that encodes an siRNA, an miRNA, an shRNA, or a ribozyme comprises one or more regulatory sequences, such as, for example, a strong constitutive pol III, e.g., human U6 snRNA promoter, the mouse U6 snRNA promoter, the human and mouse H I RNA promoter and the human tRNA-val promoter, or a strong constitutive pol II promoter.

### 2. Polypeptides

In some embodiments, the lipid particle contains a nucleic acid that encodes a protein exogenous agent (also referred to as a “payload gene encoding an exogenous agent.”). In some embodiments, a lipid particle described herein comprises an exogenous agent which is or comprises a protein.

In some embodiments, the protein may include moieties other than amino acids (e.g., may be glycoproteins, proteoglycans, etc.) and/or may be otherwise processed or modified. In some embodiments, the protein can sometimes include more than one polypeptide chain, for example linked by one or more disulfide bonds or associated by other means.

In some embodiments, the protein may contain L-amino acids, D-amino acids, or both and may contain any of a variety of amino acid modifications or analogs. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof. In some embodiments, a polypeptide may include its variants, e.g., having an overall sequence identity with a reference polypeptide of at least 85%, 86%, 87%, 88%, 89%, 90%, 91%, 92%, 93%, 94%, 95%, 96%, 97%, or 99%. In some embodiments, a variant polypeptide does not share at least one characteristic sequence element with a reference polypeptide. In some embodiments, a variant polypeptide shares one or more of the biological activities of the reference polypeptide. In some embodiments, a polypeptide variant has an amino acid sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide comprises about 10, about 9, about 8, about 7, about 6, about 5, about 4, about 3, about 2, or about 1 substituted residue as compared to a reference. In some embodiments, a variant polypeptide comprises a very small number (e.g., fewer than about 5, about 4, about 3, about 2, or about 1) number of substituted, inserted, or deleted, functional that participate in a particular biological activity relative to the reference. In some embodiments, a variant polypeptide comprises not more than about 5, about 4, about 3, about 2, or about 1 addition or deletion, and, in some embodiments, comprises no additions or deletions, as compared to the reference. In some embodiments, a variant polypeptide comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, the protein includes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, and any combination thereof. In some embodiments, the protein targets a protein in the cell for degradation. In some embodiments, the protein targets a protein in the cell for degradation by localizing the protein to the proteasome. In some embodiments, the protein is a wild-type protein. In some embodiments, the protein is a mutant protein.

Exemplary protein exogenous agents are described in the following subsections. In some embodiments, a lipid particle provided herein can include any of such exogenous agents. In particular embodiments, a lipid particle contains a nucleic acid encoding any of such exogenous agents.

a. Cytosolic Proteins

In some embodiments, the exogenous agent comprises a cytosolic protein, e.g., a protein that is produced in the recipient cell and localizes to the recipient cell cytoplasm. In some embodiments, the exogenous agent comprises a secreted protein, e.g., a protein that is produced and secreted by the recipient cell. In some embodiments, the exogenous agent comprises a nuclear protein, e.g., a protein that is produced in the recipient cell and is imported to the nucleus of the recipient cell. In some embodiments, the exogenous agent comprises an organellar protein (e.g., a mitochondrial protein), e.g., a protein that is produced in the recipient cell and is imported into an organelle (e.g., a mitochondrial) of the recipient cell. In some embodiments, the protein is a wild-type protein or a mutant protein. In some embodiments the protein is a fusion or chimeric protein.

b. Membrane Proteins

In some embodiments, the exogenous agent comprises a membrane protein. In some embodiments, the membrane protein comprises a chimeric antigen receptor (CAR), a T cell receptor, an integrin, an ion channel, a pore forming protein, a Toll-Like Receptor, an interleukin receptor, a cell adhesion protein, or a transport protein.

i. Chimeric Antigen Receptors

In certain embodiments, the payload gene may comprise an exogenous polynucleotide encoding a CAR. CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. The polycistronic vector of the present disclosure may be used to express one or more CARs in a host cell (e.g., a T cell) for use in cell-based therapies against various target antigens. The CARs expressed by the one or more expression cassettes may be the same or different. In these embodiments, the CAR may comprise an extracellular binding domain (also referred to as a “binder”) that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell. In any of these embodiments, the nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. The sequence variations may be due to codon-optimization, humanization, restriction enzyme-based cloning scars, and/or additional amino acid residues linking the functional domains, etc.

In certain embodiments, the CAR may comprise a signal peptide at the N-terminus. Non-limiting examples of signal peptides include CD8a signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 2 below.

TABLE 2
Exemplary sequences of signal peptides

| SEQ ID | | |
|---|---|---|
| NO: | Sequence | Description |
| 148 | MALPVTALLLPLA | CD8α signal peptide |
| | LLLHAARP | |
| 149 | METDTLLLWVLLL | IgK signal peptide |
| | WVPGSTG | |
| 150 | MLLLVTSLLLCEL | GMCSFR-α (CSF2RA) |
| | PHPAFLLIP | signal peptide |

In certain embodiments, the extracellular binding domain of the CAR may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (V_{H}) and a light chain variable region (V_{L}) of an antibody connected by a linker. The V_{H} and the V_{L} may be connected in either order, i.e., V_{H}-linker-V_{L} or V_{L}-linker-V_{H}. Non-limiting examples of linkers include Whitlow linker, (G4S) n (n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, and ROR1 (associated with solid tumors). In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8a hinge domain, CD28 hinge domain, IgG4 hinge domain, IgG4 hinge-CH2-CH3 domain, and variants thereof, the amino acid sequences of which are provided in Table 3 below.

TABLE 3
Exemplary sequences of hinge domains

| SEQ ID | | |
|---|---|---|
| NO: | Sequence | Description |
| 151 | TTTPAPRPPTPAPTIASQPLSL | CD8α hinge domain |
| | RPEACRPAAGGAVHTRGLDFAC | |
| | D | |
| 152 | IEVMYPPPYLDNEKSNGTIIH | CD28 hinge domain |
| | VKGKHLCPSPLFPGPSKP | |
| 153 | AAAIEVMYPPPYLDNEKSNGT | CD28 hinge domain |
| | IIHVKGKHLCPSPLFPGPSKP | |
| 154 | ESKYGPPCPPCP | IgG4 hinge domain |
| 155 | ESKYGPPCPSCP | IgG4 hinge domain |
| 156 | ESKYGPPCPPCPAPEFLGGPSV | IgG4 hinge-CH2-CH3 |
| | FLFPPKPKDTLMISRTPEVTC | domain |
| | VVVDVSQEDPEVQFNWYVDGV | |
| | EVHNAKTKPREEQFNSTYRVV | |
| | SVLTVLHQDWLNGKEYKCKVS | |
| | NKGLPSSIEKTISKAKGQPRE | |
| | PQVYTLPPSQEEMTKNQVSLT | |
| | CLVKGFYPSDIAVEWESNGQP | |
| | ENNYKTTPPVLDSDGSFFLYS | |
| | RLTVDKSRWQEGNVFSCSVMH | |
| | EALHNHYTQKSLSLSLGK | |

In certain embodiments, the transmembrane domain of the CAR may comprise a transmembrane region of the alpha, beta, or zeta chain of a T cell receptor, CD28, CD38, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or a functional variant thereof, including the human versions of each of these sequences. Table 4 provides the amino acid sequences of a few exemplary transmembrane domains.

TABLE 4
Exemplary sequences of transmembrane domains

| SEQ ID | | |
|---|---|---|
| NO: | Sequence | Description |
| 157 | IYIWAPLAGTCGV | CD8α transmembrane |
| | LLLSLVITLYC | domain |
| 158 | FWVLVVVGGVLAC | CD28 transmembrane |
| | YSLLVTVAFIIFW | domain |
| | V | |
| 159 | MFWVLVVVGGVLA | CD28 transmembrane |
| | CYSLLVTVAFIIF | domain |
| | WV | |

In certain embodiments, the intracellular signaling domain and/or intracellular costimulatory domain of the CAR may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros. Integrin alpha 4/CD49d, Integrin alpha 4 beta 1. Integrin alpha 4 beta 7/LPAM-1, LAG-3, TCLIA, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD35, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD3ζ domain, an ITAM, a CD28 domain, 4-1BB domain, or a functional variant thereof. Table 5 provides the amino acid sequences of a few exemplary intracellular costimulatory and/or signaling domains. In certain embodiments, as in the case of tisagenlecleucel as described below, the CD3ζ signaling domain of SEQ ID NO:162 may have a mutation, e.g., a glutamine (Q) to lysine (K) mutation, at amino acid position 14 (see SEQ ID NO:163).

TABLE 5
Exemplary sequences of intracellular
costimulatory and/or signaling domains

| SEQ ID | | |
|---|---|---|
| NO: | Sequence | Description |
| 160 | KRGRKKLLYIFKQPFMRPVQTTQEEDGCSC | 4-1BB costimulatory |
| | RFPEEEEGGCEL | domain |
| 161 | RSKRSRLLHSDYMNMTPRRPGPTRKHYQPY | CD28 costimulatory |
| | APPRDFAAYRS | domain |
| 162 | RVKFSRSADAPAYQQGQNQLYNELNLGRRE | CD3ζ signaling |
| | EYDVLDKRRGRDPEMGGKPRRKNPQEGLYN | domain |
| | ELQKDKMAEAYSEIGMKGERRRGKGHDGLY | |
| | QGLSTATKDTYDALHMQALPPR | |
| 163 | RVKFSRSADAPAYKQGQNQLYNELNLGRRE | CD3ζ signaling |
| | EYDVLDKRRGRDPEMGGKPRRKNPQEGLYN | domain (with Q |
| | ELQKDKMAEAYSEIGMKGERRRGKGHDGLY | to K mutation at |
| | QGLSTATKDTYDALHMQALPPR | position 14) |

In certain embodiments where the polycistronic vector encodes two or more CARs, the two or more CARs may comprise the same functional domains, or one or more different functional domains, as described. For example, the two or more CARs may comprise different signal peptides, extracellular binding domains, hinge domains, transmembrane domains, costimulatory domains, and/or intracellular signaling domains, in order to minimize the risk of recombination due to sequence similarities. Or, alternatively, the two or more CARs may comprise the same domains. In the cases where the same domain(s) and/or backbone are used, it is optional to introduce codon divergence at the nucleotide sequence level to minimize the risk of recombination.

a. CD19 CAR

In some embodiments, the CAR is a CD19 CAR (“CD19-CAR”), and in these embodiments, the polycistronic vector comprises an expression cassette that contains a nucleotide sequence encoding a CD19 CAR. In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8a signal peptide. In some embodiments, the CD8a signal peptide comprises or consists of an amino acid sequence set forth in SEQ ID NO:148 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:148. In some embodiments, the signal peptide comprises an IgK signal peptide. In some embodiments, the IgK signal peptide comprises or consists of an amino acid sequence set forth in SEQ ID NO: 149 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:149. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO: 150 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:150.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34 (16-17): 1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 6 below. In some embodiments, the CD19-specific scFv comprises or consists of an amino acid sequence set forth in SEQ ID NO: 164, 165, or 170, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 164, 165, or 170. In some embodiments, the CD19-specific scFv may comprise one or more CDRs having amino acid sequences set forth in SEQ ID NOs: 166-168 and 172-174. In some embodiments, the CD19-specific scFv may comprise a light chain with one or more CDRs having amino acid sequences set forth in SEQ ID NOs: 166-168. In some embodiments, the CD19-specific scFv may comprise a heavy chain with one or more CDRs having amino acid sequences set forth in SEQ ID NOs: 172-174. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the V_{H} and the V_{L} portions of the scFv is a Whitlow linker having an amino acid sequence set forth in SEQ ID NO:169. In some embodiments, the Whitlow linker may be replaced by a different linker, for example, a 3×G₄S linker having an amino acid sequence set forth in SEQ ID NO:175, which gives rise to a different FMC63-derived scFv having an amino acid sequence set forth in SEQ ID NO:174. In certain of these embodiments, the CD19-specific scFv comprises or consists of an amino acid sequence set forth in SEQ ID NO:174 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:174.

TABLE 6
Exemplary sequences of anti-CD19 scFv and components

| SEQ ID NO: | Amino Acid Sequence | Description |
|---|---|---|
| 164 | DIQMTQTTSSLSASLGDRVTISCRASQDI | Anti-CD19 FMC63 scFv |
| | SKYLNWYQQKPDGTVKLLIYHTSRLHS | entire sequence, with |
| | GVPSRFSGSGSGTDYSLTISNLEQEDIAT | Whitlow linker |
| | YFCQQGNTLPYTFGGGTKLEITGSTSGS | |
| | GKPGSGEGSTKGEVKLQESGPGLVAPS | |
| | QSLSVTCTVSGVSLPDYGVSWIRQPPRK | |
| | GLEWLGVIWGSETTYYNSALKSRLTIIK | |
| | DNSKSQVFLKMNSLQTDDTAIYYCAKH | |
| | YYYGGSYAMDYWGQGTSVTVSS | |
| 165 | DIQMTQTTSSLSASLGDRVTISCRASQDI | Anti-CD19 FMC63 scFv |
| | SKYLNWYQQKPDGTVKLLIYHTSRLHS | light chain variable region |
| | GVPSRFSGSGSGTDYSLTISNLEQEDIAT | |
| | YFCQQGNTLPYTFGGGTKLEIT | |
| 166 | QDISKY | Anti-CD19 FMC63 scFv |
| | | light chain CDR1 |
| 167 | HTS | Anti-CD19 FMC63 scFv |
| | | light chain CDR2 |
| 168 | QQGNTLPYT | Anti-CD19 FMC63 scFv |
| | | light chain CDR3 |
| 169 | GSTSGSGKPGSGEGSTKG | Whitlow linker |
| 170 | EVKLQESGPGLVAPSQSLSVTCTVSGVS | Anti-CD19 FMC63 scFv |
| | LPDYGVSWIRQPPRKGLEWLGVIWGSE | heavy chain variable region |
| | TTYYNSALKSRLTIIKDNSKSQVFLKMN | |
| | SLQTDDTAIYYCAKHYYYGGSYAMDY | |
| | WGQGTSVTVSS | |
| 171 | GVSLPDYG | Anti-CD19 FMC63 scFv |
| | | heavy chain CDR1 |
| 172 | IWGSETT | Anti-CD19 FMC63 scFv |
| | | heavy chain CDR2 |
| 173 | AKHYYYGGSYAMDY | Anti-CD19 FMC63 scFv |
| | | heavy chain CDR3 |
| 174 | DIQMTQTTSSLSASLGDRVTISCRASQD | Anti-CD19 FMC63 scFv |
| | ISKYLNWYQQKPDGTVKLLIYHTSRLHS | entire sequence, with 3xG4S |
| | GVPSRFSGSGSGTDYSLTISNLEQEDIA | linker |
| | TYFCQQGNTLPYTFGGGTKLEITGGGGS | |
| | GGGGSGGGGSEVKLQESGPGLVAPSQS | |
| | LSVTCTVSGVSLPDYGVSWIRQPPRKGL | |
| | EWLGVIWGSETTYYNSALKSRLTIIKDN | |
| | SKSQVFLKMNSLQTDDTAIYYCAKHYY | |
| | YGGSYAMDYWGQGTSVTVSS | |
| 175 | GGGGSGGGGSGGGGS | 3xG4S linker |

In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138 (9): 2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148 (10): 2983-2987 (1992)), and CLB-CD19 (De Rie Cell. Immunol. 118:368-381 (1989)). In any of these embodiments, the extracellular binding domain of the CD19 CAR can comprise or consist of the V_{H}, the V_{L}, and/or one or more CDRs of any of the antibodies.

In some embodiments, the hinge domain of the CD19 CAR comprises a CD8a hinge domain, for example, a human CD8a hinge domain. In some embodiments, the CD8a hinge domain comprises or consists of an amino acid sequence set forth in SEQ ID NO: 157 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:151. In some embodiments, the hinge domain comprises a CD28 hinge domain, for example, a human CD28 hinge domain. In some embodiments, the CD28 hinge domain comprises or consists of an amino acid sequence set forth in SEQ ID NO: 152 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:152. In some embodiments, the CD28 hinge domain comprises or consists of an amino acid sequence set forth in SEQ ID NO:153 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 153. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO: 155 or SEQ ID NO:154, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:155 or SEQ ID NO:154. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO: 156 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:156.

In some embodiments, the transmembrane domain of the CD19 CAR comprises a CD8a transmembrane domain, for example, a human CD8a transmembrane domain. In some embodiments, the CD8a transmembrane domain comprises or consists of an amino acid sequence set forth in SEQ ID NO: 157 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 157. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 158 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 158. In some embodiments, the CD28 transmembrane domain comprises or consists of an amino acid sequence set forth in SEQ ID NO:159 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:159.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO: 160 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:160. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO:161 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:161. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (2) signaling domain. CD3& associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In some embodiments, the CD3ζ signaling domain is human. In some embodiments, the CD32 signaling domain comprises or consists of an amino acid sequence set forth in SEQ ID NO:162 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:162.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO: 164 or SEQ ID NO: 165, the CD8a hinge domain of SEQ ID NO: 151, the CD8a transmembrane domain of SEQ ID NO: 157, the 4-1BB costimulatory domain of SEQ ID NO: 160, the CD3ζ signaling domain of SEQ ID NO: 162, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8a signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO: 164 or SEQ ID NO: 165, the IgG4 hinge domain of SEQ ID NO: 154 or SEQ ID NO: 155, the CD28 transmembrane domain of SEQ ID NO: 158, the 4-1BB costimulatory domain of SEQ ID NO: 160, the CD3ζ signaling domain of SEQ ID NO:162, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8a signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO: 164 or SEQ ID NO: 165, the CD28 hinge domain of SEQ ID NO:152, the CD28 transmembrane domain of SEQ ID NO: 158, the CD28 costimulatory domain of SEQ ID NO:161, the CD3ζ signaling domain of SEQ ID NO: 162, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8a signal peptide) as described.

In some embodiments, the polycistronic vector comprises an expression cassette that contains a nucleotide sequence encoding a CD19 CAR as set forth in SEQ ID NO:176 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 176 (see Table 8). The encoded CD19 CAR has a corresponding amino acid sequence set forth in SEQ ID NO: 177 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 177, with the following components: CD8a signal peptide, FMC63 scFv (V_{L}-Whitlow linker-V_{H}), CD8a hinge domain, CD8a transmembrane domain, 4-1BB costimulatory domain, and CD3ζ signaling domain.

In some embodiments, the polycistronic vector comprises an expression cassette that contains a nucleotide sequence encoding a commercially available embodiment of CD19 CAR. Non-limiting examples of commercially available embodiments of CD19 CARs expressed and/or encoded by T cells include tisagenlecleucel, lisocabtagene maraleucel, axicabtagene ciloleucel, and brexucabtagene autoleucel.

In some embodiments, the polycistronic vector comprises an expression cassette that contains a nucleotide sequence encoding tisagenlecleucel or portions thereof. Tisagenlecleucel comprises a CD19 CAR with the following components: CD8a signal peptide, FMC63 scFv (V_{L}-3×G₄S linker-V_{H}), CD8a hinge domain, CD8a transmembrane domain, 4-1BB costimulatory domain, and CD3ζ signaling domain. The nucleotide and amino acid sequence of the CD19 CAR in tisagenlecleucel are provided in Table 8, with annotations of the sequences provided in Table 8.

In some embodiments, the polycistronic vector comprises an expression cassette that contains a nucleotide sequence encoding lisocabtagene maraleucel or portions thereof. Lisocabtagene maraleucel comprises a CD19 CAR with the following components: GMCSFR-α or CSF2RA signal peptide, FMC63 scFv (V_{L}-Whitlow linker-V_{H}), IgG4 hinge domain, CD28 transmembrane domain, 4-1BB costimulatory domain, and CD3ζ signaling domain. The nucleotide and amino acid sequence of the CD19 CAR in lisocabtagene maraleucel are provided in Table 7, with annotations of the sequences provided in Table 9.

In some embodiments, the polycistronic vector comprises an expression cassette that contains a nucleotide sequence encoding axicabtagene ciloleucel or portions thereof. Axicabtagene ciloleucel comprises a CD19 CAR with the following components: GMCSFR-α or CSF2RA signal peptide, FMC63 scFv (V_{L}-Whitlow linker-V_{H}), CD28 hinge domain, CD28 transmembrane domain, CD28 costimulatory domain, and CD3ζ signaling domain. The nucleotide and amino acid sequence of the CD19 CAR in axicabtagene ciloleucel are provided in Table 7, with annotations of the sequences provided in Table 10.

In some embodiments, the polycistronic vector comprises an expression cassette that contains a nucleotide sequence encoding brexucabtagene autoleucel or portions thereof. Brexucabtagene autoleucel comprises a CD19 CAR with the following components: GMCSFR-α signal peptide, FMC63 scFv, CD28 hinge domain, CD28 transmembrane domain, CD28 costimulatory domain, and CD3ζ signaling domain.

In some embodiments, the polycistronic vector comprises an expression cassette that contains a nucleotide sequence encoding a CD19 CAR as set forth in SEQ ID NO: 178, 180, or 182, or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 178, 180, or 182. The encoded CD19 CAR has a corresponding amino acid sequence set forth in SEQ ID NO: 179, 181, or 183, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 179, 181, or 183, respectively.

TABLE 7
Exemplary sequences of CD19 CARs

| SEQ ID NO: | Sequence | Description |
|---|---|---|
| 176 | atggccttaccagtgaccgccttgctcctgccgctggcct | Exemplary CD19 |
| | tgctgctccacgccgccaggccggacatccagatgacaca | CAR nucleotide |
| | gactacatcctccctgtctgcctctctgggagacagagtc | sequence |
| | accatcagttgcagggcaagtcaggacattagtaaatatt | |
| | taaattggtatcagcagaaaccagatggaactgttaaact | |
| | cctgatctaccatacatcaagattacactcaggagtccca | |
| | tcaaggttcagtggcagtgggtctggaacagattattctc | |
| | tcaccattagcaacctggagcaagaagatattgccactta | |
| | cttttgccaacagggtaatacgcttccgtacacgttcgga | |
| | ggggggaccaagctggagatcacaggctccacctctggat | |
| | ccggcaagcccggatctggcgagggatccaccaagggcga | |
| | ggtgaaactgcaggagtcaggacctggcctggtggcgccc | |
| | tcacagagcctgtccgtcacatgcactgtctcaggggtct | |
| | cattacccgactatggtgtaagctggattcgccagcctcc | |
| | acgaaagggtctggagtggctgggagtaatatggggtagt | |
| | gaaaccacatactataattcagctctcaaatccagactga | |
| | ccatcatcaaggacaactccaagagccaagttttcttaaa | |
| | aatgaacagtctgcaaactgatgacacagccatttactac | |
| | tgtgccaaacattattactacggtggtagctatgctatgg | |
| | actactggggccaaggaacctcagtcaccgtctcctcaac | |
| | cacgacgccagcgccgcgaccaccaacaccggcgcccacc | |
| | atcgcgtcgcagcccctgtccctgcgcccagaggcgtgcc | |
| | ggccagcggcggggggcgcagtgcacacgagggggctgga | |
| | cttcgcctgtgatatctacatctgggcgcccttggccggg | |
| | acttgtggggtccttctcctgtcactggttatcacccttt | |
| | actgcaaacggggcagaaagaaactcctgtatatattcaa | |
| | acaaccatttatgagaccagtacaaactactcaagaggaa | |
| | gatggctgtagctgccgatttccagaagaagaagaaggag | |
| | gatgtgaactgagagtgaagttcagcaggagcgcagacgc | |
| | ccccgcgtaccagcagggccagaaccagctctataacgag | |
| | ctcaatctaggacgaagagaggagtacgatgttttggaca | |
| | agagacgtggccgggaccctgagatggggggaaagccgag | |
| | aaggaagaaccctcaggaaggcctgtacaatgaactgcag | |
| | aaagataagatggcggaggcctacagtgagattgggatga | |
| | aaggcgagcgccggaggggcaaggggcacgatggccttta | |
| | ccagggtctcagtacagccaccaaggacacctacgacgcc | |
| | cttcacatgcaggccctgccccctcgc | |
| 177 | MALPVTALLLPLALLLHAARPDIQMTQTTSSLSASLGDRV | Exemplary CD19 |
| | TISCRASQDISKYLNWYQQKPDGTVKLLIYHTSRLHSGVP | CAR amino acid |
| | SRFSGSGSGTDYSLTISNLEQEDIATYFCQQGNTLPYTFG | sequence |
| | GGTKLEITGSTSGSGKPGSGEGSTKGEVKLQESGPGLVAP | |
| | SQSLSVTCTVSGVSLPDYGVSWIRQPPRKGLEWLGVIWGS | |
| | ETTYYNSALKSRLTIIKDNSKSQVFLKMNSLQTDDTAIYY | |
| | CAKHYYYGGSYAMDYWGQGTSVTVSSTTTPAPRPPTPAPT | |
| | IASQPLSLRPEACRPAAGGAVHTRGLDFACDIYIWAPLAG | |
| | TCGVLLLSLVITLYCKRGRKKLLYIFKQPFMRPVQTTQEE | |
| | DGCSCRFPEEEEGGCELRVKFSRSADAPAYQQGQNQLYNE | |
| | LNLGRREEYDVLDKRRGRDPEMGGKPRRKNPQEGLYNELQ | |
| | KDKMAEAYSEIGMKGERRRGKGHDGLYQGLSTATKDTYDA | |
| | LHMQALPPR | |
| 178 | atggccttaccagtgaccgccttgctcctgccgctggcct | Tisagenlecleucel |
| | tgctgctccacgccgccaggccggacatccagatgacaca | CD19 CAR |
| | gactacatcctccctgtctgcctctctgggagacagagtc | nucleotide sequence |
| | accatcagttgcagggcaagtcaggacattagtaaatatt | |
| | taaattggtatcagcagaaaccagatggaactgttaaact | |
| | cctgatctaccatacatcaagattacactcaggagtccca | |
| | tcaaggttcagtggcagtgggtctggaacagattattctc | |
| | tcaccattagcaacctggagcaagaagatattgccactta | |
| | cttttgccaacagggtaatacgcttccgtacacgttcgga | |
| | ggggggaccaagctggagatcacaggtggcggtggctcgg | |
| | gcggtggtgggtcgggtggcggcggatctgaggtgaaact | |
| | gcaggagtcaggacctggcctggtggcgccctcacagagc | |
| | ctgtccgtcacatgcactgtctcaggggtctcattacccg | |
| | actatggtgtaagctggattcgccagcctccacgaaaggg | |
| | tctggagtggctgggagtaatatggggtagtgaaaccaca | |
| | tactataattcagctctcaaatccagactgaccatcatca | |
| | aggacaactccaagagccaagttttcttaaaaatgaacag | |
| | tctgcaaactgatgacacagccatttactactgtgccaaa | |
| | cattattactacggtggtagctatgctatggactactggg | |
| | gccaaggaacctcagtcaccgtctcctcaaccacgacgcc | |
| | agcgccgcgaccaccaacaccggcgcccaccatcgcgtcg | |
| | cagcccctgtccctgcgcccagaggcgtgccggccagcgg | |
| | ggggggcgcagtgcacacgagggggctggacttcgcctgt | |
| | gatatctacatctgggcgcccttggccgggacttgtgggg | |
| | tccttctcctgtcactggttatcaccctttactgcaaacg | |
| | gggcagaaagaaactcctgtatatattcaaacaaccattt | |
| | atgagaccagtacaaactactcaagaggaagatggctgta | |
| | gctgccgatttccagaagaagaagaaggaggatgtgaact | |
| | gagagtgaagttcagcaggagcgcagacgcccccgcgtac | |
| | aagcagggccagaaccagctctataacgagctcaatctag | |
| | gacgaagagaggagtacgatgttttggacaagagacgtgg | |
| | ccgggaccctgagatggggggaaagccgagaaggaagaac | |
| | cctcaggaaggcctgtacaatgaactgcagaaagataaga | |
| | tggcggaggcctacagtgagattgggatgaaaggcgagcg | |
| | ccggaggggcaaggggcacgatggcctttaccagggtctc | |
| | agtacagccaccaaggacacctacgacgcccttcacatgc | |
| | aggccctgccccctcgc | |
| 179 | MALPVTALLLPLALLLHAARPDIQMTQTTSSLSASLGDRV | Tisagenlecleucel |
| | TISCRASQDISKYLNWYQQKPDGTVKLLIYHTSRLHSGVP | CD19 CAR amino |
| | SRFSGSGSGTDYSLTISNLEQEDIATYFCQQGNTLPYTFG | acid sequence |
| | GGTKLEITGGGGSGGGGSGGGGSEVKLQESGPGLVAPSQS | |
| | LSVTCTVSGVSLPDYGVSWIRQPPRKGLEWLGVIWGSETT | |
| | YYNSALKSRLTIIKDNSKSQVFLKMNSLQTDDTAIYYCAK | |
| | HYYYGGSYAMDYWGQGTSVTVSSTTTPAPRPPTPAPTIAS | |
| | QPLSLRPEACRPAAGGAVHTRGLDFACDIYIWAPLAGTCG | |
| | VLLLSLVITLYCKRGRKKLLYIFKQPFMRPVQTTQEEDGC | |
| | SCRFPEEEEGGCELRVKFSRSADAPAYKQGQNQLYNELNL | |
| | GRREEYDVLDKRRGRDPEMGGKPRRKNPQEGLYNELQKDK | |
| | MAEAYSEIGMKGERRRGKGHDGLYQGLSTATKDTYDALHM | |
| | QALPPR | |
| 180 | atgctgctgctggtgaccagcctgctgctgtgcgagctgc | Lisocabtagene |
| | cccaccccgcctttctgctgatccccgacatccagatgac | maraleucel CD19 |
| | ccagaccacctccagcctgagcgccagcctgggcgaccgg | CAR nucleotide |
| | gtgaccatcagctgccgggccagccaggacatcagcaagt | sequence |
| | acctgaactggtatcagcagaagcccgacggcaccgtcaa | |
| | gctgctgatctaccacaccagccggctgcacagcggcgtg | |
| | cccagccggtttagcggcagcggctccggcaccgactaca | |
| | gcctgaccatctccaacctggaacaggaagatatcgccac | |
| | ctacttttgccagcagggcaacacactgccctacaccttt | |
| | ggcggcggaacaaagctggaaatcaccggcagcacctccg | |
| | gcagcggcaagcctggcagcggcgagggcagcaccaaggg | |
| | cgaggtgaagctgcaggaaagcggccctggcctggtggcc | |
| | cccagccagagcctgagcgtgacctgcaccgtgagcggcg | |
| | tgagcctgcccgactacggcgtgagctggatccggcagcc | |
| | ccccaggaagggcctggaatggctgggcgtgatctggggc | |
| | agcgagaccacctactacaacagcgccctgaagagccggc | |
| | tgaccatcatcaaggacaacagcaagagccaggtgttcct | |
| | gaagatgaacagcctgcagaccgacgacaccgccatctac | |
| | tactgcgccaagcactactactacggcggcagctacgcca | |
| | tggactactggggccagggcaccagcgtgaccgtgagcag | |
| | cgaatctaagtacggaccgccctgccccccttgccctatg | |
| | ttctgggtgctggtggtggtcggaggcgtgctggcctgct | |
| | acagcctgctggtcaccgtggccttcatcatcttttgggt | |
| | gaaacggggcagaaagaaactcctgtatatattcaaacaa | |
| | ccatttatgagaccagtacaaactactcaagaggaagatg | |
| | gctgtagctgccgatttccagaagaagaagaaggaggatg | |
| | tgaactgcgggtgaagttcagcagaagcgccgacgcccct | |
| | gcctaccagcagggccagaatcagctgtacaacgagctga | |
| | acctgggcagaagggaagagtacgacgtcctggataagcg | |
| | gagaggccgggaccctgagatgggcggcaagcctcggcgg | |
| | aagaacccccaggaaggcctgtataacgaactgcagaaag | |
| | acaagatggccgaggcctacagcgagatcggcatgaaggg | |
| | cgagcggaggcggggcaagggccacgacggcctgtatcag | |
| | ggcctgtccaccgccaccaaggatacctacgacgccctgc | |
| | acatgcaggccctgcccccaagg | |
| 181 | MLLLVTSLLLCELPHPAFLLIPDIQMTQTTSSLSASLGDR | Lisocabtagene |
| | VTISCRASQDISKYLNWYQQKPDGTVKLLIYHTSRLHSGV | maraleucel CD19 |
| | PSRFSGSGSGTDYSLTISNLEQEDIATYFCQQGNTLPYTF | CAR amino acid |
| | GGGTKLEITGSTSGSGKPGSGEGSTKGEVKLQESGPGLVA | sequence |
| | PSQSLSVTCTVSGVSLPDYGVSWIRQPPRKGLEWLGVIWG | |
| | SETTYYNSALKSRLTIIKDNSKSQVFLKMNSLQTDDTAIY | |
| | YCAKHYYYGGSYAMDYWGQGTSVTVSSESKYGPPCPPCPM | |
| | FWVLVVVGGVLACYSLLVTVAFIIFWVKRGRKKLLYIFKQ | |
| | PFMRPVQTTQEEDGCSCRFPEEEEGGCELRVKFSRSADAP | |
| | AYQQGQNQLYNELNLGRREEYDVLDKRRGRDPEMGGKPRR | |
| | KNPQEGLYNELQKDKMAEAYSEIGMKGERRRGKGHDGLYQ | |
| | GLSTATKDTYDALHMQALPPR | |
| 182 | atgcttctcctggtgacaagccttctgctctgtgagttac | Axicabtagene |
| | cacacccagcattcctcctgatcccagacatccagatgac | ciloleucel CD19 |
| | acagactacatcctccctgtctgcctctctgggagacaga | CAR nucleotide |
| | gtcaccatcagttgcagggcaagtcaggacattagtaaat | sequence |
| | atttaaattggtatcagcagaaaccagatggaactgttaa | |
| | actcctgatctaccatacatcaagattacactcaggagtc | |
| | ccatcaaggttcagtggcagtgggtctggaacagattatt | |
| | ctctcaccattagcaacctggagcaagaagatattgccac | |
| | ttacttttgccaacagggtaatacgcttccgtacacgttc | |
| | ggaggggggactaagttggaaataacaggctccacctctg | |
| | gatccggcaagcccggatctggcgagggatccaccaaggg | |
| | cgaggtgaaactgcaggagtcaggacctggcctggtggcg | |
| | ccctcacagagcctgtccgtcacatgcactgtctcagggg | |
| | tctcattacccgactatggtgtaagctggattcgccagcc | |
| | tccacgaaagggtctggagtggctgggagtaatatggggt | |
| | agtgaaaccacatactataattcagctctcaaatccagac | |
| | tgaccatcatcaaggacaactccaagagccaagttttctt | |
| | aaaaatgaacagtctgcaaactgatgacacagccatttac | |
| | tactgtgccaaacattattactacggtggtagctatgcta | |
| | tggactactggggtcaaggaacctcagtcaccgtctcctc | |
| | agcggccgcaattgaagttatgtatcctcctccttaccta | |
| | gacaatgagaagagcaatggaaccattatccatgtgaaag | |
| | ggaaacacctttgtccaagtcccctatttcccggaccttc | |
| | taagcccttttgggtgctggtggtggttgggggagtcctg | |
| | gcttgctatagcttgctagtaacagtggcctttattattt | |
| | tctgggtgaggagtaagaggagcaggctcctgcacagtga | |
| | ctacatgaacatgactccccgccgccccgggcccacccgc | |
| | aagcattaccagccctatgccccaccacgcgacttcgcag | |
| | cctatcgctccagagtgaagttcagcaggagcgcagacgc | |
| | ccccgcgtaccagcagggccagaaccagctctataacgag | |
| | ctcaatctaggacgaagagaggagtacgatgttttggaca | |
| | agagacgtggccgggaccctgagatggggggaaagccgag | |
| | aaggaagaaccctcaggaaggcctgtacaatgaactgcag | |
| | aaagataagatggcggaggcctacagtgagattgggatga | |
| | aaggcgagcgccggaggggcaaggggcacgatggccttta | |
| | ccagggtctcagtacagccaccaaggacacctacgacgcc | |
| | cttcacatgcaggccctgccccctcgc | |
| 183 | MLLLVTSLLLCELPHPAFLLIPDIQMTQTTSSLSASLGDR | Axicabtagene |
| | VTISCRASQDISKYLNWYQQKPDGTVKLLIYHTSRLHSGV | ciloleucel CD19 |
| | PSRFSGSGSGTDYSLTISNLEQEDIATYFCQQGNTLPYTF | CAR amino acid |
| | GGGTKLEITGSTSGSGKPGSGEGSTKGEVKLQESGPGLVA | sequence |
| | PSQSLSVTCTVSGVSLPDYGVSWIRQPPRKGLEWLGVIWG | |
| | SETTYYNSALKSRLTIIKDNSKSQVFLKMNSLQTDDTAIY | |
| | YCAKHYYYGGSYAMDYWGQGTSVTVSSAAAIEVMYPPPYL | |
| | DNEKSNGTIIHVKGKHLCPSPLFPGPSKPFWVLVVVGGVL | |
| | ACYSLLVTVAFIIFWVRSKRSRLLHSDYMNMTPRRPGPTR | |
| | KHYQPYAPPRDFAAYRSRVKFSRSADAPAYQQGQNQLYNE | |
| | LNLGRREEYDVLDKRRGRDPEMGGKPRRKNPQEGLYNELQ | |
| | KDKMAEAYSEIGMKGERRRGKGHDGLYQGLSTATKDTYDA | |
| | LHMQALPPR | |

TABLE 8
Annotation of tisagenlecleucel CD19 CAR sequences

| | Nucleotide | Amino Acid |
|---|---|---|
| | Sequence | Sequence |
| Feature | Position | Position |
| CD8α signal peptide | 1-63 | 1-21 |
| FMC63 scFv | 64-789 | 22-263 |
| (V_{L}-3xG₄S linker-V_{H}) | | |
| CD8α hinge domain | 790-924 | 264-308 |
| CD8α transmembrane domain | 925-996 | 309-332 |
| 4-1BB costimulatory domain | 997-1122 | 333-374 |
| CD3ζ signaling domain | 1123-1458 | 375-486 |

TABLE 9
Annotation of lisocabtagene maraleucel CD19 CAR sequences

| | Nucleotide | Amino Acid |
|---|---|---|
| | Sequence | Sequence |
| Feature | Position | Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 10
Annotation of axicabtagene ciloleucel CD19 CAR sequences

| | Nucleotide | Amino Acid |
|---|---|---|
| | Sequence | Sequence |
| Feature | Position | Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 costimulatory domain | 1009-1131 | 337-377 |
| CD3ζ signaling domain | 1132-1467 | 378-489 |

In some embodiments, the polycistronic vector comprises an expression cassette that contains a nucleotide sequence encoding CD19 CAR as set forth in SEQ ID NO: 178, 180, or 182, or at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 178, 180, or 182. The encoded CD19 CAR has a corresponding amino acid sequence set forth in SEQ ID NO: 179, 181, or 183, respectively, is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 179, 181, or 183, respectively.

b. CD20 CAR

In some embodiments, the CAR is a CD20 CAR (“CD20-CAR”), and in these embodiments, the polycistronic vector comprises an expression cassette that contains a nucleotide sequence encoding a CD20 CAR. CD20 is an antigen found on the surface of B cells as early at the pro-B phase and progressively at increasing levels until B cell maturity, as well as on the cells of most B-cell neoplasms. CD20 positive cells are also sometimes found in cases of Hodgkins disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8a signal peptide. In some embodiments, the CD8a signal peptide comprises or consists of an amino acid sequence set forth in SEQ ID NO:148 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:148. In some embodiments, the signal peptide comprises an IgK signal peptide. In some embodiments, the IgK signal peptide comprises or consists of an amino acid sequence set forth in SEQ ID NO:149 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:149. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO: 150 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:150.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IF5, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14 (12): 1025-1033 (2001). In some embodiments, the linker is a 3×G₄S linker. In other embodiments, the linker is a Whitlow linker as described herein. In some embodiments, the amino acid sequences of different portions of the entire Leu16-derived scFv (also referred to as Leu16 scFv) and its different portions are provided in Table 11 below. In some embodiments, the CD20-specific scFv comprises or consists of an amino acid sequence set forth in SEQ ID NO:184, 185, or 189, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 184, 185, or 189 In some embodiments, the CD20-specific scFv may comprise one or more CDRs having amino acid sequences set forth in SEQ ID NOs: 186-188, 190, 191, and 192. In some embodiments, the CD20-specific scFv may comprise a light chain with one or more CDRs having amino acid sequences set forth in SEQ ID NOs: 186-188. In some embodiments, the CD20-specific scFv may comprise a heavy chain with one or more CDRs having amino acid sequences set forth in SEQ ID NOs: 190, 191, and 192. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 11
Exemplary sequences of anti-CD20 scFv and components

| SEQ ID NO: | Amino Acid Sequence | Description |
|---|---|---|
| 184 | DIVLTQSPAILSASPGEKVTMTCRASS | Anti-CD20 Leu16 scFv |
| | SVNYMDWYQKKPGSSPKPWIYATSNLA | entire sequence, with |
| | SGVPARFSGSGSGTSYSLTISRVEAED | Whitlow linker |
| | AATYYCQQWSFNPPTFGGGTKLEIKGS | |
| | TSGSGKPGSGEGSTKGEVQLQQSGAEL | |
| | VKPGASVKMSCKASGYTFTSYNMHWVK | |
| | QTPGQGLEWIGAIYPGNGDTSYNQKFK | |
| | GKATLTADKSSSTAYMQLSSLTSEDSA | |
| | DYYCARSNYYGSSYWFFDVWGAGTTVT | |
| | VSS | |
| 185 | DIVLTQSPAILSASPGEKVTMTCRASS | Anti-CD20 Leu16 scFv |
| | SVNYMDWYQKKPGSSPKPWIYATSNLA | light chain variable |
| | SGVPARFSGSGSGTSYSLTISRVEAED | region |
| | AATYYCQQWSFNPPTFGGGTKLEIK | |
| 186 | RASSSVNYMD | Anti-CD20 Leu16 scFv |
| | | light chain CDR1 |
| 187 | ATSNLAS | Anti-CD20 Leu16 scFv |
| | | light chain CDR2 |
| 188 | QQWSFNPPT | Anti-CD20 Leu16 scFv |
| | | light chain CDR3 |
| 189 | EVQLQQSGAELVKPGASVKMSCKASG | Anti-CD20 Leu16 scFv |
| | YTFTSYNMHWVKQTPGQGLEWIGAIY | heavy chain |
| | PGNGDTSYNQKFKGKATLTADKSSST | |
| | AYMQLSSLTSEDSADYYCARSNYYGS | |
| | SYWFFDVWGAGTTVTVSS | |
| 190 | SYNMH | Anti-CD20 Leu16 scFv |
| | | heavy chain CDR1 |
| 191 | AIYPGNGDTSYNQKFKG | Anti-CD20 Leu16 scFv |
| | | heavy chain CDR2 |
| 192 | SNYYGSSYWFFDV | Anti-CD20 Leu16 scFv |
| | | heavy chain CDR3 |

In some embodiments, the hinge domain of the CD20 CAR comprises a CD8a hinge domain, for example, a human CD8a hinge domain. In some embodiments, the CD8a hinge domain comprises or consists of an amino acid sequence set forth in SEQ ID NO:151 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:151. In some embodiments, the hinge domain comprises a CD28 hinge domain, for example, a human CD28 hinge domain. In some embodiments, the CD28 hinge domain comprises or consists of an amino acid sequence set forth in SEQ ID NO:152 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 152. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO: 154 or SEQ ID NO:155, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:154 or SEQ ID NO:155. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:156 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:156.

In some embodiments, the transmembrane domain of the CD20 CAR comprises a CD8a transmembrane domain, for example, a human CD8a transmembrane domain. In some embodiments, the CD8a transmembrane domain comprises or consists of an amino acid sequence set forth in SEQ ID NO: 157 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 157. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:159 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:159.

In some embodiments, the intracellular costimulatory domain of the CD20 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:160 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:160. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:161 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:161.

In some embodiments, the intracellular signaling domain of the CD20 CAR comprises a CD3 zeta (ζ) signaling domain, for example, a human CD3 signaling domain. In some embodiments, the CD3& signaling domain comprises or consists of an amino acid sequence set forth in SEQ ID NO: 162 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:162.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:184, the CD8a hinge domain of SEQ ID NO: 151, the CD8a transmembrane domain of SEQ ID NO: 157, the 4-1BB costimulatory domain of SEQ ID NO: 160, the CD3ζ signaling domain of SEQ ID NO: 162, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO: 184, the CD28 hinge domain of SEQ ID NO: 151, the CD8a transmembrane domain of SEQ ID NO: 157, the 4-1BB costimulatory domain of SEQ ID NO: 160, the CD3ζ signaling domain of SEQ ID NO:162, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO: 184, the IgG4 hinge domain of SEQ ID NO:154 or SEQ ID NO: 155, the CD8a transmembrane domain of SEQ ID NO: 157, the 4-1BB costimulatory domain of SEQ ID NO: 160, the CD3ζ signaling domain of SEQ ID NO: 161, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:184, the CD8a hinge domain of SEQ ID NO: 151, the CD28 transmembrane domain of SEQ ID NO: 153, the 4-1BB costimulatory domain of SEQ ID NO: 160, the CD3& signaling domain of SEQ ID NO: 161, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO: 184, the CD28 hinge domain of SEQ ID NO: 153, the CD28 transmembrane domain of SEQ ID NO: 159, the 4-1BB costimulatory domain of SEQ ID NO:160, the CD3ζ signaling domain of SEQ ID NO:161, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:184, the IgG4 hinge domain of SEQ ID NO: 154 or SEQ ID NO: 155, the CD28 transmembrane domain of SEQ ID NO: 158, the 4-1BB costimulatory domain of SEQ ID NO:160, the CD3ζ signaling domain of SEQ ID NO: 161, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

c. CD22 CAR

In some embodiments, the CAR is a CD22 CAR (“CD22-CAR”), and in these embodiments, the polycistronic vector comprises an expression cassette that contains a nucleotide sequence encoding a CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8α signal peptide. In some embodiments, the CD8α signal peptide comprises or consists of an amino acid sequence set forth in SEQ ID NO209 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO209. In some embodiments, the signal peptide comprises an IgK signal peptide. In some embodiments, the IgK signal peptide comprises or consists of an amino acid sequence set forth in SEQ ID NO:210 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:210. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:211 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:211.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3×G₄S linker. In other embodiments, the Whitlow linker may be used instead. In some embodiments, the amino acid sequences of the entire m971-derived scFv (also referred to as m971 scFv) and its different portions are provided in Table 13 below. In some embodiments, the CD22-specific scFv comprises or consists of an amino acid sequence set forth in SEQ ID NO:128, 129, or 133, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 128, 129, or 133. In some embodiments, the CD22-specific scFv may comprise one or more CDRs having amino acid sequences set forth in SEQ ID NOs: 130-132 and 134-136. In some embodiments, the CD22-specific scFv may comprise a heavy chain with one or more CDRs having amino acid sequences set forth in SEQ ID NOs: 130-132. In some embodiments, the CD22-specific scFv may comprise a light chain with one or more CDRs having amino acid sequences set forth in SEQ ID NOs: 134-136. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 pM). In some embodiments, the scFv derived from m971-L7 comprises the V_{H} and the V_{L} of m971-L7 connected by a 3×G₄S linker. In other embodiments, the Whitlow linker may be used instead. In some embodiments, the amino acid sequences of the entire m971-L7-derived scFv (also referred to as m971-L7 scFv) and its different portions are provided in Table 12 below. In some embodiments, the CD22-specific scFv comprises or consists of an amino acid sequence set forth in SEQ ID NO:193, 194, or 202, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 193, 194, or 202. In some embodiments, the CD22-specific scFv may comprise one or more CDRs having amino acid sequences set forth in SEQ ID NOs: 195-197, 199-201, 204-206, or 208-210. In some embodiments, the CD22-specific scFv may comprise a heavy chain with one or more CDRs having amino acid sequences set forth in SEQ ID NOs: 195-197 or 204-206. In some embodiments, the CD22-specific scFv may comprise a light chain with one or more CDRs having amino acid sequences set forth in SEQ ID NOs: 199-201 or 208-210. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 12
Exemplary sequences of anti-CD22 scFv and components

| SEQ ID NO: | Amino Acid Sequence | Description |
|---|---|---|
| 193 | QVQLQQSGPGLVKPSQTLSLTCAISGDS | Anti-CD22 m971 scFv |
| | VSSNSAAWNWIRQSPSRGLEWLGRTYY | entire sequence, with |
| | RSKWYNDYAVSVKSRITINPDTSKNQFS | 3xG4S linker |
| | LQLNSVTPEDTAVYYCAREVTGDLEDA | |
| | FDIWGQGTMVTVSSGGGGSGGGGSGG | |
| | GGSDIQMTQSPSSLSASVGDRVTITCRA | |
| | SQTIWSYLNWYQQRPGKAPNLLIYAAS | |
| | SLQSGVPSRFSGRGSGTDFTLTISSLQAE | |
| | DFATYYCQQSYSIPQTFGQGTKLEIK | |
| 194 | QVQLQQSGPGLVKPSQTLSLTCAISGDS | Anti-CD22 m971 scFv |
| | VSSNSAAWNWIRQSPSRGLEWLGRTYY | heavy chain variable |
| | RSKWYNDYAVSVKSRITINPDTSKNQFS | region |
| | LQLNSVTPEDTAVYYCAREVTGDLEDA | |
| | FDIWGQGTMVTVSS | |
| 195 | GDSVSSNSAA | Anti-CD22 m971 scFv |
| | | heavy chain CDR1 |
| 196 | TYYRSKWYN | Anti-CD22 m971 scFv |
| | | heavy chain CDR2 |
| 197 | AREVTGDLEDAFDI | Anti-CD22 m971 scFv |
| | | heavy chain CDR3 |
| 198 | DIQMTQSPSSLSASVGDRVTITCRASQTI | Anti-CD22 m971 scFv |
| | WSYLNWYQQRPGKAPNLLIYAASSLQS | light chain |
| | GVPSRFSGRGSGTDFTLTISSLQAEDFAT | |
| | YYCQQSYSIPQTFGQGTKLEIK | |
| 199 | QTIWSY | Anti-CD22 m971 scFv |
| | | light chain CDR1 |
| 200 | AAS | Anti-CD22 m971 scFv |
| | | light chain CDR2 |
| 201 | QQSYSIPQT | Anti-CD22 m971 scFv |
| | | light chain CDR3 |
| 202 | QVQLQQSGPGMVKPSQTLSLTCAISGD | Anti-CD22 m971-L7 scFv |
| | SVSSNSVAWNWIRQSPSRGLEWLGRTY | entire sequence, with |
| | YRSTWYNDYAVSMKSRITINPDTNKNQ | 3xG4S linker |
| | FSLQLNSVTPEDTAVYYCAREVTGDLE | |
| | DAFDIWGQGTMVTVSSGGGGSGGGGSG | |
| | GGGSDIQMIQSPSSLSASVGDRVTITC | |
| | RASQTIWSYLNWYRQRPGEAPNLLIYA | |
| | ASSLQSGVPSRFSGRGSGTDFTLTISS | |
| | LQAEDFATYYCQQSYSIPQTFGQGTKL | |
| | EIK | |
| 203 | QVQLQQSGPGMVKPSQTLSLTCAISGD | Anti-CD22 m971-L7 scFv |
| | SVSSNSVAWNWIRQSPSRGLEWLGRTY | heavy chain variable |
| | YRSTWYNDYAVSMKSRITINPDTNKNQ | region |
| | FSLQLNSVTPEDTAVYYCAREVTGDLE | |
| | DAFDIWGQGTMVTVSS | |
| 204 | GDSVSSNSVA | Anti-CD22 m971-L7 scFv |
| | | heavy chain CDR1 |
| 205 | TYYRSTWYN | Anti-CD22 m971-L7 scFv |
| | | heavy chain CDR2 |
| 206 | AREVTGDLEDAFDI | Anti-CD22 m971-L7 scFv |
| | | heavy chain CDR3 |
| 207 | DIQMIQSPSSLSASVGDRVTITCRASQTI | Anti-CD22 m971-L7 scFv |
| | WSYLNWYRQRPGEAPNLLIYAASSLQS | light chain variable region |
| | GVPSRFSGRGSGTDFTLTISSLQAEDFAT | |
| | YYCQQSYSIPQTFGQGTKLEIK | |
| 208 | QTIWSY | Anti-CD22 m971-L7 scFv |
| | | light chain CDR1 |
| 209 | AAS | Anti-CD22 m971-L7 scFv |
| | | light chain CDR2 |
| 210 | QQSYSIPQT | Anti-CD22 m971-L7 scFv |
| | | light chain CDR3 |

In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280 (1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8α hinge domain, for example, a human CD8α hinge domain. In some embodiments, the CD8α hinge domain comprises or consists of an amino acid sequence set forth in SEQ ID NO:212 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:212. In some embodiments, the hinge domain comprises a CD28 hinge domain, for example, a human CD28 hinge domain. In some embodiments, the CD28 hinge domain comprises or consists of an amino acid sequence set forth in SEQ ID NO:213 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:213. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:215 or SEQ ID NO:216, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:215 or SEQ ID NO:216. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:217 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:217.

In some embodiments, the transmembrane domain of the CD22 CAR comprises a CD8α transmembrane domain, for example, a human CD8α transmembrane domain. In some embodiments, the CD8α transmembrane domain comprises or consists of an amino acid sequence set forth in SEQ ID NO: 218 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:218. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO219 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO219.

In some embodiments, the intracellular costimulatory domain of the CD22 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:221 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:221. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:222 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:222.

In some embodiments, the intracellular signaling domain of the CD22 CAR comprises a CD3 zeta (ζ) signaling domain, for example, a human CD3 signaling domain. In some embodiments, the CD3ζ signaling domain comprises or consists of an amino acid sequence set forth in SEQ ID NO: 223 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:223.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO: 128 or SEQ ID NO: 137, the CD8α hinge domain of SEQ ID NO:212, the CD8α transmembrane domain of SEQ ID NO: 218, the 4-1BB costimulatory domain of SEQ ID NO:221, the CD3ζ signaling domain of SEQ ID NO: 223, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO: 128 or SEQ ID NO: 137, the CD28 hinge domain of SEQ ID NO:213, the CD8α transmembrane domain of SEQ ID NO: 218, the 4-1BB costimulatory domain of SEQ ID NO:221, the CD3ζ signaling domain of SEQ ID NO: 223, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO: 128 or SEQ ID NO: 137, the IgG4 hinge domain of SEQ ID NO:215 or SEQ ID NO:216, the CD8α transmembrane domain of SEQ ID NO:218, the 4-1BB costimulatory domain of SEQ ID NO:221, the CD3ζ signaling domain of SEQ ID NO:223, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO: 128 or SEQ ID NO: 137, the CD8α hinge domain of SEQ ID NO:8, the CD28 transmembrane domain of SEQ ID NO219, the 4-1BB costimulatory domain of SEQ ID NO:221, the CD3ζ signaling domain of SEQ ID NO: 223, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:193 or SEQ ID NO: 202, the CD28 hinge domain of SEQ ID NO:213, the CD28 transmembrane domain of SEQ ID NO219, the 4-1BB costimulatory domain of SEQ ID NO:221, the CD3ζ signaling domain of SEQ ID NO: 223, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO: 193 or SEQ ID NO: 202, the IgG4 hinge domain of SEQ ID NO:215 or SEQ ID NO:216, the CD28 transmembrane domain of SEQ ID NO219, the 4-1BB costimulatory domain of SEQ ID NO:221, the CD3& signaling domain of SEQ ID NO:223, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

d. BCMA CAR

In some embodiments, the CAR is a BCMA CAR (“BCMA-CAR”), and in these embodiments, the polycistronic vector comprises an expression cassette that contains a nucleotide sequence encoding a BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8α signal peptide. In some embodiments, the CD8α signal peptide comprises or consists of an amino acid sequence set forth in SEQ ID NO209 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO209. In some embodiments, the signal peptide comprises an IgK signal peptide. In some embodiments, the IgK signal peptide comprises or consists of an amino acid sequence set forth in SEQ ID NO:210 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:210. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:211 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:211.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, and ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19 (8): 2048-2060 (2013). See also PCT Application Publication No. WO2010/104949. The C11D5.3-derived scFv may comprise the heavy chain variable region (V_{H}) and the light chain variable region (V_{L}) of C11D5.3 connected by the Whitlow linker, the amino acid sequences of which is provided in Table 13 below. In some embodiments, the BCMA-specific extracellular binding domain comprises or consists of an amino acid sequence set forth in SEQ ID NO:211, 212, or 216 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 211, 212, or 216. In some embodiments, the BCMA-specific extracellular binding domain may comprise one or more CDRs having amino acid sequences set forth in SEQ ID NOs: 213-215 or 217-219. In some embodiments, the BCMA-specific extracellular binding domain may comprise a light chain with one or more CDRs having amino acid sequences set forth in SEQ ID NOs: 213-215. In some embodiments, the BCMA-specific extracellular binding domain may comprise a heavy chain with one or more CDRs having amino acid sequences set forth in SEQ ID NOs: 217-219. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19 (8): 2048-2060 (2013) and PCT Application Publication No. WO2010/104949, the amino acid sequence of which is also provided in Table 13 below. In some embodiments, the BCMA-specific extracellular binding domain comprises or consists of an amino acid sequence set forth in SEQ ID NO:220, 221, or 225, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:220, 221, or 225. In some embodiments, the BCMA-specific extracellular binding domain may comprise one or more CDRs having amino acid sequences set forth in SEQ ID NOs: 222-224 and 226-229. In some embodiments, the BCMA-specific extracellular binding domain may comprise a light chain with one or more CDRs having amino acid sequences set forth in SEQ ID NOs: 222-224. In some embodiments, the BCMA-specific extracellular binding domain may comprise a heavy chain with one or more CDRs having amino acid sequences set forth in SEQ ID NOs: 226-229. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29 (5): 585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11 (1): 141 (2018), also referred to as LCAR-B38M. See also, PCT Application Publication No. WO2018/028647.

In some embodiments, the extracellular binding domain of the BCMA CAR comprises a fully human heavy-chain variable domain (FHVH) as described in Lam et al., Nat. Commun. 11 (1): 283 (2020), also referred to as FHVH33. See also, PCT Application Publication No. WO2019/006072. The amino acid sequences of FHVH33 and its CDRs are provided in Table 13 below. In some embodiments, the BCMA-specific extracellular binding domain comprises or consists of an amino acid sequence set forth in SEQ ID NO:229 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 229. In some embodiments, the BCMA-specific extracellular binding domain may comprise one or more CDRs having amino acid sequences set forth in SEQ ID NOs: 230-232. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 13 below. In some embodiments, the BCMA-specific extracellular binding domain comprises or consists of an amino acid sequence set forth in SEQ ID NO: 233, 234, or 238, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 233, 234, or 238. In some embodiments, the BCMA-specific extracellular binding domain may comprise one or more CDRs having amino acid sequences set forth in SEQ ID NOs: 235-237 and 239-241. In some embodiments, the BCMA-specific extracellular binding domain may comprise a light chain with one or more CDRs having amino acid sequences set forth in SEQ ID NOs: 235-237. In some embodiments, the BCMA-specific extracellular binding domain may comprise a heavy chain with one or more CDRs having amino acid sequences set forth in SEQ ID NOs: 239-241. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 13
Exemplary sequences of anti-BCMA binder and components

| SEQ ID NO: | Amino Acid Sequence | Description |
|---|---|---|
| 211 | DIVLTQSPASLAMSLGKRATISCRASES | Anti-BCMA C11D5.3 |
| | VSVIGAHLIHWYQQKPGQPPKLLIYLAS | scFv entire sequence, with |
| | NLETGVPARFSGSGSGTDFTLTIDPVEE | Whitlow linker |
| | DDVAIYSCLQSRIFPRTFGGGTKLEIKGS | |
| | TSGSGKPGSGEGSTKGQIQLVQSGPELK | |
| | KPGETVKISCKASGYTFTDYSINWVKR | |
| | APGKGLKWMGWINTETREPAYAYDFR | |
| | GRFAFSLETSASTAYLQINNLKYEDTAT | |
| | YFCALDYSYAMDYWGQGTSVTVSS | |
| 212 | DIVLTQSPASLAMSLGKRATISCRASES | Anti-BCMA C11D5.3 |
| | VSVIGAHLIHWYQQKPGQPPKLLIYLAS | scFv light chain variable |
| | NLETGVPARFSGSGSGTDFTLTIDPVEE | region |
| | DDVAIYSCLQSRIFPRTFGGGTKLEIK | |
| 213 | RASESVSVIGAHLIH | Anti-BCMA C11D5.3 |
| | | scFv light chain CDR1 |
| 214 | LASNLET | Anti-BCMA C11D5.3 |
| | | scFv light chain CDR2 |
| 215 | LQSRIFPRT | Anti-BCMA C11D5.3 |
| | | scFv light chain CDR3 |
| 216 | QIQLVQSGPELKKPGETVKISCKASGYT | Anti-BCMA C11D5.3 |
| | FTDYSINWVKRAPGKGLKWMGWINTE | scFv heavy chain variable |
| | TREPAYAYDFRGRFAFSLETSASTAYLQ | region |
| | INNLKYEDTATYFCALDYSYAMDYWG | |
| | QGTSVTVSS | |
| 217 | DYSIN | Anti-BCMA C11D5.3 |
| | | scFv heavy chain CDR1 |
| 218 | WINTETREPAYAYDFRG | Anti-BCMA C11D5.3 |
| | | scFv heavy chain CDR2 |
| 219 | DYSYAMDY | Anti-BCMA C11D5.3 |
| | | scFv heavy chain CDR3 |
| 220 | DIVLTQSPPSLAMSLGKRATISCRASESV | Anti-BCMA C12A3.2 |
| | TILGSHLIYWYQQKPGQPPTLLIQLASN | scFv entire sequence, with |
| | VQTGVPARFSGSGSRTDFTLTIDPVEED | Whitlow linker |
| | DVAVYYCLQSRTIPRTFGGGTKLEIKGS | |
| | TSGSGKPGSGEGSTKGQIQLVQSGPELK | |
| | KPGETVKISCKASGYTFRHYSMNWVK | |
| | QAPGKGLKWMGRINTESGVPIYADDFK | |
| | GRFAFSVETSASTAYLVINNLKDEDTAS | |
| | YFCSNDYLYSLDFWGQGTALTVSS | |
| 221 | DIVLTQSPPSLAMSLGKRATISCRASESV | Anti-BCMA C12A3.2 |
| | TILGSHLIYWYQQKPGQPPTLLIQLASN | scFv light chain variable |
| | VQTGVPARFSGSGSRTDFTLTIDPVEED | region |
| | DVAVYYCLQSRTIPRTFGGGTKLEIK | |
| 222 | RASESVTILGSHLIY | Anti-BCMA C12A3.2 |
| | | scFv light chain CDR1 |
| 223 | LASNVQT | Anti-BCMA C12A3.2 |
| | | scFv light chain CDR2 |
| 224 | LQSRTIPRT | Anti-BCMA C12A3.2 |
| | | scFv light chain CDR3 |
| 225 | QIQLVQSGPELKKPGETVKISCKASGYT | Anti-BCMA C12A3.2 |
| | FRHYSMNWVKQAPGKGLKWMGRINTE | scFv heavy chain variable |
| | SGVPIYADDFKGRFAFSVETSASTAYLV | region |
| | INNLKDEDTASYFCSNDYLYSLDFWGQ | |
| | GTALTVSS | |
| 226 | HYSMN | Anti-BCMA C12A3.2 |
| | | scFv heavy chain CDR1 |
| 227 | RINTESGVPIYADDFKG | Anti-BCMA C12A3.2 |
| | | scFv heavy chain CDR2 |
| 228 | DYLYSLDF | Anti-BCMA C12A3.2 |
| | | scFv heavy chain CDR3 |
| 229 | EVQLLESGGGLVQPGGSLRLSCAASGF | Anti-BCMA FHVH33 |
| | TFSSYAMSWVRQAPGKGLEWVSSISGS | entire sequence |
| | GDYIYYADSVKGRFTISRDISKNTLYLQ | |
| | MNSLRAEDTAVYYCAKEGTGANSSLA | |
| | DYRGQGTLVTVSS | |
| 230 | GFTFSSYA | Anti-BCMA FHVH33 |
| | | CDR1 |
| 231 | ISGSGDYI | Anti-BCMA FHVH33 |
| | | CDR2 |
| 232 | AKEGTGANSSLADY | Anti-BCMA FHVH33 |
| | | CDR3 |
| 233 | DIQMTQSPSSLSASVGDRVTITCRASQS | Anti-BCMA CT103A |
| | ISSYLNWYQQKPGKAPKLLIYAASSLQS | scFv entire sequence, with |
| | GVPSRFSGSGSGTDFTLTISSLQPEDFA | Whitlow linker |
| | TYYCQQKYDLLTFGGGTKVEIKGSTSGS | |
| | GKPGSGEGSTKGQLQLQESGPGLVKPSE | |
| | TLSLTCTVSGGSISSSSYYWGWIRQPPG | |
| | KGLEWIGSISYSGSTYYNPSLKSRVTIS | |
| | VDTSKNQFSLKLSSVTAADTAVYYCAR | |
| | DRGDTILDVWGQGTMVTVSS | |
| 234 | DIQMTQSPSSLSASVGDRVTITCRASQS | Anti-BCMA CT103A |
| | ISSYLNWYQQKPGKAPKLLIYAASSLQS | scFv light chain variable |
| | GVPSRFSGSGSGTDFTLTISSLQPEDFA | region |
| | TYYCQQKYDLLTFGGGTKVEIK | |
| 235 | QSISSY | Anti-BCMA CT103A |
| | | scFv light chain CDR1 |
| 236 | AAS | Anti-BCMA CT103A |
| | | scFv light chain CDR2 |
| 237 | QQKYDLLT | Anti-BCMA CT103A |
| | | scFv light chain CDR3 |
| 238 | QLQLQESGPGLVKPSETLSLTCTVSGGS | Anti-BCMA CT103A |
| | ISSSSYYWGWIRQPPGKGLEWIGSISYS | scFv heavy chain variable |
| | GSTYYNPSLKSRVTISVDTSKNQFSLKL | region |
| | SSVTAADTAVYYCARDRGDTILDVWG | |
| | QGTMVTVSS | |
| 239 | GGSISSSSYY | Anti-BCMA CT103A |
| | | scFv heavy chain CDR1 |
| 240 | ISYSGST | Anti-BCMA CT103A |
| | | scFv heavy chain CDR2 |
| 241 | ARDRGDTILDV | Anti-BCMA CT103A |
| | | scFv heavy chain CDR3 |

In some embodiments, the hinge domain of the BCMA CAR comprises a CD8α hinge domain, for example, a human CD8α hinge domain. In some embodiments, the CD8α hinge domain comprises or consists of an amino acid sequence set forth in SEQ ID NO:212 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:212. In some embodiments, the hinge domain comprises a CD28 hinge domain, for example, a human CD28 hinge domain. In some embodiments, the CD28 hinge domain comprises or consists of an amino acid sequence set forth in SEQ ID NO:213 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:213. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:215 or SEQ ID NO:216, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:215 or SEQ ID NO:216. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:217 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:217.

In some embodiments, the transmembrane domain of the BCMA CAR comprises a CD8α transmembrane domain, for example, a human CD8α transmembrane domain. In some embodiments, the CD8α transmembrane domain comprises or consists of an amino acid sequence set forth in SEQ ID NO: 218 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:218. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO219 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO219.

In some embodiments, the intracellular costimulatory domain of the BCMA CAR comprises a 4-1BB costimulatory domain, for example, a human 4-1BB costimulatory domain. In some embodiments, the 4-1BB costimulatory domain comprises or consists of an amino acid sequence set forth in SEQ ID NO:221 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:221. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:222 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:222.

In some embodiments, the intracellular signaling domain of the BCMA CAR comprises a CD3 zeta (ζ) signaling domain, for example, a human CD3ζ signaling domain. In some embodiments, the CD3ζ signaling domain comprises or consists of an amino acid sequence set forth in SEQ ID NO: 223 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:223.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:212, the CD8α transmembrane domain of SEQ ID NO:218, the 4-1BB costimulatory domain of SEQ ID NO:221, the CD3& signaling domain of SEQ ID NO:223, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8α signal peptide) as described.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:212, the CD8α transmembrane domain of SEQ ID NO:218, the CD28 costimulatory domain of SEQ ID NO:222, the CD3ζ signaling domain of SEQ ID NO:223, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the polycistronic vector comprises an expression cassette that contains a nucleotide sequence encoding a BCMA CAR as set forth in SEQ ID NO:242 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:242 (see Table 14). The encoded BCMA CAR has a corresponding amino acid sequence set forth in SEQ ID NO: 243 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:242, with the following components: CD8α signal peptide, CT103A scFv (V_{L}-Whitlow linker-V_{H}), CD8α hinge domain, CD8α transmembrane domain, 4-1BB costimulatory domain, and CD3ζ signaling domain.

In some embodiments, the polycistronic vector comprises an expression cassette that contains a nucleotide sequence encoding a commercially available embodiment of BCMA CAR, including, for example, idecabtagene vicleucel (ide-cel, also called bb2121). In some embodiments, the polycistronic vector comprises an expression cassette that contains a nucleotide sequence encoding idecabtagene vicleucel or portions thereof. Idecabtagene vicleucel comprises a BCMA CAR with the following components: the BB2121 binder, CD8α hinge domain, CD8α transmembrane domain, 4-1BB costimulatory domain, and CD3ζ signaling domain.

TABLE 14
Exemplary sequences of BCMA CARs

| SEQ | | |
|---|---|---|
| ID | | |
| NO: | Sequence | Description |
| 242 | atggccttaccagtgaccgccttgctcctg | Exemplary |
| | ccgctggccttgctgctccacgccgccagg | BCMA |
| | ccggacatccagatgacccagtctccatcc | CAR |
| | tccctgtctgcatctgtaggagacagagtc | nucleotide |
| | accatcacttgccgggcaagtcagagcatt | sequence |
| | agcagctatttaaattggtatcagcagaaa | |
| | ccagggaaagcccctaagctcctgatctat | |
| | gctgcatccagtttgcaaagtggggtccca | |
| | tcaaggttcagtggcagtggatctgggaca | |
| | gatttcactctcaccatcagcagtctgcaa | |
| | cctgaagattttgcaacttactactgtcag | |
| | caaaaatacgacctcctcacttttggcgga | |
| | gggaccaaggttgagatcaaaggcagcacc | |
| | agcggctccggcaagcctggctctggcgag | |
| | ggcagcacaaagggacagctgcagctgcag | |
| | gagtcgggcccaggactggtgaagccttcg | |
| | gagaccctgtccctcacctgcactgtctct | |
| | ggtggctccatcagcagtagtagttactac | |
| | tggggctggatccgccagcccccagggaag | |
| | gggctggagtggattgggagtatctcctat | |
| | agtgggagcacctactacaacccgtccctc | |
| | aagagtcgagtcaccatatccgtagacacg | |
| | tccaagaaccagttctccctgaagctgagt | |
| | tctgtgaccgccgcagacacggcggtgtac | |
| | tactgcgccagagatcgtggagacaccata | |
| | ctagacgtatggggtcagggtacaatggtc | |
| | accgtcagctcattcgtgcccgtgttcctg | |
| | cccgccaaacctaccaccacccctgcccct | |
| | agacctcccaccccagccccaacaatcgcc | |
| | agccagcctctgtctctgcggcccgaagcc | |
| | tgtagacctgctgccggcggagccgtgcac | |
| | accagaggcctggacttcgcctgcgacatc | |
| | tacatctgggcccctctggccggcacctgt | |
| | ggcgtgctgctgctgagcctggtgatcacc | |
| | ctgtactgcaaccaccggaacaaacggggc | |
| | agaaagaaactcctgtatatattcaaacaa | |
| | ccatttatgagaccagtacaaactactcaa | |
| | gaggaagatggctgtagctgccgatttcca | |
| | gaagaagaagaaggaggatgtgaactgaga | |
| | gtgaagttcagcagatccgccgacgcccct | |
| | gcctaccagcagggacagaaccagctgtac | |
| | aacgagctgaacctgggcagacgggaagag | |
| | tacgacgtgctggacaagcggagaggccgg | |
| | gaccccgagatgggcggaaagcccagacgg | |
| | aagaacccccaggaaggcctgtataacgaa | |
| | ctgcagaaagacaagatggccgaggcctac | |
| | agcgagatcggcatgaagggcgagcggagg | |
| | cgcggcaagggccacgatggcctgtaccag | |
| | ggcctgagcaccgccaccaaggacacctac | |
| | gacgccctgcacatgcaggccctgcccccc | |
| | aga | |
| 243 | MALPVTALLLPLALLLHAARPDIQMTQSPS | Exemplary |
| | SLSASVGDRVTITCRASQSISSYLNWYQQK | BCMA |
| | PGKAPKLLIYAASSLQSGVPSRFSGSGSGT | CAR |
| | DFTLTISSLQPEDFATYYCQQKYDLLTFGG | amino |
| | GTKVEIKGSTSGSGKPGSGEGSTKGQLQLQ | acid |
| | ESGPGLVKPSETLSLTCTVSGGSISSSSYY | sequence |
| | WGWIRQPPGKGLEWIGSISYSGSTYYNPSL | |
| | KSRVTISVDTSKNQFSLKLSSVTAADTAVY | |
| | YCARDRGDTILDVWGQGTMVTVSSFVPVFL | |
| | PAKPTTTPAPRPPTPAPTIASQPLSLRPEA | |
| | CRPAAGGAVHTRGLDFACDIYIWAPLAGTC | |
| | GVLLLSLVITLYCNHRNKRGRKKLLYIFKQ | |
| | PFMRPVQTTQEEDGCSCRFPEEEEGGCELR | |
| | VKFSRSADAPAYQQGQNQLYNELNLGRREE | |
| | YDVLDKRRGRDPEMGGKPRRKNPQEGLYNE | |
| | LQKDKMAEAYSEIGMKGERRRGKGHDGLYQ | |
| | GLSTATKDTYDALHMQALPPR | |

### 3. Genome Modifying Enzymes

In some embodiments, the heterologous protein is associated with a genome editing technology. Any of a variety of agents associated with gene editing technologies can be included as the heterologous protein, such as for delivery of gene editing machinery to a cell. In some embodiments, the gene editing technology can include systems involving nuclease, nickase, homing, integrase, transposase, recombinase, and/or reverse transcriptase activity. In some embodiments, the gene editing technologies can be used for knock-out or knock-down of genes. In some embodiments, the gene-editing technologies can be used for knock-in or integration of DNA into a region of the genome. In some embodiments, the heterologous protein mediates single-strand breaks (SSB). In some embodiments, the heterologous protein mediates double-strand breaks (DSB), including in connection with non-homologous end-joining (NHEJ) or homology-directed repair (HDR). In some embodiments, the heterologous protein does not mediate SSB. In some embodiments, the heterologous protein does not mediate DSB. In some embodiments, the heterologous protein can be used for DNA base editing or prime-editing. In some embodiments, the heterologous protein can be used for Programmable Addition via Site-specific Targeting Elements (PASTE).

In some embodiments, the heterologous protein is a nuclease for use in gene editing methods. In some embodiments, the heterologous protein is a zinc-finger nucleases (ZFNs), transcription-activator like effector nucleases (TALENs), or a CRISPR-associated (Cas) protein. In some embodiments, the Cas protein is selected from the group consisting of Cas3, Cas9, Cas10, Cas12, and Cas13. In some embodiments, the Cas is a Cas12a (also known as cpf1) from a Prevotella, Francisella novicida, Acidaminococcus sp., Lachnospiraceae bacterium, or Francisella bacteria. In some embodiments, the Cas is a Cas12b from a Bacillus, optionally Bacillus hisashii. In some embodiments, the Cas is Cas9 from Streptococcus pyogenes (SpCas). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9). The Cas9 nuclease can, in some embodiments, be a Cas9 or functional fragment thereof from any bacterial species. Sec, e.g., Makarova et al. Nature Reviews, Microbiology, 9:467-477 (2011), including supplemental information, hereby incorporated by reference in its entirety.

In some embodiments, the Cas is wild-type Cas9, which can site-specifically cleave double-stranded DNA, resulting in the activation of the double-strand break (DSB) repair machinery. DSBs can be repaired by the cellular Non-Homologous End Joining (NHEJ) pathway (Overballe-Petersen et al., 2013, Proc Natl Acad Sci USA, Vol. 110:19860-19865), resulting in insertions and/or deletions (indels) which disrupt the targeted locus. Alternatively, if a donor template with homology to the targeted locus is supplied, the DSB may be repaired by the homology-directed repair (HDR) pathway allowing for precise replacement mutations to be made (Overballe-Petersen et al., 2013, Proc Natl Acad Sci USA, Vol. 110:19860-19865; Gong et al., 2005, Nat. Struct Mol Biol, Vol. 12:304-312). In some embodiments, the Cas is mutant form, known as Cas9 D10A, with only nickase activity. This means that Cas9D10A cleaves only one DNA strand, and does not activate NHEJ. Instead, when provided with a homologous repair template, DNA repairs are conducted via the high-fidelity HDR pathway only, resulting in reduced indel mutations (Cong et al., 2013, Science, Vol. 339:819-823; Jinek et al., 2012, Science, Vol. 337:816-821; Qi et al., 2013 Cell, Vol. 152:1173-1183), Cas9D10A is even more appealing in terms of target specificity when loci are targeted by paired Cas9 complexes designed to generate adjacent DNA nicks (Ran et al., 2013, Cell, Vol. 154:1380-1389). In some embodiments, the Cas is a nuclease-deficient Cas9 (Qi et al., 2013 Cell, Vol. 152:1173-1183). For instance, mutations H840A in the HNH domain and D10A in the RuvC domain inactivate cleavage activity, but do not prevent DNA binding. Therefore, this variant can be used to target in a sequence-specific manner any region of the genome without cleavage. Instead, by fusing with various effector domains, dCas9 can be used either as a gene silencing or activation tools. Furthermore, it can be used as a visualization tool by coupling the guide RNA or the Cas9 protein to a fluorophore or a fluorescent protein. In some embodiments, the Cas protein comprises one or more mutations such that the Cas protein is converted into a nickase that is able to cleave only one strand of a double stranded DNA molecule (e.g., a SSB). In some embodiments, the Cas protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Csc2, Csf1, Csm2, Csn2, Csx10, Csx11, Csy1, Csy2, Csy3, and Mad7. In some embodiments, the Cas protein is Cas9. In some embodiments, the Cas9 is from a bacteria selected from the group consisting of Streptococcus pyogenes, Staphylococcus aureus, Neisseria meningitides, Campylobacter jejuni, and Streptococcus thermophilis. In some embodiments, the Cas9 is from Streptococcus pyogenes. In some embodiments, the Cas9 is from Streptococcus pyogenes and comprises one or more mutations in the RuvC I, RuvC II, or RuvC III motifs. In some embodiments, the Cas9 is from Streptococcus pyogenes and comprises a D10A mutation in the RuvC I motif. In some embodiments, the Cas9 is from Streptococcus pyogenes and comprises one or more mutations in the HNH catalytic domain. In some embodiments, the Cas9 is from Streptococcus pyogenes and comprises one or more mutations in the HNH catalytic domain selected from the group consisting of H840A, H854A, and H863A. In some embodiments, the Cas9 is from Streptococcus pyogenes and comprises a H840A mutation in the HNH catalytic domain. In some embodiments, the Cas9 is from Streptococcus pyogenes and comprises a mutation selected from the group consisting of D10A. H840A, H854A, and H863A.

In some embodiments, the Cas protein is selected from the group consisting of Cas3, Cas9, Cas10, Cas12, and Cas13. In some embodiments, the Cas protein is Cas9. In some embodiments, the one or more agent(s) (e.g., the heterologous protein) capable of inducing a DSB comprise Cas9 or a functional fragment thereof, and a first guide RNA, e.g., a first sgRNA, and a second guide RNA, e.g., a second sgRNA. The guide RNA, e.g., the first guide RNA or the second guide RNA, in some embodiments, binds to the recombinant nuclease and targets the recombinant nuclease to a specific location within the target gene such as at a location within the sense strand or the antisense strand of the target gene that is or includes the cleavage site. In some embodiments, the recombinant nuclease is a Cas protein from any bacterial species, or is a functional fragment thereof. In some embodiments, the Cas protein is Cas9 nuclease, Cas9 can, in some embodiments, be a Cas9 or functional fragment thereof from any bacterial species. See, e.g., Makarova et al. Nature Reviews, Microbiology, 9:467-477 (2011), including supplemental information, hereby incorporated by reference in its entirety. In some embodiments, the Cas9 is from Streptococcus pyogenes (SpCas9). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9).

In some embodiments, the Cas9 is from Streptococcus pyogenes (SpCas9) and comprises one or more mutations in the RuvC catalytic domain or the HNH catalytic domain. In some embodiments, the one or more mutations in the RuvC catalytic domain or the HNH catalytic domain inactivates the catalytic activity of the domain. In some embodiments, the recombinant nuclease has RuvC activity but does not have HNH activity. In some embodiments, the recombinant nuclease does not have RuvC activity but does have HNH activity. In some embodiments, the Cas9 is from Streptococcus pyogenes (SpCas9) and comprises one or more mutations selected from the group consisting of D10A, H840A, H854A, and H863A. In some embodiments, the Cas9 is from Streptococcus pyogenes (SpCas9) and comprises one or more mutations in the RuvC I, RuvC II, or RuvC III motifs. In some embodiments, the Cas9 is from Streptococcus pyogenes (SpCas9) and comprises a mutation in the RuvC I motif. In some embodiments, the Cas9 is from Streptococcus pyogenes (SpCas9) and comprises a D10A mutation in the RuvC I motif. In some embodiments, the Cas9 is from Streptococcus pyogenes (SpCas9) and comprises one or more mutations in the HNH catalytic domain. In some embodiments, the one or more mutations in the HNH catalytic domain is selected from the group consisting of H840A, H854A, and H863A. In some embodiments, the Cas9 is from Streptococcus pyogenes (SpCas9) and comprises a H840A mutation in the HNH catalytic domain. In some embodiments, the Cas9 is from Streptococcus pyogenes (SpCas9) and comprises a H840A mutation. In some embodiments, the Cas9 is from Streptococcus pyogenes (SpCas9) and comprises a D10A mutation. In some embodiments, the Cas9 is from Streptococcus pyogenes (SpCas9) and comprises one or more mutations selected from the group consisting of N497A, R661A, Q695A, and Q926A. In some embodiments, the Cas9 is from Streptococcus pyogenes (SpCas9) and comprises one or more mutations selected from the group consisting of R780A, K810A, K855A, H982A, K1003A, R1060A, and K848A. In some embodiments, the Cas9 is from Streptococcus pyogenes (SpCas9) and comprises one or more mutations selected from the group consisting of N692A, M694A, Q695A, and H698A. In some embodiments, the Cas9 is from Streptococcus pyogenes (SpCas9) and comprises one or more mutations selected from the group consisting of M495V, Y515N, K526E, and R661Q. In some embodiments, the Cas9 is from Streptococcus pyogenes (SpCas9) and comprises one or more mutations selected from the group consisting of F539S, M763I, and K890N. In some embodiments, the Cas9 is from Streptococcus pyogenes (SpCas9) and comprises one or more mutations selected from the group consisting of E480K, E543D, E1219V, A262T, S409I, M694I, E108G, S217A.

In some embodiments, the Cas9 is from Streptococcus pyogenes (SaCas9). In some embodiments, the SaCas9 is wild type SaCas9. In some embodiments, the SaCas9 comprises one or more mutations in REC3 domain. In some embodiments, the SaCas9 comprises one or more mutations in RECI domain. In some embodiments, the SaCas9 comprises one or more mutations selected from the group consisting of N260D, N260Q, N260E, Q414A, Q414L. In some embodiments, the SaCas9 comprises one or more mutations in the recognition lobe. In some embodiments, the SaCas9 comprises one or more mutations selected from the group consisting of R245A, N413A, N419A. In some embodiments, the SaCas9 comprises one or more mutations in the RuvC-III domain. In some embodiments, the SaCas9 comprises a R654A mutation.

In some embodiments, the Cas protein is Cas12. In some embodiments, the Cas protein is Cas12a (i.e. cpf1). In some embodiments, the Cas12a is from the group consisting of Francisella novicida U112 (FnCas12a), Acidaminococcus sp. BV3L6 (AsCas12a), Moraxella bovoculi AAX11_00205 (Mb3Cas12a), Lachnospiraceae bacterium ND2006 (LbCas12a), Thiomicrospira sp. Xs5 (TsCas12a), Moraxella bovoculi AAX08_00205 (Mb2Cas12a), and Butyrivibrio sp. NC3005 (BsCas12a). In some embodiments, the Cas12a recognizes a T-rich 5′ protospacer adjacent motif (PAM). In some embodiments, the Cas12a processes its own crRNA without requiring a transactivating crRNA (tracrRNA). In some embodiments, the Cas12a processes both RNase and DNase activity. In some embodiments, the Cas12a is a split Cas12a platform, consisting of N-terminal and C-terminal fragments of Cas12a. In some embodiments, the split Cas12a platform is from Lachnospiraceae bacterium.

In some embodiments, the lipid particle further comprises a polynucleotide per se, i.e. a polynucleotide that does not encode for a heterologous protein. In some embodiments, the polynucleotide per se is associated with a gene editing system. For example, a lipid particle may comprise a guide RNA (gRNA), such as a single guide RNA (sgRNA).

In some embodiments, the one or more agent(s) (e.g., the heterologous protein) comprise, or are used in combination with, a guide RNA, e.g., single guide RNA (sgRNA), for inducing a DSB at the cleavage site. In some embodiments, the one or more agent(s) comprise, or are used in combination with, more than one guide RNA, e.g., a first sgRNA and a second sgRNA, for inducing a DSB at the cleavage site through a SSB on each strand. In some embodiments, the one or more agent(s) (e.g., the heterologous protein) can be used in combination with a donor template, e.g., a single-stranded DNA oligonucleotide (ssODN), for HDR-mediated integration of the donor template into the target gene, such as at the targeting sequence. In some embodiments, the one or more agent(s) (e.g., the heterologous protein) can be used in combination with a donor template, e.g., an ssODN, and a guide RNA, e.g., a sgRNA, for HDR-mediated integration of the donor template into the target gene, such as at the targeting sequence. In some embodiments, the one or more agent(s) (e.g., the heterologous protein) can be used in combination with a donor template, e.g., an ssODN, and a first guide RNA, e.g., a first sgRNA, and a second guide RNA, e.g., a second sgRNA, for HDR-mediated integration of the donor template into the target gene, such as at the targeting sequence.

In particular embodiments, the genome-modifying agent is a Cas protein, such as Cas9. In some embodiments, delivery of the CRISPR/Cas can be used to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. In some embodiments, a dCas9 version of the CRISPR/Cas9 system can be used to target protein domains for transcriptional regulation, epigenetic modification, and microscopic visualization of specific genome loci.

In some embodiments, the genome-modifying agent, e.g., Cas9, is targeted to the cleavage site by interacting with a guide RNA, e.g., sgRNA, that hybridizes to a DNA sequence that immediately precedes a Protospacer Adjacent Motif (PAM) sequence. In general, a guide RNA, e.g., sgRNA, is any nucleotide sequence comprising a sequence, e.g., a crRNA sequence, that has sufficient complementarity with a target gene sequence to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., sgRNA. In some embodiments, the cleavage site is situated at a site within the target gene that is homologous to the sequence of the guide RNA, e.g., sgRNA. In some embodiments, the cleavage site is situated approximately 3 nucleotides upstream of the PAM sequence. In some embodiments, the cleavage site is situated approximately 3 nucleotides upstream of the juncture between the guide RNA and the PAM sequence. In some embodiments, the cleavage site is situated 3 nucleotides upstream of the PAM sequence. In some embodiments, the cleavage site is situated 4 nucleotides upstream of the PAM sequence.

In some embodiments, the one or more agent(s) (e.g., the heterologous protein) capable of inducing a DSB comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fok1 nuclease domain. In some embodiments, the TAL effector DNA binding domain is engineered to target a specific target sequence, e.g., a portion of a target gene that includes a cleavage site.

In some embodiments, the fusion protein is a zinc finger nuclease (ZFN) comprising a zinc finger DNA binding domain and a DNA cleavage domain. In some embodiments, the DNA cleavage domain is a Fok1 nuclease domain. In some embodiments, the zinc finger DNA binding domain is engineered to target a specific target sequence, e.g., a portion of a target gene, that includes a cleavage site, such as the targeting sequence.

In some embodiments, the method involves introducing, into a cell, one or more agent(s) (e.g., the heterologous protein) capable of inducing a SSB at a cleavage site within the sense strand and a SSB at a cleavage site within the antisense strand of an endogenous target gene in the cell.

In some embodiments, the cleavage site in the sense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the antisense strand. In some embodiments, the cleavage site in the antisense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the sense strand. In some embodiments, the cleavage site in the sense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the antisense strand. In some embodiments, the cleavage site in the antisense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the sense strand.

In some embodiments, the one or more agent(s) (e.g., the heterologous protein) capable of inducing a SSB at a cleavage site within the sense strand and a SSB at a cleavage site within the antisense strand comprise a recombinant nuclease. In some embodiments, the recombinant nuclease includes a recombinant nuclease that induces the SSB in the sense strand, and a recombinant nuclease that induced the SSB in the antisense strand, and both of which recombinant nucleases are referred to as the recombinant nuclease. Accordingly, in some embodiments, the method involves introducing, into a cell, one or more agent(s) (e.g., the heterologous protein) comprising a recombinant nuclease for inducing a SSB at a cleavage site in the sense strand and a SSB at a cleavage site in the antisense strand within an endogenous target gene in the cell. Although, in some embodiments, it is described that “a” “the” recombinant nuclease induces a SSB in the antisense strand a SSB in the sense strand, it is to be understood that this includes situations where two of the same recombinant nuclease is used, such that one of the recombinant nuclease induces the SSB in the sense strand and the other recombinant nuclease induces the SSB in the antisense strand. In some embodiments, the recombinant nuclease that induces the SSB lacks the ability to induce a DSB by cleaving both strands of double stranded DNA.

In some embodiments, the one or more agent(s) capable of inducing a SSB comprise a recombinant nuclease and a first guide RNA, e.g., a first sgRNA, and a second guide RNA, e.g., a second sgRNA.

In some embodiments, the genome-modifying agent is a Cas protein, a transcription activator-like effector nuclease (TALEN), or a zinc finger nuclease (ZFN). In some embodiments, the recombinant nuclease is a Cas nuclease. In some embodiments, the recombinant nuclease is a TALEN. In some embodiments, the recombinant nuclease is a ZFN.

In some embodiments, the one or more agent(s) capable of inducing a SSB at a cleavage site within the sense strand and a SSB at a cleavage site within the antisense strand comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fok1 nuclease domain. In some embodiments, the TAL effector DNA binding domain is engineered to target a specific target sequence, e.g., a portion of a target gene that includes a cleavage site. In some embodiments, the fusion protein is a zinc finger nuclease (ZFN) comprising a zinc finger DNA binding domain and a DNA cleavage domain. In some embodiments, the DNA cleavage domain is a Fok1 nuclease domain. In some embodiments, the zinc finger DNA binding domain is engineered to target a specific target sequence, e.g., a portion of a target gene that includes a cleavage site, such as the targeting sequence.

In some embodiments, the one or more agent(s) capable of inducing a SSB at a cleavage site within the sense strand and a SSB at a cleavage site within the antisense strand involve use of the CRISPR/Cas gene editing system. In some embodiments, the one or more agent(s) comprise a recombinant nuclease.

In some embodiments, the genome-modifying agent is a Cas protein. In some embodiments, the Cas protein comprises one or more mutations such that the Cas protein is converted into a nickase that lacks the ability to cleave both strands of a double stranded DNA molecule. In some embodiments, the Cas protein comprises one or more mutations such that the Cas protein is converted into a nickase that is able to cleave only one strand of a double stranded DNA molecule. For example, Cas9, which is normally capable of inducing a double strand break, can be converted into a Cas9 nickase, which is capable of inducing a single strand break, by mutating one of two Cas9 catalytic domains: the RuvC domain, which comprises the RuvC I, RuvC II, and RuvC III motifs, or the NHN domain. In some embodiments, the Cas protein comprises one or more mutations in the RuvC catalytic domain or the HNH catalytic domain. In some embodiments, the genome-modifying protein is a recombinant nuclease that has been modified to have nickase activity. In some embodiments, the recombinant nuclease cleaves the strand to which the guide RNA, e.g., sgRNA, hybridizes, but does not cleave the strand that is complementary to the strand to which the guide RNA. e.g., sgRNA, hybridizes. In some embodiments, the recombinant nuclease does not cleave the strand to which the guide RNA, e.g., sgRNA, hybridizes, but does cleave the strand that is complementary to the strand to which the guide RNA, e.g., sgRNA, hybridizes.

In some embodiments, the lipid particle further comprises a guide RNA (gRNA), such as a single guide RNA (sgRNA). Thus, in some embodiments, the heterologous agent comprises a guide RNA (gRNA). In some embodiments, the gRNA is a single guide RNA (sgRNA).

In some embodiments, the genome-modifying protein, e.g., Cas9, is targeted to the cleavage site by interacting with a guide RNA, e.g., a first guide RNA, such as a first sgRNA, or a second guide RNA, such as a second sgRNA, that hybridizes to a DNA sequence on the sense strand or the antisense strand that immediately precedes a Protospacer Adjacent Motif (PAM) sequence.

In some embodiments, the genome-modifying agent, e.g., Cas9, is targeted to the cleavage site on the sense strand by interacting with a first guide RNA, e.g., first sgRNA, that hybridizes to a sequence on the sense strand that immediately precedes a PAM sequence. In some embodiments, the genome-modifying agent, e.g., Cas9, is targeted to the cleavage site on the antisense strand by interacting with a second guide RNA, e.g., second sgRNA, that hybridizes to a sequence on the antisense strand that immediately precedes a PAM sequence.

In some embodiments, the first guide RNA, e.g., first sgNA, that is specific to the sense strand of a target gene of interest is used to target the recombinant nuclease, e.g., Cas9, to induce a SSB at a cleavage site within the sense strand of the target gene. In some embodiments, the first guide RNA, e.g., first sgNA, that is specific to the antisense strand of a target gene of interest is used to target the recombinant nuclease, e.g., Cas9, to induce a SSB at a cleavage site within the antisense strand of the target gene.

In some embodiments, the second guide RNA, e.g., second sgNA, that is specific to the sense strand of a target gene of interest used to target the recombinant nuclease, e.g., Cas9, to induce a SSB at a cleavage site within the sense strand of the target gene. In some embodiments, the second guide RNA, e.g., second sgNA, that is specific to the antisense strand of a target gene of interest is used to target the recombinant nuclease, e.g., Cas9, to induce a SSB at a cleavage site within the antisense strand of the target gene.

In some embodiments, the first guide RNA, e.g., first sgNA, that is specific to the sense strand of a target gene of interest is used to target the recombinant nuclease, e.g., Cas9, to induce a SSB at a cleavage site within the sense strand of the target gene; and the second guide RNA, e.g., second sgNA, that is specific to the antisense strand of a target gene of interest is used to target the recombinant nuclease, e.g., Cas9, to induce a SSB at a cleavage site within the antisense strand of the target gene.

In some embodiments, the first guide RNA, e.g., first sgNA, that is specific to the antisense strand of a target gene of interest is used to target the recombinant nuclease, e.g., Cas9, to induce a SSB at a cleavage site within the antisense strand of the target gene; and the second guide RNA, e.g., second sgNA, that is specific to the sense strand of a target gene of interest is used to target the recombinant nuclease, e.g., Cas9, to induce a SSB at a cleavage site within the sense strand of the target gene. In general, a guide RNA, e.g., a first guide RNA, such as a first sgRNA, or a second guide RNA, such as a second sgRNA, is any nucleotide sequence comprising a sequence, e.g., a crRNA sequence, that has sufficient complementarity with a target gene sequence to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., the first guide RNA, such as the first sgRNA, or the second guide RNA, such as the second sgRNA.

In some embodiments, the cleavage site is situated at a site within the target gene that is homologous to a sequence comprised within the guide RNA, e.g., sgRNA. In some embodiments, the cleavage site of the sense strand is situated at a site within the sense strand of the target gene that is homologous to a sequence comprised within the first guide RNA, e.g., the first sgRNA. In some embodiments, the cleavage site of the antisense strand is situated at a site within the antisense strand of the target gene that is homologous to a sequence comprised within the first guide RNA, e.g., the first sgRNA. In some embodiments, the cleavage site of the sense strand is situated at a site within the sense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the second guide RNA, e.g., the second sgRNA. In some embodiments, the cleavage site of the sense strand is situated at a site within the sense strand of the target gene that is homologous to a sequence comprised within the first guide RNA, e.g., the first sgRNA; and the cleavage site of the antisense strand is situated at a site within the antisense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the first guide RNA, e.g., the first sgRNA; and the cleavage site of the sense strand is situated at a site within the sense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the second guide RNA, e.g., the second sgRNA; and the cleavage site of the sense strand is situated at a site within the sense strand of the target gene that is homologous to a sequence comprised within the first guide RNA, e.g., the first sgRNA.

In some embodiments, the sense strand comprises the targeting sequence, and the targeting sequence includes the SNP and a protospacer adjacent motif (PAM) sequence. In some embodiments, the sense strand comprises the targeting sequence, and the targeting sequence includes the SNP and a protospacer adjacent motif (PAM) sequence; and the antisense strand comprises a sequence that is complementary to the targeting sequence and includes a PAM sequence. In some embodiments, the antisense strand comprises the targeting sequence, and the targeting sequence includes the SNP and a protospacer adjacent motif (PAM) sequence. In some embodiments, the antisense strand comprises the targeting sequence, and the targeting sequence includes the SNP and a protospacer adjacent motif (PAM) sequence; and the sense strand comprises a sequence that is complementary to the targeting sequence and includes a PAM sequence.

In some embodiments, the cleavage site on the sense strand and/or the antisense strand is situated approximately 3 nucleotides upstream of the PAM sequence. In some embodiments, the cleavage site on the sense strand and/or the antisense strand is situated approximately 3 nucleotides upstream of the juncture between the guide RNA and the PAM sequence. In some embodiments, the cleavage site on the sense strand and/or the antisense strand is situated 3 nucleotides upstream of the PAM sequence. In some embodiments, the cleavage site on the sense strand and/or the antisense strand is situated 4 nucleotides upstream of the PAM sequence.

In some embodiments, the PAM sequence that is recognized by a recombinant nuclease is in the sense strand. In some embodiments, the PAM sequence that is recognized by a recombinant nuclease is in the antisense strand. In some embodiments, the PAM sequence that is recognized by a recombinant nuclease is in the sense strand and is in the antisense strand. In some embodiments, the PAM sequence on the sense strand and the PAM sequence on the antisense strand are outwardly facing. In some embodiments, the the PAM sequence on the sense strand and the PAM sequence on the antisense strand comprise the same nucleic acid sequence, which can be any PAM sequence disclosed herein. In some embodiments, the the PAM sequence on the sense strand and the PAM sequence on the antisense strand each comprise a different nucleic acid sequence, each of which can be any of the PAM sequences disclosed herein.

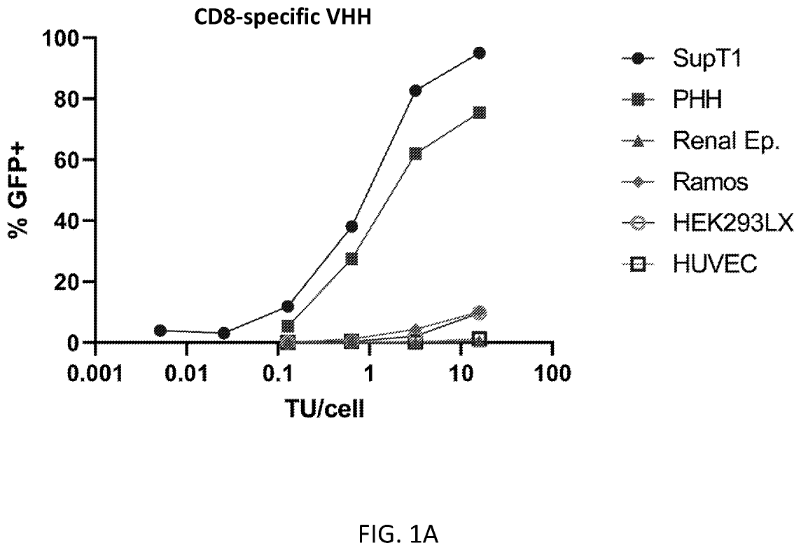

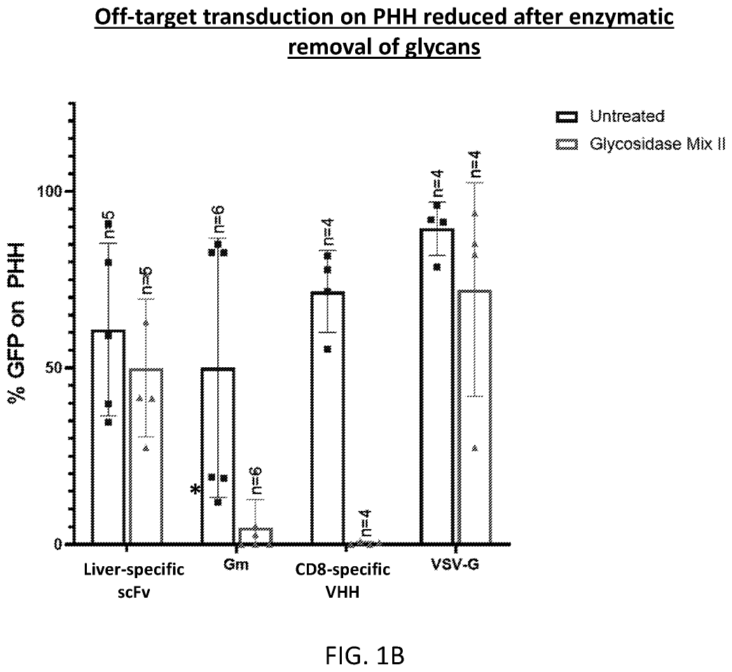

In some embodiments, the PAM sequence that is recognized by a recombinant nuclease, e.g., Cas9, differs depending on the particular recombinant nuclease and the bacterial species it is from Methods for designing guide RNAs, e.g., sgRNAs, and their exemplary targeting sequences, e.g., crRNA sequences, can include those described in, e.g., International PCT Pub. Nos. WO2015/161276, WO2017/193107, and WO2017/093969. Exemplary guide RNA structures, including particular domains, are described in WO2015/161276, e.g., in FIGS. 1A-1G therein. Since guide RNA is an RNA molecule, it will comprise the base uracil (U), while any DNA encoding the guide RNA molecule will comprise the base thymine (T). In some embodiments, the guide RNA, e.g., sgRNA, comprises a CRISPR targeting RNA sequence (crRNA) and a trans-activating crRNA sequence (tracrRNA). In some embodiments, the first guide RNA, e.g., the first sgRNA, and the second guide RNA, e.g., the second sgRNA, each comprise a crRNA and a tracrRNA. In some embodiments, the guide RNA, e.g., sgRNA, is an RNA comprising, from 5′ to 3′: a crRNA sequence and a tracrRNA sequence. In some embodiments, each of the first guide RNA, e.g., first sgRNA, and the second guide RNA, e.g., second sgRNA, is an RNA comprising, from 5′ to 3′: a crRNA sequence and a tracrRNA sequence. In some embodiments, the crRNA and tracrRNA do not naturally occur together in the same sequence.

In some embodiments, the crRNA comprises a nucleotide sequence that is homologous, e.g., is at least 80%, 85%, 90%, 95%, 96%, 97%, 98%, or 99% homologous, or is 100% homologous, to a portion of the target gene that includes the cleavage site. In some embodiments, the crRNA comprises a nucleotide sequence that is 100% homologous to a portion of the target gene that includes the cleavage site. In some embodiments, the portion of the target gene that includes the cleavage site is a portion of the sense strand of the target gene that includes the cleavage site. In some embodiments, the portion of the target gene that includes the cleavage site is a portion of the antisense strand of the target gene that includes the cleavage site.

In some embodiments, the sgRNA comprises a crRNA sequence that is homologous to a sequence in the target gene that includes the cleavage site. In some embodiments, the first sgRNA comprises a crRNA sequence that is homologous to a sequence in the sense strand of the target gene that includes the cleavage site; and/or the second sgRNA comprises a crRNA sequence that is homologous to a sequence in the antisense strand of the target gene that includes the cleavage site. In some embodiments, the first sgRNA comprises a crRNA sequence that is homologous to a sequence in the antisense strand of the target gene that includes the cleavage site; and/or the second sgRNA comprises a crRNA sequence that is homologous to a sequence in the sense strand of the target gene that includes the cleavage site.

In some embodiments, the crRNA sequence has 100% sequence identity to a sequence in the target gene that includes the cleavage site. In some embodiments, the crRNA sequence of the first sgRNA has 100% sequence identity to a sequence in the sense strand of the target gene that includes the cleavage site; and/or the crRNA sequence of the second sgRNA has 100% sequence identity to a sequence in the antisense strand of the target gene that includes the cleavage site. In some embodiments, the crRNA sequence of the first sgRNA has 100% sequence identity to a sequence in the antisense strand of the target gene that includes the cleavage site; and/or the crRNA sequence of the second sgRNA has 100% sequence identity to a sequence in the sense strand of the target gene that includes the cleavage site.

Guidance on the selection of crRNA sequences can be found, e.g., in Fu Y et al., Nat Biotechnol 2014 (doi: 10.1038/nbt.2808) and Sternberg S H et al., Nature 2014 (doi: 10.1038/nature13011). Examples of the placement of crRNA sequences within the guide RNA, e.g., sgRNA, structure include those described in WO2015/161276, e.g., in FIGS. 1A-1G therein.

Reference to “the crRNA” is to be understood as also including reference to the crRNA of the first sgRNA and the crRNA of the second sgRNA, each independently. Thus, embodiments referring to “the crRNA” is to be understood as independently referring to embodiments of (i) the crRNA. (ii) the crRNA of the first sgRNA, and (iii) the crRNA of the second sgRNA. In some embodiments, the crRNA is 15-27 nucleotides in length, i.e., the crRNA is 15, 16, 17, 18, 19, 20, 21, 22, 23, 24, 25, 26, or 27 nucleotides in length. In some embodiments, the crRNA is 18-22 nucleotides in length. In some embodiments, the crRNA is 19-21 nucleotides in length. In some embodiments, the crRNA is 20 nucleotides in length.

In some embodiments, the crRNA is homologous to a portion of a target gene that includes the cleavage site. In some embodiments, the crRNA is homologous to a portion of the sense strand of the target gene that includes the cleavage site. In some embodiments, the crRNA is homologous to a portion of the antisense strand of the target gene that includes the cleavage site. In some embodiments, the crRNA of the first sgRNA is homologous to a portion of the sense strand of the target gene that includes the cleavage site; and the crRNA of the second sgRNA is homologous to a portion of the antisense strand of the target gene that includes the cleavage site.

In some embodiments, the crRNA is homologous to a portion of the antisense strand of a target gene that includes the cleavage site. In some embodiments, the crRNA is homologous to a portion of the sense strand of the target gene that includes the cleavage site. In some embodiments, the crRNA of the first sgRNA is homologous to a portion of the antisense strand of the target gene that includes the cleavage site; and the crRNA of the second sgRNA is homologous to a portion of the sense strand of the target gene that includes the cleavage site.

In some embodiments, the crRNA is homologous to a portion of a target gene that includes the cleavage site, and is 15-27 nucleotides in length, i.e., the crRNA is 15, 16, 17, 18, 19, 20, 21, 22, 23, 24, 25, 26, or 27 nucleotides in length. In some embodiments, the portion of the target gene that includes the cleavage site is on the sense strand. In some embodiments, the portion of the target gene that includes the cleavage site is on the antisense strand.

In some embodiments, the crRNA is homologous to a portion, i.e., sequence, in the sense strand or the antisense strand of the target gene that includes the cleavage site and is immediately upstream of the PAM sequence.

In some embodiments, the tracrRNA sequence may be or comprise any sequence for tracrRNA that is used in any CRISPR/Cas9 system known in the art. Reference to “the tracrRNA” is to be understood as also including reference to the tracrRNA of the first sgRNA and the tracrRNA of the second sgRNA, each independently. Thus, embodiments referring to “the tracrRNA” is to be understood as independently referring to embodiments of (i) the tracrRNA, (ii) the tracrRNA of the first sgRNA, and (iii) the tracrRNA of the second sgRNA. Exemplary CRISPR/Cas9 systems, sgRNA, crRNA, and tracrRNA, and their manufacturing process and use include those described in, e.g., International PCT Pub. Nos. WO2015/161276, WO2017/193107 and WO2017/093969, and those described in, e.g., U.S. Patent Application Publication Nos. 20150232882, 20150203872, 20150184139, 20150079681, 20150073041, 20150056705, 20150031134, 20150020223, 20140357530, 20140335620, 20140310830, 20140273234, 20140273232, 20140273231, 20140256046, 20140248702, 20140242700, 20140242699, 20140242664, 20140234972, 20140227787, 20140189896, 20140186958, 20140186919, 20140186843, 20140179770, 20140179006, 20140170753, 20140093913, and 20140080216.

In some embodiments, the heterologous protein is associated with base editing. Base editors (BEs) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some cases, base editors may also include proteins or domains that alter cellular DNA repair processes to increase the efficiency and/or stability of the resulting single-nucleotide change.

In some aspects, currently available base editors include cytidine base editors (e.g., BE4) that convert target C⋅G to T⋅A and adenine base editors (e.g., ABE7.10) that convert target A⋅T to G⋅C. In some aspects, Cas9-targeted deamination was first demonstrated in connection with a Base Editor (BE) system designed to induce base changes without introducing double-strand DNA breaks. Further Rat deaminase APOBEC1 (rAPOBEC1) fused to deactivated Cas9 (dCas9) was used to successfully convert cytidines to thymidines upstream of the PAM of the sgRNA. In some aspects, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U: A base pair, which is then converted to T: A during DNA replication.

In some embodiments, the heterologous protein is or encodes a base editor (e.g., a nucleobase editor). In some embodiments, the heterologous protein is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, the base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, the base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, the base editor is a adenine-to-thymine or “ATBE” (or thymine-to-adenine or “TABE”) transversion base editors. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, the heterologous protein is one for use in target-primed reverse transcription (TPRT) or “prime editing”. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors search and locate the desired target site to be edited, and encode a replacement strand containing a desired edit which is installed in place of the corresponding target site endogenous DNA strand at the same time. For example, prime editing can be adapted for conducting precision CRISPR/Cas-based genome editing in order to bypass double stranded breaks. In some embodiments, the heterologous protein is or encodes for a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. In some embodiments, the prime editor protein is paired with two prime editing guide RNAs (pegRNAs) that template the synthesis of complementary DNA flaps on opposing strands of genomic DNA, resulting in the replacement of endogenous DNA sequence between the PE-induced nick sites with pegRNA-encoded sequences.

In some embodiments, the heterologous protein is or encodes for a primer editor that is a reverse transcriptase, or any DNA polymerase known in the art. Thus, in one aspect, the prime editor may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. Such methods include any disclosed in Anzalone et al., (doi.org/10.1038/s41586-019-1711-4), or in PCT publication Nos. WO2020191248, WO2021226558, or WO2022067130, which are hereby incorporated in their entirety.

In some embodiments, the heterologous protein is for use in Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks, but allowed for integration of sequences as large as ˜36 kb. In some embodiments, the serine integrase can be any known in the art. In some embodiments, the serine integrase has sufficient orthogonality such that PASTE can be used for multiplexed gene integration, simultaneously integrating at least two different genes at at least two genomic loci. In some embodiments, PASTE has editing efficiencies comparable to or better than those of homology directed repair or non-homologous end joining based integration, with activity in nondividing cells and fewer detectable off-target events.

In some embodiments, the heterologous protein is or encodes one or more polypeptides having an activity selected from the group consisting of: nuclease activity (e.g., programmable nuclease activity); nickase activity (e.g., programmable nickase activity); homing activity (e.g., programmable DNA binding activity); nucleic acid polymerase activity (e.g., DNA polymerase or RNA polymerase activity); integrase activity; recombinase activity; or base editing activity (e.g., cytidine deaminase or adenosine deaminase activity).

In some embodiments, delivery of the nuclease is by a provided vector encoding the nuclease (e.g, Cas).

In some embodiments, the provided lipid particles contain a nuclease protein and the nuclease protein is directly delivered to a target cell. Methods of delivering a nuclease protein include those as described, for example, in Cai et al. Elife, 2014, 3: e01911 and International patent publication No. WO2017068077. For instance, provided lipid particles comprise one or more Cas protein(s), such as Cas9. In some embodiments, the nuclease protein (e.g, Cas, such as Cas 9) is engineered as a chimeric nuclease protein with a viral structural protein (e.g. GAG) for packaging into the lipid particle (e.g. lentiviral vector particle, VLP, or gesicle). For instance, a chimeric Cas9-protein fusion with the structural GAG protein can be packaged inside a lipid particle. In some embodiments, the fusion protein is a cleavable fusion protein between (i) a viral structural protein (e.g. GAG) and (ii) a nuclease protein (e.g, Cas protein, such as Cas9). In some embodiments, the fusion protein is a cleavable fusion protein between (i) a viral matrix (MA) protein and (ii) a nuclease protein (e.g, Cas protein, such as Cas9). In some embodiments, the particle contains a nuclease protein (e.g., Cas protein, such as Cas 9) immediately downstream of the gag start codon.

In some embodiments, the provided lipid particles contain mRNA encoding a Cas nuclease (e.g., Cas9). In some embodiments, the provided lipid particles contain guide RNA (gRNA), such as a single guide RNA (sgRNA).

In some embodiments, the provided lipidparticles (e.g. lentiviral particles, VLPs, or gesicles) containing a Cas nuclease (e.g, Cas9) further comprise, or is further complexed with, one or more CRISPR-Cas system guide RNA(s) for targeting a desired target gene. In some embodiments, the CRISPR guide RNAs are efficiently encapsulated in the CAS-containing lipid particles. In some embodiments, the provided lipid particles (e.g. lentiviral particles, VLPs, or gesicles) further comprises, or is further complexed with a targeting nucleic acid.

### 4. Small Molecules

In some embodiments, the exogenous agent includes a small molecule, e.g., ions (e.g. Ca²⁺, Cl—, Fe²⁺), carbohydrates, lipids, reactive oxygen species, reactive nitrogen species, isoprenoids, signaling molecules, heme, polypeptide cofactors, electron accepting compounds, electron donating compounds, metabolites, ligands, and any combination thereof. In some embodiments the small molecule is a pharmaceutical that interacts with a target in the cell. In some embodiments the small molecule targets a protein in the cell for degradation. In some embodiments the small molecule targets a protein in the cell for degradation by localizing the protein to the proteasome. In some embodiments that small molecule is a proteolysis targeting chimera molecule (PROTAC).

In some embodiments, the exogenous agent includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g, Cas9-gRNA complex); multiple transcription factors, multiple epigenetic factors, reprogramming factors (e.g. Oct4, Sox2, cMyc, and Klf4); multiple regulatory RNAs; and any combination thereof.

### IV. Pharmaceutical Compositions

Also provided are compositions comprising the lipid particles herein comprising a variant Paramyxovirus G/H or F or polynucleotides encoding the variant Paramyxovirus G/H or F, including pharmaceutical compositions and formulations. The pharmaceutical compositions can include any of the described variant Paramyxovirus G/H or F containing lipid particles.

The present disclosure also provides, in some aspects, a pharmaceutical composition comprising the composition described herein and pharmaceutically acceptable carrier.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular lipid particle and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some embodiments, the lipid particle meets a pharmaceutical or good manufacturing practices (GMP) standard. In some embodiments, the lipid particle is made according to good manufacturing practices (GMP). In some embodiments, the lipid particle has a pathogen level below a predetermined reference value, e.g., is substantially free of pathogens. In some embodiments, the lipid particle has a contaminant level below a predetermined reference value, e.g., is substantially free of contaminants. In some embodiments, the lipid particle has low immunogenicity.

In some embodiments, formulations of the pharmaceutical compositions described herein may be prepared by any method known or hereafter developed in the art of pharmacology. In some embodiments, preparatory methods include the step of bringing the active ingredient into association with a carrier or one or more other accessory ingredients, and then, if necessary or desirable, shaping or packaging the product into a desired single- or multi-dose unit.

In some embodiments, a “unit dose” is a discrete amount of the pharmaceutical composition comprising a predetermined amount of the active ingredient. In some embodiments, the amount of the active ingredient is generally equal to the dosage of the active ingredient that would be administered to a subject or a convenient fraction of such a dosage such as, for example, one-half or one-third of such a dosage. In some embodiments, the unit dosage form may be for a single daily dose or one of multiple daily doses (e.g., about 1 to 4 or more times per day). In some embodiments, when multiple daily doses are used, the unit dosage form may be the same or different for each dose.

In some embodiments, the lipid particle containing the variant Paramyxovirus G/H and/or F protein is a viral vector or virus-like particle (e.g., Section III). In some embodiments, the compositions provided herein can be formulated in dosage units of genome copies (GC). Suitable method for determining GC have been described and include, e.g., qPCR or digital droplet PCR (ddPCR) as described in, e.g., M. Lock et al, Hu Gene Therapy Methods, Hum Gene Ther Methods 25 (2): 115-25, 2014, which is incorporated herein by reference. In some embodiments, the dosage of administration of a viral vector or virus-like particle is from about 10⁴ to about 10¹⁰ GC units, inclusive. In some embodiments, the dosage of administration of a viral vector or virus-like particle is from about 10⁹ to about 10¹⁵ GC units, inclusive. In some embodiments, the dosage of administration of a viral vector or virus-like particle is from about 10⁵ to about 10⁹ GC units, inclusive. In some embodiments, the dosage of administration of a viral vector or virus-like particle is from about 10⁶ to about 10⁹ GC units, inclusive. In some embodiments, the dosage of administration of a viral vector or virus-like particle is from about 10¹² to about 10¹⁴ GC units, inclusive. In some embodiments, the dosage of administration is 1.0×10⁹ GC units, 5.0×10⁹ GC units, 1.0×10¹⁰ GC units, 5.0×10¹⁰ GC units, 1.0×10¹¹ GC units, 5.0×10¹¹ GC units, 1.0×10¹² GC units, 5.0×10¹² GC units, or 1.0×10¹³ GC units, 5.0×10¹³ GC units, 1.0×10¹⁴ GC units, 5.0×10¹⁴ GC units, or 1.0×10¹⁵ GC units.

In some embodiments, the dosage of administration of a viral vector or virus-like particle is from about 10⁴ to about 10¹⁰ infectious units, inclusive. In some embodiments, the dosage of administration of a viral vector or virus-like particle is from about 10⁹ to about 10¹⁵ infectious units, inclusive. In some embodiments, the dosage of administration of a viral vector or virus-like particle is from about 10⁵ to about 10⁹ infectious units. In some embodiments, the dosage of administration of a viral vector or virus-like particle is from about 10⁶ to about 10⁹ infectious units. In some embodiments, the dosage of administration of a viral vector or virus-like particle is from about 10¹² to about 10¹⁴ infectious units, inclusive. In some embodiments, the dosage of administration is 1.0×10⁹ infectious units, 5.0×10¹¹ infectious units, 1.0×10¹⁰ infectious units, 5.0×10¹⁰ infectious units, 1.0×10¹¹ infectious units, 5.0×10¹¹ infectious units, 1.0×10¹² infectious units, 5.0×10¹² infectious units, or 1.0×10¹³ infectious units, 5.0×10¹³ infectious units, 1.0×10¹⁴ infectious units, 5.0×10¹⁴ infectious units, or 1.0×10¹⁵ infectious units. The techniques available for quantifying infectious units are routine in the art and include viral particle number determination, fluorescence microscopy, and titer by plaque assay. For example, the number of adenovirus particles can be determined by measuring the absorbance at A260. Similarly, infectious units can also be determined by quantitative immunofluorescence of vector specific proteins using monoclonal antibodies or by plaque assay.

In some embodiments, methods that calculate the infectious units include the plaque assay, in which titrations of the virus are grown on cell monolayers and the number of plaques is counted after several days to several weeks. For example, the infectious titer is determined, such as by plaque assay, for example an assay to assess cytopathic effects (CPE). In some embodiments, a CPE assay is performed by serially diluting virus on monolayers of cells, such as HFF cells, that are overlaid with agarose. After incubation for a time period to achieve a cytopathic effect, such as for about 3 to 28 days, generally 7 to 10 days, the cells can be fixed and foci of absent cells visualized as plaques are determined. In some embodiments, infectious units can be determined using an endpoint dilution (TCID50) method, which determines the dilution of virus at which 50% of the cell cultures are infected and hence, generally, can determine the titer within a certain range, such as one log.

In some embodiments, the dosage of administration of a viral vector or virus-like particle is from about 10⁴ to about 10¹⁰ plaque forming units (pfu), inclusive. In some embodiments, the dosage of administration of a viral vector or virus-like particle is from about 10⁹ to about 10¹⁵ pfu, inclusive In some embodiments, the dosage of administration of a viral vector or virus-like particle is from about 10⁵ to about 10⁹ pfu. In some embodiments, the dosage of administration of a viral vector or virus-like particle is from about 10⁶ to about 10⁹ pfu. In some embodiments, the dosage of administration of a viral vector or virus-like particle is from about 10¹² to about 10¹⁴ pfu, inclusive. In some embodiments, the dosage of administration is 1.0×10⁹ pfu, 5.0×10⁹ pfu, 1.0×10¹⁰ pfu, 5.0×10¹⁰ pfu, 1.0×10¹¹ pfu, 5.0×10¹¹ pfu, 1.0×10¹² pfu, 5.0×10¹² pfu, or 1.0×10¹³ pfu, 5.0×10¹³ pfu, 1.0×10¹⁴ pfu, 5.0×10¹⁴ pfu, or 1.0×10¹⁵ pfu.

In some embodiments, the subject will receive a single injection. In some embodiments, administration can be repeated at daily/weekly/monthly intervals for an indefinite period and/or until the efficacy of the treatment has been established. As set forth herein, the efficacy of treatment can be determined by evaluating the symptoms and clinical parameters described herein and/or by detecting a desired response.

The exact amount of vehicle provided lipid particle required will vary from subject to subject, depending on the species, age, weight and general condition of the subject, the particular polynucleic acid, polypeptide, or vector used, its mode of administration etc. TAn appropriate amount can be determined by one of ordinary skill in the art using only routine experimentation given the teachings herein.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the lipid particles in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Injectables can be prepared in conventional forms, either as liquid solutions or suspensions, solid forms suitable for solution of suspension in liquid prior to injection, or as emulsions. As used herein, “parenteral administration” includes intradermal, intranasal, subcutaneous, intramuscular, intraperitoneal, intravenous and intratracheal routes, as well as a slow release or sustained release system such that a constant dosage is maintained.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

In some embodiments, vehicle formulations may comprise cyroprotectants. As used herein, there term “cryoprotectant” refers to one or more agent that when combined with a given substance, helps to reduce or eliminate damage to that substance that occurs upon freezing. In some embodiments, cryoprotectants are combined with vector vehicles in order to stabilize them during freezing. In some aspects, Frozen storage of RNA between −20° C., and −80° C. may be advantageous for long term (e.g. 36 months) stability of polynucleotide. In some embodiments, the RNA species is mRNA. In some embodiments, cryoprotectants are included in vehicle formulations to stabilize polynucleotide through freeze/thaw cycles and under frozen storage conditions. Cryoprotectants of the provided embodiments may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

The formulations to be used for in vivo administration are generally sterile. Sterility may be readily accomplished, e.g., by filtration through sterile filtration membranes.

### V. Methods of Use

In some embodiments, the lipid particles provided herein or pharmaceutical compositions containing same can be administered to a subject, e.g. a mammal, e.g. a human. In such embodiments, the subject may be at risk of, may have a symptom of, or may be diagnosed with or identified as having, a particular disease or condition. In one embodiment, the subject has cancer. In one embodiment, the subject has an infectious disease. In some embodiments, the lipid particle contains nucleic acid sequences encoding an exogenous agent for treating the disease or condition in the subject. For example, the exogenous agent is one that targets or is specific for a protein of a neoplastic cells and the lipid particle is administered to a subject for treating a tumor or cancer in the subject. In another example, the exogenous agent is an inflammatory mediator or immune molecule, such as a cytokine, and lipid particle is administered to a subject for treating any condition in which it is desired to modulate (e.g. increase) the immune response, such as a cancer or infectious disease. In some embodiments, the lipid particle is administered in an effective amount or dose to effect treatment of the disease, condition or disorder. Provided herein are uses of any of the provided lipid particles in such methods and treatments, and in the preparation of a medicament in order to carry out such therapeutic methods. In some embodiments, the methods are carried out by administering the lipid particle or compositions comprising the same, to the subject having, having had, or suspected of having the disease or condition or disorder. In some embodiments, the methods thereby treat the disease or condition or disorder in the subject. Also provided herein are uses of any of the compositions, such as pharmaceutical compositions provided herein, for the treatment of a disease, condition or disorder associated with a particular gene or protein targeted by or provided by the exogenous agent.

In some embodiments, the provided methods or uses involve administration of a pharmaceutical composition comprising oral, inhaled, transdermal or parenteral (including intravenous, intratumoral, intraperitoneal, intramuscular, intracavity, intranodal and subcutaneous) administration. In some embodiments, the lipid particle may be administered alone or formulated as a pharmaceutical composition. In some embodiments, the lipid particle or compositions described herein can be administered to a subject, e.g., a mammal, e.g., a human. In some of any embodiments, the subject may be at risk of, may have a symptom of, or may be diagnosed with or identified as having, a particular disease or condition (e.g., a disease or condition described herein). In some embodiments, the disease is a disease or disorder.

In some embodiments, the lipid particles may be administered in the form of a unit-dose composition, such as a unit dose oral, parenteral, transdermal or inhaled composition. In some embodiments, the compositions are prepared by admixture and are adapted for oral, inhaled, transdermal or parenteral administration, and as such may be in the form of tablets, capsules, oral liquid preparations, powders, granules, lozenges, reconstitutable powders, injectable and infusable solutions or suspensions or suppositories or aerosols.

In some embodiments, the regimen of administration may affect what constitutes an effective amount. In some embodiments, the therapeutic formulations may be administered to the subject either prior to or after a diagnosis of disease. In some embodiments, several divided dosages, as well as staggered dosages may be administered daily or sequentially, or the dose may be continuously infused, or may be a bolus injection. In some embodiments, the dosages of the therapeutic formulations may be proportionally increased or decreased as indicated by the exigencies of the therapeutic or prophylactic situation.

In some embodiments, the administration of the compositions of the present invention to a subject, preferably a mammal, more preferably a human, may be carried out using known procedures, at dosages and for periods of time effective to prevent or treat disease. In some embodiments, an effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. In some embodiments, the dosage regimens may be adjusted to provide the optimum therapeutic response. In some embodiments, several divided doses may be administered daily or the dose may be proportionally reduced as indicated by the exigencies of the therapeutic situation. In some embodiments, the effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

In some embodiments, the compound may be administered to a subject as frequently as several times daily, or it may be administered less frequently, such as once a day, once a week, once every two weeks, once a month, or even less frequently, such as once every several months or even once a year or less. In some embodiments, the amount of compound dosed per day may be administered, in non-limiting examples, every day, every other day, every 2 days, every 3 days, every 4 days, or every 5 days. In some embodiments, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

In some embodiments, dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. In some embodiments, the physician or veterinarian could start doses of the compounds of the invention employed in the pharmaceutical composition at levels lower than that required in order to achieve the desired therapeutic effect and gradually increase the dosage until the desired effect is achieved.

In some embodiments, it is especially advantageous to formulate the compound in dosage unit form for ease of administration and uniformity of dosage. In some embodiments, dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. In some embodiments, the dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In some embodiments, the term “container” includes any receptacle for holding the pharmaceutical composition. In some embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. It should be understood that the instructions for use of the pharmaceutical composition may be contained on the packaging containing the pharmaceutical composition, and as such the instructions form an increased functional relationship to the packaged product. In some embodiments, instructions may contain information pertaining to the compound's ability to perform its intended function, e.g., treating or preventing a disease in a subject, or delivering an imaging or diagnostic agent to a subject.

In some embodiments, routes of administration of any of the compositions disclosed herein include oral, nasal, rectal, parenteral, sublingual, transdermal, transmucosal (e.g., sublingual, lingual, (trans) buccal, (trans) urethral, vaginal (e.g., trans- and perivaginally), (intra) nasal, and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

In some of any embodiments, suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like.

In some embodiments, the lipid particle composition comprising an exogenous agent or cargo, may be used to deliver such exogenous agent or cargo to a cell tissue or subject. In some embodiments, delivery of a cargo by administration of a lipid particle composition described herein may modify cellular protein expression levels. In certain embodiments, the administered composition directs upregulation of (via expression in the cell, delivery in the cell, or induction within the cell) of one or more cargo (e.g., a polypeptide or mRNA) that provide a functional activity which is substantially absent or reduced in the cell in which the polypeptide is delivered. In some embodiments, the missing functional activity may be enzymatic, structural, or regulatory in nature. In some embodiments, the administered composition directs up-regulation of one or more polypeptides that increases (e.g., synergistically) a functional activity which is present but substantially deficient in the cell in which the polypeptide is upregulated. In some of any embodiments, the administered composition directs downregulation of (via expression in the cell, delivery in the cell, or induction within the cell) of one or more cargo (e.g., a polypeptide, siRNA, or miRNA) that repress a functional activity which is present or upregulated in the cell in which the polypeptide, siRNA, or miRNA is delivered. In some of any embodiments, the upregulated functional activity may be enzymatic, structural, or regulatory in nature. In some embodiments, the administered composition directs down-regulation of one or more polypeptides that decreases (e.g., synergistically) a functional activity which is present or upregulated in the cell in which the polypeptide is downregulated. In some embodiments, the administered composition directs upregulation of certain functional activities and downregulation of other functional activities.

In some of any embodiments, the lipid particle composition (e.g., one comprising mitochondria or DNA) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the lipid particle composition comprises an exogenous protein), the effect lasts for less than 1, 2, 3, 4, 5, 6, or 7 days, 2, 3, or 4 weeks, or 1, 2, 3, 6, or 12 months.

In some of any embodiments, the lipid particle composition described herein is delivered ex-vivo to a cell or tissue, e.g., a human cell or tissue. In embodiments, the composition improves function of a cell or tissue ex-vivo, e.g., improves cell viability, respiration, or other function (e.g., another function described herein).

In some embodiments, the composition is delivered to an ex vivo tissue that is in an injured state (e.g., from trauma, disease, hypoxia, ischemia or other damage).

In some embodiments, the composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the composition is delivered to the tissue or organ before, during and/or after transplantation.

In some embodiments, the composition is delivered, administered or contacted with a cell, e.g., a cell preparation. In some embodiments, the cell preparation may be a cell therapy preparation (a cell preparation intended for administration to a human subject). In embodiments, the cell preparation comprises cells expressing a chimeric antigen receptor (CAR), e.g., expressing a recombinant CAR. The cells expressing the CAR may be, e.g., T cells, Natural Killer (NK) cells, cytotoxic T lymphocytes (CTL), regulatory T cells. In embodiments, the cell preparation is a neural stem cell preparation. In embodiments, the cell preparation is a mesenchymal stem cell (MSC) preparation. In embodiments, the cell preparation is a hematopoietic stem cell (HSC) preparation. In embodiments, the cell preparation is an islet cell preparation.

In some embodiments, the lipid particle compositions described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein).

In some embodiments, the source of lipid particles are from the same subject that is administered a lipid particle composition. In other embodiments, they are different. In some embodiments, the source of lipid particles and recipient tissue may be autologous (from the same subject) or heterologous (from different subjects). In some embodiments, the donor tissue for lipid particle compositions described herein may be a different tissue type than the recipient tissue. In some embodiments, the donor tissue may be muscular tissue and the recipient tissue may be connective tissue (e.g., adipose tissue). In other embodiments, the donor tissue and recipient tissue may be of the same or different type, but from different organ systems.

In some embodiments, the lipid particle composition described herein may be administered to a subject having a cancer, an autoimmune disease, an infectious disease, a metabolic disease, a neurodegenerative disease, or a genetic disease (e.g., enzyme deficiency). In some embodiments, the subject is in need of regeneration.

In some embodiments, the lipid particle is co-administered with an inhibitor of a protein that inhibits membrane fusion. For example, Suppressyn is a human protein that inhibits cell-cell fusion (Sugimoto et al., “A novel human endogenous retroviral protein inhibits cell-cell fusion” Scientific Reports 3:1462 (DOI: 10.1038/srep01462)). In some embodiments, the lipid particle particles is co-administered with an inhibitor of sypressyn, e.g., a siRNA or inhibitory antibody.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1. Glycan Removal of Nipah-Retargeted Fusogens Results in Decreased Off-Target Transduction with No Decrease in On-Target Transduction

Nipah-retargeted or VSV-G pseudotyped lentiviral vectors for expression of GFP were treated with Protein Deglycosylation Mix II (New England Biolabs, Ipswich, MA), which includes the enzymes PNGaseF, O-glycosidase, α2-3,6,8,9 neuraminidase, β 1-4 Galactosidase S, and β-N-Acetylhexosaminidase F. Briefly, concentrated vector was diluted 5-10 fold in PBS, an appropriate volume of 10× digestion buffer was added, and 2-5 μL of the enzyme mixture was added. The vectors and enzymes were incubated at room temperature for 30 minutes, followed by 2 hours at 37C. The reactions were quenched with an equal volume of serum containing media. An untreated vector control underwent the incubations and additions of enzyme buffer, but no enzyme was added. Primary Human Hepatocytes (plated 2 hours prior to transduction) were transduced with enzyme-treated and untreated virus for 4 days before being analyzed via Cytation5. SupT1 cells were also transduced for 4 days and analyzed by flow cytometry to show on target transduction was maintained.

In pseudotyped vectors comprising Nipah F and G proteins retargeted with a CD8-specific VHH, off-target transduction was observed in primary human hepatocyte (PHH) cells, though no significant off-target transduction was observed in renal epithelial cells, Ramos cells, HEK293X cells, or HUVECs at similar dilutions (FIG. 1A). To examine whether removing glycans from these proteins affected transduction, fusogens comprising Nipah-F and CD8-retargeted Nipah-G proteins were treated with deglycosylation mixture prior to transduction. As shown in FIG. 1B, fusogens comprising re-targeted Nipah proteins showed a significant reduction in off-target transduction of PHH cells after treatment with deglycosylation mixture.

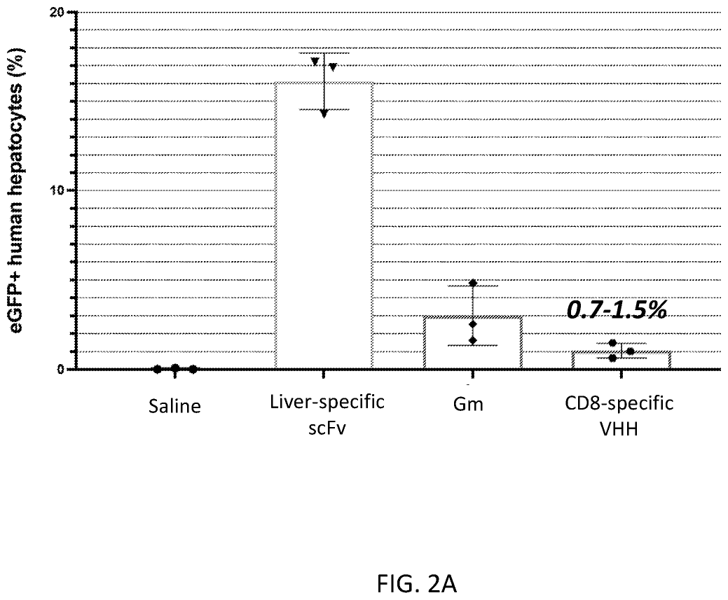

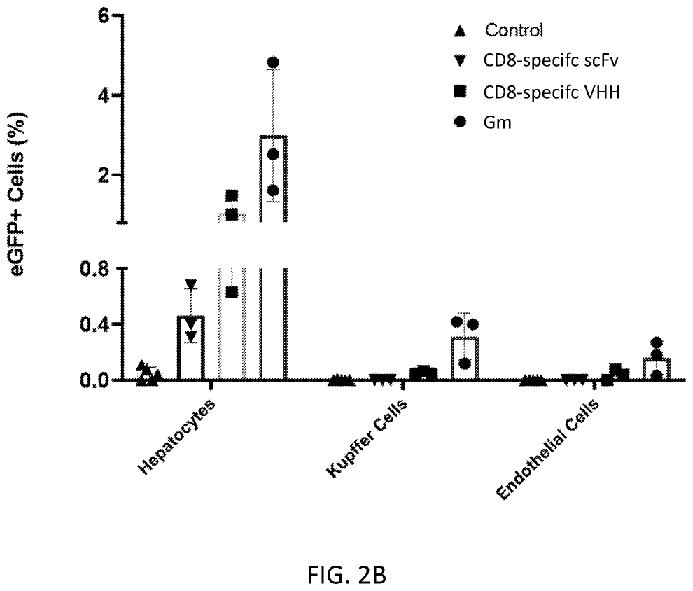

To examine whether off-target transduction could be recapitulated in vivo, vectors pseudotyped with Nipah fusogens retargeted against a CD8-specific VHH, a CD8-specific scFv, a liver-specific scFv, or blinded yet untargeted (GM) were transduced into mice with a humanized liver (PHH). As shown in FIGS. 2A-2B, retargeted and blinded fusogens transduced human liver cells in vivo, with the highest transduction observed in hepatocytes and lower transduction observed in Kupffer cells and endothelial cells.

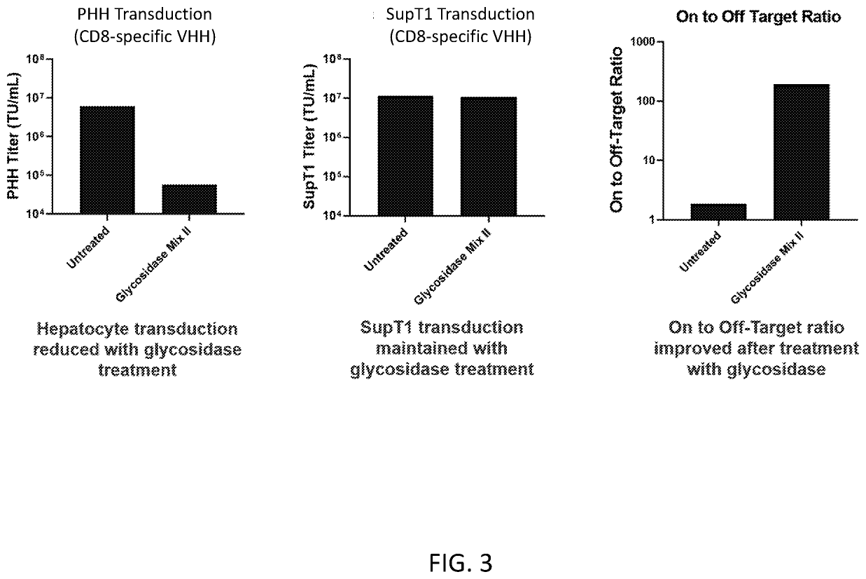

Notably, vectors pseudotyped with Nipah fusogens retargeted with a VHH against CD8 treated with deglycosylation mixture did not display a reduction in on-target transduction of on-target SupT1 cells, as shown in FIG. 3. In particular, the deglycosylation enzyme treated lipid particles displayed a substantial increase in the ratio of on-target to off-target transduction (FIG. 3, right plot).

### Example 2. Screening and Testing of N-Glycan Mutations Having Decreased Off-Target Transduction

To examine whether mutations in NiV-G at amino acid positions predicted to be sites of glycosylation could reduce off-target transduction, a library of Niv-G N-linked glycosylation was constructed, where predicted N-glycosylation sites were substituted with glutamine, either individually or in combination. Individual mutations were screened via an adherent. 24-well lentivirus production system. Lentivirus from these productions were titered on primary human hepatocytes. SupT1 cells, or ASGR1-overexpressing cells. Lead mutations were produced using a suspension lentivirus production system and these lentivirus preparations were concentrated 100 to 200-fold. These concentrated preparations were titered on primary human hepatocytes or SupT1 cells.

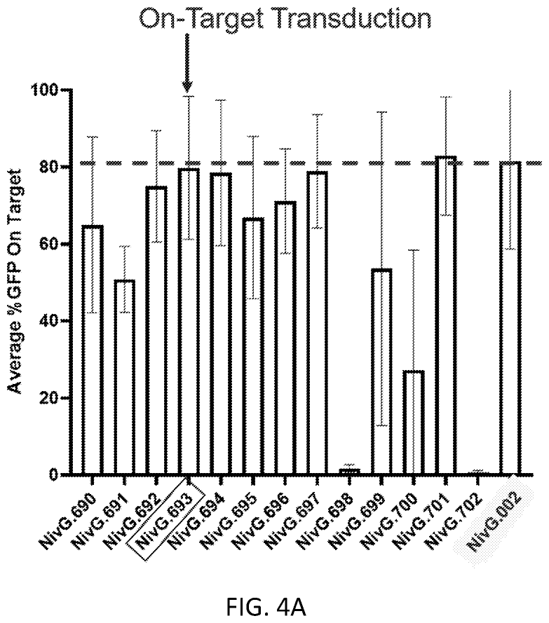

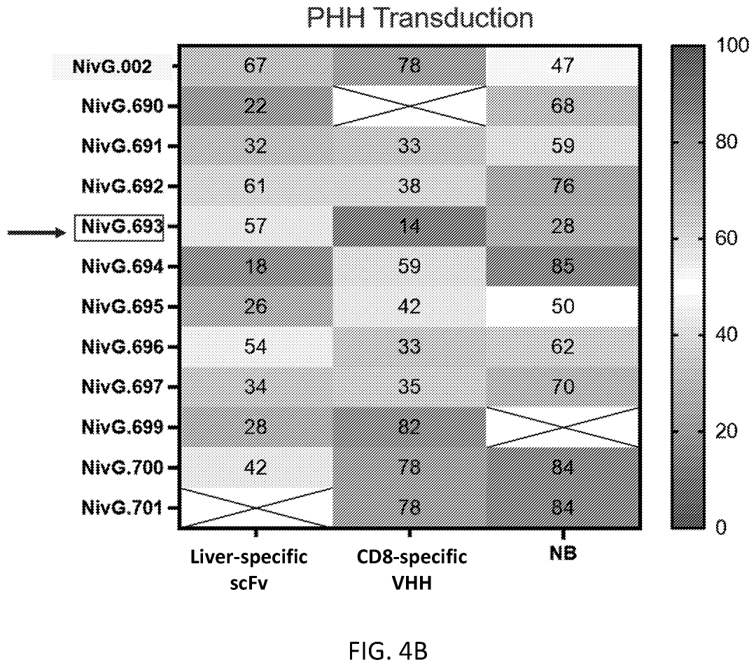

As shown in FIG. 4A, several of the mutant NiV-G proteins had similar on-target transduction efficiencies, as compared to a non-mutated control (NivG.002. SEQ ID NO: 1) in FIG. 4A). Several of these mutations, however, showed reduced PHH transduction as compared to the control. In particular, several mutants had an on-target transduction efficiency of nearly 80%, but had appreciably lower off-target transduction (FIG. 4B, 5A, and 5B), either when fused to a CD8-specific VHH or comprising only the blinding mutations (FIG. 4B).

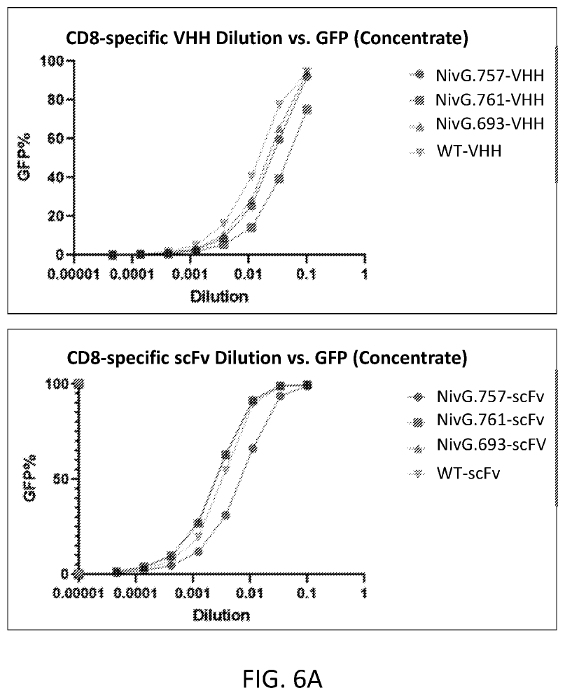

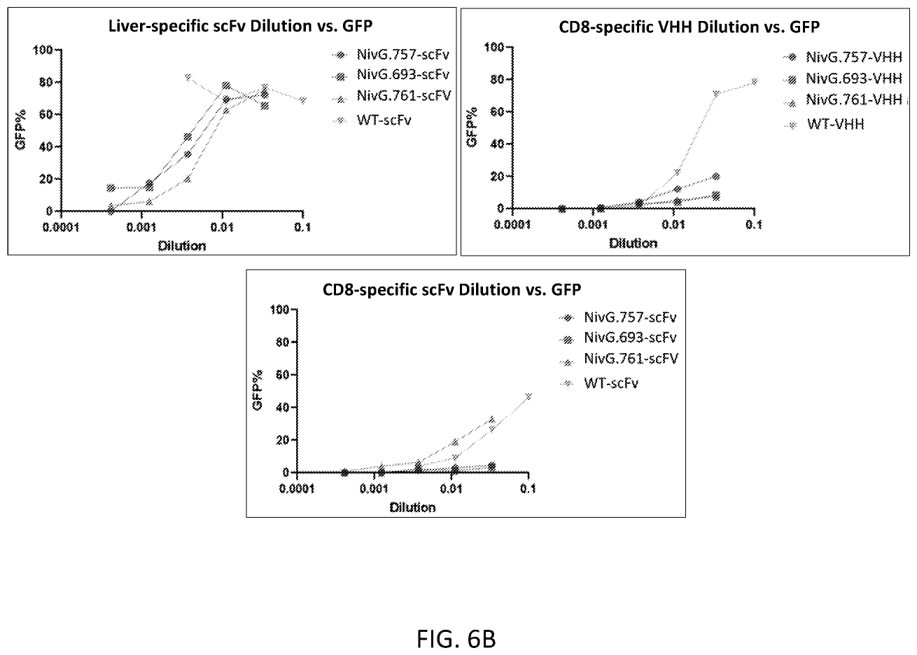

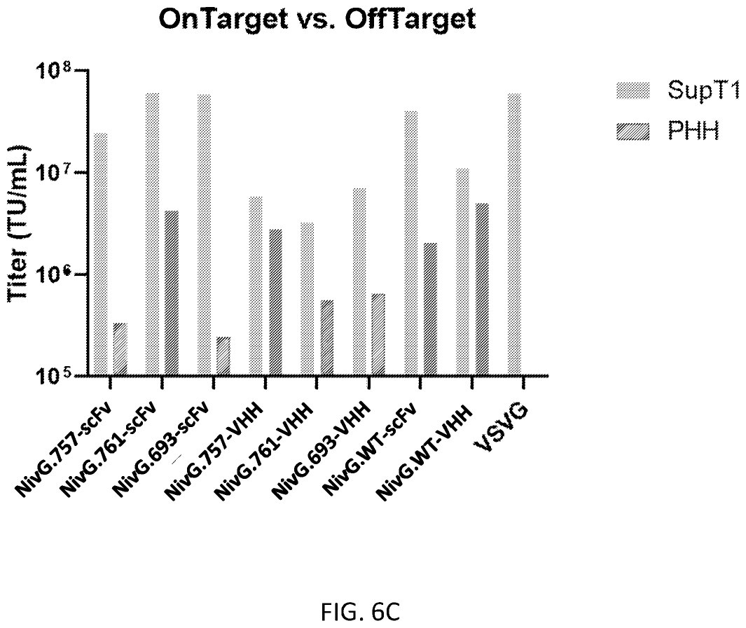

Three mutant constructs were chosen for further analysis. NivG.693 comprises a single glycosylation mutation. N273Q (SEQ ID NO: 581); NivG.757 comprises three mutations. N273Q. N384Q, and N496Q (SEQ ID NO: 602); NivG.761 also comprises three mutations, N273Q. N345Q. and N496Q (SEQ ID NO: 606). As shown in FIG. 6A, all three variant G proteins displayed similar on-target transduction of SupT1 cells as compared to a wild-type, across various dilutions.

In contrast, however, the mutated constructs displayed a significant decrease in titers in off-target transduction of PHH. (FIG. 6B). Moreover, several of the constructs displayed improved on-target vs. off-target titers as compared to wild-type (FIG. 6C).

### Example 3. In Vivo Transduction in Humanized Mouse Liver Model (FRG)

Transduction efficiency of viral vector pseudotyped with N-glycan mutation NiV-G was also assessed in vivo. Specifically, a mouse model was generated to determine the ability of a lentiviral vector carrying a GFP transgene and pseudotyped with a N-glycan glycan mutation NiV-G (NivG.693; N273Q; SEQ ID NO: 581) to transduce off-target primary human hepatocytes (PHHs) in vivo. In addition to the N-glycan mutation, the NiV-G also contained the blinding mutations (E501A. W504A, Q530A and E533A with reference to numbering of SEQ ID NO: 13) which reduce or eliminate binding to the native Ephrin B2 or B3 (NiVG Blinded+Glycan Mutation). For comparison, transduction of a lentiviral vector carrying the GFP transgene and pseudotyped with a blinded NiV-G without the N-glycan mutation also was tested (NiVG Blinded).

Chimeric Fah−/−/Rag2−/−/12rg−/− (FRG) mice were used, which allows repopulation with wild-type human liver cells in the immunodeficient mice (Wilson, E. M. et al. Extensive double humanization of both liver and hematopoiesis in FRGN mice. Stem Cell Res 13, 404-412, 2014). Without wishing to be bound by theory, this enables targeting of human hepatocytes in cases where mouse hepatocytes may not harbor similar surface proteome or permissivity to the delivery modality. In some aspects, the FRG model enables targeted delivery into human tissues and supports detectable off-target delivery in vitro (in human hepatocytes, such as in the Examples above).

TABLE E2
FRG in vivo Glycan Mutation Study Arms

| Arm | Lentiviral Vector | Dose | # of Doses | mL/kg |
|---|---|---|---|---|
| 1 | NivG Blinded | MAX | 3 | 10 |
| 2 | NivG Blinded + GlycanMutation | MAX | 3 | 10 |

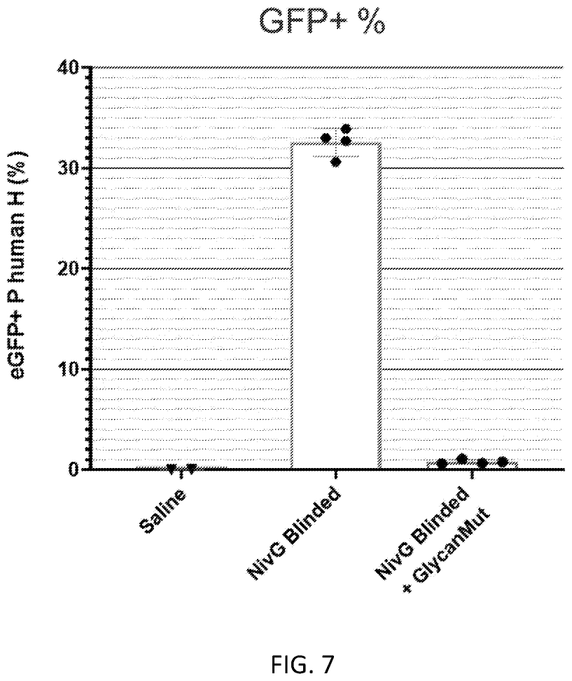

FRG mice engrafted with primary human hepatocytes were injected with three doses of lentiviral vector per one of the two study arms described in Table E1 over 3 days. GFP positive PHH cells were then detected by flow cytometry 7 days following the final injection. As shown in FIG. 7, the addition of the glycosylation mutant NivG.693 (N273Q; SEQ ID NO: 581) resulted in a decrease in off target transduction compared to a lentiviral vector pseudotyped with NiV-G without the glycosylation mutation.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

| SEQ | | |
|---|---|---|
| ID NO | SEQUENCES | ANNOTATION |
| 1 | MKKINEGLLDSKILSAFNTVIALLGSIVIIVMNIM | NIV-G |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 2 | MKKINEGLLDSKILSAFNTVIALLGSIVIIVMNIM | NIV-G N39Q |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 3 | MKKINEGLLDSKILSAFNTVIALLGSIVIIVMNIM | NIV-G N126Q |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 4 | MKKINEGLLDSKILSAFNTVIALLGSIVIIVMNIM | NV-GS128A |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNIACPNPLPFREYRP | |
| | QTEGVSNLVGLPNNICLQKTSNQILKPKLISYTL | |
| | PVVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 5 | MKKINEGLLDSKILSAFNTVIALLGSIVIIVMNIM | NIV-G N273Q |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRENTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 6 | MKKINEGLLDSKILSAFNTVIALLGSIVIIVMNIM | NIV-G N345Q |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 7 | MKKINEGLLDSKILSAFNTVIALLGSIVIIVMNIM | NiV-G N384Q |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 8 | MKKINEGLLDSKILSAFNTVIALLGSIVIIVMNIM | NiV-G N448Q |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 9 | MKKINEGLLDSKILSAFNTVIALLGSIVIIVMNIM | NIV-G N496Q |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 10 | MKKINEGLLDSKILSAFNTVIALLGSIVIIVMNIM | NiV-G N345Q, |
| | IIQNYTRSTDNQAVIKDALQGIQQQIKGLADKIG | N384Q, N448Q |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 11 | MKKINEGLLDSKILSAFNTVIALLGSIVIIVMNIM | NiV-G N345Q, |
| | IIQNYTRSTDNQAVIKDALQGIQQQIKGLADKIG | N448Q, N496Q |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 12 | MKKINEGLLDSKILSAFNTVIALLGSIVIIVMNIM | NiV-G N345Q, |
| | IIQNYTRSTDNQAVIKDALQGIQQQIKGLADKIG | N496Q |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 13 | MPAENKKVRFENTTSDKGKIPSKVIKSYYGTMD | NiVG protein |
| | IKKINEGLLDSKILSAFNTVIALLGSIVIIVMNIMII | attachment |
| | QNYTRSTDNQAVIKDALQGIQQQIKGLADKIGT | glycoprotein (602 |
| | EIGPKVSLIDTSSTITIPANIGLLGSKISQSTASINE | aa) |
| | NVNEKCKFTLPPLKIHECNISCPNPLPFREYRPQT | Uniprot Q9IH62 |
| | EGVSNLVGLPNNICLQKTSNQILKPKLISYTLPV | Cytoplasmic tail |
| | VGQSGTCITDPLLAMDEGYFAYSHLERIGSCSR | 1-49 |
| | GVSKQRIIGVGEVLDRGDEVPSLFMTNVWTPPN | Transmembrane |
| | PNTVYHCSAVYNNEFYYVLCAVSTVGDPILNST | 50-70 |
| | YWSGSLMMTRLAVKPKSNGGGYNQHQLALRSI | Ectodomain 71- |
| | EKGRYDKVMPYGPSGIKQGDTLYFPAVGFLVR | 602; Stalk domain |
| | TEFKYNDSNCPITKCQYSKPENCRLSMGIRPNSH | 71-187; head |
| | YILRSGLLKYNLSDGENPKVVFIEISDQRLSIGSP | domain 188-602) |
| | SKIYDSLGQPVFYQASFSWDTMIKFGDVLTVNP | |
| | LVVNWRNNTVISRPGQSQCPRFNTCPEICWEGV | |
| | YNDAFLIDRINWISAGVFLDSNQTAENPVFTVFK | |
| | DNEILYRAQLASEDTNAQKTITNCFLLKNKIWCI | |
| | SLVEIYDTGDNVIRPKLFAVKIPEQCT | |
| 14 | ILSAFNTVIALLGSIVIIVMNIMIIQNYTRSTDNQA | NiV-G backbone |
| | VIKDALQGIQQQIKGLADKIGTEIGPKVSLIDTSS | ΔCT |
| | TITIPANIGLLGSKISQSTASINENVNEKCKFTLPP | |
| | LKIHECNISCPNPLPFREYRPQTEGVSNLVGLPN | |
| | NICLQKTSNQILKPKLISYTLPVVGQSGTCITDPL | |
| | LAMDEGYFAYSHLERIGSCSRGVSKQRIIGVGE | |
| | VLDRGDEVPSLFMTNVWTPPNPNTVYHCSAVY | |
| | NNEFYYVLCAVSTVGDPILNSTYWSGSLMMTR | |
| | LAVKPKSNGGGYNQHQLALRSIEKGRYDKVMP | |
| | YGPSGIKQGDTLYFPAVGFLVRTEFKYNDSNCPI | |
| | TKCQYSKPENCRLSMGIRPNSHYILRSGLLKYNL | |
| | SDGENPKVVFIEISDQRLSIGSPSKIYDSLGQPVF | |
| | YQASFSWDTMIKFGDVLTVNPLVVNWRNNTVI | |
| | SRPGQSQCPRFNTCPEICWEGVYNDAFLIDRINW | |
| | ISAGVFLDSNQTAENPVFTVFKDNEILYRAQLAS | |
| | EDTNAQKTITNCFLLKNKIWCISLVEIYDTGDNV | |
| | IRPKLFAVKIPEQCT | |
| 15 | ILSAFNTVIALLGSIVIIVMNIMIIQNYTRSTDNQA | NiV-G backbone |
| | VIKDALQGIQQQIKGLADKIGTEIGPKVSLIDTSS | ΔCT and mutated |
| | TITIPANIGLLGSKISQSTASINENVNEKCKFTLPP | (E501A, W504A, |
| | LKIHECNISCPNPLPFREYRPQTEGVSNLVGLPN | Q530A, E533A) |
| | NICLQKTSNQILKPKLISYTLPVVGQSGTCITDPL | |
| | LAMDEGYFAYSHLERIGSCSRGVSKQRIIGVGE | |
| | VLDRGDEVPSLFMTNVWTPPNPNTVYHCSAVY | |
| | NNEFYYVLCAVSTVGDPILNSTYWSGSLMMTR | |
| | LAVKPKSNGGGYNQHQLALRSIEKGRYDKVMP | |
| | YGPSGIKQGDTLYFPAVGFLVRTEFKYNDSNCPI | |
| | TKCQYSKPENCRLSMGIRPNSHYILRSGLLKYNL | |
| | SDGENPKVVFIEISDQRLSIGSPSKIYDSLGQPVF | |
| | YQASFSWDTMIKFGDVLTVNPLVVNWRNNTVI | |
| | SRPGQSQCPRFNTCPAICAEGVYNDAFLIDRINW | |
| | ISAGVFLDSNATAANPVFTVFKDNEILYRAQLAS | |
| | EDTNAQKTITNCFLLKNKIWCISLVEIYDTGDNV | |
| | IRPKLFAVKIPEQCT | |
| 16 | MPAENKKVRFENTTSDKGKIPSKVIKSYYGTMD | NiVG with NiVG |
| | IKKINEGLLDSKILSAFNTVIALLGSIVIIVMNIMII | CT_45_FL, |
| | QNYTRSTDNQAVIKDALQGIQQQIKGLADKIGT | mutated (E501A, |
| | EIGPKVSLIDTSSTITIPANIGLLGSKISQSTASINE | W504A, Q530A, |
| | NVNEKCKFTLPPLKIHECNISCPNPLPFREYRPQT | E533A) |
| | EGVSNLVGLPNNICLQKTSNQILKPKLISYTLPV | |
| | VGQSGTCITDPLLAMDEGYFAYSHLERIGSCSR | |
| | GVSKQRIIGVGEVLDRGDEVPSLFMTNVWTPPN | |
| | PNTVYHCSAVYNNEFYYVLCAVSTVGDPILNST | |
| | YWSGSLMMTRLAVKPKSNGGGYNQHQLALRSI | |
| | EKGRYDKVMPYGPSGIKQGDTLYFPAVGFLVR | |
| | TEFKYNDSNCPITKCQYSKPENCRLSMGIRPNSH | |
| | YILRSGLLKYNLSDGENPKVVFIEISDQRLSIGSP | |
| | SKIYDSLGQPVFYQASFSWDTMIKFGDVLTVNP | |
| | LVVNWRNNTVISRPGQSQCPRFNTCPAICAEGV | |
| | YNDAFLIDRINWISAGVFLDSNATAANPVFTVF | |
| | KDNEILYRAQLASEDTNAQKTITNCFLLKNKIW | |
| | CISLVEIYDTGDNVIRPKLFAVKIPEQCT | |
| | EVQLVESGGGLVQSGGSLRLSCAASGFTFSGYW | VHH |
| | MYWVRQAPGKGLEWVSAISPGGGSTYYPDSVK | |
| | GRFTISRDNAKNTLYLQMNSLEPEDTALYYCAS | |
| | SLTATHTYEYDYWGQGTQVTVSS | |
| 17 | GYTFSNYW | CDR-H1 |
| 18 | ILPGSGST | CDR-H2 |
| 19 | ARRGYGYDEGFDY | CDR-H3 |
| 20 | QDINSY | CDR-L1 |
| 21 | RAN | CDR-L2 |
| 22 | LQYDEFPPT | CDR-L3 |
| 23 | QVQLQQSGAELMKPGASVKMSCKATGYTFSNY | VH |
| | WIEWVKQRPGHGLEWIGEILPGSGSTSYNEKFK | |
| | GKATFTADTSSSTAYMQLSSLTSEDSAVYYCAR | |
| | RGYGYDEGFDYWGQGSTLTVSS | |
| 24 | DIKMTQSPSSMYASLGERVTITCKASQDINSYLS | VL |
| | WFQQKPGKSPKTLIYRANRLVDGVPSRFSGSGS | |
| | GQDYSLTISSLEYEDMGIYYCLQYDEFPPTFGAG | |
| | TKLELKR | |
| 25 | QVQLQQSGAELMKPGASVKMSCKATGYTFSNY | scFv |
| | WIEWVKQRPGHGLEWIGEILPGSGSTSYNEKFK | |
| | GKATFTADTSSSTAYMQLSSLTSEDSAVYYCAR | |
| | RGYGYDEGFDYWGQGSTLTVSSGGGGSGGGGS | |
| | GGGGSDIKMTQSPSSMYASLGERVTITCKASQDI | |
| | NSYLSWFQQKPGKSPKTLIYRANRLVDGVPSRF | |
| | SGSGSGQDYSLTISSLEYEDMGIYYCLQYDEFPP | |
| | TFGAGTKLELKR | |
| 26 | GYTFTDYV | CDR-H1 |
| 27 | IYPGSGSS | CDR-H2 |
| 28 | ARPGDLGFAY | CDR-H3 |
| 29 | QSVDYDGDSY | CDR-L1 |
| 30 | AAS | CDR-L2 |
| 31 | QQSNKDPFT | CDR-L3 |
| 32 | QVQLQQSGPELVKPGASVKMSCKASGYTFTDY | VH |
| | VISWVRQAPGQGLEWIGEIYPGSGSSYYNEKFK | |
| | GRATLTADKSSNTAYMQLSSLRSEDSAVYFCAR | |
| | PGDLGFAYWGQGTLVTVSS | |
| 33 | DIVLTQSPSSLAVSLGQRATISCKASQSVDYDGD | VL |
| | SYMNWYQQKPGQPPKLLIYAASNLESGIPARFS | |
| | GSGSGTDFTLTIHPVEEEDAATYYCQQSNKDPF | |
| | TFGGGTKLELKR | |
| 34 | QVQLQQSGPELVKPGASVKMSCKASGYTFTDY | scFv |
| | VISWVRQAPGQGLEWIGEIYPGSGSSYYNEKFK | |
| | GRATLTADKSSNTAYMQLSSLRSEDSAVYFCAR | |
| | PGDLGFAYWGQGTLVTVSSGGGGSGGGGSGGG | |
| | GSDIVLTQSPSSLAVSLGQRATISCKASQSVDYD | |
| | GDSYMNWYQQKPGQPPKLLIYAASNLESGIPAR | |
| | FSGSGSGTDFTLTIHPVEEEDAATYYCQQSNKDP | |
| | FTFGGGTKLELKR | |
| 35 | GGTFSSYA | CDR-H1 |
| 36 | INPNSGGT | CDR-H2 |
| 37 | ARDGYSGSYSD | CDR-H3 |
| 38 | QSVLSSSYNKNY | CDR-L1 |
| 39 | WAS | CDR-L2 |
| 40 | QQYYSTPWT | CDR-L3 |
| 41 | QVQLVQSGAEVKKPGASVKVSCKASGGTFSSY | VH |
| | AISWVRQAPGQGLEWMGWINPNSGGTNYAQK | |
| | FQGRVTMTRDTSTSTVYMELSSLRSEDTAVYYC | |
| | ARDGYSGSYSDWGQGTLVTVSS | |
| 42 | DIVMTQSPDSLAVSLGERATINCKSSQSVLSSSY | VL |
| | NKNYLAWYQQKPGQPPKLLIYWASTRESGVPD | |
| | RFSGSGSGTDFTLTISSLQAEDVAVYYCQQYYS | |
| | TPWTFGQGTKVEIK | |
| 43 | QVQLVQSGAEVKKPGASVKVSCKASGGTFSSY | scFv |
| | AISWVRQAPGQGLEWMGWINPNSGGTNYAQK | |
| | FQGRVTMTRDTSTSTVYMELSSLRSEDTAVYYC | |
| | ARDGYSGSYSDWGQGTLVTVSSGGGGSGGGGS | |
| | GGGGSDIVMTQSPDSLAVSLGERATINCKSSQS | |
| | VLSSSYNKNYLAWYQQKPGQPPKLLIYWASTR | |
| | ESGVPDRFSGSGSGTDFTLTISSLQAEDVAVYYC | |
| | QQYYSTPWTFGQGTKVEIK | |
| 44 | GYTFTSYD | CDR-H1 |
| 45 | IIPLSGAP | CDR-H2 |
| 46 | ARGALYNWNDGWFDP | CDR-H3 |
| 47 | QDIGDY | CDR-L1 |
| 48 | DAS | CDR-L2 |
| 49 | QQANSFPLT | CDR-L3 |
| 50 | QVQLVQSGAEVKKPGSSVKVSCKASGYTFTSY | VH |
| | DINWVRQAPGQGLEWMGGIIPLSGAPNYAHKF | |
| | QGRVTITADESTSTAYMELSSLRSEDTAVYYCA | |
| | RGALYNWNDGWFDPWGQGTLVTVSS | |
| 51 | DIQMTQSPSSLSASVGDRVTITCRASQDIGDYLA | VL |
| | WYQQKPGKAPKLLIYDASSLQSGVPSRFSGSGS | |
| | GTDFTLTISSLQPEDFATYYCQQANSFPLTFGGG | |
| | TKVEIK | |
| 52 | QVQLVQSGAEVKKPGSSVKVSCKASGYTFTSY | scFv |
| | DINWVRQAPGQGLEWMGGIIPLSGAPNYAHKF | |
| | QGRVTITADESTSTAYMELSSLRSEDTAVYYCA | |
| | RGALYNWNDGWFDPWGQGTLVTVSSGGGGSG | |
| | GGGSGGGGSDIQMTQSPSSLSASVGDRVTITCR | |
| | ASQDIGDYLAWYQQKPGKAPKLLIYDASSLQSG | |
| | VPSRFSGSGSGTDFTLTISSLQPEDFATYYCQQA | |
| | NSFPLTFGGGTKVEIK | |
| 53 | GDTFSDHA | CDR-H1 |
| 54 | MNPKIGNT | CDR-H2 |
| 55 | VYDSSGYDAFDI | CDR-H3 |
| 56 | QSVLSTSYNRNF | CDR-L1 |
| 57 | QQYYSTPYT | CDR-L3 |
| 58 | QVQLVQSGAEVKKPGASVKVSCKASGDTFSDH | VH |
| | AINWVRQAPGQGLEWMGWMNPKIGNTGYAQK | |
| | FQGRVTMTRDTSTSTVYMELSSLRSEDTAVYYC | |
| | VYDSSGYDAFDIWGQGTTVTVSS | |
| 59 | DIVMTQSPDSLAVSLGERATINCKSSQSVLSTSY | VL |
| | NRNFLAWYQQKPGQPPKLLIYWASTRQSGVPD | |
| | RFSGSGSGTDFTLTISSLQAEDVAVYYCQQYYS | |
| | TPYTFGQGTKLEIK | |
| 60 | QVQLVQSGAEVKKPGASVKVSCKASGDTFSDH | scFv |
| | AINWVRQAPGQGLEWMGWMNPKIGNTGYAQK | |
| | FQGRVTMTRDTSTSTVYMELSSLRSEDTAVYYC | |
| | VYDSSGYDAFDIWGQGTTVTVSSGGGGSGGGG | |
| | SGGGGSDIVMTQSPDSLAVSLGERATINCKSSQS | |
| | VLSTSYNRNFLAWYQQKPGQPPKLLIYWASTR | |
| | QSGVPDRFSGSGSGTDFTLTISSLQAEDVAVYYC | |
| | QQYYSTPYTFGQGTKLEIK | |
| 61 | GYSLITHW | CDR-H1 |
| 62 | INPSDGVT | CDR-H2 |
| 63 | AREYYGEGFDY | CDR-H3 |
| 64 | QGISNY | CDR-L1 |
| 65 | SAS | CDR-L2 |
| 66 | QQSYSTPLT | CDR-L3 |
| 67 | QVQLVQSGAEVKKPGASVKVSCKASGYSLITH | VH |
| | WMHWVRQAPGQGLEWMGMINPSDGVTYYAQ | |
| | TFQGRVTMTRDTSTSTVYMELSSLRSEDTAVYY | |
| | CAREYYGEGFDYWGQGTLVTVSS | |
| 68 | DIQMTQSPSSLSASVGDRVTITCRASQGISNYLA | VL |
| | WYQQKPGKAPKLLIYSASNLQSGVPSRFSGSGS | |
| | GTDFTLTISSLQPEDFATYYCQQSYSTPLTFGGG | |
| | TKVEIKR | |
| 69 | QVQLVQSGAEVKKPGASVKVSCKASGYSLITH | scFv |
| | WMHWVRQAPGQGLEWMGMINPSDGVTYYAQ | |
| | TFQGRVTMTRDTSTSTVYMELSSLRSEDTAVYY | |
| | CAREYYGEGFDYWGQGTLVTVSSGGGGSGGG | |
| | GSGGGGSDIQMTQSPSSLSASVGDRVTITCRASQ | |
| | GISNYLAWYQQKPGKAPKLLIYSASNLQSGVPS | |
| | RFSGSGSGTDFTLTISSLQPEDFATYYCQQSYSTP | |
| | LTFGGGTKVEIKR | |
| 70 | GYWMY | CDR-H1 |
| 71 | AISPGGGSTYYPDSVKG | CDR-H2 |
| 72 | SLTATHTYEYDY | CDR-H3 |
| 73 | GFTFSGY | CDR-H1 |
| 74 | SPGGGS | CDR-H2 |
| 75 | NYWIE | CDR-H1 |
| 76 | EILPGSGSTSYNEKFKG | CDR-H2 |
| 77 | RGYGYDEGFDY | CDR-H3 |
| 78 | KASQDINSYLS | CDR-L1 |
| 79 | RANRLVD | CDR-L2 |
| 80 | GYTFSNY | CDR-H1 |
| 81 | LPGSGS | CDR-H2 |
| 82 | DYVIS | CDR-H1 |
| 83 | EIYPGSGSSYYNEKFKG | CDR-H2 |
| 84 | PGDLGFAY | CDR-H3 |
| 85 | KASQSVDYDGDSYMN | CDR-L1 |
| 86 | AASNLES | CDR-L2 |
| 87 | GYTFTDY | CDR-H1 |
| 88 | YPGSGS | CDR-H2 |
| 89 | SYAIS | CDR-H1 |
| 90 | WINPNSGGTNYAQKFQG | CDR-H2 |
| 91 | DGYSGSYSD | CDR-H3 |
| 92 | KSSQSVLSSSYNKNYLA | CDR-L1 |
| 93 | WASTRES | CDR-L2 |
| 94 | GGTFSSY | CDR-H1 |
| 95 | NPNSGG | CDR-H2 |
| 96 | SYDIN | CDR-H1 |
| 97 | GIIPLSGAPNYAHKFQG | CDR-H2 |
| 98 | GALYNWNDGWFDP | CDR-H3 |
| 99 | RASQDIGDYLA | CDR-L1 |
| 100 | DASSLQS | CDR-L2 |
| 101 | GYTFTSY | CDR-H1 |
| 102 | IPLSGA | CDR-H2 |
| 103 | DHAIN | CDR-H1 |
| 104 | WMNPKIGNTGYAQKFQG | CDR-H2 |
| 105 | DSSGYDAFDI | CDR-H3 |
| 106 | KSSQSVLSTSYNRNFLA | CDR-L1 |
| 107 | WASTRQS | CDR-L2 |
| 108 | GDTFSDH | CDR-H1 |
| 109 | NPKIGN | CDR-H2 |
| 110 | THWMH | CDR-H1 |
| 111 | MINPSDGVTYYAQTFQG | CDR-H2 |
| 112 | EYYGEGFDY | CDR-H3 |
| 113 | RASQGISNYLA | CDR-L1 |
| 114 | SASNLQS | CDR-L2 |
| 115 | GYSLITH | CDR-H1 |
| 116 | NPSDGV | CDR-H2 |
| 117 | QVQLVQSGAEVKKPGASVKVSCKASGGTFSSY | CD8_1 VH |
| | AISWVRQAPGQGLEWMGIIDPSDGNTNYAQNF | |
| | QGRVTMTRDTSTSTVYMELSSLRSEDTAVYYC | |
| | AKERAAAGYYYYMDVWGQGTTVTVSS | |
| 118 | DIQMTQSPSSLSASVGDRVTITCRASQSISSYLN | CD8_1 VL |
| | WYQQKPGKAPKLLIYAASSLQSGVPSRFSGSGS | |
| | GTDFTLTISSLQPEDFATYYCQQSYSTPLTFGGG | |
| | TKVEIKR | |
| 119 | QVQLVQSGAEVKKPGASVKVSCKASGYTFTDY | CD8_2 VH |
| | YIQWVRQAPGQGLEWMGWINPNSGGTSYAQK | |
| | FQGRVTMTRDTSTSTVYMELSSLRSEDTAVYYC | |
| | AKEGDYYYGMDAWGQGTMVTVSS | |
| 120 | DIVMTQSPLSLPVTPGEPASISCRSSQSLLHSNGY | CD8_2 VL |
| | NYLDWYLQKPGQSPQLLIYLGSNRASGVPDRFS | |
| | GSGSGTDFTLKISRVEAEDVGVYYCMQGLQTPH | |
| | TFGQGTKVEIKR | |
| 121 | QVQLVQSGAEVKKPGASVKVSCKASGYTFTSY | CD8_3 VH |
| | YMHWVRQAPGQGLEWMGGFDPEDGETIYAQK | |
| | FQGRVTMTRDTSTSTVYMELSSLRSEDTAVYYC | |
| | ARDQGWGMDVWGQGTTVTVSS | |
| 122 | DIQMTQSPSSLSASVGDRVTITCRASQSISSYLN | CD8_3 VL |
| | WYQQKPGKAPKLLIYAASSLQSGVPSRFSGSGS | |
| | GTDFTLTISSLQPEDFATYYCQQTYSTPYTFGQG | |
| | TKLEIKR | |
| 123 | QVQLVQSGAEVKKPGASVKVSCKASGYTFTNH | CD8_4 VH |
| | YMHWVRQAPGQGLEWMGWMNPNSGNTGYA | |
| | QKFQGRVTMTRDTSTSTVYMELSSLRSEDTAVY | |
| | YCASSESGSDLDYWGQGTLVTVSS | |
| 124 | DIQMTQSPSSLSASVGDRVTITCRASQTIGNYVN | CD8_4 VL |
| | WYQQKPGKAPKLLIYGASNLHTGVPSRFSGSGS | |
| | GTDFTLTISSLQPEDFATYYCQQTYSAPLTFGGG | |
| | TKVEIKR | |
| 125 | QVQLVESGGGLVQAGGSLRLSCAASGRTFSGY | CD8 VHH |
| | VMGWFRQAPGKQRKFVAAISRGGLSTSYADSV | |
| | KGRFTISRDNAKNTVFLQMNTLKPEDTAVYYC | |
| | AADRSDLYEITAASNIDSWGQGTLVTVSS | |
| 126 | GGGGS | |
| 127 | GGGGGS | |
| 128 | (GGGGS)n; wherein n is 1-10 | |
| 129 | (GGGGGS)n; wherein n is 1-6 | |
| 130 | MATQEVRLKCLLCGIIVLVLSLEGLGILHYEKLS | Hendra F |
| | KIGLVKGITRKYKIKSNPLTKDIVIKMIPNVSNVS | |
| | KCTGTVMENYKSRLTGILSPIKGAIELYNNNTH | |
| | DLVGDVKLAGVVMAGIAIGIATAAQITAGVALY | |
| | EAMKNADNINKLKSSIESTNEAVVKLQETAEKT | |
| | VYVLTALQDYINTNLVPTIDQISCKQTELALDLA | |
| | LSKYLSDLLFVFGPNLQDPVSNSMTIQAISQAFG | |
| | GNYETLLRTLGYATEDFDDLLESDSIAGQIVYV | |
| | DLSSYYIIVRVYFPILTEIQQAYVQELLPVSFNND | |
| | NSEWISIVPNFVLIRNTLISNIEVKYCLITKKSVIC | |
| | NQDYATPMTASVRECLTGSTDKCPRELVVSSHV | |
| | PRFALSGGVLFANCISVTCQCQTTGRAISQSGEQ | |
| | TLLMIDNTTCTTVVLGNIIISLGKYLGSINYNSESI | |
| | AVGPPVYTDKVDISSQISSMNQSLQQSKDYIKEA | |
| | QKILDTVNPSLISMLSMIILYVLSIAALCIGLITFIS | |
| | FVIVEKKRGNYSRLDDRQVRPVSNGDLYYIGT | |
| 131 | MVVILDKRCYCNLLILILMISECSVGILHYEKLS | Nipah F0 |
| | KIGLVKGVTRKYKIKSNPLTKDIVIKMIPNVSNM | |
| | SQCTGSVMENYKTRLNGILTPIKGALEIYKNNT | |
| | HDLVGDVRLAGVIMAGVAIGIATAAQITAGVAL | |
| | YEAMKNADNINKLKSSIESTNEAVVKLQETAEK | |
| | TVYVLTALQDYINTNLVPTIDKISCKQTELSLDL | |
| | ALSKYLSDLLFVFGPNLQDPVSNSMTIQAISQAF | |
| | GGNYETLLRTLGYATEDFDDLLESDSITGQIIYV | |
| | DLSSYYIIVRVYFPILTEIQQAYIQELLPVSFNND | |
| | NSEWISIVPNFILVRNTLISNIEIGFCLITKRSVICN | |
| | QDYATPMTNNMRECLTGSTEKCPRELVVSSHV | |
| | PRFALSNGVLFANCISVTCQCQTTGRAISQSGEQ | |
| | TLLMIDNTTCPTAVLGNVIISLGKYLGSVNYNSE | |
| | GIAIGPPVFTDKVDISSQISSMNQSLQQSKDYIKE | |
| | AQRLLDTVNPSLISMLSMIILYVLSIASLCIGLITF | |
| | ISFIIVEKKRNTYSRLEDRRVRPTSSGDLYYIGT | |
| | MSNKRTTVLIIISYTLFYLNNAAIVGFDFDKLNKI | |
| 132 | GVVQGRVLNYKIKGDPMTKDLVLKFIPNIVNIT | CedV F |
| | ECVREPLSRYNETVRRLLLPIHNMLGLYLNNTN | |
| | AKMTGLMIAGVIMGGIAIGIATAAQITAGFALYE | |
| | AKKNTENIQKLTDSIMKTQDSIDKLTDSVGTSILI | |
| | LNKLQTYINNQLVPNLELLSCRQNKIEFDLMLT | |
| | KYLVDLMTVIGPNINNPVNKDMTIQSLSLLFDG | |
| | NYDIMMSELGYTPQDFLDLIESKSITGQIIYVDM | |
| | ENLYVVIRTYLPTLIEVPDAQIYEFNKITMSSNG | |
| | GEYLSTIPNFILIRGNYMSNIDVATCYMTKASVI | |
| | CNQDYSLPMSQNLRSCYQGETEYCPVEAVIASH | |
| | SPRFALTNGVIFANCINTICRCQDNGKTITQNINQ | |
| | FVSMIDNSTCNDVMVDKFTIKVGKYMGRKDIN | |
| | NINIQIGPQIIIDKVDLSNEINKMNQSLKDSIFYLR | |
| | EAKRILDSVNISLISPSVQLFLIIISVLSFIILLIIIVY | |
| | LYCKSKHSYKYNKFIDDPDYYNDYKRERINGK | |
| | ASKSNNIYYVGD | |
| 133 | MALNKNMFSSLFLGYLLVYATTVQSSIHYDSLS | Mojiang F |
| | KVGVIKGLTYNYKIKGSPSTKLMVVKLIPNIDSV | |
| | KNCTQKQYDEYKNLVRKALEPVKMAIDTMLN | |
| | NVKSGNNKYRFAGAIMAGVALGVATAATVTA | |
| | GIALHRSNENAQAIANMKSAIQNTNEAVKQLQL | |
| | ANKQTLAVIDTIRGEINNNIIPVINQLSCDTIGLSV | |
| | GIRLTQYYSEIITAFGPALQNPVNTRITIQAISSVF | |
| | NGNFDELLKIMGYTSGDLYEILHSELIRGNIIDV | |
| | DVDAGYIALEIEFPNLTLVPNAVVQELMPISYNI | |
| | DGDEWVTLVPRFVLTRTTLLSNIDTSRCTITDSS | |
| | VICDNDYALPMSHELIGCLQGDTSKCAREKVVS | |
| | SYVPKFALSDGLVYANCLNTICRCMDTDTPISQS | |
| | LGATVSLLDNKRCSVYQVGDVLISVGSYLGDGE | |
| | YNADNVELGPPIVIDKIDIGNQLAGINQTLQEAE | |
| | DYIEKSEEFLKGVNPSIITLGSMVVLYIFMILIAIV | |
| | SVIALVLSIKLTVKGNVVRQQFTYTQHVPSMENI | |
| | NYVSH | |
| 134 | MKKKTDNPTISKRGHNHSRGIKSRALLRETDNY | Bat PV F |
| | SNGLIVENLVRNCHHPSKNNLNYTKTQKRDSTI | |
| | PYRVEERKGHYPKIKHLIDKSYKHIKRGKRRNG | |
| | HNGNIITIILLLILILKTQMSEGAIHYETLSKIGLIK | |
| | GITREYKVKGTPSSKDIVIKLIPNVTGLNKCTNIS | |
| | MENYKEQLDKILIPINNIIELYANSTKSAPGNARF | |
| | AGVIIAGVALGVAAAAQITAGIALHEARQNAER | |
| | INLLKDSISATNNAVAELQEATGGIVNVITGMQ | |
| | DYINTNLVPQIDKLQCSQIKTALDISLSQYYSEIL | |
| | TVFGPNLQNPVTTSMSIQAISQSFGGNIDLLLNL | |
| | LGYTANDLLDLLESKSITGQITYINLEHYFMVIR | |
| | VYYPIMTTISNAYVQELIKISFNVDGSEWVSLVP | |
| | SYILIRNSYLSNIDISECLITKNSVICRHDFAMPMS | |
| | YTLKECLTGDTEKCPREAVVTSYVPRFAISGGVI | |
| | YANCLSTTCQCYQTGKVIAQDGSQTLMMIDNQ | |
| | TCSIVRIEEILISTGKYLGSQEYNTMHVSVGNPVF | |
| | TDKLDITSQISNINQSIEQSKFYLDKSKAILDKINL | |
| | NLIGSVPISILFIIAILSLILSIITFVIVMIIVRRYNKY | |
| | TPLINSDPSSRRSTIQDVYIIPNPGEHSIRSAARSID | |
| | RDRD | |
| 135 | ILHYEKLSKIGLVKGITRKYKIKSNPLTKDIVIKM | Hendra virus F |
| | IPNVSNVSKCTGTVMENYKSRLTGILSPIKGAIEL | Protein, Without |
| | YNNNTHDLVGDVKLAGVVMAGIAIGIATAAQIT | signal sequence |
| | AGVALYEAMKNADNINKLKSSIESTNEAVVKL | |
| | QETAEKTVYVLTALQDYINTNLVPTIDQISCKQT | |
| | ELALDLALSKYLSDLLFVFGPNLQDPVSNSMTIQ | |
| | AISQAFGGNYETLLRTLGYATEDFDDLLESDSIA | |
| | GQIVYVDLSSYYIIVRVYFPILTEIQQAYVQELLP | |
| | VSFNNDNSEWISIVPNFVLIRNTLISNIEVKYCLIT | |
| | KKSVICNQDYATPMTASVRECLTGSTDKCPREL | |
| | VVSSHVPRFALSGGVLFANCISVTCQCQTTGRAI | |
| | SQSGEQTLLMIDNTTCTTVVLGNIIISLGKYLGSI | |
| | NYNSESIAVGPPVYTDKVDISSQISSMNQSLQQS | |
| | KDYIKEAQKILDTVNPSLISMLSMIILYVLSIAAL | |
| | CIGLITFISFVIVEKKRGNYSRLDDRQVRPVSNG | |
| | DLYYIGT | |
| 136 | ILHYEKLSKIGLVKGVTRKYKIKSNPLTKDIVIK | Nipah virus F |
| | MIPNVSNMSQCTGSVMENYKTRLNGILTPIKGA | Protein, without |
| | LEIYKNNTHDLVGDVRLAGVIMAGVAIGIATAA | signal sequence |
| | QITAGVALYEAMKNADNINKLKSSIESTNEAVV | |
| | KLQETAEKTVYVLTALQDYINTNLVPTIDKISCK | |
| | QTELSLDLALSKYLSDLLFVFGPNLQDPVSNSM | |
| | TIQAISQAFGGNYETLLRTLGYATEDFDDLLESD | |
| | SITGQIIYVDLSSYYIIVRVYFPILTEIQQAYIQELL | |
| | PVSFNNDNSEWISIVPNFILVRNTLISNIEIGFCLIT | |
| | KRSVICNQDYATPMTNNMRECLTGSTEKCPREL | |
| | VVSSHVPRFALSNGVLFANCISVTCQCQTTGRAI | |
| | SQSGEQTLLMIDNTTCPTAVLGNVIISLGKYLGS | |
| | VNYNSEGIAIGPPVFTDKVDISSQISSMNQSLQQS | |
| | KDYIKEAQRLLDTVNPSLISMLSMIILYVLSIASL | |
| | CIGLITFISFIIVEKKRNTYSRLEDRRVRPTSSGDL | |
| | YYIGT | |
| 137 | TVLIIISYTLFYLNNAAIVGFDFDKLNKIGVVQG | Cedar Virus F |
| | RVLNYKIKGDPMTKDLVLKFIPNIVNITECVREP | Protein, without |
| | LSRYNETVRRLLLPIHNMLGLYLNNTNAKMTG | signal sequence |
| | LMIAGVIMGGIAIGIATAAQITAGFALYEAKKNT | |
| | ENIQKLTDSIMKTQDSIDKLTDSVGTSILILNKLQ | |
| | TYINNQLVPNLELLSCRQNKIEFDLMLTKYLVD | |
| | LMTVIGPNINNPVNKDMTIQSLSLLFDGNYDIM | |
| | MSELGYTPQDFLDLIESKSITGQIIYVDMENLYV | |
| | VIRTYLPTLIEVPDAQIYEFNKITMSSNGGEYLST | |
| | IPNFILIRGNYMSNIDVATCYMTKASVICNQDYS | |
| | LPMSQNLRSCYQGETEYCPVEAVIASHSPRFALT | |
| | NGVIFANCINTICRCQDNGKTITQNINQFVSMID | |
| | NSTCNDVMVDKFTIKVGKYMGRKDINNINIQIG | |
| | PQIIIDKVDLSNEINKMNQSLKDSIFYLREAKRIL | |
| | DSVNISLISPSVQLFLIIISVLSFIILLIIIVYLYCKSK | |
| | HSYKYNKFIDDPDYYNDYKRERINGKASKSNNI | |
| | YYVGD | |
| 138 | IHYDSLSKVGVIKGLTYNYKIKGSPSTKLMVVK | Mojiang virus, |
| | LIPNIDSVKNCTQKQYDEYKNLVRKALEPVKM | Tongguan 1 F |
| | AIDTMLNNVKSGNNKYRFAGAIMAGVALGVAT | Protein, without |
| | AATVTAGIALHRSNENAQAIANMKSAIQNTNEA | signal sequence |
| | VKQLQLANKQTLAVIDTIRGEINNNIIPVINQLSC | |
| | DTIGLSVGIRLTQYYSEIITAFGPALQNPVNTRITI | |
| | QAISSVFNGNFDELLKIMGYTSGDLYEILHSELIR | |
| | GNIIDVDVDAGYIALEIEFPNLTLVPNAVVQELM | |
| | PISYNIDGDEWVTLVPRFVLTRTTLLSNIDTSRCT | |
| | ITDSSVICDNDYALPMSHELIGCLQGDTSKCARE | |
| | KVVSSYVPKFALSDGLVYANCLNTICRCMDTDT | |
| | PISQSLGATVSLLDNKRCSVYQVGDVLISVGSYL | |
| | GDGEYNADNVELGPPIVIDKIDIGNQLAGINQTL | |
| | QEAEDYIEKSEEFLKGVNPSIITLGSMVVLYIFMI | |
| | LIAIVSVIALVLSIKLTVKGNVVRQQFTYTQHVP | |
| | SMENINYVSH | |
| 139 | SRALLRETDNYSNGLIVENLVRNCHHPSKNNLN | Bat Paramyxovirus |
| | YTKTQKRDSTIPYRVEERKGHYPKIKHLIDKSYK | F Protein, without |
| | HIKRGKRRNGHNGNIITIILLLILILKTQMSEGAIH | signal sequence |
| | YETLSKIGLIKGITREYKVKGTPSSKDIVIKLIPNV | |
| | TGLNKCTNISMENYKEQLDKILIPINNIIELYANS | |
| | TKSAPGNARFAGVIIAGVALGVAAAAQITAGIA | |
| | LHEARQNAERINLLKDSISATNNAVAELQEATG | |
| | GIVNVITGMQDYINTNLVPQIDKLQCSQIKTALD | |
| | ISLSQYYSEILTVFGPNLQNPVTTSMSIQAISQSF | |
| | GGNIDLLLNLLGYTANDLLDLLESKSITGQITYI | |
| | NLEHYFMVIRVYYPIMTTISNAYVQELIKISFNV | |
| | DGSEWVSLVPSYILIRNSYLSNIDISECLITKNSVI | |
| | CRHDFAMPMSYTLKECLTGDTEKCPREAVVTS | |
| | YVPRFAISGGVIYANCLSTTCQCYQTGKVIAQD | |
| | GSQTLMMIDNQTCSIVRIEEILISTGKYLGSQEYN | |
| | TMHVSVGNPVFTDKLDITSQISNINQSIEQSKFYL | |
| | DKSKAILDKINLNLIGSVPISILFIIAILSLILSIITFV | |
| | IVMIIVRRYNKYTPLINSDPSSRRSTIQDVYIIPNP | |
| | GEHSIRSAARSIDRDRD | |
| 140 | MVVILDKRCY CNLLILILMI SECSVGILHY | Truncated NiV |
| | EKLSKIGLVK GVTRKYKIKS NPLTKDIVIK | fusion |
| | MIPNVSNMSQ CTGSVMENYK TRLNGILTPI | glycoprotein |
| | KGALEIYKNN THDLVGDVRL AGVIMAGVAI | (FcDelta22) at |
| | GIATAAQITA GVALYEAMKN ADNINKLKSS | cytoplasmic tail |
| | IESTNEAVVK LQETAEKTVY VLTALQDYIN | (with signal |
| | TNLVPTIDKI SCKQTELSLD LALSKYLSDL | sequence) |
| | LFVFGPNLQD PVSNSMTIQA ISQAFGGNYE | |
| | TLLRTLGYAT EDFDDLLESD SITGQIIYVD | |
| | LSSYYIIVRV YFPILTEIQQ AYIQELLPVS | |
| | FNNDNSEWIS IVPNFILVRN TLISNIEIGF | |
| | CLITKRSVIC NQDYATPMTN NMRECLTGST | |
| | EKCPRELVVS SHVPRFALSN GVLFANCISV | |
| | TCQCQTTGRA ISQSGEQTLL MIDNTTCPTA | |
| | VLGNVIISLG KYLGSVNYNS EGIAIGPPVF | |
| | TDKVDISSQI SSMNQSLQQS KDYIKEAQRL | |
| | LDTVNPSLIS MLSMIILYVL SIASLCIGLI | |
| | TFISFIIVEK KRNT | |
| 141 | ILHY EKLSKIGLVK GVTRKYKIKS | Truncated mature |
| | NPLTKDIVIK MIPNVSNMSQ CTGSVMENYK | NiV fusion |
| | TRLNGILTPI KGALEIYKNN THDLVGDVRL | glycoprotein |
| | AGVIMAGVAI GIATAAQITA GVALYEAMKN | (FcDelta22) at |
| | ADNINKLKSS IESTNEAVVK LQETAEKTVY | cytoplasmic tail |
| | VLTALQDYIN TNLVPTIDKI SCKQTELSLD | |
| | LALSKYLSDL LFVFGPNLQD PVSNSMTIQA | |
| | ISQAFGGNYE TLLRTLGYAT EDFDDLLESD | |
| | SITGQIIYVD LSSYYIIVRV YFPILTEIQQ | |
| | AYIQELLPVS FNNDNSEWIS IVPNFILVRN | |
| | TLISNIEIGF CLITKRSVIC NQDYATPMTN | |
| | NMRECLTGST EKCPRELVVS SHVPRFALSN | |
| | GVLFANCISV TCQCQTTGRA ISQSGEQTLL | |
| | MIDNTTCPTA VLGNVIISLG KYLGSVNYNS | |
| | EGIAIGPPVF TDKVDISSQI SSMNQSLQQS | |
| | KDYIKEAQRL LDTVNPSLIS MLSMIILYVL | |
| | SIASLCIGLI TFISFIIVEK KRNT | |
| 142 | ENLYFQS | |
| 143 | MMADSKLVSLNNNLSGKIKDQGKVIKNYYGTM | gb: AF017149\| |
| | DIKKINDGLLDSKILGAFNTVIALLGSIIIIVMNIM | Organism: Hendra |
| | IIQNYTRTTDNQALIKESLQSVQQQIKALTDKIG | virus\| |
| | TEIGPKVSLIDTSSTITIPANIGLLGSKISQSTSSIN | Strain Name: UNKNOWN- |
| | ENVNDKCKFTLPPLKIHECNISCPNPLPFREYRPI | AF017149\|Protein |
| | SQGVSDLVGLPNQICLQKTTSTILKPRLISYTLPI | Name: glycoprotein\| |
| | NTREGVCITDPLLAVDNGFFAYSHLEKIGSCTRG | Gene Symbol: G |
| | IAKQRIIGVGEVLDRGDKVPSMFMTNVWTPPNP | |
| | STIHHCSSTYHEDFYYTLCAVSHVGDPILNSTSW | |
| | TESLSLIRLA VRPKSDSGDYNQKYIAITKVERGK | |
| | YDKVMPYGPSGIKQGDTLYFPAVGFLPRTEFQY | |
| | NDSNCPIIHCKYSKAENCRLSMGVNSKSHYILRS | |
| | GLLKYNLSLGGDIILQFIEIADNRLTIGSPSKIYNS | |
| | LGQPVFYQASYSWDTMIKLGDVDTVDPLRVQW | |
| | RNNSVISRPGQSQCPRFNVCPEVCWEGTYNDAF | |
| | LIDRLNWVSAGVYLNSNQTAENPVFAVFKDNEI | |
| | LYQVPLAEDDTNAQKTITDCFLLENVIWCISLVE | |
| | IYDTGDSVIRPKLFAVKIPAQCSES | |
| 144 | MLSQLQKNYLDNSNQQGDKMNNPDKKLSVNF | gb: JQ001776: |
| | NPLELDKGQKDLNKSYYVKNKNYNVSNLLNES | 8170-110275\| |
| | LHDIKFCIYCIFSLLIIITIINIITISIVITRLKVHEEN | Organism: |
| | NGMESPNLQSIQDSLSSLTNMINTEITPRIGILVT | Cedar virus\|Strain |
| | ATSVTLSSSINYVGTKTNQLVNELKDYITKSCGF | Name: CG1a\| |
| | KVPELKLHECNISCADPKISKSAMYSTNAYAEL | Protein |
| | AGPPKIFCKSVSKDPDFRLKQIDYVIPVQQDRSIC | Name: attachment |
| | MNNPLLDISDGFFTYIHYEGINSCKKSDSFKVLL | glycoprotein\|Gene |
| | SHGEIVDRGDYRPSLYLLSSHYHPYSMQVINCV | Symbol: G |
| | PVTCNQSSFVFCHISNNTKTLDNSDYSSDEYYIT | |
| | YFNGIDRPKTKKIPINNMTADNRYIHFTFSGGGG | |
| | VCLGEEFIIPVTTVINTDVFTHDYCESFNCSVQT | |
| | GKSLKEICSESLRSPTNSSRYNLNGIMIISQNNMT | |
| | DFKIQLNGITYNKLSFGSPGRLSKTLGQVLYYQS | |
| | SMSWDTYLKAGFVEKWKPFTPNWMNNTVISRP | |
| | NQGNCPRYHKCPEICYGGTYNDIAPLDLGKDM | |
| | YVSVILDSDQLAENPEITVENSTTILYKERVSKD | |
| | ELNTRSTTTSCFLFLDEPWCISVLETNRFNGKSIR | |
| | PEIYSYKIPKYC | |
| 145 | MPQKTVEFINMNSPLERGVSTLSDKKTLNQSKIT | gb: NC_025256: |
| | KQGYFGLGSHSERNWKKQKNQNDHYMTVSTM | 9117-11015\| |
| | ILEILVVLGIMFNLIVLTMVYYQNDNINQRMAEL | Organism: |
| | TSNITVLNLNLNQLTNKIQREIIPRITLIDTATTITI | Bat Paramyxovirus |
| | PSAITYILATLTTRISELLPSINQKCEFKTPTLVLN | Eid_hel/GH- |
| | DCRINCTPPLNPSDGVKMSSLATNLVAHGPSPC | M74a/GHA/2009\| |
| | RNFSSVPTIYYYRIPGLYNRTALDERCILNPRLTI | Strain |
| | SSTKFAYVHSEYDKNCTRGFKYYELMTFGEILE | Name: BatPV/Eid |
| | GPEKEPRMFSRSFYSPTNAVNYHSCTPIVTVNEG | hel/GH- |
| | YFLCLECTSSDPLYKANLSNSTFHLVILRHNKDE | M74a/GHA/2009\| |
| | KIVSMPSFNLSTDQEYVQIIPAEGGGTAESGNLY | Protein |
| | FPCIGRLLHKRVTHPLCKKSNCSRTDDESCLKSY | Name: glycoprotein\| |
| | YNQGSPQHQVVNCLIRIRNAQRDNPTWDVITVD | Gene Symbol: G |
| | LTNTYPGSRSRIFGSFSKPMLYQSSVSWHTLLQV | |
| | AEITDLDKYQLDWLDTPYISRPGGSECPFGNYCP | |
| | TVCWEGTYNDVYSLTPNNDLFVTVYLKSEQVA | |
| | ENPYFAIFSRDQILKEFPLDAWISSARTTTISCFM | |
| | FNNEIWCIAALEITRLNDDIIRPIYYSFWLPTDCR | |
| | TPYPHTGKMTRVPLRSTYNY | |
| 146 | MATNRDNTITSAEVSQEDKVKKYYGVETAEKV | gb: NC_025352: |
| | ADSISGNKVFILMNTLLILTGAIITITLNITNLTAA | 8716-11257\| |
| | KSQQNMLKIIQDDVNAKLEMFVNLDQLVKGEI | Organism: |
| | KPKVSLINTAVSVSIPGQISNLQTKFLQKYVYLE | Mojiang |
| | ESITKQCTCNPLSGIFPTSGPTYPPTDKPDDDTTD | virus\|Strain |
| | DDKVDTTIKPIEYPKPDGCNRTGDHFTMEPGAN | Name: Tongguan1\| |
| | FYTVPNLGPASSNSDECYTNPSFSIGSSIYMFSQE | Protein |
| | IRKTDCTAGEILSIQIVLGRIVDKGQQGPQASPLL | Name: attachment |
| | VWAVPNPKIINSCAVAAGDEMGWVLCSVTLTA | glycoprotein\|Gene |
| | ASGEPIPHMFDGFWLYKLEPDTEVVSYRITGYA | Symbol: G |
| | YLLDKQYDSVFIGKGGGIQKGNDLYFQMYGLS | |
| | RNRQSFKALCEHGSCLGTGGGGYQVLCDRAVM | |
| | SFGSEESLITNAYLKVNDLASGKPVIIGQTFPPSD | |
| | SYKGSNGRMYTIGDKYGLYLAPSSWNRYLRFGI | |
| | TPDISVRSTTWLKSQDPIMKILSTCTNTDRDMCP | |
| | EICNTRGYQDIFPLSEDSEYYTYIGITPNNGGTKN | |
| | FVAVRDSDGHIASIDILQNYYSITSATISCFMYKD | |
| | EIWCIAITEGKKQKDNPQRIYAHSYKIRQMCYN | |
| | MKSATVTVGNAKNITIRRY | |
| 147 | MPAENKKVRFENTTSDKGKIPSKVIKSYYGTMD | gb: AF212302\| |
| | IKKINEGLLDSKILSAFNTVIALLGSIVIIVMNIMII | Organism: Nipah |
| | QNYTRSTDNQAVIKDALQGIQQQIKGLADKIGT | virus\|Strain |
| | EIGPKVSLIDTSSTITIPANIGLLGSKISQSTASINE | Name: UNKNOWN- |
| | NVNEKCKFTLPPLKIHECNISCPNPLPFREYRPQT | AF212302\|Protein |
| | EGVSNLVGLPNNICLQKTSNQILKPKLISYTLPV | Name: attachment |
| | VGQSGTCITDPLLAMDEGYFAYSHLERIGSCSR | glycoprotein\|Gene |
| | GVSKQRIIGVGEVLDRGDEVPSLFMTNVWTPPN | Symbol: G |
| | PNTVYHCSAVYNNEFYYVLCAVSTVGDPILNST | |
| | YWSGSLMMTRLAVKPKSNGGGYNQHQLALRSI | |
| | EKGRYDKVMPYGPSGIKQGDTLYFPAVGFLVR | |
| | TEFKYNDSNCPITKCQYSKPENCRLSMGIRPNSH | |
| | YILRSGLLKYNLSDGENPKVVFIEISDQRLSIGSP | |
| | SKIYDSLGQPVFYQASFSWDTMIKFGDVLTVNP | |
| | LVVNWRNNTVISRPGQSQCPRFNTCPEICWEGV | |
| | YNDAFLIDRINWISAGVFLDSNQTAENPVFTVFK | |
| | DNEILYRAQLASEDTNAQKTITNCFLLKNKIWCI | |
| | SLVEIYDTGDNVIRPKLFAVKIPEQCT | |
| 148 | MALPVTALLLPLALLLHAARP | CD8α signal |
| | | peptide |
| 149 | METDTLLLWVLLLWVPGSTG | IgK signal peptide |
| 150 | MLLLVTSLLLCELPHPAFLLIP | GMCSFR-α |
| | | (CSF2RA) signal |
| | | peptide |
| 151 | TTTPAPRPPTPAPTIASQPLSLRPEACRPAAGGA | CD8α hinge |
| | VHTRGLDFACD | domain |
| 152 | IEVMYPPPYLDNEKSNGTIIHVKGKHLCPSPLFP | CD28 hinge |
| | GPSKP | domain |
| 153 | AAAIEVMYPPPYLDNEKSNGTIIHVKGKHLCPSP | CD28 hinge |
| | LFPGPSKP | domain |
| 154 | ESKYGPPCPPCP | IgG4 hinge domain |
| 155 | ESKYGPPCPSCP | IgG4 hinge domain |
| 156 | ESKYGPPCPPCPAPEFLGGPSVFLFPPKPKDTLMI | IgG4 hinge-CH2- |
| | SRTPEVTCVVVDVSQEDPEVQFNWYVDGVEVH | CH3 domain |
| | NAKTKPREEQFNSTYRVVSVLTVLHQDWLNGK | |
| | EYKCKVSNKGLPSSIEKTISKAKGQPREPQVYTL | |
| | PPSQEEMTKNQVSLTCLVKGFYPSDIAVEWESN | |
| | GQPENNYKTTPPVLDSDGSFFLYSRLTVDKSRW | |
| | QEGNVFSCSVMHEALHNHYTQKSLSLSLGK | |
| 157 | IYIWAPLAGTCGVLLLSLVITLYC | CD8α |
| | | transmembrane |
| | | domain |
| 158 | FWVLVVVGGVLACYSLLVTVAFIIFWV | CD28 |
| | | transmembrane |
| | | domain |
| 159 | MFWVLVVVGGVLACYSLLVTVAFIIFWV | CD28 |
| | | transmembrane |
| | | domain |
| 160 | KRGRKKLLYIFKQPFMRPVQTTQEEDGCSCRFP | 4-1BB |
| | EEEEGGCEL | costimulatory |
| | | domain |
| 161 | RSKRSRLLHSDYMNMTPRRPGPTRKHYQPYAPP | CD28 |
| | RDFAAYRS | costimulatory |
| | | domain |
| 162 | RVKFSRSADAPAYQQGQNQLYNELNLGRREEY | CD3ζ signaling |
| | DVLDKRRGRDPEMGGKPRRKNPQEGLYNELQK | domain |
| | DKMAEAYSEIGMKGERRRGKGHDGLYQGLSTA | |
| | TKDTYDALHMQALPPR | |
| 163 | RVKFSRSADAPAYKQGQNQLYNELNLGRREEY | CD3ζ signaling |
| | DVLDKRRGRDPEMGGKPRRKNPQEGLYNELQK | domain (with Q to |
| | DKMAEAYSEIGMKGERRRGKGHDGLYQGLSTA | K mutation at |
| | TKDTYDALHMQALPPR | position 14) |
| 164 | DIQMTQTTSSLSASLGDRVTISCRASQDISKYLN | Anti-CD19 |
| | WYQQKPDGTVKLLIYHTSRLHSGVPSRFSGSGS | FMC63 scFv entire |
| | GTDYSLTISNLEQEDIATYFCQQGNTLPYTFGGG | sequence, with |
| | TKLEITGSTSGSGKPGSGEGSTKGEVKLQESGPG | Whitlow linker |
| | LVAPSQSLSVTCTVSGVSLPDYGVSWIRQPPRK | |
| | GLEWLGVIWGSETTYYNSALKSRLTIIKDNSKS | |
| | QVFLKMNSLQTDDTAIYYCAKHYYYGGSYAM | |
| | DYWGQGTSVTVSS | |
| 165 | DIQMTQTTSSLSASLGDRVTISCRASQDISKYLN | Anti-CD19 |
| | WYQQKPDGTVKLLIYHTSRLHSGVPSRFSGSGS | FMC63 scFv light |
| | GTDYSLTISNLEQEDIATYFCQQGNTLPYTFGGG | chain variable |
| | TKLEIT | region |
| 166 | QDISKY | Anti-CD19 |
| | | FMC63 scFv light |
| | | chain CDR1 |
| 167 | HTS | Anti-CD19 |
| | | FMC63 scFv light |
| | | chain CDR2 |
| 168 | QQGNTLPYT | Anti-CD19 |
| | | FMC63 scFv light |
| | | chain CDR3 |
| 169 | GSTSGSGKPGSGEGSTKG | Whitlow linker |
| 170 | EVKLQESGPGLVAPSQSLSVTCTVSGVSLPDYG | Anti-CD19 |
| | VSWIRQPPRKGLEWLGVIWGSETTYYNSALKSR | FMC63 scFv |
| | LTIIKDNSKSQVFLKMNSLQTDDTAIYYCAKHY | heavy chain |
| | YYGGSYAMDYWGQGTSVTVSS | variable region |
| 171 | GVSLPDYG | Anti-CD19 |
| | | FMC63 scFv |
| | | heavy chain CDR1 |
| 172 | IWGSETT | Anti-CD19 |
| | | FMC63 scFv |
| | | heavy chain CDR2 |
| 173 | AKHYYYGGSYAMDY | Anti-CD19 |
| | | FMC63 scFv |
| | | heavy chain CDR3 |
| 174 | DIQMTQTTSSLSASLGDRVTISCRASQDISKYLN | Anti-CD19 |
| | WYQQKPDGTVKLLIYHTSRLHSGVPSRFSGSGS | FMC63 scFv entire |
| | GTDYSLTISNLEQEDIATYFCQQGNTLPYTFGGG | sequence, with |
| | TKLEITGGGGSGGGGSGGGGSEVKLQESGPGLV | 3xG₄S linker |
| | APSQSLSVTCTVSGVSLPDYGVSWIRQPPRKGLE | |
| | WLGVIWGSETTYYNSALKSRLTIIKDNSKSQVFL | |
| | KMNSLQTDDTAIYYCAKHYYYGGSYAMDYWG | |
| | QGTSVTVSS | |
| 175 | GGGGSGGGGSGGGGS | 3xG₄S linker |
| 176 | atggccttaccagtgaccgccttgctcctgccgctggccttgctgctccacg | Exemplary CD19 |
| | ccgccaggccggacatccagatgacacagactacatcctccctgtctgcct | CAR nucleotide |
| | ctctgggagacagagtcaccatcagttgcagggcaagtcaggacattagta | sequence |
| | aatatttaaattggtatcagcagaaaccagatggaactgttaaactcctgatct | |
| | accatacatcaagattacactcaggagtcccatcaaggttcagtggcagtg | |
| | ggtctggaacagattattctctcaccattagcaacctggagcaagaagatatt | |
| | gccacttacttttgccaacagggtaatacgcttccgtacacgttcggagggg | |
| | ggaccaagctggagatcacaggctccacctctggatccggcaagcccgg | |
| | atctggcgagggatccaccaagggcgaggtgaaactgcaggagtcagga | |
| | cctggcctggtggcgccctcacagagcctgtccgtcacatgcactgtctca | |
| | ggggtctcattacccgactatggtgtaagctggattcgccagcctccacgaa | |
| | agggtctggagtggctgggagtaatatggggtagtgaaaccacatactata | |
| | attcagctctcaaatccagactgaccatcatcaaggacaactccaagagcc | |
| | aagttttcttaaaaatgaacagtctgcaaactgatgacacagccatttactact | |
| | gtgccaaacattattactacggtggtagctatgctatggactactggggcca | |
| | aggaacctcagtcaccgtctcctcaaccacgacgccagcgccgcgaccac | |
| | caacaccggcgcccaccatcgcgtcgcagcccctgtccctgcgcccaga | |
| | ggcgtgccggccagcggcggggggcgcagtgcacacgagggggctgg | |
| | acttcgcctgtgatatctacatctgggcgcccttggccgggacttgtggggt | |
| | ccttctcctgtcactggttatcaccctttactgcaaacggggcagaaagaaac | |
| | tcctgtatatattcaaacaaccatttatgagaccagtacaaactactcaagag | |
| | gaagatggctgtagctgccgatttccagaagaagaagaaggaggatgtga | |
| | actgagagtgaagttcagcaggagcgcagacgcccccgcgtaccagcag | |
| | ggccagaaccagctctataacgagctcaatctaggacgaagagaggagta | |
| | cgatgttttggacaagagacgtggccgggaccctgagatggggggaaag | |
| | ccgagaaggaagaaccctcaggaaggcctgtacaatgaactgcagaaag | |
| | ataagatggcggaggcctacagtgagattgggatgaaaggcgagcgccg | |
| | gaggggcaaggggcacgatggcctttaccagggtctcagtacagccacc | |
| | aaggacacctacgacgcccttcacatgcaggccctgccccctcgc | |
| 177 | MALPVTALLLPLALLLHAARPDIQMTQTTSSLS | Exemplary CD19 |
| | ASLGDRVTISCRASQDISKYLNWYQQKPDGTVK | CAR amino acid |
| | LLIYHTSRLHSGVPSRFSGSGSGTDYSLTISNLEQ | sequence |
| | EDIATYFCQQGNTLPYTFGGGTKLEITGSTSGSG | |
| | KPGSGEGSTKGEVKLQESGPGLVAPSQSLSVTC | |
| | TVSGVSLPDYGVSWIRQPPRKGLEWLGVIWGSE | |
| | TTYYNSALKSRLTIIKDNSKSQVFLKMNSLQTD | |
| | DTAIYYCAKHYYYGGSYAMDYWGQGTSVTVS | |
| | STTTPAPRPPTPAPTIASQPLSLRPEACRPAAGGA | |
| | VHTRGLDFACDIYIWAPLAGTCGVLLLSLVITLY | |
| | CKRGRKKLLYIFKQPFMRPVQTTQEEDGCSCRF | |
| | PEEEEGGCELRVKFSRSADAPAYQQGQNQLYN | |
| | ELNLGRREEYDVLDKRRGRDPEMGGKPRRKNP | |
| | QEGLYNELQKDKMAEAYSEIGMKGERRRGKG | |
| | HDGLYQGLSTATKDTYDALHMQALPPR | |
| 178 | atggccttaccagtgaccgccttgctcctgccgctggccttgctgctccacg | Tisagenlecleucel |
| | ccgccaggccggacatccagatgacacagactacatcctccctgtctgcct | CD19 CAR |
| | ctctgggagacagagtcaccatcagttgcagggcaagtcaggacattagta | nucleotide |
| | aatatttaaattggtatcagcagaaaccagatggaactgttaaactcctgatct | sequence |
| | accatacatcaagattacactcaggagtcccatcaaggttcagtggcagtg | |
| | ggtctggaacagattattctctcaccattagcaacctggagcaagaagatatt | |
| | gccacttacttttgccaacagggtaatacgcttccgtacacgttcggagggg | |
| | ggaccaagctggagatcacaggtggcggtggctcgggcggtggtgggtc | |
| | gggtggcggcggatctgaggtgaaactgcaggagtcaggacctggcctg | |
| | gtggcgccctcacagagcctgtccgtcacatgcactgtctcaggggtctcat | |
| | tacccgactatggtgtaagctggattcgccagcctccacgaaagggtctgg | |
| | agtggctgggagtaatatggggtagtgaaaccacatactataattcagctct | |
| | caaatccagactgaccatcatcaaggacaactccaagagccaagttttctta | |
| | aaaatgaacagtctgcaaactgatgacacagccatttactactgtgccaaac | |
| | attattactacggtggtagctatgctatggactactggggccaaggaacctc | |
| | agtcaccgtctcctcaaccacgacgccagcgccgcgaccaccaacaccg | |
| | gcgcccaccatcgcgtcgcagcccctgtccctgcgcccagaggcgtgcc | |
| | ggccagcggggggggcgcagtgcacacgagggggctggacttcgcct | |
| | gtgatatctacatctgggcgcccttggccgggacttgtggggtccttctcctg | |
| | tcactggttatcaccctttactgcaaacggggcagaaagaaactcctgtatat | |
| | attcaaacaaccatttatgagaccagtacaaactactcaagaggaagatggc | |
| | tgtagctgccgatttccagaagaagaagaaggaggatgtgaactgagagt | |
| | gaagttcagcaggagcgcagacgcccccgcgtacaagcagggccagaa | |
| | ccagctctataacgagctcaatctaggacgaagagaggagtacgatgttttg | |
| | gacaagagacgtggccgggaccctgagatggggggaaagccgagaag | |
| | gaagaaccctcaggaaggcctgtacaatgaactgcagaaagataagatgg | |
| | cggaggcctacagtgagattgggatgaaaggcgagcgccggaggggca | |
| | aggggcacgatggcctttaccagggtctcagtacagccaccaaggacacc | |
| | tacgacgcccttcacatgcaggccctgccccctcgc | |
| 179 | MALPVTALLLPLALLLHAARPDIQMTQTTSSLS | Tisagenlecleucel |
| | ASLGDRVTISCRASQDISKYLNWYQQKPDGTVK | CD19 CAR amino |
| | LLIYHTSRLHSGVPSRFSGSGSGTDYSLTISNLEQ | acid sequence |
| | EDIATYFCQQGNTLPYTFGGGTKLEITGGGGSG | |
| | GGGSGGGGSEVKLQESGPGLVAPSQSLSVTCTV | |
| | SGVSLPDYGVSWIRQPPRKGLEWLGVIWGSETT | |
| | YYNSALKSRLTIIKDNSKSQVFLKMNSLQTDDT | |
| | AIYYCAKHYYYGGSYAMDYWGQGTSVTVSST | |
| | TTPAPRPPTPAPTIASQPLSLRPEACRPAAGGAV | |
| | HTRGLDFACDIYIWAPLAGTCGVLLLSLVITLYC | |
| | KRGRKKLLYIFKQPFMRPVQTTQEEDGCSCRFP | |
| | EEEEGGCELRVKFSRSADAPAYKQGQNQLYNE | |
| | LNLGRREEYDVLDKRRGRDPEMGGKPRRKNPQ | |
| | EGLYNELQKDKMAEAYSEIGMKGERRRGKGH | |
| | DGLYQGLSTATKDTYDALHMQALPPR | |
| 180 | atgctgctgctggtgaccagcctgctgctgtgcgagctgccccaccccgcc | Lisocabtagene |
| | tttctgctgatccccgacatccagatgacccagaccacctccagcctgagc | maraleucel CD19 |
| | gccagcctgggcgaccgggtgaccatcagctgccgggccagccaggac | CAR nucleotide |
| | atcagcaagtacctgaactggtatcagcagaagcccgacggcaccgtcaa | sequence |
| | gctgctgatctaccacaccagccggctgcacagcggcgtgcccagccggt | |
| | ttagcggcagcggctccggcaccgactacagcctgaccatctccaacctg | |
| | gaacaggaagatatcgccacctacttttgccagcagggcaacacactgccc | |
| | tacacctttggcggcggaacaaagctggaaatcaccggcagcacctccgg | |
| | cagcggcaagcctggcagcggcgagggcagcaccaagggcgaggtga | |
| | agctgcaggaaagcggccctggcctggtggcccccagccagagcctga | |
| | gcgtgacctgcaccgtgagcggcgtgagcctgcccgactacggcgtgag | |
| | ctggatccggcagccccccaggaagggcctggaatggctgggcgtgatc | |
| | tggggcagcgagaccacctactacaacagcgccctgaagagccggctga | |
| | ccatcatcaaggacaacagcaagagccaggtgttcctgaagatgaacagc | |
| | ctgcagaccgacgacaccgccatctactactgcgccaagcactactactac | |
| | ggcggcagctacgccatggactactggggccagggcaccagcgtgacc | |
| | gtgagcagegaatctaagtacggaccgccctgccccccttgccctatgttct | |
| | gggtgctggtggtggtcggaggcgtgctggcctgctacagcctgctggtc | |
| | accgtggccttcatcatcttttgggtgaaacggggcagaaagaaactcctgt | |
| | atatattcaaacaaccatttatgagaccagtacaaactactcaagaggaagat | |
| | ggctgtagctgccgatttccagaagaagaagaaggaggatgtgaactgcg | |
| | ggtgaagttcagcagaagcgccgacgcccctgcctaccagcagggccag | |
| | aatcagctgtacaacgagctgaacctgggcagaagggaagagtacgacg | |
| | tcctggataagcggagaggccgggaccctgagatgggcggcaagcctcg | |
| | gcggaagaacccccaggaaggcctgtataacgaactgcagaaagacaag | |
| | atggccgaggcctacagcgagatcggcatgaagggcgagcggaggcgg | |
| | ggcaagggccacgacggcctgtatcagggcctgtccaccgccaccaagg | |
| | atacctacgacgccctgcacatgcaggccctgcccccaagg | |
| 181 | MLLLVTSLLLCELPHPAFLLIPDIQMTQTTSSLSA | Lisocabtagene |
| | SLGDRVTISCRASQDISKYLNWYQQKPDGTVKL | maraleucel CD19 |
| | LIYHTSRLHSGVPSRFSGSGSGTDYSLTISNLEQE | CAR amino acid |
| | DIATYFCQQGNTLPYTFGGGTKLEITGSTSGSGK | sequence |
| | PGSGEGSTKGEVKLQESGPGLVAPSQSLSVTCT | |
| | VSGVSLPDYGVSWIRQPPRKGLEWLGVIWGSET | |
| | TYYNSALKSRLTIIKDNSKSQVFLKMNSLQTDD | |
| | TAIYYCAKHYYYGGSYAMDYWGQGTSVTVSS | |
| | ESKYGPPCPPCPMFWVLVVVGGVLACYSLLVT | |
| | VAFIIFWVKRGRKKLLYIFKQPFMRPVQTTQEE | |
| | DGCSCRFPEEEEGGCELRVKFSRSADAPAYQQG | |
| | QNQLYNELNLGRREEYDVLDKRRGRDPEMGG | |
| | KPRRKNPQEGLYNELQKDKMAEAYSEIGMKGE | |
| | RRRGKGHDGLYQGLSTATKDTYDALHMQALPP | |
| | R | |
| 182 | atgcttctcctggtgacaagccttctgctctgtgagttaccacacccagcattc | Axicabtagene |
| | ctcctgatcccagacatccagatgacacagactacatcctccctgtctgcctc | ciloleucel CD19 |
| | tctgggagacagagtcaccatcagttgcagggcaagtcaggacattagtaa | CAR nucleotide |
| | atatttaaattggtatcagcagaaaccagatggaactgttaaactcctgatcta | sequence |
| | ccatacatcaagattacactcaggagtcccatcaaggttcagtggcagtgg | |
| | gtctggaacagattattctctcaccattagcaacctggagcaagaagatattg | |
| | ccacttacttttgccaacagggtaatacgcttccgtacacgttcggaggggg | |
| | gactaagttggaaataacaggctccacctctggatccggcaagcccggatc | |
| | tggcgagggatccaccaagggcgaggtgaaactgcaggagtcaggacct | |
| | ggcctggtggcgccctcacagagcctgtccgtcacatgcactgtctcaggg | |
| | gtctcattacccgactatggtgtaagctggattcgccagcctccacgaaagg | |
| | gtctggagtggctgggagtaatatggggtagtgaaaccacatactataattc | |
| | agctctcaaatccagactgaccatcatcaaggacaactccaagagccaagt | |
| | tttcttaaaaatgaacagtctgcaaactgatgacacagccatttactactgtgc | |
| | caaacattattactacggtggtagctatgctatggactactggggtcaagga | |
| | acctcagtcaccgtctcctcageggccgcaattgaagttatgtatcctcctcc | |
| | ttacctagacaatgagaagagcaatggaaccattatccatgtgaaagggaa | |
| | acacctttgtccaagtcccctatttcccggaccttctaagcccttttgggtgct | |
| | ggtggtggttgggggagtcctggcttgctatagcttgctagtaacagtggcc | |
| | tttattattttctgggtgaggagtaagaggagcaggctcctgcacagtgacta | |
| | catgaacatgactccccgccgccccgggcccacccgcaagcattaccagc | |
| | cctatgccccaccacgcgacttcgcagcctatcgctccagagtgaagttca | |
| | gcaggagcgcagacgcccccgcgtaccagcagggccagaaccagctct | |
| | ataacgagctcaatctaggacgaagagaggagtacgatgttttggacaaga | |
| | gacgtggccgggaccctgagatggggggaaagccgagaaggaagaac | |
| | cctcaggaaggcctgtacaatgaactgcagaaagataagatggcggagg | |
| | cctacagtgagattgggatgaaaggcgagcgccggaggggcaaggggc | |
| | acgatggcctttaccagggtctcagtacagccaccaaggacacctacgac | |
| | gcccttcacatgcaggccctgccccctcgc | |
| 183 | MLLLVTSLLLCELPHPAFLLIPDIQMTQTTSSLSA | Axicabtagene |
| | SLGDRVTISCRASQDISKYLNWYQQKPDGTVKL | ciloleucel CD19 |
| | LIYHTSRLHSGVPSRFSGSGSGTDYSLTISNLEQE | CAR amino acid |
| | DIATYFCQQGNTLPYTFGGGTKLEITGSTSGSGK | sequence |
| | PGSGEGSTKGEVKLQESGPGLVAPSQSLSVTCT | |
| | VSGVSLPDYGVSWIRQPPRKGLEWLGVIWGSET | |
| | TYYNSALKSRLTIIKDNSKSQVFLKMNSLQTDD | |
| | TAIYYCAKHYYYGGSYAMDYWGQGTSVTVSS | |
| | AAAIEVMYPPPYLDNEKSNGTIIHVKGKHLCPSP | |
| | LFPGPSKPFWVLVVVGGVLACYSLLVTVAFIIF | |
| | WVRSKRSRLLHSDYMNMTPRRPGPTRKHYQPY | |
| | APPRDFAAYRSRVKFSRSADAPAYQQGQNQLY | |
| | NELNLGRREEYDVLDKRRGRDPEMGGKPRRKN | |
| | PQEGLYNELQKDKMAEAYSEIGMKGERRRGKG | |
| | HDGLYQGLSTATKDTYDALHMQALPPR | |
| 184 | DIVLTQSPAILSASPGEKVTMTCRASSSVNYMD | Anti-CD20 Leu16 |
| | WYQKKPGSSPKPWIYATSNLASGVPARFSGSGS | scFv entire |
| | GTSYSLTISRVEAEDAATYYCQQWSFNPPTFGG | sequence, with |
| | GTKLEIKGSTSGSGKPGSGEGSTKGEVQLQQSG | Whitlow linker |
| | AELVKPGASVKMSCKASGYTFTSYNMHWVKQ | |
| | TPGQGLEWIGAIYPGNGDTSYNQKFKGKATLTA | |
| | DKSSSTAYMQLSSLTSEDSADYYCARSNYYGSS | |
| | YWFFDVWGAGTTVTVSS | |
| 185 | DIVLTQSPAILSASPGEKVTMTCRASSSVNYMD | Anti-CD20 Leu16 |
| | WYQKKPGSSPKPWIYATSNLASGVPARFSGSGS | scFv light chain |
| | GTSYSLTISRVEAEDAATYYCQQWSFNPPTFGG | variable region |
| | GTKLEIK | |
| 186 | RASSSVNYMD | Anti-CD20 Leu16 |
| | | scFv light chain |
| | | CDR1 |
| 187 | ATSNLAS | Anti-CD20 Leu16 |
| | | scFv light chain |
| | | CDR2 |
| 188 | QQWSFNPPT | Anti-CD20 Leu16 |
| | | scFv light chain |
| | | CDR3 |
| 189 | EVQLQQSGAELVKPGASVKMSCKASGYTFTSY | Anti-CD20 Leu16 |
| | NMHWVKQTPGQGLEWIGAIYPGNGDTSYNQKF | scFv heavy chain |
| | KGKATLTADKSSSTAYMQLSSLTSEDSADYYCA | |
| | RSNYYGSSYWFFDVWGAGTTVTVSS | |
| 190 | SYNMH | Anti-CD20 Leu16 |
| | | scFv heavy chain |
| | | CDR1 |
| 191 | AIYPGNGDTSYNQKFKG | Anti-CD20 Leu16 |
| | | scFv heavy chain |
| | | CDR2 |
| 192 | SNYYGSSYWFFDV | Anti-CD20 Leu16 |
| | | scFv heavy chain |
| | | CDR3 |
| 193 | QVQLQQSGPGLVKPSQTLSLTCAISGDSVSSNSA | Anti-CD22 m971 |
| | AWNWIRQSPSRGLEWLGRTYYRSKWYNDYAV | scFv entire |
| | SVKSRITINPDTSKNQFSLQLNSVTPEDTAVYYC | sequence, with |
| | AREVTGDLEDAFDIWGQGTMVTVSSGGGGSGG | 3xG₄S linker |
| | GGSGGGGSDIQMTQSPSSLSASVGDRVTITCRAS | |
| | QTIWSYLNWYQQRPGKAPNLLIYAASSLQSGVP | |
| | SRFSGRGSGTDFTLTISSLQAEDFATYYCQQSYSI | |
| | PQTFGQGTKLEIK | |
| 194 | QVQLQQSGPGLVKPSQTLSLTCAISGDSVSSNSA | Anti-CD22 m971 |
| | AWNWIRQSPSRGLEWLGRTYYRSKWYNDYAV | scFv heavy chain |
| | SVKSRITINPDTSKNQFSLQLNSVTPEDTAVYYC | variable region |
| | AREVTGDLEDAFDIWGQGTMVTVSS | |
| 195 | GDSVSSNSAA | Anti-CD22 m971 |
| | | scFv heavy chain |
| | | CDR1 |
| 196 | TYYRSKWYN | Anti-CD22 m971 |
| | | scFv heavy chain |
| | | CDR2 |
| 197 | AREVTGDLEDAFDI | Anti-CD22 m971 |
| | | scFv heavy chain |
| | | CDR3 |
| 198 | DIQMTQSPSSLSASVGDRVTITCRASQTIWSYLN | Anti-CD22 m971 |
| | WYQQRPGKAPNLLIYAASSLQSGVPSRFSGRGS | scFv light chain |
| | GTDFTLTISSLQAEDFATYYCQQSYSIPQTFGQG | |
| | TKLEIK | |
| 199 | QTIWSY | Anti-CD22 m971 |
| | | scFv light chain |
| | | CDR1 |
| 200 | AAS | Anti-CD22 m971 |
| | | scFv light chain |
| | | CDR2 |
| 201 | QQSYSIPQT | Anti-CD22 m971 |
| | | scFv light chain |
| | | CDR3 |
| 202 | QVQLQQSGPGMVKPSQTLSLTCAISGDSVSSNS | Anti-CD22 m971- |
| | VAWNWIRQSPSRGLEWLGRTYYRSTWYNDYA | L7 scFv entire |
| | VSMKSRITINPDTNKNQFSLQLNSVTPEDTAVY | sequence, with |
| | YCAREVTGDLEDAFDIWGQGTMVTVSSGGGGS | 3xG₄S linker |
| | GGGGSGGGGSDIQMIQSPSSLSASVGDRVTITCR | |
| | ASQTIWSYLNWYRQRPGEAPNLLIYAASSLQSG | |
| | VPSRFSGRGSGTDFTLTISSLQAEDFATYYCQQS | |
| | YSIPQTFGQGTKLEIK | |
| 203 | QVQLQQSGPGMVKPSQTLSLTCAISGDSVSSNS | Anti-CD22 m971- |
| | VAWNWIRQSPSRGLEWLGRTYYRSTWYNDYA | L7 scFv heavy |
| | VSMKSRITINPDTNKNQFSLQLNSVTPEDTAVY | chain variable |
| | YCAREVTGDLEDAFDIWGQGTMVTVSS | region |
| 204 | GDSVSSNSVA | Anti-CD22 m971- |
| | | L7 scFv heavy |
| | | chain CDR1 |
| 205 | TYYRSTWYN | Anti-CD22 m971- |
| | | L7 scFv heavy |
| | | chain CDR2 |
| 206 | AREVTGDLEDAFDI | Anti-CD22 m971- |
| | | L7 scFv heavy |
| | | chain CDR3 |
| 207 | DIQMIQSPSSLSASVGDRVTITCRASQTIWSYLN | Anti-CD22 m971- |
| | WYRQRPGEAPNLLIYAASSLQSGVPSRFSGRGS | L7 scFv light chain |
| | GTDFTLTISSLQAEDFATYYCQQSYSIPQTFGQG | variable region |
| | TKLEIK | |
| 208 | QTIWSY | Anti-CD22 m971- |
| | | L7 scFv light chain |
| | | CDR1 |
| 209 | AAS | Anti-CD22 m971- |
| | | L7 scFv light chain |
| | | CDR2 |
| 210 | QQSYSIPQT | Anti-CD22 m971- |
| | | L7 scFv light chain |
| | | CDR3 |
| 211 | DIVLTQSPASLAMSLGKRATISCRASESVSVIGA | Anti-BCMA |
| | HLIHWYQQKPGQPPKLLIYLASNLETGVPARFS | C11D5.3 scFv |
| | GSGSGTDFTLTIDPVEEDDVAIYSCLQSRIFPRTF | entire sequence, |
| | GGGTKLEIKGSTSGSGKPGSGEGSTKGQIQLVQS | with Whitlow |
| | GPELKKPGETVKISCKASGYTFTDYSINWVKRA | linker |
| | PGKGLKWMGWINTETREPAYAYDFRGRFAFSL | |
| | ETSASTAYLQINNLKYEDTATYFCALDYSYAMD | |
| | YWGQGTSVTVSS | |
| 212 | DIVLTQSPASLAMSLGKRATISCRASESVSVIGA | Anti-BCMA |
| | HLIHWYQQKPGQPPKLLIYLASNLETGVPARFS | C11D5.3 scFv |
| | GSGSGTDFTLTIDPVEEDDVAIYSCLQSRIFPRTF | light chain variable |
| | GGGTKLEIK | region |
| 213 | RASESVSVIGAHLIH | Anti-BCMA |
| | | C11D5.3 scFv |
| | | light chain CDR1 |
| 214 | LASNLET | Anti-BCMA |
| | | C11D5.3 scFv |
| | | light chain CDR2 |
| 215 | LQSRIFPRT | Anti-BCMA |
| | | C11D5.3 scFv |
| | | light chain CDR3 |
| 216 | QIQLVQSGPELKKPGETVKISCKASGYTFTDYSI | Anti-BCMA |
| | NWVKRAPGKGLKWMGWINTETREPAYAYDFR | C11D5.3 scFv |
| | GRFAFSLETSASTAYLQINNLKYEDTATYFCAL | heavy chain |
| | DYSYAMDYWGQGTSVTVSS | variable region |
| 217 | DYSIN | Anti-BCMA |
| | | C11D5.3 scFv |
| | | heavy chain CDR1 |
| 218 | WINTETREPAYAYDFRG | Anti-BCMA |
| | | C11D5.3 scFv |
| | | heavy chain CDR2 |
| 219 | DYSYAMDY | Anti-BCMA |
| | | C11D5.3 scFv |
| | | heavy chain CDR3 |
| 220 | DIVLTQSPPSLAMSLGKRATISCRASESVTILGSH | Anti-BCMA |
| | LIYWYQQKPGQPPTLLIQLASNVQTGVPARFSG | C12A3.2 scFv |
| | SGSRTDFTLTIDPVEEDDVAVYYCLQSRTIPRTF | entire sequence, |
| | GGGTKLEIKGSTSGSGKPGSGEGSTKGQIQLVQS | with Whitlow |
| | GPELKKPGETVKISCKASGYTFRHYSMNWVKQ | linker |
| | APGKGLKWMGRINTESGVPIYADDFKGRFAFSV | |
| | ETSASTAYLVINNLKDEDTASYFCSNDYLYSLD | |
| | FWGQGTALTVSS | |
| 221 | DIVLTQSPPSLAMSLGKRATISCRASESVTILGSH | Anti-BCMA |
| | LIYWYQQKPGQPPTLLIQLASNVQTGVPARFSG | C12A3.2 scFv |
| | SGSRTDFTLTIDPVEEDDVAVYYCLQSRTIPRTF | light chain variable |
| | GGGTKLEIK | region |
| 222 | RASESVTILGSHLIY | Anti-BCMA |
| | | C12A3.2 scFv |
| | | light chain CDR1 |
| 223 | LASNVQT | Anti-BCMA |
| | | C12A3.2 scFv |
| | | light chain CDR2 |
| 224 | LQSRTIPRT | Anti-BCMA |
| | | C12A3.2 scFv |
| | | light chain CDR3 |
| 225 | QIQLVQSGPELKKPGETVKISCKASGYTFRHYS | Anti-BCMA |
| | MNWVKQAPGKGLKWMGRINTESGVPIYADDF | C12A3.2 scFv |
| | KGRFAFSVETSASTAYLVINNLKDEDTASYFCS | heavy chain |
| | NDYLYSLDFWGQGTALTVSS | variable region |
| 226 | HYSMN | Anti-BCMA |
| | | C12A3.2 scFv |
| | | heavy chain CDR1 |
| 227 | RINTESGVPIYADDFKG | Anti-BCMA |
| | | C12A3.2 scFv |
| | | heavy chain CDR2 |
| 228 | DYLYSLDF | Anti-BCMA |
| | | C12A3.2 scFv |
| | | heavy chain CDR3 |
| 229 | EVQLLESGGGLVQPGGSLRLSCAASGFTFSSYA | Anti-BCMA |
| | MSWVRQAPGKGLEWVSSISGSGDYIYYADSVK | FHVH33 entire |
| | GRFTISRDISKNTLYLQMNSLRAEDTAVYYCAK | sequence |
| | EGTGANSSLADYRGQGTLVTVSS | |
| 230 | GFTFSSYA | Anti-BCMA |
| | | FHVH33 CDR1 |
| 231 | ISGSGDYI | Anti-BCMA |
| | | FHVH33 CDR2 |
| 232 | AKEGTGANSSLADY | Anti-BCMA |
| | | FHVH33 CDR3 |
| 233 | DIQMTQSPSSLSASVGDRVTITCRASQSISSYLN | Anti-BCMA |
| | WYQQKPGKAPKLLIYAASSLQSGVPSRFSGSGS | CT103A scFv |
| | GTDFTLTISSLQPEDFATYYCQQKYDLLTFGGGT | entire sequence, |
| | KVEIKGSTSGSGKPGSGEGSTKGQLQLQESGPG | with Whitlow |
| | LVKPSETLSLTCTVSGGSISSSSYYWGWIRQPPG | linker |
| | KGLEWIGSISYSGSTYYNPSLKSRVTISVDTSKN | |
| | QFSLKLSSVTAADTAVYYCARDRGDTILDVWG | |
| | QGTMVTVSS | |
| 234 | DIQMTQSPSSLSASVGDRVTITCRASQSISSYLN | Anti-BCMA |
| | WYQQKPGKAPKLLIYAASSLQSGVPSRFSGSGS | CT103A scFv light |
| | GTDFTLTISSLQPEDFATYYCQQKYDLLTFGGGT | chain variable |
| | KVEIK | region |
| 235 | QSISSY | Anti-BCMA |
| | | CT103A scFv light |
| | | chain CDR1 |
| 236 | AAS | Anti-BCMA |
| | | CT103A scFv light |
| | | chain CDR2 |
| 237 | QQKYDLLT | Anti-BCMA |
| | | CT103A scFv light |
| | | chain CDR3 |
| 238 | QLQLQESGPGLVKPSETLSLTCTVSGGSISSSSYY | Anti-BCMA |
| | WGWIRQPPGKGLEWIGSISYSGSTYYNPSLKSR | CT103A scFv |
| | VTISVDTSKNQFSLKLSSVTAADTAVYYCARDR | heavy chain |
| | GDTILDVWGQGTMVTVSS | variable region |
| 239 | GGSISSSSYY | Anti-BCMA |
| | | CT103A scFv |
| | | heavy chain CDR1 |
| 240 | ISYSGST | Anti-BCMA |
| | | CT103A scFv |
| | | heavy chain CDR2 |
| 241 | ARDRGDTILDV | Anti-BCMA |
| | | CT103A scFv |
| | | heavy chain CDR3 |
| 242 | atggccttaccagtgaccgccttgctcctgccgctggccttgctgctccacg | Exemplary BCMA |
| | ccgccaggccggacatccagatgacccagtctccatcctccctgtctgcat | CAR nucleotide |
| | ctgtaggagacagagtcaccatcacttgccgggcaagtcagagcattagc | sequence |
| | agctatttaaattggtatcagcagaaaccagggaaagcccctaagctcctga | |
| | tctatgctgcatccagtttgcaaagtggggtcccatcaaggttcagtggcagt | |
| | ggatctgggacagatttcactctcaccatcagcagtctgcaacctgaagattt | |
| | tgcaacttactactgtcagcaaaaatacgacctcctcacttttggcggaggg | |
| | accaaggttgagatcaaaggcagcaccagcggctccggcaagcctggct | |
| | ctggcgagggcagcacaaagggacagctgcagctgcaggagtcgggcc | |
| | caggactggtgaagccttcggagaccctgtccctcacctgcactgtctctgg | |
| | tggctccatcagcagtagtagttactactggggctggatccgccagccccc | |
| | agggaaggggctggagtggattgggagtatctcctatagtgggagcacct | |
| | actacaacccgtccctcaagagtcgagtcaccatatccgtagacacgtcca | |
| | agaaccagttctccctgaagctgagttctgtgaccgccgcagacacggcg | |
| | gtgtactactgcgccagagatcgtggagacaccatactagacgtatggggt | |
| | cagggtacaatggtcaccgtcagctcattcgtgcccgtgttcctgcccgcca | |
| | aacctaccaccacccctgcccctagacctcccaccccagccccaacaatc | |
| | gccagccagcctctgtctctgcggcccgaagcctgtagacctgctgccgg | |
| | cggagccgtgcacaccagaggcctggacttcgcctgcgacatctacatct | |
| | gggcccctctggccggcacctgtggcgtgctgctgctgagcctggtgatca | |
| | ccctgtactgcaaccaccggaacaaacggggcagaaagaaactcctgtat | |
| | atattcaaacaaccatttatgagaccagtacaaactactcaagaggaagatg | |
| | gctgtagctgccgatttccagaagaagaagaaggaggatgtgaactgaga | |
| | gtgaagttcagcagatccgccgacgcccctgcctaccagcagggacaga | |
| | accagctgtacaacgagctgaacctgggcagacgggaagagtacgacgt | |
| | gctggacaagcggagaggccgggaccccgagatgggcggaaagccca | |
| | gacggaagaacccccaggaaggcctgtataacgaactgcagaaagacaa | |
| | gatggccgaggcctacagcgagatcggcatgaagggcgagcggaggcg | |
| | cggcaagggccacgatggcctgtaccagggcctgagcaccgccaccaa | |
| | ggacacctacgacgccctgcacatgcaggccctgccccccaga | |
| 243 | MALPVTALLLPLALLLHAARPDIQMTQSPSSLSA | Exemplary BCMA |
| | SVGDRVTITCRASQSISSYLNWYQQKPGKAPKL | CAR amino acid |
| | LIYAASSLQSGVPSRFSGSGSGTDFTLTISSLQPE | sequence |
| | DFATYYCQQKYDLLTFGGGTKVEIKGSTSGSGK | |
| | PGSGEGSTKGQLQLQESGPGLVKPSETLSLTCTV | |
| | SGGSISSSSYYWGWIRQPPGKGLEWIGSISYSGS | |
| | TYYNPSLKSRVTISVDTSKNQFSLKLSSVTAADT | |
| | AVYYCARDRGDTILDVWGQGTMVTVSSFVPVF | |
| | LPAKPTTTPAPRPPTPAPTIASQPLSLRPEACRPA | |
| | AGGAVHTRGLDFACDIYIWAPLAGTCGVLLLSL | |
| | VITLYCNHRNKRGRKKLLYIFKQPFMRPVQTTQ | |
| | EEDGCSCRFPEEEEGGCELRVKFSRSADAPAYQ | |
| | QGQNQLYNELNLGRREEYDVLDKRRGRDPEM | |
| | GGKPRRKNPQEGLYNELQKDKMAEAYSEIGMK | |
| | GERRRGKGHDGLYQGLSTATKDTYDALHMQA | |
| | LPPR | |
| 244 | MSPQRDRINAFYKDNPHPKGSRIVINREHLMIDR | Measles virus H |
| | PYVLLAVLFVMFLSLIGLLAIAGIRLHRAAIYTA | protein |
| | EIHKSLSTNLDVTNSIEHQVKDVLTPLFKIIGDEV | |
| | GLRTPQRFTDLVKFISDKIKFLNPDREYDFRDLT | |
| | WCINPPERIKLDYDQYCADVAAEELMNALVNS | |
| | TLLETRTTNQFLAVSKGNCSGPTTIRGQFSNMSL | |
| | SLLDLYLGRGYNVSSIVTMTSQGMYGGTYLVE | |
| | KPNLSSKRSELSQLSMYRVFEVGVIRNPGLGAP | |
| | VFHMTNYLEQPVSNDLSNCMVALGELKLAALC | |
| | HGEDSITIPYQGSGKGVSFQLVKLGVWKSPTDM | |
| | QSWVPLSTDD- | |
| | PVIDRLYLSSHRGVIADNQAKWAVPTTRTDDKL | |
| | RMETCFQQACKGKIQALCENPEWAPLKDNRIPS | |
| | YGVLSVDLSLTVELKIKIASGFGPLITHGSGMDL | |
| | YKSNHNNVYWLTIPPMKNLALGVINTLEWIPRF | |
| | KVSPYLFNVPIKEAGEDCHAPTYLPAEVDGDVK | |
| | LSSNLVILPGQDLQYVLATYDTSRVEHAVVYYV | |
| | YSPSRSFSYFYPFRLPIKGVPIELQVECFTWDQKL | |
| | WCRHFCVLADSESGGHITHSGMEGMGVSCTVT | |
| | REDGTNRR | |
| 245 | MLPYQDKVGAFYKDNARANSTKLSLVTEGHGG | Canine distemper |
| | RRPPYLLFVLLILLVGILALLAITGVRFHQVSTSN | virus H protein |
| | MEFSRLLKEDMEKSEAVHHQVIDVLTPLFKIIGD | |
| | EIGLRLPQKLNEIKQFILQKTNFFNPNREFDFRDL | |
| | HWCINPPSTVKVNFTNYCESIGIRKAIASAANPIL | |
| | LSALSGGRGDIFPPHRCSGATTSVGKVFPLSVSL | |
| | SMSLISRTSEVINMLTAISDGVYGKTYLLVPDDI | |
| | EREFDTREIRVFEIGFIKRWLNDMPLLQTTNYM | |
| | VLPKNSKAKVCTIAVGELTLASLCVEESTVLLY | |
| | HDSSGSQDGILVVTLGIFWATPMDHIEEVIPVAH | |
| | PSMKKIHITNHRGFIKDSIATWMVPALASEKQEE | |
| | QKGCLESACQRKTYPMCNQASWEPFGGRQLPS | |
| | YGRLTLPLDASVDLQLNISFTYGPVILNGDGMD | |
| | YYESPLLNSGWLTIPPKDGTISGLINKAGRGDQF | |
| | TVLPHVLTFAPRESSGNCYLPIQTSQIRDRDVLIE | |
| | SNIVVLPTQSIRYVIATYDISRSDHAIVYYVYDPI | |
| | RTISYTHPFRLTTKGRPDFLRIECFVWDDNLWC | |
| | HQF | |
| 246 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N N N N Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 247 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N N N Q N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 248 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N N N Q Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 249 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N N N Q Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 250 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N N Q N Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 251 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N N Q N Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 252 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N N Q Q N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 253 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N N Q Q N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 254 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N N Q Q Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 255 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N Q N N N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 256 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N Q N N Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 257 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N Q N N Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 258 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N Q N Q N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 259 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N Q N Q N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 260 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N Q N Q Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 261 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N Q N Q Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCTGGGG | |
| | SGGGGSGGGGS | |
| 262 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N Q Q N N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 263 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N Q Q N N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 264 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N Q Q N Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 265 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N Q Q N Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 266 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N Q Q Q N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 267 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N Q Q Q N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 268 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N Q Q Q Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 269 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | N N Q Q Q Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCTGGGG | |
| | SGGGGSGGGGS | |
| 270 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q N N N N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCTGGGG | |
| | SGGGGSGGGGS | |
| 271 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q N N N Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 272 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q N N N Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCTGGGG | |
| | SGGGGSGGGGS | |
| 273 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q N N Q N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 274 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q N N Q N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCTGGGG | |
| | SGGGGSGGGGS | |
| 275 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q N N Q Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 276 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q N N Q Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 277 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q N Q N N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 278 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q N Q N N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 279 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q N Q N Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 280 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q N Q N Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 281 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q N Q Q N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 282 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q N Q Q Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 283 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q N Q Q Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 284 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q Q N N N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 285 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q Q N N N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 286 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q Q N N Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 287 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q Q N N Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 288 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q Q N Q N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 289 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q Q N Q N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 290 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q Q N Q Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 291 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q Q N Q Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 292 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q Q Q N N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 293 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q Q Q N N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 294 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q Q Q N Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 295 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q Q Q N Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 296 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q Q Q Q N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 297 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q Q Q Q N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 298 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q Q Q Q Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 299 | MKKINEGLLDSKILSAFNTVIALLGSIVIIVMNIM | Amino acid at |
| | IIQNYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | N Q Q Q Q Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCTGGGG | |
| | SGGGGSGGGGS | |
| 300 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N N N N N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 301 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N N N N Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCTGGGG | |
| | SGGGGSGGGGS | |
| 302 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N N N N Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 303 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N N N Q N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCTGGGG | |
| | SGGGGSGGGGS | |
| 304 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N N N Q N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 305 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N N N Q Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 306 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N N Q N N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 307 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N N Q N N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 308 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N N Q N Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCTGGGG | |
| | SGGGGSGGGGS | |
| 309 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N N Q Q N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 310 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N N Q Q N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 311 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N N Q Q Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCTGGGG | |
| | SGGGGSGGGGS | |
| 312 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N N Q Q Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 313 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N Q N N N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 314 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N Q N N N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 315 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N Q N N Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 316 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N Q N N Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCTGGGG | |
| | SGGGGSGGGGS | |
| 317 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N Q N Q N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 318 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N Q N Q N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 319 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N Q N Q Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 320 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N Q N Q Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 321 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N Q Q N N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 322 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N Q Q N N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 323 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N Q Q N Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 324 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N Q Q N Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 325 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N Q Q Q N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 326 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N Q Q Q N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 327 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N Q Q Q Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 328 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q N Q Q Q Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 329 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q N N N N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 330 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q N N N N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 331 | MKKINEGXLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q N N N Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCTGGGG | |
| | SGGGGSGGGGS | |
| 332 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q N N N Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 333 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q N N Q N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCTGGGG | |
| | SGGGGSGGGGS | |
| 334 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q N N Q Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 335 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q N N Q Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 336 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q N Q N N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 337 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q N Q N N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 338 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q N Q N Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 339 | MKKINEGXLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q N Q N Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCTGGGG | |
| | SGGGGSGGGGS | |
| 340 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q N Q Q N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 341 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q N Q Q N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 342 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q N Q Q Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 343 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q Q N N N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 344 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q Q N N N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 345 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q Q N N Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 346 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q Q N Q N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 347 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q Q N Q N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 348 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q Q N Q Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCTGGGG | |
| | SGGGGSGGGGS | |
| 349 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q Q Q N N N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 350 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q Q Q N N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 351 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q Q Q N Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 352 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q Q Q Q N Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCTGGGG | |
| | SGGGGSGGGGS | |
| 353 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECNISCPNPLPFREYRPQ | 496, respectively: |
| | TEGVSNLVGLPNNICLQKTSNQILKPKLISYTLP | Q Q Q Q Q Q N |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 354 | MKKINEGLLDSKILSAFNTVIALLGSIVIIVMNIM | Amino acid at |
| | IIQQYTRSTDNQAVIKDALQGIQQQIKGLADKIG | positions 39, 126, |
| | TEIGPKVSLIDTSSTITIPANIGLLGSKISQSTASIN | 273, 345, 384, 448, |
| | ENVNEKCKFTLPPLKIHECQISCPNPLPFREYRPQ | 496, respectively: |
| | TEGVSNLVGLPNNICLQKTSNQILKPKLISYTLP | Q Q Q Q Q Q Q |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 355 | MPAENKKVRFENTTSDKGKIPSKVIKSYYGTMD | NivG CT_45_FL |
| | IKKINEGLLDSK | |
| 356 | MLDSK | NivG_CT_05 |
| | | cytoplasmic tail |
| 357 | MLLDSK | NivG_CT_06 |
| | | cytoplasmic tail |
| 358 | MGLLDSK | NivG_CT_07 |
| | | cytoplasmic tail |
| 359 | MEGLLDSK | NivG_CT_08 |
| | | cytoplasmic tail |
| 360 | MNEGLLDSK | NivG_CT_09 |
| | | cytoplasmic tail |
| 361 | MINEGLLDSK | NivG_CT_10 |
| | | cytoplasmic tail |
| 362 | MKINEGLLDSK | NivG_CT_11 |
| | | cytoplasmic tail |
| 363 | MIKKINEGLLDSK | NivG_CT_13 |
| | | cytoplasmic tail |
| 364 | MDIKKINEGLLDSK | NivG_CT_14 |
| | | cytoplasmic tail |
| 365 | MMDIKKINEGLLDSK | NivG_CT_15 |
| | | cytoplasmic tail |
| 366 | MTMDIKKINEGLLDSK | NivG_CT_16 |
| | | cytoplasmic tail |
| 367 | MGTMDIKKINEGLLDSK | NivG_CT_17 |
| | | cytoplasmic tail |
| 368 | MYGTMDIKKINEGLLDSK | NivG_CT_18 |
| | | cytoplasmic tail |
| 369 | MYYGTMDIKKINEGLLDSK | NivG_CT 19 |
| | | cytoplasmic tail |
| 370 | MSYYGTMDIKKINEGLLDSK | NivG_CT_20 |
| | | cytoplasmic tail |
| 371 | MKSYYGTMDIKKINEGLLDSK | NivG_CT_21 |
| | | cytoplasmic tail |
| 372 | MIKSYYGTMDIKKINEGLLDSK | NivG_CT_22 |
| | | cytoplasmic tail |
| 373 | MVIKSYYGTMDIKKINEGLLDSK | NivG_CT_23 |
| | | cytoplasmic tail |
| 374 | MKVIKSYYGTMDIKKINEGLLDSK | NivG_CT_24 |
| | | cytoplasmic tail |
| 375 | MSKVIKSYYGTMDIKKINEGLLDSK | NivG_CT_25 |
| | | cytoplasmic tail |
| 376 | MKGKNPSKVIKSYYGTMDIKKINEGLLDSK | NivG_CT_30 |
| | | cytoplasmic tail |
| 377 | MNTTSDKGKNPSKVIKSYYGTMDIKKINEGLLD | NivG_CT_35 |
| | SK | cytoplasmic tail |
| 378 | MKVRFENTTSDKGKNPSKVIKSYYGTMDIKKIN | NivG_CT_40 |
| | EGLLDSK | cytoplasmic tail |
| 379 | MIIKSYYGTMDIKKINEGLLDSK | NivG_CSUR38_C |
| | | T_23 cytoplasmic |
| | | tail |
| 380 | MGKIPSKIIKSYYGTMDIKKINEGLLDSK | NivG_CSUR38_C |
| | | T_29 cytoplasmic |
| | | tail |
| 381 | MNTTSDKGKIPSKIIKSYYGTMDIKKINEGLLDS | NivG_CSUR38_C |
| | K | T_35 cytoplasmic |
| | | tail |
| 382 | MSKKVRFENTTSDKGKIPSKIIKSYYGTMDIKKI | NivG_CSUR38_C |
| | NEGLLDSK | T_42 cytoplasmic |
| | | tail |
| 383 | MPAESKKVRFENTTSDKGKIPSKIIKSYYGTMDI | NivG_CSUR38_C |
| | KKINEGLLDSK | T_45_FL |
| | | cytoplasmic tail |
| 384 | MNPSKVIKSYYGTMDIKKINEGLLDSK | NivG_VRI- |
| | | 0626_CT_27 |
| | | cytoplasmic tail |
| 385 | MDKGKNPSKVIKSYYGTMDIKKINEGLLDSK | NivG_VRI- |
| | | 0626_CT_31 |
| | | cytoplasmic tail |
| 386 | MNTTSDKGKNPSKVIKSYYGTMDIKKINEGLLD | NivG_VRI- |
| | SK | 0626_CT_35 |
| 387 | MKVRFENTTSDKGKNPSKVIKSYYGTMDIKKIN | NivG_VRI- |
| | EGLLDSK | 0626_CT_40 |
| | | cytoplasmic tail |
| 388 | MPAENKKVRFENTTSDKGKNPSKVIKSYYGTM | NivG_VRI- |
| | DIKKINEGLLDSK | 0626_CT_45_FL |
| | | cytoplasmic tail |
| 389 | MKKINEGLLDSK | NivG_CT_12 |
| | | cytoplasmic tail |
| 390 | MDGLLDSK | HevG_CT_08 |
| 391 | MNDGLLDSK | HevG_CT_09 |
| 392 | MINDGLLDSK | HevG_CT_10 |
| 393 | MKINDGLLDSK | HevG_CT_11 |
| 394 | MKKINDGLLDSK | HevG_CT_12 |
| 395 | MIKKINDGLLDSK | HevG_CT_13 |
| 396 | MDIKKINDGLLDSK | HevG_CT_14 |
| 397 | MMDIKKINDGLLDSK | HevG_CT_15 |
| 398 | MTMDIKKINDGLLDSK | HevG_CT_16 |
| 399 | MGTMDIKKINDGLLDSK | HevG_CT_17 |
| 400 | MYGTMDIKKINDGLLDSK | HevG_CT_18 |
| 401 | MYYGTMDIKKINDGLLDSK | HevG_CT_19 |
| 402 | MNYYGTMDIKKINDGLLDSK | HevG_CT_20 |
| 403 | MKNYYGTMDIKKINDGLLDSK | HevG_CT_21 |
| 404 | MIKNYYGTMDIKKINDGLLDSK | HevG_CT_22 |
| 405 | MVIKNYYGTMDIKKINDGLLDSK | HevG_CT_23 |
| 406 | MKVIKNYYGTMDIKKINDGLLDSK | HevG_CT_24 |
| 407 | MMADSKLVSLNNNLSGKIKDQGKVIKNYYGTM | HevG_CT_45_FL |
| | DIKKINDGLLDSK | |
| 408 | MQNDHY | BatPV_G_CT_6 |
| 409 | MNQNDHY | BatPV_G_CT_7 |
| 410 | MKNQNDHY | BatPV_G_CT_8 |
| 411 | MQKNQNDHY | BatPV_G_CT_9 |
| 412 | MKQKNQNDHY | BatPV_G_CT_10 |
| 413 | MKKQKNQNDHY | BatPV_G_CT_11 |
| 414 | MWKKQKNQNDHY | BatPV_G_CT_12 |
| 415 | MNWKKQKNQNDHY | BatPV_G_CT_13 |
| 416 | MRNWKKQKNQNDHY | BatPV_G_CT_14 |
| 417 | MERNWKKQKNQNDHY | BatPV_G_CT_15 |
| 418 | MSERNWKKQKNQNDHY | BatPV_G_CT_16 |
| 419 | MHSERNWKKQKNQNDHY | BatPV_G_CT_17 |
| 420 | MSHSERNWKKQKNQNDHY | BatPV_G_CT_18 |
| 421 | MGSHSERNWKKQKNQNDHY | BatPV_G_CT_19 |
| 422 | MLGSHSERNWKKQKNQNDHY | BatPV_G_CT_20 |
| 423 | MGLGSHSERNWKKQKNQNDHY | BatPV_G_CT_21 |
| 424 | MFGLGSHSERNWKKQKNQNDHY | BatPV_G_CT_22 |
| 425 | MYFGLGSHSERNWKKQKNQNDHY | BatPV_G_CT_23 |
| 426 | MGYFGLGSHSERNWKKQKNQNDHY | BatPV_G_CT_24 |
| 427 | MKITKQGYFGLGSHSERNWKKQKNQNDHY | BatPV_G_CT_29 |
| 428 | MPQKTVEFINMNSPLERGVSTLSDKKTLNQSKIT | BatPV_G_CT_59_ |
| | KQGYFGLGSHSERNWKKQKNQNDHY | FL |
| 429 | MLHDIK | CedarV_G_CT_6 |
| 430 | MSLHDIK | CedarV_G_CT_7 |
| 431 | MESLHDIK | CedarV_G_CT_8 |
| 432 | MNESLHDIK | CedarV_G_CT_9 |
| 433 | MLNESLHDIK | CedarV_G_CT_10 |
| 434 | MLLNESLHDIK | CedarV_G_CT_11 |
| 435 | MNLLNESLHDIK | CedarV_G_CT_12 |
| 436 | MSNLLNESLHDIK | CedarV_G_CT_13 |
| 437 | MVSNLLNESLHDIK | CedarV_G_CT_14 |
| 438 | MNVSNLLNESLHDIK | CedarV_G_CT_15 |
| 439 | MYNVSNLLNESLHDIK | CedarV_G_CT_16 |
| 440 | MNYNVSNLLNESLHDIK | CedarV_G_CT_17 |
| 441 | MKNYNVSNLLNESLHDIK | CedarV_G_CT_18 |
| 442 | MNKNYNVSNLLNESLHDIK | CedarV_G_CT_19 |
| 443 | MKNKNYNVSNLLNESLHDIK | CedarV_G_CT_20 |
| 444 | MVKNKNYNVSNLLNESLHDIK | CedarV_G_CT_21 |
| 445 | MYVKNKNYNVSNLLNESLHDIK | CedarV_G_CT_22 |
| 446 | MYYVKNKNYNVSNLLNESLHDIK | CedarV_G_CT_23 |
| 447 | MSYYVKNKNYNVSNLLNESLHDIK | CedarV_G_CT_24 |
| 448 | MQKDLNKSYYVKNKNYNVSNLLNESLHDIK | CedarV_G_CT_30 |
| 449 | MLSQLQKNYLDNSNQQGDKMNNPDKKLSVNF | CedarV_G_CT_68_ |
| | NPLELDKGQKDLNKSY | FL |
| | YVKNKNYNVSNLLNESLHDIK | |
| 450 | MQGGRR | CDV-H_CT_06 |
| 451 | MEQGGRR | CDV-H_CT_07 |
| 452 | MEEQGGRR | CDV-H_CT_08 |
| 453 | MTEEQGGRR | CDV-H_CT_09 |
| 454 | MVTEEQGGRR | CDV-H_CT_10 |
| 455 | MLVTEEQGGRR | CDV-H_CT_11 |
| 456 | MSLVTEEQGGRR | CDV-H_CT_12 |
| 457 | MLSLVTEEQGGRR | CDV-H_CT_13 |
| 458 | MKLSLVTEEQGGRR | CDV-H_CT_14 |
| 459 | MSKLSLVTEEQGGRR | CDV-H_CT_15 |
| 460 | MSSKLSLVTEEQGGRR | CDV-H_CT_16 |
| 461 | MNSSKLSLVTEEQGGRR | CDV-H_CT_17 |
| 462 | MANSSKLSLVTEEQGGRR | CDV-H_CT_18 |
| 463 | MRANSSKLSLVTEEQGGRR | CDV-H_CT_19 |
| 464 | MARANSSKLSLVTEEQGGRR | CDV-H_CT_20 |
| 465 | MLSYQDKVGAFYKDNARANSSKLSLVTEEQGG | CDV- |
| | RR | H_CT_34_FL |
| 466 | MRIIFR | HPIV2- |
| | | HN_CT_06 |
| 467 | MIPKRTCRIIFR | HPIV2- |
| | | HN_CT_12 |
| 468 | MEDYSNLSLKSIPKRTCRIIFR | HPIV2- |
| | | HN_CT_22_FL |
| 469 | MLMIDR | MvH_CT_06 |
| 470 | MHLMIDR | MvH_CT_07 |
| 471 | MEHLMIDR | MvH_CT_08 |
| 472 | MREHLMIDR | MvH_CT_09 |
| 473 | MNREHLMIDR | MvH_CT_10 |
| 474 | MINREHLMIDR | MvH_CT_11 |
| 475 | MVINREHLMIDR | MvH_CT_12 |
| 476 | MIVINREHLMIDR | MvH_CT_13 |
| 477 | MRIVINREHLMIDR | MvH_CT_14 |
| 478 | MSRIVINREHLMIDR | MvH_CT_15 |
| 479 | MGSRIVINREHLMIDR | MvH_CT_16 |
| 480 | MKGSRIVINREHLMIDR | MvH_CT_17 |
| 481 | MPKGSRIVINREHLMIDR | MvH_CT_18 |
| 482 | MHPKGSRIVINREHLMIDR | MvH_CT_19 |
| 483 | MPHPKGSRIVINREHLMIDR | MvH_CT_20 |
| 484 | MSPQRDRINAFYKDNPHPKGSRIVINREHLMIDR | MvH_CT_34_FL |
| 485 | MRLVFR | NDV-HN_CT_06 |
| 486 | MWRLVFR | NDV-HN_CT_07 |
| 487 | MTWRLVFR | NDV-HN_CT_08 |
| 488 | MNTWRLVFR | NDV-HN_CT_09 |
| 489 | MKNTWRLVFR | NDV-HN_CT_10 |
| 490 | MAKNTWRLVFR | NDV-HN_CT_11 |
| 491 | MEAKNTWRLVFR | NDV-HN_CT_12 |
| 492 | MREAKNTWRLVFR | NDV-HN_CT_13 |
| 493 | MEREAKNTWRLVFR | NDV-HN_CT_14 |
| 494 | MDEREAKNTWRLVFR | NDV-HN_CT_15 |
| 495 | MNDEREAKNTWRLVFR | NDV-HN_CT_16 |
| 496 | MENDEREAKNTWRLVFR | NDV-HN_CT_17 |
| 497 | MLENDEREAKNTWRLVFR | NDV-HN_CT_18 |
| 498 | MALENDEREAKNTWRLVFR | NDV-HN_CT_19 |
| 499 | MVALENDEREAKNTWRLVFR | NDV-HN_CT_20 |
| 500 | MERGVSQVALENDEREAKNTWRLVFR | NDV- |
| | | HN_CT_26_FL |
| 501 | MERSGK | SeV_CT_06 |
| 502 | MSERSGK | SeV_CT_07 |
| 503 | MDSERSGK | SeV_CT_08 |
| 504 | MSDSERSGK | SeV_CT_09 |
| 505 | MVSDSERSGK | SeV_CT_10 |
| 506 | MLVSDSERSGK | SeV_CT_11 |
| 507 | MKLVSDSERSGK | SeV_CT_12 |
| 508 | MTKLVSDSERSGK | SeV_CT_13 |
| 509 | MTTKLVSDSERSGK | SeV_CT_14 |
| 510 | MSTTKLVSDSERSGK | SeV_CT_15 |
| 511 | MGSTTKLVSDSERSGK | SeV_CT_16 |
| 512 | MGGSTTKLVSDSERSGK | SeV_CT_17 |
| 513 | MPGGSTTKLVSDSERSGK | SeV_CT_18 |
| 514 | MSPGGSTTKLVSDSERSGK | SeV_CT_19 |
| 515 | MTSPGGSTTKLVSDSERSGK | SeV_CT_20 |
| 516 | MDGDRSKRDSYWSTSPGGSTTKLVSDSERSGK | SeV_CT_32_FL |
| 517 | MDQAEEDTRLVQYQQTLVMAHIINLKDNIFATL | BaEVwt_CT |
| | RN | |
| 518 | MAHIINLKDNIFATLRNKKINEGLLDSK | BaEVRLess_CT |
| 519 | MAHIINLKDNIFATLRN | BaEVRLess_CT |
| 520 | MKRFRSMEIDNYIRKNNSGQYRYR | Cocal_CT |
| 521 | MKRFRSMEIDNYIRKNNSGQYRYRKKINEGLLD | Cocal_CT |
| | SK | |
| 522 | MFVFKC | EboV-GP_CT |
| 523 | MFVFKCKKINEGLLDSK | EboV-GP_CT |
| 524 | MLNGENELAQYKQRLVLIKVASIRDNIFQVLKK | GaLV_CT |
| | KINEGLLDSK | |
| 525 | MLNGENELAQYKQRLVLIKVASIRDNIFQVLK | GaLV_CT |
| 526 | MLIKVASIRDNIFQVLKKKINEGLLDSK | GaLV-RLess_CT |
| 527 | MLIKVASIRDNIFQVLK | GaLV-RLess_CT |
| 528 | MRRKWVTKVGPVKFAGCSCIGKNTLRHPKPCS | LCMV_GP_CT |
| | GGKIHRHT | |
| 529 | MPEYEIPKLQHYQQTLVLAQVVSIRDKVFQVLR | MLV-A_CT |
| | KKINEGLLDSK | |
| 530 | MPEYEIPKLQHYQQTLVLAQVVSIRDKVFQVLR | MLV-A_CT |
| 531 | MLAQVVSIRDKVFQVLRKKINEGLLDSK | MLV-A- |
| | | RLess_CT |
| 532 | MLAQVVSIRDKVFQVLR | MLV-A- |
| | | RLess_CT |
| 533 | MKGLRNMEIDTYIQRKKTHKLKICLHIGVR | VSV-G_CT |
| 534 | MKGLRNMEIDTYIQRKKTHKLKICLHIGVRKKI | VSV-G_CT |
| | NEGLLDSK | |
| 535 | MLLIRELGQRIRRPIHRIARYAAQLVEIVRDTGE | HIV- |
| | AVAIATANLLNVAS | 1_gp41_MLLIR- |
| | NKLEQSWYQLLNWWYKLAEWGRRGLLEVIRT | LLP1-LLP3- |
| | VILLLDRLRHYSFL | LLP2-gp41-CT-N |
| | CLSRLDDWILALSGNVLRISRDRDREGGEEEIGE | |
| | PRDPGRPIPLHTQF | |
| | SLPSYGQRVRN | |
| 536 | MLLIRELGQRIRRPIHRIARYAAQLVEIVRDTGE | HIV- |
| | AVAIATANLLNVASNKL | 1_gp41_MLLIR- |
| | EQSWYQLLNWWYKLAEWGRRGLLEVIRTVILL | LLP1-LLP3- |
| | LDRLRHYSFLCLSRLD | LLP2-gp41-CT-N |
| | DWILALSGNVLRISRDRDREGGEEEIGEPRDPGR | |
| | PIPLHTQFSLPSYGQRV | |
| | RNKKINEGLLDSK | |
| 537 | MAVAIATANLLNVASNKLEQSWYQLLNWWYK | HIV- |
| | LAEWGRRGLLEVIRTVI | 1_gp41_LLP3- |
| | LLLDRLRHYSFLCLSRLDDWILALSGNVLRISRD | LLP2-gp41-CT-N |
| | RDREGGEEEIGEPRDPG | |
| | RPIPLHTQFSLPSYGQRVRN | |
| 538 | MLLEVIRTVILLLDRLRHYSFLCLSRLDDWILAL | HIV- |
| | SGNVLRISRDRDREGGEE | 1_gp41_LLP2- |
| | EIGEPRDPGRPIPLHTQFSLPSYGQRVRN | gp41-CT-N |
| 539 | MSGNVLRISRDRDREGGEEEIGEPRDPGRPIPLH | HIV- |
| | TQFSLPSYGQRVRN | 1_gp41_gp41-CT- |
| | | N |
| 540 | MLLIRELGQRIRRPIHRIARYAAQLVEIVRDTGE | HIV- |
| | AVAIATANLLNVASNKLE | 1_gp41_MLLIR- |
| | QSWYQLLNWWYKLAEWGRRGLLEVIRTVILLL | LLP1-LLP3-LLP2 |
| | DRLRHYSFLCLSRLDDWILAL | |
| 541 | MELGQRIRRPIHRIARYAAQLVEIVRDTGEAVAI | HIV- |
| | ATANLLNVASNKLEQSW | 1_gp41_LLP1- |
| | YQLLNWWYKLAEWGRRGLLEVIRTVILLLDRL | LLP3-LLP2 |
| | RHYSFLCLSRLDDWILAL | |
| 542 | MAVAIATANLLNVASNKLEQSWYQLLNWWYK | HIV- |
| | LAEWGRRGLLEVIRTVI | 1_gp41_LLP3- |
| | LLLDRLRHYSFLCLSRLDDWILAL | LLP2 |
| 543 | MLLEVIRTVILLLDRLRHYSFLCLSRLDDWILAL | HIV- |
| | | 1_gp41_LLP2 |
| 544 | MLLIRELGQRIRRPIHRIARYAAQLVEIVRDTGE | HIV- |
| | AVAIATANLLNVASNKLE | 1_gp41_MLLIR- |
| | QSWYQLLNWWYKLAEW | LLP1-LLP3 |
| 545 | MELGQRIRRPIHRIARYAAQLVEIVRDTGEAVAI | HIV- |
| | ATANLLNVASNKLEQSW | 1_gp41_LLP1- |
| | YQLLNWWYKLAEW | LLP3 |
| 546 | MAVAIATANLLNVASNKLEQSWYQLLNWWYK | HIV- |
| | LAEW | 1_gp41_LLP3 |
| 547 | MLLIRELGQRIRRPIHRIARYAAQLVEIVR | HIV- |
| | | 1_gp41_MLLIR- |
| | | LLP1 |
| 548 | MELGQRIRRPIHRIARYAAQLVEIVR | HIV- |
| | | 1_gp41_LLP1 |
| 549 | MLLIR | HIV- |
| | | 1_gp41_MLLIR |
| 550 | MAVEGGMKCVK | CD63_CT_1to11 |
| 551 | MCGACKENYC | CD63_CT_73to81 |
| 552 | MVEYGSRISKVLCC | CD63_CT_225to238 |
| 553 | MAVEGGMKCVKKKINEGLLDSK | CD63_CT_1to11 |
| 554 | MCGACKENYCKKINEGLLDSK | CD63_CT_73to81 |
| 555 | MVEYGSRISKVLCCKKINEGLLDSK | CD63_CT_225to238 |
| 556 | MKGEYKPNVVTTVASKYIPNEGTDWKANMKE | CD29_CT |
| | KEFKAFERRDHIIMLLK | |
| 557 | MKGEYKPNVVTTVASKYIPNEGTDWKANMKE | CD29_CT |
| | KEFKAFERRDHIIMLLKK | |
| | KINEGLLDSK | |
| 558 | MVINREHLMIDRILSAFNTVIALLGSIVIIVMNIMI | MvH_CT_12 |
| | IQNYTRSTDNQAVIKDALQ | |
| | GIQQQIKGLADKIGTEIGPKVSLIDTSSTITIPANI | |
| | GLLGSKISQSTASINENVNEK | |
| | CKFTLPPLKIHECNISCPNPLPFREYRPQTEGVSN | |
| | LVGLPNNICLQKTSNQILKP | |
| | KLISYTLPVVGQSGTCITDPLLAMDEGYFAYSHL | |
| | ERIGSCSRGVSKQRIIGVGE | |
| | VLDRGDEVPSLFMTNVWTPPNPNTVYHCSAVY | |
| | NNEFYYVLCAVSTVGDPILN | |
| | STYWSGSLMMTRLAVKPKSNGGGYNQHQLAL | |
| | RSIEKGRYDKVMPYGPSGIK | |
| | QGDTLYFPAVGFLVRTEFKYNDSNCPITKCQYS | |
| | KPENCRLSMGIRPNSHYILR | |
| | SGLLKYNLSDGENPKVVFIEISDQRLSIGSPSKIY | |
| | DSLGQPVFYQASFSWDTMI | |
| | KFGDVLTVNPLVVNWRNNTVISRPGQSQCPRFN | |
| | TCPAICAEGVYNDAFLIDR | |
| | INWISAGVFLDSNATAANPVFTVFKDNEILYRA | |
| | QLASEDTNAQKTITNCFLLK | |
| | NKIWCISLVEIYDTGDNVIRPKLFAVKIPEQCT | |
| 559 | MGSRIVINREHLMIDRILSAFNTVIALLGSIVIIVM | MvH_CT_16 |
| | NIMIIQNYTRSTDNQAVIKD | |
| | ALQGIQQQIKGLADKIGTEIGPKVSLIDTSSTITIP | |
| | ANIGLLGSKISQSTASINENV | |
| | NEKCKFTLPPLKIHECNISCPNPLPFREYRPQTEG | |
| | VSNLVGLPNNICLQKTSNQI | |
| | LKPKLISYTLPVVGQSGTCITDPLLAMDEGYFAY | |
| | SHLERIGSCSRGVSKQRIIG | |
| | VGEVLDRGDEVPSLFMTNVWTPPNPNTVYHCS | |
| | AVYNNEFYYVLCAVSTVGD | |
| | PILNSTYWSGSLMMTRLAVKPKSNGGGYNQHQ | |
| | LALRSIEKGRYDKVMPYGP | |
| | SGIKQGDTLYFPAVGFLVRTEFKYNDSNCPITKC | |
| | QYSKPENCRLSMGIRPNSH | |
| | YILRSGLLKYNLSDGENPKVVFIEISDQRLSIGSP | |
| | SKIYDSLGQPVFYQASFSWD | |
| | TMIKFGDVLTVNPLVVNWRNNTVISRPGQSQCP | |
| | RFNTCPAICAEGVYNDAFLI | |
| | DRINWISAGVFLDSNATAANPVFTVFKDNEILY | |
| | RAQLASEDTNAQKTITNCFL | |
| | LKNKIWCISLVEIYDTGDNVIRPKLFAVKIPEQC | |
| | T | |
| 560 | MPHPKGSRIVINREHLMIDRILSAFNTVIALLGSI | MvH_CT_20 |
| | VIIVMNIMIIQNYTRSTDN | |
| | QAVIKDALQGIQQQIKGLADKIGTEIGPKVSLID | |
| | TSSTITIPANIGLLGSKISQ | |
| | STASINENVNEKCKFTLPPLKIHECNISCPNPLPF | |
| | REYRPQTEGVSNLVGLP | |
| | NNICLQKTSNQILKPKLISYTLPVVGQSGTCITDP | |
| | LLAMDEGYFAYSHLER | |
| | IGSCSRGVSKQRIIGVGEVLDRGDEVPSLFMTNV | |
| | WTPPNPNTVYHCSAVY | |
| | NNEFYYVLCAVSTVGDPILNSTYWSGSLMMTR | |
| | LAVKPKSNGGGYNQHQ | |
| | LALRSIEKGRYDKVMPYGPSGIKQGDTLYFPAV | |
| | GFLVRTEFKYNDSNCPI | |
| | TKCQYSKPENCRLSMGIRPNSHYILRSGLLKYNL | |
| | SDGENPKVVFIE | |
| | ISDQRLSIGSPSKIYDSLGQPVFYQASFSWDTMIK | |
| | FGDVLTVNPLVVNWR | |
| | NNTVISRPGQSQCPRFNTCPAICAEGVYNDAFLI | |
| | DRINWISAGVFLDSNAT | |
| | AANPVFTVFKDNEILYRAQLASEDTNAQKTITN | |
| | CFLLKNKIWCISLVEIY | |
| | DTGDNVIRPKLFAVKIPEQCT | |
| 561 | MYYGTMDIKKINEGLLDSKILSAFNTVIALLGSI | NivG_CT_19 |
| | VIIVMNIMIIQNYTRSTDNQ | |
| | AVIKDALQGIQQQIKGLADKIGTEIGPKVSLIDTS | |
| | STITIPANIGLLGSKISQSTA | |
| | SINENVNEKCKFTLPPLKIHECNISCPNPLPFREY | |
| | RPQTEGVSNLVGLPNNICL | |
| | QKTSNQILKPKLISYTLPVVGQSGTCITDPLLAM | |
| | DEGYFAYSHLERIGSCSRG | |
| | VSKQRIIGVGEVLDRGDEVPSLFMTNVWTPPNP | |
| | NTVYHCSAVYNNEFYYVL | |
| | CAVSTVGDPILNSTYWSGSLMMTRLAVKPKSN | |
| | GGGYNQHQLALRSIEKGR | |
| | YDKVMPYGPSGIKQGDTLYFPAVGFLVRTEFKY | |
| | NDSNCPITKCQYSKPENC | |
| | RLSMGIRPNSHYILRSGLLKYNLSDGENPKVVFI | |
| | EISDQRLSIGSPSKIYDSLG | |
| | QPVFYQASFSWDTMIKFGDVLTVNPLVVNWRN | |
| | NTVISRPGQSQCPRFNTCP | |
| | AICAEGVYNDAFLIDRINWISAGVFLDSNATAA | |
| | NPVFTVFKDNEILYRAQLA | |
| | SEDTNAQKTITNCFLLKNKIWCISLVEIYDTGDN | |
| | VIRPKLFAVKIPEQCT | |
| 562 | MKNQNDHYILSAFNTVIALLGSIVIIVMNIMIIQN | BatPV_G_CT_8 |
| | YTRSTDNQAVIKDALQGIQQQIKGLADKIGTEIG | |
| | PKVSLIDTSSTITIPANIGLLGSKISQSTASINENV | |
| | NEKCKFTLPPLKIHECNISCPNPLPFREYRPQTEG | |
| | VSNLVGLPNNICLQKTSNQILKPKLISYTLPVVG | |
| | QSGTCITDPLLAMDEGYFAYSHLERIGSCSRGVS | |
| | KQRIIGVGEVLDRGDEVPSLFMTNVWTPPNPNT | |
| | VYHCSAVYNNEFYYVLCAVSTVGDPILNSTYW | |
| | SGSLMMTRLAVKPKSNGGGYNQHQLALRSIEK | |
| | GRYDKVMPYGPSGIKQGDTLYFPAVGFLVRTEF | |
| | KYNDSNCPITKCQYSKPENCRLSMGIRPNSHYIL | |
| | RSGLLKYNLSDGENPKVVFIEISDQRLSIGSPSKI | |
| | YDSLGQPVFYQASFSWDTMIKFGDVLTVNPLV | |
| | VNWRNNTVISRPGQSQCPRFNTCPAICAEGVYN | |
| | DAFLIDRINWISAGVFLDSNATAANPVFTVFKD | |
| | NEILYRAQLASEDTNAQKTITNCFLLKNKIWCIS | |
| | LVEIYDTGDNVIRPKLFAVKIPEQCT | |
| 563 | MDSERSGKILSAFNTVIALLGSIVIIVMNIMIIQN | SeV_CT_08 |
| | YTRSTDNQAVIKDALQGIQQQIKGLADKIGTEIG | |
| | PKVSLIDTSSTITIPANIGLLGSKISQSTASINENV | |
| | NEKCKFTLPPLKIHECNISCPNPLPFREYRPQTEG | |
| | VSNLVGLPNNICLQKTSNQILKPKLISYTLPVVG | |
| | QSGTCITDPLLAMDEGYFAYSHLERIGSCSRGVS | |
| | KQRIIGVGEVLDRGDEVPSLFMTNVWTPPNPNT | |
| | VYHCSAVYNNEFYYVLCAVSTVGDPILNSTYW | |
| | SGSLMMTRLAVKPKSNGGGYNQHQLALRSIEK | |
| | GRYDKVMPYGPSGIKQGDTLYFPAVGFLVRTEF | |
| | KYNDSNCPITKCQYSKPENCRLSMGIRPNSHYIL | |
| | RSGLLKYNLSDGENPKVVFIEISDQRLSIGSPSKI | |
| | YDSLGQPVFYQASFSWDTMIKFGDVLTVNPLV | |
| | VNWRNNTVISRPGQSQCPRFNTCPAICAEGVYN | |
| | DAFLIDRINWISAGVFLDSNATAANPVFTVFKD | |
| | NEILYRAQLASEDTNAQKTITNCFLLKNKIWCIS | |
| | LVEIYDTGDNVIRPKLFAVKIPEQCT | |
| 564 | MLAQVVSIRDKVFQVLRILSAFNTVIALLGSIVII | MLV-A- |
| | VMNIMIIQNYTRSTDNQAVIKDALQGIQQQIKGL | RLess_CT |
| | ADKIGTEIGPKVSLIDTSSTITIPANIGLLGSKISQS | |
| | TASINENVNEKCKFTLPPLKIHECNISCPNPLPFR | |
| | EYRPQTEGVSNLVGLPNNICLQKTSNQILKPKLI | |
| | SYTLPVVGQSGTCITDPLLAMDEGYFAYSHLERI | |
| | GSCSRGVSKQRIIGVGEVLDRGDEVPSLFMTNV | |
| | WTPPNPNTVYHCSAVYNNEFYYVLCAVSTVGD | |
| | PILNSTYWSGSLMMTRLAVKPKSNGGGYNQHQ | |
| | LALRSIEKGRYDKVMPYGPSGIKQGDTLYFPAV | |
| | GFLVRTEFKYNDSNCPITKCQYSKPENCRLSMGI | |
| | RPNSHYILRSGLLKYNLSDGENPKVVFIEISDQR | |
| | LSIGSPSKIYDSLGQPVFYQASFSWDTMIKFGDV | |
| | LTVNPLVVNWRNNTVISRPGQSQCPRFNTCPAI | |
| | CAEGVYNDAFLIDRINWISAGVFLDSNATAANP | |
| | VFTVFKDNEILYRAQLASEDTNAQKTITNCFLLK | |
| | NKIWCISLVEIYDTGDNVIRPKLFAVKIPEQCT | |
| 565 | MLLIRILSAFNTVIALLGSIVIIVMNIMIIQNYTRS | HIV- |
| | TDNQAVIKDALQGIQQQIKGLADKIGTEIGPKVS | 1_gp41_MLLIR |
| | LIDTSSTITIPANIGLLGSKISQSTASINENVNEKC | |
| | KFTLPPLKIHECNISCPNPLPFREYRPQTEGVSNL | |
| | VGLPNNICLQKTSNQILKPKLISYTLPVVGQSGT | |
| | CITDPLLAMDEGYFAYSHLERIGSCSRGVSKQRI | |
| | IGVGEVLDRGDEVPSLFMTNVWTPPNPNTVYH | |
| | CSAVYNNEFYYVLCAVSTVGDPILNSTYWSGSL | |
| | MMTRLAVKPKSNGGGYNQHQLALRSIEKGRYD | |
| | KVMPYGPSGIKQGDTLYFPAVGFLVRTEFKYND | |
| | SNCPITKCQYSKPENCRLSMGIRPNSHYILRSGL | |
| | LKYNLSDGENPKVVFIEISDQRLSIGSPSKIYDSL | |
| | GQPVFYQASFSWDTMIKFGDVLTVNPLVVNWR | |
| | NNTVISRPGQSQCPRFNTCPAICAEGVYNDAFLI | |
| | DRINWISAGVFLDSNATAANPVFTVFKDNEILY | |
| | RAQLASEDTNAQKTITNCFLLKNKIWCISLVEIY | |
| | DTGDNVIRPKLFAVKIPEQCT | |
| 566 | MAVEGGMKCVKILSAFNTVIALLGSIVIIVMNIM | CD63_CT_1to11 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 567 | MCGACKENYCILSAFNTVIALLGSIVIIVMNIMII | CD63_CT_73to81 |
| | QNYTRSTDNQAVIKDALQGIQQQIKGLADKIGT | |
| | EIGPKVSLIDTSSTITIPANIGLLGSKISQSTASINE | |
| | NVNEKCKFTLPPLKIHECNISCPNPLPFREYRPQT | |
| | EGVSNLVGLPNNICLQKTSNQILKPKLISYTLPV | |
| | VGQSGTCITDPLLAMDEGYFAYSHLERIGSCSR | |
| | GVSKQRIIGVGEVLDRGDEVPSLFMTNVWTPPN | |
| | PNTVYHCSAVYNNEFYYVLCAVSTVGDPILNST | |
| | YWSGSLMMTRLAVKPKSNGGGYNQHQLALRSI | |
| | EKGRYDKVMPYGPSGIKQGDTLYFPAVGFLVR | |
| | TEFKYNDSNCPITKCQYSKPENCRLSMGIRPNSH | |
| | YILRSGLLKYNLSDGENPKVVFIEISDQRLSIGSP | |
| | SKIYDSLGQPVFYQASFSWDTMIKFGDVLTVNP | |
| | LVVNWRNNTVISRPGQSQCPRFNTCPAICAEGV | |
| | YNDAFLIDRINWISAGVFLDSNATAANPVFTVF | |
| | KDNEILYRAQLASEDTNAQKTITNCFLLKNKIW | |
| | CISLVEIYDTGDNVIRPKLFAVKIPEQCT | |
| 568 | MKKINDGLLDSKILSAFNTVIALLGSIVIIVMNIM | HevG_CT_12 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 569 | MPEYEIPKLQHYQQTLVLAQVVSIRDKVFQVLR | MLV-A_CT |
| | ILSAFNTVIALLGSIVIIVMNIMIIQNYTRSTDNQA | |
| | VIKDALQGIQQQIKGLADKIGTEIGPKVSLIDTSS | |
| | TITIPANIGLLGSKISQSTASINENVNEKCKFTLPP | |
| | LKIHECNISCPNPLPFREYRPQTEGVSNLVGLPN | |
| | NICLQKTSNQILKPKLISYTLPVVGQSGTCITDPL | |
| | LAMDEGYFAYSHLERIGSCSRGVSKQRIIGVGE | |
| | VLDRGDEVPSLFMTNVWTPPNPNTVYHCSAVY | |
| | NNEFYYVLCAVSTVGDPILNSTYWSGSLMMTR | |
| | LAVKPKSNGGGYNQHQLALRSIEKGRYDKVMP | |
| | YGPSGIKQGDTLYFPAVGFLVRTEFKYNDSNCPI | |
| | TKCQYSKPENCRLSMGIRPNSHYILRSGLLKYNL | |
| | SDGENPKVVFIEISDQRLSIGSPSKIYDSLGQPVF | |
| | YQASFSWDTMIKFGDVLTVNPLVVNWRNNTVI | |
| | SRPGQSQCPRFNTCPAICAEGVYNDAFLIDRINW | |
| | ISAGVFLDSNATAANPVFTVFKDNEILYRAQLAS | |
| | EDTNAQKTITNCFLLKNKIWCISLVEIYDTGDNV | |
| | IRPKLFAVKIPEQCT | |
| 570 | MLLDSKILSAFNTVIALLGSIVIIVMNIMIIQNYTR | NivG_CT_06 |
| | STDNQAVIKDALQGIQQQIKGLADKIGTEIGPKV | |
| | SLIDTSSTITIPANIGLLGSKISQSTASINENVNEK | |
| | CKFTLPPLKIHECNISCPNPLPFREYRPQTEGVSN | |
| | LVGLPNNICLQKTSNQILKPKLISYTLPVVGQSG | |
| | TCITDPLLAMDEGYFAYSHLERIGSCSRGVSKQR | |
| | IIGVGEVLDRGDEVPSLFMTNVWTPPNPNTVYH | |
| | CSAVYNNEFYYVLCAVSTVGDPILNSTYWSGSL | |
| | MMTRLAVKPKSNGGGYNQHQLALRSIEKGRYD | |
| | KVMPYGPSGIKQGDTLYFPAVGFLVRTEFKYND | |
| | SNCPITKCQYSKPENCRLSMGIRPNSHYILRSGL | |
| | LKYNLSDGENPKVVFIEISDQRLSIGSPSKIYDSL | |
| | GQPVFYQASFSWDTMIKFGDVLTVNPLVVNWR | |
| | NNTVISRPGQSQCPRFNTCPAICAEGVYNDAFLI | |
| | DRINWISAGVFLDSNATAANPVFTVFKDNEILY | |
| | RAQLASEDTNAQKTITNCFLLKNKIWCISLVEIY | |
| | DTGDNVIRPKLFAVKIPEQCT | |
| 571 | MIKKINEGLLDSKILSAFNTVIALLGSIVIIVMNI | NivG_CT_13 |
| | MIIQNYTRSTDNQAVIKDALQGIQQQIKGLADKI | |
| | GTEIGPKVSLIDTSSTITIPANIGLLGSKISQSTASI | |
| | NENVNEKCKFTLPPLKIHECNISCPNPLPFREYRP | |
| | QTEGVSNLVGLPNNICLQKTSNQILKPKLISYTL | |
| | PVVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 572 | MWKKQKNQNDHYILSAFNTVIALLGSIVIIVMNI | BatPV_G_CT_12 |
| | MIIQNYTRSTDNQAVIKDALQGIQQQIKGLADKI | |
| | GTEIGPKVSLIDTSSTITIPANIGLLGSKISQSTASI | |
| | NENVNEKCKFTLPPLKIHECNISCPNPLPFREYRP | |
| | QTEGVSNLVGLPNNICLQKTSNQILKPKLISYTL | |
| | PVVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 573 | MRIIFRILSAFNTVIALLGSIVIIVMNIMIIQNYTRS | HPIV2- |
| | TDNQAVIKDALQGIQQQIKGLADKIGTEIGPKVS | HN_CT_06 |
| | LIDTSSTITIPANIGLLGSKISQSTASINENVNEKC | |
| | KFTLPPLKIHECNISCPNPLPFREYRPQTEGVSNL | |
| | VGLPNNICLQKTSNQILKPKLISYTLPVVGQSGT | |
| | CITDPLLAMDEGYFAYSHLERIGSCSRGVSKQRI | |
| | IGVGEVLDRGDEVPSLFMTNVWTPPNPNTVYH | |
| | CSAVYNNEFYYVLCAVSTVGDPILNSTYWSGSL | |
| | MMTRLAVKPKSNGGGYNQHQLALRSIEKGRYD | |
| | KVMPYGPSGIKQGDTLYFPAVGFLVRTEFKYND | |
| | SNCPITKCQYSKPENCRLSMGIRPNSHYILRSGL | |
| | LKYNLSDGENPKVVFIEISDQRLSIGSPSKIYDSL | |
| | GQPVFYQASFSWDTMIKFGDVLTVNPLVVNWR | |
| | NNTVISRPGQSQCPRFNTCPAICAEGVYNDAFLI | |
| | DRINWISAGVFLDSNATAANPVFTVFKDNEILY | |
| | RAQLASEDTNAQKTITNCFLLKNKIWCISLVEIY | |
| | DTGDNVIRPKLFAVKIPEQCT | |
| 574 | MIPKRTCRIIFRILSAFNTVIALLGSIVIIVMNIMII | HPIV2- |
| | QNYTRSTDNQAVIKDALQGIQQQIKGLADKIGT | HN_CT_12 |
| | EIGPKVSLIDTSSTITIPANIGLLGSKISQSTASINE | |
| | NVNEKCKFTLPPLKIHECNISCPNPLPFREYRPQT | |
| | EGVSNLVGLPNNICLQKTSNQILKPKLISYTLPV | |
| | VGQSGTCITDPLLAMDEGYFAYSHLERIGSCSR | |
| | GVSKQRIIGVGEVLDRGDEVPSLFMTNVWTPPN | |
| | PNTVYHCSAVYNNEFYYVLCAVSTVGDPILNST | |
| | YWSGSLMMTRLAVKPKSNGGGYNQHQLALRSI | |
| | EKGRYDKVMPYGPSGIKQGDTLYFPAVGFLVR | |
| | TEFKYNDSNCPITKCQYSKPENCRLSMGIRPNSH | |
| | YILRSGLLKYNLSDGENPKVVFIEISDQRLSIGSP | |
| | SKIYDSLGQPVFYQASFSWDTMIKFGDVLTVNP | |
| | LVVNWRNNTVISRPGQSQCPRFNTCPAICAEGV | |
| | YNDAFLIDRINWISAGVFLDSNATAANPVFTVF | |
| | KDNEILYRAQLASEDTNAQKTITNCFLLKNKIW | |
| | CISLVEIYDTGDNVIRPKLFAVKIPEQCT | |
| 575 | MKLVSDSERSGKILSAFNTVIALLGSIVIIVMNIM | CD63_CT_1to11 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 576 | EVKLVESGPELVKPGETVKISCKASGYTFTDYS | CD133.S.001.02 |
| | MHWVRQAPGKGLEWMGWINTETGEPSYADDF | |
| | KGRFAFSLDTSASTAYLQINSLRSEDTATYFCAT | |
| | DYGDYFDYWGQGTTVTVSSGGGGSGGGGSGG | |
| | GGSDIVLSQSPAIMSASPGEKVTISCSASSSVSYM | |
| | YWYQQKPGSSPKPWIYRTSNLASGVPARFSGSG | |
| | SGTSYSLTISSMEPEDAATYYCQQYHSYPPTFGA | |
| | GTKLELK | |
| 577 | DVQLQESGPGLVKPSQSLSLTCSVTGYSITSGYY | CD133.s.004.01 |
| | WSWIRQFPGNKLEWMGYISYDGSNNYNPSLKN | |
| | RISITRDTSKNQIFLKLNSVTTEDTATYYCTRVY | |
| | FGHPYYYGMDYWGQGISVTVGGGGSGGGGSG | |
| | GGGSQIVLTQSPAIMSASLGERVTMTCTASSSVS | |
| | SFYLHWYRQKPGSSPKLWIYSTSNLASGVPARF | |
| | SGSGSGTSYSLTISSMEAEDAATYYCHQYHRSP | |
| | YMYMFGGGTKLEIK | |
| 578 | EVQLVESGGGLVQPGGSLRLSCAASGFNIYYYG | CD133.s.006.01 |
| | SMHWVRQAPGKGLEWVASISPYYGSTYYADSV | |
| | KGRFTISADTSKNTAYLQMNSLRAEDTAVYYC | |
| | ARHASSGYGHYAVYGIDYWGQGTLVTVSSGGG | |
| | GSGGGGSGGGGSDIQMTQSPSSLSASVGDRVTI | |
| | TCRASQGSSYVAWYQQKPGKAPKLLIYSASYL | |
| | YSGVPSRFSGSRSGTDFTLTISSLQPEDFATYYC | |
| | QQGVWSLITFGQGTKVEIK | |
| 579 | MKKINEGLLDSKILSAFNTVIALLGSIVIIVMNIM | NivG.690 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 580 | MKKINEGLLDSKILSAFNTVIALLGSIVIIVMNIM | NivG.691 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 581 | MKKINEGLLDSKILSAFNTVIALLGSIVIIVMNIM | NivG.693 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 582 | MKKINEGLLDSKILSAFNTVIALLGSIVIIVMNIM | NivG.694 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 583 | MKKINEGLLDSKILSAFNTVIALLGSIVIIVMNIM | NivG.695 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 584 | MKKINEGLLDSKILSAFNTVIALLGSIVIIVMNIM | NivG.696 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | NivG.697 |
| | FKDNEILYRAQLASEDTNAQKTITNCFLLKNKI | |
| | WCISLVEIYDTGDNVIRPKLFAVKIPEQCT | |
| 585 | MKKINEGLLDSKILSAFNTVIALLGSIVIIVMNIM | |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 586 | MKKINEGLLDSKILSAFNTVIALLGSIVIIVMNIM | NivG.699 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 587 | MKKINEGLLDSKILSAFNTVIALLGSIVIIVMNIM | NivG.700 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 588 | MKKINEGLLDSKILSAFNTVIALLGSIVIIVMNIM | NivG.701 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 589 | MKKINEGLLDSKILSAFNTVIALLGSIVIIVMNIM | NivG.702 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 590 | MKKINEGLLDSKILSAFNTVIALLGSIVIIVMNIM | NivG.740 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 591 | MKKINEGLLDSKILSAFNTVIALLGSIVIIVMNIM | NivG.742 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 592 | MKKINEGLLDSKILSAFNTVIALLGSIVIIVMNIM | NivG.743 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 593 | MKKINEGLLDSKILSAFNTVIALLGSIVIIVMNIM | NivG.744 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | NivG.748 |
| | WCISLVEIYDTGDNVIRPKLFAVKIPEQCT | |
| 594 | MKKINEGLLDSKILSAFNTVIALLGSIVIIVMNIM | |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 595 | MKKINEGLLDSKILSAFNTVIALLGSIVIIVMNIM | NivG.749 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 596 | MKKINEGLLDSKILSAFNTVIALLGSIVIIVMNIM | NivG.750 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 597 | MKKINEGLLDSKILSAFNTVIALLGSIVIIVMNIM | NivG.752 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 598 | MKKINEGLLDSKILSAFNTVIALLGSIVIIVMNIM | NivG.753 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 599 | MKKINEGLLDSKILSAFNTVIALLGSIVIIVMNIM | NivG.754 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 600 | MKKINEGLLDSKILSAFNTVIALLGSIVIIVMNIM | NivG.755 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 601 | MKKINEGLLDSKILSAFNTVIALLGSIVIIVMNIM | NivG.756 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 602 | MKKINEGLLDSKILSAFNTVIALLGSIVIIVMNIM | NivG.757 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 603 | MKKINEGLLDSKILSAFNTVIALLGSIVIIVMNIM | NivG.758 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 604 | MKKINEGLLDSKILSAFNTVIALLGSIVIIVMNIM | NivG.759 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 605 | MKKINEGLLDSKILSAFNTVIALLGSIVIIVMNIM | NivG.760 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 606 | MKKINEGLLDSKILSAFNTVIALLGSIVIIVMNIM | NivG.761 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 607 | MKKINEGLLDSKILSAFNTVIALLGSIVIIVMNIM | NivG.762 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 608 | MKKINEGLLDSKILSAFNTVIALLGSIVIIVMNIM | NivG.763 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 609 | MKKINEGLLDSKILSAFNTVIALLGSIVIIVMNIM | NivG.764 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 610 | MKKINEGLLDSKILSAFNTVIALLGSIVIIVMNIM | NivG.765 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 611 | MKKINEGLLDSKILSAFNTVIALLGSIVIIVMNIM | NivG.766 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 612 | MKKINEGLLDSKILSAFNTVIALLGSIVIIVMNIM | NivG.767 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 613 | MKKINEGLLDSKILSAFNTVIALLGSIVIIVMNIM | NivG.768 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 614 | MKKINEGLLDSKILSAFNTVIALLGSIVIIVMNIM | NivG.769 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 615 | MKKINEGLLDSKILSAFNTVIALLGSIVIIVMNIM | NivG.770 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 616 | MKKINEGLLDSKILSAFNTVIALLGSIVIIVMNIM | NivG.771 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 617 | MKKINEGLLDSKILSAFNTVIALLGSIVIIVMNIM | NivG.772 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 618 | MKKINEGLLDSKILSAFNTVIALLGSIVIIVMNIM | NivG.773 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 619 | MKKINEGLLDSKILSAFNTVIALLGSIVIIVMNIM | NivG.774 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 620 | MKKINEGLLDSKILSAFNTVIALLGSIVIIVMNIM | NivG.775 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 621 | MKKINEGLLDSKILSAFNTVIALLGSIVIIVMNIM | NivG.776 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 622 | MKKINEGLLDSKILSAFNTVIALLGSIVIIVMNIM | NivG.777 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 623 | MKKINEGLLDSKILSAFNTVIALLGSIVIIVMNIM | NivG.778 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 624 | MKKINEGLLDSKILSAFNTVIALLGSIVIIVMNIM | NivG.779 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 625 | MKKINEGLLDSKILSAFNTVIALLGSIVIIVMNIM | NivG.780 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 626 | MKKINEGLLDSKILSAFNTVIALLGSIVIIVMNIM | NivG.781 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISVEIYDTGDNVIRPKLFAVKIPEQCT | |
| 627 | MKKINEGLLDSKILSAFNTVIALLGSIVIIVMNIM | NivG.782 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 628 | MKKINEGLLDSKILSAFNTVIALLGSIVIIVMNIM | NivG.783 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 629 | MKKINEGLLDSKILSAFNTVIALLGSIVIIVMNIM | NivG.784 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 630 | MKKINEGLLDSKILSAFNTVIALLGSIVIIVMNIM | NivG.785 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 631 | MKKINEGLLDSKILSAFNTVIALLGSIVIIVMNIM | NivG.786 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 632 | MKKINEGLLDSKILSAFNTVIALLGSIVIIVMNIM | NivG.787 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 633 | MKKINEGLLDSKILSAFNTVIALLGSIVIIVMNIM | NivG.788 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | NivG.789 |
| | WCISLVEIYDTGDNVIRPKLFAVKIPEQCT | |
| 634 | MKKINEGLLDSKILSAFNTVIALLGSIVIIVMNIM | |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 635 | MKKINEGLLDSKILSAFNTVIALLGSIVIIVMNIM | NivG.790 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 636 | MKKINEGLLDSKILSAFNTVIALLGSIVIIVMNIM | NivG.791 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 637 | MKKINEGLLDSKILSAFNTVIALLGSIVIIVMNIM | NivG.792 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 638 | MKKINEGLLDSKILSAFNTVIALLGSIVIIVMNIM | NivG.793 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 639 | MKKINEGLLDSKILSAFNTVIALLGSIVIIVMNIM | NivG.794 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 640 | MKKINEGLLDSKILSAFNTVIALLGSIVIIVMNIM | NivG.795 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 641 | MKKINEGLLDSKILSAFNTVIALLGSIVIIVMNIM | NivG.796 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 642 | MKKINEGLLDSKILSAFNTVIALLGSIVIIVMNIM | NivG.797 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 643 | MKKINEGLLDSKILSAFNTVIALLGSIVIIVMNIM | NivG.798 |
| | IIQNYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 644 | MKKINEGLLDSKILSAFNTVIALLGSIVIIVMNIM | NivG.799 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 645 | MKKINEGLLDSKILSAFNTVIALLGSIVIIVMNIM | NivG.800 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 646 | MKKINEGLLDSKILSAFNTVIALLGSIVIIVMNIM | NivG.801 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 647 | MKKINEGLLDSKILSAFNTVIALLGSIVIIVMNIM | NivG.802 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 648 | MKKINEGLLDSKILSAFNTVIALLGSIVIIVMNIM | NivG.803 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 649 | MKKINEGLLDSKILSAFNTVIALLGSIVIIVMNIM | NivG.804 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 650 | MKKINEGLLDSKILSAFNTVIALLGSIVIIVMNIM | NivG.805 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 651 | MKKINEGLLDSKILSAFNTVIALLGSIVIIVMNIM | NivG.806 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 652 | MKKINEGLLDSKILSAFNTVIALLGSIVIIVMNIM | NivG.807 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 653 | MKKINEGLLDSKILSAFNTVIALLGSIVIIVMNIM | NivG.808 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 654 | MKKINEGLLDSKILSAFNTVIALLGSIVIIVMNIM | NivG.809 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 655 | MKKINEGLLDSKILSAFNTVIALLGSIVIIVMNIM | NivG.810 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 656 | MKKINEGLLDSKILSAFNTVIALLGSIVIIVMNIM | NivG.811 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 657 | MKKINEGLLDSKILSAFNTVIALLGSIVIIVMNIM | NivG.812 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 658 | MKKINEGLLDSKILSAFNTVIALLGSIVIIVMNIM | NivG.813 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 659 | MKKINEGLLDSKILSAFNTVIALLGSIVIIVMNIM | NivG.814 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 660 | MKKINEGLLDSKILSAFNTVIALLGSIVIIVMNIM | NivG.815 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 661 | MKKINEGLLDSKILSAFNTVIALLGSIVIIVMNIM | NivG.816 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 662 | MKKINEGLLDSKILSAFNTVIALLGSIVIIVMNIM | NivG.817 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 663 | MKKINEGLLDSKILSAFNTVIALLGSIVIIVMNIM | NivG.818 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 664 | MKKINEGLLDSKILSAFNTVIALLGSIVIIVMNIM | NivG.819 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 665 | MKKINEGLLDSKILSAFNTVIALLGSIVIIVMNIM | NivG.820 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 666 | MKKINEGLLDSKILSAFNTVIALLGSIVIIVMNIM | NivG.821 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 667 | MKKINEGLLDSKILSAFNTVIALLGSIVIIVMNIM | NivG.822 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 668 | MKKINEGLLDSKILSAFNTVIALLGSIVIIVMNIM | NivG.823 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 669 | MKKINEGLLDSKILSAFNTVIALLGSIVIIVMNIM | NivG.824 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 670 | MKKINEGLLDSKILSAFNTVIALLGSIVIIVMNIM | NivG.825 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 671 | MKKINEGLLDSKILSAFNTVIALLGSIVIIVMNIM | NivG.826 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 672 | MKKINEGLLDSKILSAFNTVIALLGSIVIIVMNIM | NivG.827 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 673 | MKKINEGLLDSKILSAFNTVIALLGSIVIIVMNIM | NivG.828 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 674 | MKKINEGLLDSKILSAFNTVIALLGSIVIIVMNIM | NivG.829 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECNISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 675 | MKKINEGLLDSKILSAFNTVIALLGSIVIIVMNIM | NivG.830 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 676 | MKKINEGLLDSKILSAFNTVIALLGSIVIIVMNIM | NivG.831 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 677 | MKKINEGLLDSKILSAFNTVIALLGSIVIIVMNIM | NivG.832 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 678 | MKKINEGLLDSKILSAFNTVIALLGSIVIIVMNIM | NivG.833 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 679 | MKKINEGLLDSKILSAFNTVIALLGSIVIIVMNIM | NivG.834 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 680 | MKKINEGLLDSKILSAFNTVIALLGSIVIIVMNIM | NivG.835 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 681 | MKKINEGLLDSKILSAFNTVIALLGSIVIIVMNIM | NivG.836 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 682 | MKKINEGLLDSKILSAFNTVIALLGSIVIIVMNIM | NivG.837 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 683 | MKKINEGLLDSKILSAFNTVIALLGSIVIIVMNIM | NivG.838 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 684 | MKKINEGLLDSKILSAFNTVIALLGSIVIIVMNIM | NivG.839 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 685 | MKKINEGLLDSKILSAFNTVIALLGSIVIIVMNIM | NivG.840 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 686 | MKKINEGLLDSKILSAFNTVIALLGSIVIIVMNIM | NivG.841 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 687 | MKKINEGLLDSKILSAFNTVIALLGSIVIIVMNIM | NivG.842 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 688 | MKKINEGLLDSKILSAFNTVIALLGSIVIIVMNIM | NivG.843 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 689 | MKKINEGLLDSKILSAFNTVIALLGSIVIIVMNIM | NivG.844 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 690 | MKKINEGLLDSKILSAFNTVIALLGSIVIIVMNIM | NivG.845 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILNS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 691 | MKKINEGLLDSKILSAFNTVIALLGSIVIIVMNIM | NivG.846 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 692 | MKKINEGLLDSKILSAFNTVIALLGSIVIIVMNIM | NivG.847 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 693 | MKKINEGLLDSKILSAFNTVIALLGSIVIIVMNIM | NivG.848 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 694 | MKKINEGLLDSKILSAFNTVIALLGSIVIIVMNIM | NivG.849 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 695 | MKKINEGLLDSKILSAFNTVIALLGSIVIIVMNIM | NivG.850 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 696 | MKKINEGLLDSKILSAFNTVIALLGSIVIIVMNIM | NivG.851 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 697 | MKKINEGLLDSKILSAFNTVIALLGSIVIIVMNIM | NivG.852 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 698 | MKKINEGLLDSKILSAFNTVIALLGSIVIIVMNIM | NivG.853 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYNDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 699 | MKKINEGLLDSKILSAFNTVIALLGSIVIIVMNIM | NivG.854 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 700 | MKKINEGLLDSKILSAFNTVIALLGSIVIIVMNIM | NivG.855 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 701 | MKKINEGLLDSKILSAFNTVIALLGSIVIIVMNIM | NivG.856 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 702 | MKKINEGLLDSKILSAFNTVIALLGSIVIIVMNIM | NivG.857 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYNLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 703 | MKKINEGLLDSKILSAFNTVIALLGSIVIIVMNIM | NivG.858 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 704 | MKKINEGLLDSKILSAFNTVIALLGSIVIIVMNIM | NivG.859 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRNNTVISRPGQSQCPRFNTCPAICAEG | |
| | VYNDAFLIDRINWISAGVFLDSQATAANPVFTV | |
| | FKDNEILYRAQLASEDTNAQKTITNCFLLKNKI | |
| | WCISLVEIYDTGDNVIRPKLFAVKIPEQCT | |
| 705 | MKKINEGLLDSKILSAFNTVIALLGSIVIIVMNIM | NivG.860 |
| | IIQQYTRSTDNQAVIKDALQGIQQQIKGLADKIG | |
| | TEIGPKVSLIDTSSTITIPANIGLLGSKISQSTASIN | |
| | ENVNEKCKFTLPPLKIHECQISCPNPLPFREYRPQ | |
| | TEGVSNLVGLPNNICLQKTSNQILKPKLISYTLP | |
| | VVGQSGTCITDPLLAMDEGYFAYSHLERIGSCS | |
| | RGVSKQRIIGVGEVLDRGDEVPSLFMTNVWTPP | |
| | NPNTVYHCSAVYNNEFYYVLCAVSTVGDPILQS | |
| | TYWSGSLMMTRLAVKPKSNGGGYNQHQLALR | |
| | SIEKGRYDKVMPYGPSGIKQGDTLYFPAVGFLV | |
| | RTEFKYQDSNCPITKCQYSKPENCRLSMGIRPNS | |
| | HYILRSGLLKYQLSDGENPKVVFIEISDQRLSIGS | |
| | PSKIYDSLGQPVFYQASFSWDTMIKFGDVLTVN | |
| | PLVVNWRQNTVISRPGQSQCPRFNTCPAICAEG | |
| | VYNDAFLIDRINWISAGVFLDSNATAANPVFTV | |
| | FKDNEILYRAQLASEDTNAQKTITNCFLLKNKI | |
| | WCISLVEIYDTGDNVIRPKLFAVKIPEQCT | |
| 706 | EVQLVESGGG LVQSGGSLRL SCAASGFTFS | VHH |
| | GYWMYWVRQA PGKGLEWVSA ISPGGGSTYY | |
| | PDSVKGRFTI SRDNAKNTLY LQMNSLEPED | |
| | TALYYCASSL TATHTYEYDY WGQGTQVTVS | |

1. A pseudotyped viral particle or virus-like particle comprising a Paramyxovirus glycoprotein (F) and a Paramyxovirus G/H glycoprotein, wherein the G/H protein has reduced glycosylation as compared to a wild-type G/H protein.

2. A lipid particle, comprising a lipid bilayer, a Paramyxovirus glycoprotein F, and a Paramyxovirus G/H glycoprotein, wherein the G/H protein is exposed on the outside of the lipid bilayer, and wherein the G/H protein has reduced glycosylation as compared to a wild-type G/H protein.

3. The pseudotyped viral particle or virus-like particle of claim 1 or the lipid particle of claim 2, wherein the G/H protein is a variant G/H protein.

4. The pseudotyped viral particle, virus-like particle, or lipid particle of claim 3, wherein the variant G/H protein comprises an amino acid substitution at one or more amino acid positions that reduce glycosylation of the G/H protein.

5. The pseudotyped viral particle, virus-like particle, or lipid particle of claim 4, wherein the variant G/H protein is a variant of a truncated paramyxovirus G/H glycoprotein in which is present the amino acid substitution at one or more amino acid positions.

6. The pseudotyped viral particle, virus-like particle, or lipid particle of claim 5, wherein the truncated paramyxovirus G/H glycoprotein has a truncated cytoplasmic tail as compared to the full-length G/H glycoprotein from the same Paramyxovirus, optionally wherein the full-length G/H glycoprotein is set forth in SEQ ID NO: 13.

7. The pseudotyped viral particle, virus-like particle, or lipid particle of claim 6, wherein the truncated paramyxovirus G/H glycoprotein has a truncated cytoplasmic that is set forth by any one of SEQ ID NOS: 355-557.

8. The pseudotyped viral particle, virus-like particle, or lipid particle of claim 5-7, wherein the truncated paramyxovirus G/H glycoprotein is a truncated Nipah G (NiV-G).

9. The pseudotyped viral particle, virus-like particle, or lipid particle of any of claims 5-8, wherein the truncated paramyxovirus G/H glycoprotein lacks up to 40, up to 30, up to 20, or up to 10 contiguous amino acids at the N-terminus.

10. The pseudotyped viral particle, virus-like particle, or lipid particle of any of claims 5-9, wherein the truncated paramyxovirus G/H glycoprotein lacks amino acid residues 2-34.

11. The pseudotyped viral particle, virus-like particle, or lipid particle of any of claims 5-10, wherein the truncated paramyxovirus G/H glycoprotein is set forth by any one of SEQ ID NOS: 1, 558-575.

12. The pseudotyped viral particle, virus-like particle, or lipid particle of any of claims 5-11, wherein the truncated G/H is set forth by a SEQ ID NO:1.

13. The pseudotyped viral particle, virus-like particle, or lipid particle of any of claims 4-12, wherein the one or more amino acid substitutions disrupts an N-linked glycosylation site.

14. The pseudotyped viral particle, virus-like particle, or lipid particle of any of claims 4-12, wherein the one or more amino acid substitutions disrupts an O-linked glycosylation site.

15. The pseudotyped viral particle, virus-like particle, or lipid particle of any one of claims 4-14, wherein the one or more amino acid substitutions are at positions corresponding to positions selected from the group consisting of 39, 126, 128, 273, 345, 384, 448, and 496 of SEQ ID NO:1.

16. The pseudotyped viral particle, virus-like particle, or lipid particle of any one of claims 4-15, wherein the amino acid substitution is a substitution at position 273.

17. The pseudotyped viral particle, virus-like particle, or lipid particle of any one of claims 4-15, wherein the protein comprises at least three amino acid substitutions.

18. The pseudotyped viral particle, virus-like particle, or lipid particle of claim 17, wherein the amino acid substitutions are at positions 273, 384, and 496.

19. The pseudotyped viral particle, virus-like particle, or lipid particle of claim 17, wherein the amino acid substitution are at positions 273, 345, and 496.

20. The pseudotyped viral particle, virus-like particle, or lipid particle of any one of claims 4-19, wherein the amino acid substitution is an asparagine substituted with a glutamine.

21. The pseudotyped viral particle, virus-like particle, or lipid particle of any one of claims 4-20, wherein the G/H protein is a NiV-G protein comprising a substitution at amino acid position 273 (N273Q) with reference to numbering of positions of SEQ ID NO: 1.

22. The pseudotyped viral particle, virus-like particle, or lipid particle of any one of claims 4-20, wherein the G/H protein is a NiV-G protein comprising a substitution at amino acid position 273 (N273Q), a substitution at amino acid position 384 (N384Q), and a substitution at amino acid position 496 (N496Q), each with reference to numbering of positions of SEQ ID NO: 1.

23. The pseudotyped viral particle, virus-like particle, or lipid particle of any one of claims 4-20, wherein the G/H protein is a NiV-G protein comprising a substitution at amino acid position 273 (N273Q), a substitution at amino acid position 345 (N345Q), and a substitution at amino acid position 496 (N496Q), each with reference to numbering of positions of SEQ ID NO: 1.

24. The pseudotyped viral particle, virus-like particle, or lipid particle of any one of claims 4-20, wherein the variant G/H protein comprises the sequence selected from the group consisting of SEQ ID Nos: 579-705.

25. The pseudotyped viral particle, virus-like particle, or lipid particle of any one of claims 1-21 and 13, wherein the variant G/H protein comprises the sequence of SEQ ID NO: 581.

26. The pseudotyped viral particle, virus-like particle, or lipid particle of any one of claims 1-20, 22 and 24, wherein the variant G/H protein comprises the sequence of SEQ ID NO: 602.

27. The pseudotyped viral particle, virus-like particle, or lipid particle of any one of claims 1-20, 23 and 24, wherein the variant G/H protein comprises the sequence of SEQ ID NO: 606.

28. The pseudotyped viral particle, virus-like particle, or lipid particle of any one of claims 4-27, wherein the paramyxovirus G/H protein comprises a truncation at the N-terminus.

29. The pseudotyped viral particle, virus-like particle, or lipid particle of claim 28, wherein the truncation comprises a truncation corresponding to deletion of amino acids 2-34 of SEQ ID NO: 13.

30. The pseudotyped viral particle, virus-like particle, or lipid particle of any one of claims 1-29, wherein the paramyxovirus G/H protein further comprises one or more amino acid mutations that reduce or abrogate native receptor tropism.

31. The pseudotyped viral particle, virus-like particle, or lipid particle of claim 30, wherein the amino acid mutations comprise mutations at positions corresponding to E501, W504, Q530, and E533 of SEQ ID NO: 13.

32. The pseudotyped viral particle, virus-like particle, or lipid particle of any one of claims 1-31, wherein the paramyxovirus G/H protein is fused to a targeting domain.

33. The pseudotyped viral particle, virus-like particle, or lipid particle of claim 32, wherein the targeting domain comprises a VHH or a scFv domain.

34. The pseudotyped viral particle, virus-like particle, or lipid particle of any one of claims 1-33, wherein the paramyxovirus G/H protein is a Nipah virus G protein (NiV-G).

35. The pseudotyped viral particle, virus-like particle, or lipid particle of any one of claims 1-34, wherein the pseudotyped viral particle, virus-like particle, or lipid particle further comprises a paramyxovirus F protein.

36. The pseudotyped viral particle, virus-like particle, or lipid particle of claim 35, wherein the F protein comprises a 22 amino acid truncation at the C-terminus.

37. The pseudotyped viral particle, virus-like particle, or lipid particle of claim 35 or 36, wherein the F protein is a Nipah virus F glycoprotein (NiV-F).

38. The pseudotyped viral particle, virus-like particle, or lipid particle of any one of claims 1-37, wherein the pseudotyped viral particle, virus-like particle, or lipid particle is generated from a producer cell comprising one or more mutations that disrupts protein glycosylation.

39. The pseudotyped viral particle, virus-like particle, or lipid particle of any one of claims 1-38, wherein the pseudotyped viral particle, virus-like particle, or lipid particle is generated from a producer cell contacted with an inhibitor of glycosylation.

40. The pseudotyped viral particle, virus-like particle, or lipid particle of any one of claims 1-39, wherein the pseudotyped viral particle, virus-like particle, or lipid particle is contacted with one or more glycosidases after generation.

41. The pseudotyped viral particle, virus-like particle, or lipid particle of any one of claims 1-40, further comprising an exogenous agent.

42. The pseudotyped viral particle, virus-like particle, or lipid particle of claim 41, wherein the exogenous agent is a protein or a nucleic acid.

43. The pseudotyped viral particle, virus-like particle, or lipid particle of claim 42, wherein the exogenous agent is a nucleic acid encoding a cargo for delivery to the target cell.

44. The pseudotyped viral particle, virus-like particle, or lipid particle of claim 42 or 43, wherein the exogenous agent is or encodes a therapeutic agent or a diagnostic agent.

45. The pseudotyped viral particle, virus-like particle, or lipid particle of any one of claims 41-44, wherein the exogenous agent encodes a chimeric antigen receptor (CAR).

46. The pseudotyped viral particle, virus-like particle, or lipid particle of any one of claims 41-44, wherein the exogenous agent encodes a genome modifying enzyme.

47. The pseudotyped viral particle, virus-like particle, or lipid particle of any one of claims 1-46, wherein the pseudotyped viral particle or virus-like particle or the lipid particle has reduced transduction of one or more off-target cell types.

48. The pseudotyped viral particle, virus-like particle, or lipid particle of claim 47, wherein the off-target cell type is a hepatocyte.

49. The pseudotyped viral particle, virus-like particle, or lipid particle of claim 47, wherein the off-target cell type is a hematopoietic stem cell.

50. The pseudotyped viral particle, virus-like particle, or lipid particle of any one of claims 1-49, wherein the pseudotyped viral particle or virus-like particle or the lipid particle does not have reduced transduction of on-target cell types.

51. The pseudotyped viral particle, virus-like particle, or lipid particle of claim 50, wherein the on target cell types comprise an immune cell.

52. A variant paramyxovirus G/H protein comprising one or more amino acid substitutions at positions 39, 126, 128, 273, 345, 384, 448, and 496 corresponding to SEQ ID NO: 1

53. The variant paramyxovirus G/H protein of claim 52, further comprising a truncation of amino acids 2-34 corresponding to SEQ ID NO: 13.

54. The variant paramyxovirus G/H protein of claim 52 or 53, wherein the variant protein is fused to a CD8 binding domain.

55. The variant paramyxovirus G/H protein of claim 52 or 53, wherein the variant protein is fused to a CD4 binding domain.

56. A polynucleotide comprising a nucleic acid molecule encoding a variant paramyxovirus G protein comprising an amino acid substitution at one or more amino acid positions that reduce glycosylation of the variant G/H protein.

57. The polynucleotide of claim 56, wherein the variant G/H protein comprises an amino acid sequence selected from the group consisting of SEQ ID NO: 579-705.

58. The polynucleotide of claim 57, wherein the variant G/H protein comprises an amino acid sequence SEQ ID NO: 581.

59. The polynucleotide of claim 57, wherein the variant G/H protein comprises an amino acid sequence SEQ ID NO: 602.

60. The polynucleotide of claim 57, wherein the variant G/H protein comprises an amino acid sequence SEQ ID NO: 606.

61. A vector comprising the polynucleotide of any one of claims 56-60.

62. The vector of claim 61, wherein the vector is a mammalian vector, viral vector or artificial chromosome, optionally wherein the artificial chromosome is a bacterial artificial chromosome (BAC).

63. A plasmid comprising the polynucleotide of any one of claims 56-60.

64. The plasmid of claim 63, further comprising one or more nucleic acids encoding proteins for lentivirus production.

65. A cell comprising the polynucleotide of any one of claims 56-60, the vector of claim 61 or 62, or the plasmid of claim 63 or 64.

66. A method of making a pseudotyped viral particle or virus-like particle comprising a variant paramyxovirus F and/or paramyxovirus G/H protein having reduced glycosylation, comprising:
a) providing a producer cell that comprises a viral particle or virus-like viral nucleic acid(s), and the polynucleotide of any one of claims 56-60, the vector of claim 61 or 62, or the plasmid of claim 63 or 64;
b) culturing the cell under conditions that allow for production of the viral particle or virus-like particle, and
c) separating, enriching, or purifying the viral particle or virus-like particle from the cell, thereby making the pseudotyped viral particle or virus-like particle.

67. A method of making a lipid particle comprising a variant paramyxovirus F and/or paramyxovirus G/H protein having reduced glycosylation, comprising:
a) providing a cell that comprises the polynucleotide of any one of claims 56-60, the vector of claim 61 or 62, or the plasmid of claim 63 or 64;
b) culturing the cell under conditions that allow for production of a lipid particle, and
c) separating, enriching, or purifying the lipid particle from the cell, thereby making the lipid particle.

68. A method of making a pseudotyped viral particle or virus-like particle comprising a paramyxovirus F and/or paramyxovirus G/H protein having reduced glycosylation, comprising separating, enriching, or purifying the pseudotyped viral particle or virus-like particle, wherein the separating, enriching, or purifying further comprises enzymatic removal of glycans attached to the paramyxovirus F protein and/or paramyxovirus G/H protein.

69. A pseudotyped viral particle or virus-like particle made according to the method of claim 68.

70. A method of making a lipid particle comprising a paramyxovirus F and/or paramyxovirus G/H protein having reduced glycosylation, comprising separating, enriching, or purifying the lipid particle, wherein the separating, enriching, or purifying further comprises enzymatic removal of glycans attached to the paramyxovirus F protein and/or paramyxovirus G/H protein.

71. A lipid particle made according to the method of claim 70.

72. A method of making a pseudotyped viral particle or virus-like particle comprising a paramyxovirus F and/or paramyxovirus G/H protein having reduced glycosylation, comprising:
a) providing a producer cell that comprises a viral particle or virus-like viral nucleic acid(s) and paramyxovirus F and/or paramyxovirus G/H nucleic acid(s);
b) culturing the cell under conditions that allow for production of the viral particle or virus-like particle, wherein the culturing comprises treating the cell with a modulator of glycosylation; and
c) separating, enriching, or purifying the viral particle or virus-like particle from the cell, thereby making the pseudotyped viral particle or virus-like particle.

73. A pseudotyped viral particle or virus-like particle according to the method of claim 72.

74. A method of making a lipid particle comprising a paramyxovirus F and/or paramyxovirus G/H protein having reduced glycosylation, comprising:
a) providing a cell that comprises a paramyxovirus F and/or paramyxovirus G/H nucleic acid(s);
b) culturing the cell under conditions that allow for production of a lipid particle, wherein the culturing comprises treating the cell with a modulator of glycosylation; and
c) separating, enriching, or purifying the lipid particle from the cell, thereby making the lipid particle.

75. A lipid particle according to the method of claim 74.

76. A composition comprising a producer cell, cell medium, and one or more modulators of glycosylation.

77. A method of making a pseudotyped viral particle or virus-like particle comprising a paramyxovirus F and/or paramyxovirus G/H protein having reduced glycosylation, comprising:
a) providing a producer cell that comprises a viral particle or virus-like viral nucleic acid(s) and paramyxovirus F and/or paramyxovirus G/H nucleic acid(s), wherein the producer cell comprises one or more genetic modifications that modulate glycosylation;
b) culturing the cell under conditions that allow for production of the viral particle or virus-like particle, and
c) separating, enriching, or purifying the viral particle or virus-like particle from the cell, thereby making the pseudotyped viral particle or virus-like particle.

78. A composition comprising a producer cell, wherein the producer cell comprises one or more nucleic acids for producing a viral particle or virus-like particle comprising a paramyxovirus F and/or paramyxovirus G/H protein, and wherein the producer cell comprises one or more genetic modifications that modulate glycosylation.

79. A method of making a lipid particle comprising a variant paramyxovirus F and/or paramyxovirus G/H protein having reduced glycosylation, comprising:
a) providing a cell paramyxovirus F and/or paramyxovirus G/H nucleic acid(s), wherein the cell comprises one or more genetic modifications that modulate glycosylation;
b) culturing the cell under conditions that allow for production of a lipid particle, and
c) separating, enriching, or purifying the lipid particle from the cell, thereby making the lipid particle.

80. A composition comprising a producer cell, wherein the producer cell comprises one or more nucleic acids for producing a lipid particle comprising a paramyxovirus F and/or paramyxovirus G/H protein, and wherein the producer cell comprises one or more genetic modifications that modulate glycosylation.

81. A method of transducing a cell with a with a pseudotyped viral particle, virus-like particle, or lipid particle, comprising contacting the cell with the pseudotyped viral particle, virus-like particle, or lipid particle of any one of claims 1-51, 69, 71, 73 and 75.

82. A method of delivering an exogenous agent to a cell, comprising contacting the cell with the pseudotyped viral particle, virus-like particle, or lipid particle of any one of claims 1-51, 69, 71, 73 and 75, wherein the pseudotyped viral particle, virus-like particle, or lipid particle comprises the exogenous agent.

83. The method of claim 82, wherein the contacting transduces the cell with the pseudotyped viral particle, virus-like particle, or lipid particle.

84. The method of claim 82 or 83, wherein the contacting is in vivo in a subject.

85. A method of reducing off-target transduction of a pseudotyped viral particle, virus-like particle, or lipid particle comprising administering the pseudotyped viral particle, virus-like particle, or lipid particle of any one of claims 1-51, 69, 71, 73 and 75.

86. The method of claim 85, wherein on-target transduction is not reduced.

87. A method of treating a disease or disorder in a subject, comprising administering to the subject the pseudotyped viral particle, virus-like particle, or lipid particle of any one of claims 1-51, 69, 71, 73 and 75.


SEQUENCE LISTING (706 sequences)

SEQ ID NO: 1 - protein, 569 aa, synthetic construct
  REGION 1..569: note = NiV-G
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 2 - protein, 569 aa, synthetic construct
  REGION 1..569: note = NiV-G N39Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 3 - protein, 569 aa, synthetic construct
  REGION 1..569: note = NiV-G N126Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 4 - protein, 569 aa, synthetic construct
  REGION 1..569: note = NiV-G N126Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNIACPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 5 - protein, 569 aa, synthetic construct
  REGION 1..569: note = NiV-G N273Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 6 - protein, 569 aa, synthetic construct
  REGION 1..569: note = NiV-G N345Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 7 - protein, 569 aa, synthetic construct
  REGION 1..569: note = NiV-G N384Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 8 - protein, 569 aa, synthetic construct
  REGION 1..569: note = NiV-G N448Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 9 - protein, 569 aa, synthetic construct
  REGION 1..569: note = NiV-G N496Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 10 - protein, 569 aa, synthetic construct
  REGION 1..569: note = NiV-G N345Q, N384Q, N448Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 11 - protein, 569 aa, synthetic construct
  REGION 1..569: note = NiV-G N345Q, N448Q, N496Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 12 - protein, 569 aa, synthetic construct
  REGION 1..569: note = NiV-G N345Q, N496Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 13 - protein, 602 aa, synthetic construct
  REGION 1..602: note = NiVG protein attachment glycoprotein (602 aa) Uniprot Q9IH62
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 14 - protein, 557 aa, synthetic construct
  REGION 1..557: note = NiV-G backbone DeltaCT
ILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 15 - protein, 557 aa, synthetic construct
  REGION 1..557: note = NiV-G backbone DeltaCT and mutated (E501 A, W504A, Q530A, E533A)
ILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 16 - protein, 602 aa, synthetic construct
  REGION 1..602: note = NiVG with NiVG CT_45_FL, mutated (E501 A, W504A, Q530A, E533A)
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 17 - protein, 8 aa, synthetic construct
  REGION 1..8: note = CDR-H1
GYTFSNYW

SEQ ID NO: 18 - protein, 8 aa, synthetic construct
  REGION 1..8: note = CDR-H2
ILPGSGST

SEQ ID NO: 19 - protein, 13 aa, synthetic construct
  REGION 1..13: note = CDR-H3
ARRGYGYDEGFDY

SEQ ID NO: 20 - protein, 6 aa, synthetic construct
  REGION 1..6: note = CDR-L1
QDINSY

SEQ ID NO: 21 - skipped

SEQ ID NO: 22 - protein, 9 aa, synthetic construct
  REGION 1..9: note = CDR-L3
LQYDEFPPT

SEQ ID NO: 23 - protein, 120 aa, synthetic construct
  REGION 1..120: note = VH
QVQLQQSGAELMKPGASVKMSCKATGYTFSNYWIEWVKQRPGHGLEWIGEILPGSGSTSYNEKFKGKATFTADTSSSTAYMQLSSLTSEDSAVYYCARRGYGYDEGFDYWGQGSTLTVSS

SEQ ID NO: 24 - protein, 108 aa, synthetic construct
  REGION 1..108: note = VL
DIKMTQSPSSMYASLGERVTITCKASQDINSYLSWFQQKPGKSPKTLIYRANRLVDGVPSRFSGSGSGQDYSLTISSLEYEDMGIYYCLQYDEFPPTFGAGTKLELKR

SEQ ID NO: 25 - protein, 243 aa, synthetic construct
  REGION 1..243: note = scFv
QVQLQQSGAELMKPGASVKMSCKATGYTFSNYWIEWVKQRPGHGLEWIGEILPGSGSTSYNEKFKGKATFTADTSSSTAYMQLSSLTSEDSAVYYCARRGYGYDEGFDYWGQGSTLTVSSGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDEFPPTFGAGTKLELKR

SEQ ID NO: 26 - protein, 8 aa, synthetic construct
  REGION 1..8: note = CDR-H1
GYTFTDYV

SEQ ID NO: 27 - protein, 8 aa, synthetic construct
  REGION 1..8: note = CDR-H2
IYPGSGSS

SEQ ID NO: 28 - protein, 10 aa, synthetic construct
  REGION 1..10: note = CDR-H3
ARPGDLGFAY

SEQ ID NO: 29 - protein, 10 aa, synthetic construct
  REGION 1..10: note = CDR-L1
QSVDYDGDSY

SEQ ID NO: 30 - skipped

SEQ ID NO: 31 - protein, 9 aa, synthetic construct
  REGION 1..9: note = CDR-L3
QQSNKDPFT

SEQ ID NO: 32 - protein, 117 aa, synthetic construct
  REGION 1..117: note = VH
QVQLQQSGPELVKPGASVKMSCKASGYTFTDYVISWVRQAPGQGLEWIGEIYPGSGSSYYNEKFKGRATLTADKSSNTAYMQLSSLRSEDSAVYFCARPGDLGFAYWGQGTLVTVSS

SEQ ID NO: 33 - protein, 112 aa, synthetic construct
  REGION 1..112: note = VL
DIVLTQSPSSLAVSLGQRATISCKASQSVDYDGDSYMNWYQQKPGQPPKLLIYAASNLESGIPARFSGSGSGTDFTLTIHPVEEEDAATYYCQQSNKDPFTFGGGTKLELKR

SEQ ID NO: 34 - protein, 244 aa, synthetic construct
  REGION 1..244: note = scFv
QVQLQQSGPELVKPGASVKMSCKASGYTFTDYVISWVRQAPGQGLEWIGEIYPGSGSSYYNEKFKGRATLTADKSSNTAYMQLSSLRSEDSAVYFCARPGDLGFAYWGQGTLVTVSSGGGGSGGGGSGGGGSDIVLTQSPSSLAVSLGQRATISCKASQSVDYDGDSYMNWYQQKPGQPPKLLIYAASNLESGIPARFSGSGSGTDFTLTIHPVEEEDAATYYCQQSNKDPFTFGGGTKLELKR

SEQ ID NO: 35 - protein, 8 aa, synthetic construct
  REGION 1..8: note = CDR-H1
GGTFSSYA

SEQ ID NO: 36 - protein, 8 aa, synthetic construct
  REGION 1..8: note = CDR-H2
INPNSGGT

SEQ ID NO: 37 - protein, 11 aa, synthetic construct
  REGION 1..11: note = CDR-H3
ARDGYSGSYSD

SEQ ID NO: 38 - protein, 12 aa, synthetic construct
  REGION 1..12: note = CDR-L1
QSVLSSSYNKNY

SEQ ID NO: 39 - skipped

SEQ ID NO: 40 - protein, 9 aa, synthetic construct
  REGION 1..9: note = CDR-L3
QQYYSTPWT

SEQ ID NO: 41 - protein, 118 aa, synthetic construct
  REGION 1..118: note = VH
QVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCARDGYSGSYSDWGQGTLVTVSS

SEQ ID NO: 42 - protein, 113 aa, synthetic construct
  REGION 1..113: note = VL
DIVMTQSPDSLAVSLGERATINCKSSQSVLSSSYNKNYLAWYQQKPGQPPKLLIYWASTRESGVPDRFSGSGSGTDFTLTISSLQAEDVAVYYCQQYYSTPWTFGQGTKVEIK

SEQ ID NO: 43 - protein, 246 aa, synthetic construct
  REGION 1..246: note = scFv
QVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCARDGYSGSYSDWGQGTLVTVSSGGGGSGGGGSGGGGSDIVMTQSPDSLAVSLGERATINCKSSQSVLSSSYNKNYLAWYQQKPGQPPKLLIYWASTRESGVPDRFSGSGSGTDFTLTISSLQAEDVAVYYCQQYYSTPWTFGQGTKVEIK

SEQ ID NO: 44 - protein, 8 aa, synthetic construct
  REGION 1..8: note = CDR-H1
GYTFTSYD

SEQ ID NO: 45 - protein, 8 aa, synthetic construct
  REGION 1..8: note = CDR-H2
IIPLSGAP

SEQ ID NO: 46 - protein, 15 aa, synthetic construct
  REGION 1..15: note = CDR-H3
ARGALYNWNDGWFDP

SEQ ID NO: 47 - protein, 6 aa, synthetic construct
  REGION 1..6: note = CDR-L1
QDIGDY

SEQ ID NO: 48 - skipped

SEQ ID NO: 49 - protein, 9 aa, synthetic construct
  REGION 1..9: note = CDR-L3
QQANSFPLT

SEQ ID NO: 50 - protein, 122 aa, synthetic construct
  REGION 1..122: note = VH
QVQLVQSGAEVKKPGSSVKVSCKASGYTFTSYDINWVRQAPGQGLEWMGGIIPLSGAPNYAHKFQGRVTITADESTSTAYMELSSLRSEDTAVYYCARGALYNWNDGWFDPWGQGTLVTVSS

SEQ ID NO: 51 - protein, 107 aa, synthetic construct
  REGION 1..107: note = VL
DIQMTQSPSSLSASVGDRVTITCRASQDIGDYLAWYQQKPGKAPKLLIYDASSLQSGVPSRFSGSGSGTDFTLTISSLQPEDFATYYCQQANSFPLTFGGGTKVEIK

SEQ ID NO: 52 - protein, 244 aa, synthetic construct
  REGION 1..244: note = scFv
QVQLVQSGAEVKKPGSSVKVSCKASGYTFTSYDINWVRQAPGQGLEWMGGIIPLSGAPNYAHKFQGRVTITADESTSTAYMELSSLRSEDTAVYYCARGALYNWNDGWFDPWGQGTLVTVSSGGGGSGGGGSGGGGSDIQMTQSPSSLSASVGDRVTITCRASQDIGDYLAWYQQKPGKAPKLLIYDASSLQSGVPSRFSGSGSGTDFTLTISSLQPEDFATYYCQQANSFPLTFGGGTKVEIK

SEQ ID NO: 53 - protein, 8 aa, synthetic construct
  REGION 1..8: note = CDR-H1
GDTFSDHA

SEQ ID NO: 54 - protein, 8 aa, synthetic construct
  REGION 1..8: note = CDR-H2
MNPKIGNT

SEQ ID NO: 55 - protein, 12 aa, synthetic construct
  REGION 1..12: note = CDR-H3
VYDSSGYDAFDI

SEQ ID NO: 56 - protein, 12 aa, synthetic construct
  REGION 1..12: note = CDR-L1
QSVLSTSYNRNF

SEQ ID NO: 57 - protein, 9 aa, synthetic construct
  REGION 1..9: note = CDR-L3
QQYYSTPYT

SEQ ID NO: 58 - protein, 119 aa, synthetic construct
  REGION 1..119: note = VH
QVQLVQSGAEVKKPGASVKVSCKASGDTFSDHAINWVRQAPGQGLEWMGWMNPKIGNTGYAQKFQGRVTMTRDTSTSTVYMELSSLRSEDTAVYYCVYDSSGYDAFDIWGQGTTVTVSS

SEQ ID NO: 59 - protein, 113 aa, synthetic construct
  REGION 1..113: note = VL
DIVMTQSPDSLAVSLGERATINCKSSQSVLSTSYNRNFLAWYQQKPGQPPKLLIYWASTRQSGVPDRFSGSGSGTDFTLTISSLQAEDVAVYYCQQYYSTPYTFGQGTKLEIK

SEQ ID NO: 60 - protein, 247 aa, synthetic construct
  REGION 1..247: note = scFv
QVQLVQSGAEVKKPGASVKVSCKASGDTFSDHAINWVRQAPGQGLEWMGWMNPKIGNTGYAQKFQGRVTMTRDTSTSTVYMELSSLRSEDTAVYYCVYDSSGYDAFDIWGQGTTVTVSSGGGGSGGGGSGGGGSDIVMTQSPDSLAVSLGERATINCKSSQSVLSTSYNRNFLAWYQQKPGQPPKLLIYWASTRQSGVPDRFSGSGSGTDFTLTISSLQAEDVAVYYCQQYYSTPYTFGQGTKLEIK

SEQ ID NO: 61 - protein, 8 aa, synthetic construct
  REGION 1..8: note = CDR-H1
GYSLITHW

SEQ ID NO: 62 - protein, 8 aa, synthetic construct
  REGION 1..8: note = CDR-H2
INPSDGVT

SEQ ID NO: 63 - protein, 11 aa, synthetic construct
  REGION 1..11: note = CDR-H3
AREYYGEGFDY

SEQ ID NO: 64 - protein, 6 aa, synthetic construct
  REGION 1..6: note = CDR-L1
QGISNY

SEQ ID NO: 65 - skipped

SEQ ID NO: 66 - protein, 9 aa, synthetic construct
  REGION 1..9: note = CDR-L3
QQSYSTPLT

SEQ ID NO: 67 - protein, 118 aa, synthetic construct
  REGION 1..118: note = VH
QVQLVQSGAEVKKPGASVKVSCKASGYSLITHWMHWVRQAPGQGLEWMGMINPSDGVTYYAQTFQGRVTMTRDTSTSTVYMELSSLRSEDTAVYYCAREYYGEGFDYWGQGTLVTVSS

SEQ ID NO: 68 - protein, 108 aa, synthetic construct
  REGION 1..108: note = VL
DIQMTQSPSSLSASVGDRVTITCRASQGISNYLAWYQQKPGKAPKLLIYSASNLQSGVPSRFSGSGSGTDFTLTISSLQPEDFATYYCQQSYSTPLTFGGGTKVEIKR

SEQ ID NO: 69 - protein, 241 aa, synthetic construct
  REGION 1..241: note = scFv
QVQLVQSGAEVKKPGASVKVSCKASGYSLITHWMHWVRQAPGQGLEWMGMINPSDGVTYYAQTFQGRVTMTRDTSTSTVYMELSSLRSEDTAVYYCAREYYGEGFDYWGQGTLVTVSSGGGGSGGGGSGGGGSDIQMTQSPSSLSASVGDRVTITCRASQGISNYLAWYQQKPGKAPKLLIYSASNLQSGVPSRFSGSGSGTDFTLTISSLQPEDFATYYCQQSYSTPLTFGGGTKVEIKR

SEQ ID NO: 70 - protein, 5 aa, synthetic construct
  REGION 1..5: note = CDR-H1
GYWMY

SEQ ID NO: 71 - protein, 17 aa, synthetic construct
  REGION 1..17: note = CDR-H2
AISPGGGSTYYPDSVKG

SEQ ID NO: 72 - protein, 12 aa, synthetic construct
  REGION 1..12: note = CDR-H3
SLTATHTYEYDY

SEQ ID NO: 73 - protein, 7 aa, synthetic construct
  REGION 1..7: note = CDR-H1
GFTFSGY

SEQ ID NO: 74 - protein, 6 aa, synthetic construct
  REGION 1..6: note = CDR-H2
SPGGGS

SEQ ID NO: 75 - protein, 5 aa, synthetic construct
  REGION 1..5: note = CDR-H1
NYWIE

SEQ ID NO: 76 - protein, 17 aa, synthetic construct
  REGION 1..17: note = CDR-H2
EILPGSGSTSYNEKFKG

SEQ ID NO: 77 - protein, 11 aa, synthetic construct
  REGION 1..11: note = CDR-H3
RGYGYDEGFDY

SEQ ID NO: 78 - protein, 11 aa, synthetic construct
  REGION 1..11: note = CDR-L1
KASQDINSYLS

SEQ ID NO: 79 - protein, 7 aa, synthetic construct
  REGION 1..7: note = CDR-L2
RANRLVD

SEQ ID NO: 80 - protein, 7 aa, synthetic construct
  REGION 1..7: note = CDR-H1
GYTFSNY

SEQ ID NO: 81 - protein, 6 aa, synthetic construct
  REGION 1..6: note = CDR-H2
LPGSGS

SEQ ID NO: 82 - protein, 5 aa, synthetic construct
  REGION 1..5: note = CDR-H1
DYVIS

SEQ ID NO: 83 - protein, 17 aa, synthetic construct
  REGION 1..17: note = CDR-H2
EIYPGSGSSYYNEKFKG

SEQ ID NO: 84 - protein, 8 aa, synthetic construct
  REGION 1..8: note = CDR-H3
PGDLGFAY

SEQ ID NO: 85 - protein, 15 aa, synthetic construct
  REGION 1..15: note = CDR-L1
KASQSVDYDGDSYMN

SEQ ID NO: 86 - protein, 7 aa, synthetic construct
  REGION 1..7: note = CDR-L2
AASNLES

SEQ ID NO: 87 - protein, 7 aa, synthetic construct
  REGION 1..7: note = CDR-H1
GYTFTDY

SEQ ID NO: 88 - protein, 6 aa, synthetic construct
  REGION 1..6: note = CDR-H2
YPGSGS

SEQ ID NO: 89 - protein, 5 aa, synthetic construct
  REGION 1..5: note = CDR-H1
SYAIS

SEQ ID NO: 90 - protein, 17 aa, synthetic construct
  REGION 1..17: note = CDR-H2
WINPNSGGTNYAQKFQG

SEQ ID NO: 91 - protein, 9 aa, synthetic construct
  REGION 1..9: note = CDR-H3
DGYSGSYSD

SEQ ID NO: 92 - protein, 17 aa, synthetic construct
  REGION 1..17: note = CDR-L1
KSSQSVLSSSYNKNYLA

SEQ ID NO: 93 - protein, 7 aa, synthetic construct
  REGION 1..7: note = CDR-L2
WASTRES

SEQ ID NO: 94 - protein, 7 aa, synthetic construct
  REGION 1..7: note = CDR-H1
GGTFSSY

SEQ ID NO: 95 - protein, 6 aa, synthetic construct
  REGION 1..6: note = CDR-H2
NPNSGG

SEQ ID NO: 96 - protein, 5 aa, synthetic construct
  REGION 1..5: note = CDR-H1
SYDIN

SEQ ID NO: 97 - protein, 17 aa, synthetic construct
  REGION 1..17: note = CDR-H2
GIIPLSGAPNYAHKFQG

SEQ ID NO: 98 - protein, 13 aa, synthetic construct
  REGION 1..13: note = CDR-H3
GALYNWNDGWFDP

SEQ ID NO: 99 - protein, 11 aa, synthetic construct
  REGION 1..11: note = CDR-L1
RASQDIGDYLA

SEQ ID NO: 100 - protein, 7 aa, synthetic construct
  REGION 1..7: note = CDR-L2
DASSLQS

SEQ ID NO: 101 - protein, 7 aa, synthetic construct
  REGION 1..7: note = CDR-H1
GYTFTSY

SEQ ID NO: 102 - protein, 6 aa, synthetic construct
  REGION 1..6: note = CDR-H2
IPLSGA

SEQ ID NO: 103 - protein, 5 aa, synthetic construct
  REGION 1..5: note = CDR-H1
DHAIN

SEQ ID NO: 104 - protein, 17 aa, synthetic construct
  REGION 1..17: note = CDR-H2
WMNPKIGNTGYAQKFQG

SEQ ID NO: 105 - protein, 10 aa, synthetic construct
  REGION 1..10: note = CDR-H3
DSSGYDAFDI

SEQ ID NO: 106 - protein, 17 aa, synthetic construct
  REGION 1..17: note = CDR-L1
KSSQSVLSTSYNRNFLA

SEQ ID NO: 107 - protein, 7 aa, synthetic construct
  REGION 1..7: note = CDR-L2
WASTRQS

SEQ ID NO: 108 - protein, 7 aa, synthetic construct
  REGION 1..7: note = CDR-H1
GDTFSDH

SEQ ID NO: 109 - protein, 6 aa, synthetic construct
  REGION 1..6: note = CDR-H2
NPKIGN

SEQ ID NO: 110 - protein, 5 aa, synthetic construct
  REGION 1..5: note = CDR-H1
THWMH

SEQ ID NO: 111 - protein, 17 aa, synthetic construct
  REGION 1..17: note = CDR-H2
MINPSDGVTYYAQTFQG

SEQ ID NO: 112 - protein, 9 aa, synthetic construct
  REGION 1..9: note = CDR-H3
EYYGEGFDY

SEQ ID NO: 113 - protein, 11 aa, synthetic construct
  REGION 1..11: note = CDR-L1
RASQGISNYLA

SEQ ID NO: 114 - protein, 7 aa, synthetic construct
  REGION 1..7: note = CDR-L2
SASNLQS

SEQ ID NO: 115 - protein, 7 aa, synthetic construct
  REGION 1..7: note = CDR-H1
GYSLITH

SEQ ID NO: 116 - protein, 6 aa, synthetic construct
  REGION 1..6: note = CDR-H2
NPSDGV

SEQ ID NO: 117 - protein, 122 aa, synthetic construct
  REGION 1..122: note = CD8_1 VH
QVQLVQSGAEVKKPGASVKVSCKASGGTFSSYAISWVRQAPGQGLEWMGIIDPSDGNTNYAQNFQGRVTMTRDTSTSTVYMELSSLRSEDTAVYYCAKERAAAGYYYYMDVWGQGTTVTVSS

SEQ ID NO: 118 - protein, 108 aa, synthetic construct
  REGION 1..108: note = CD8_1 VL
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 119 - protein, 119 aa, synthetic construct
  REGION 1..119: note = CD8_2 VH
QVQLVQSGAEVKKPGASVKVSCKASGYTFTDYYIQWVRQAPGQGLEWMGWINPNSGGTSYAQKFQGRVTMTRDTSTSTVYMELSSLRSEDTAVYYCAKEGDYYYGMDAWGQGTMVTVSS

SEQ ID NO: 120 - protein, 113 aa, synthetic construct
  REGION 1..113: note = CD8_2 VL
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPHTFGQGTKVEIKR

SEQ ID NO: 121 - protein, 117 aa, synthetic construct
  REGION 1..117: note = CD8_3 VH
QVQLVQSGAEVKKPGASVKVSCKASGYTFTSYYMHWVRQAPGQGLEWMGGFDPEDGETIYAQKFQGRVTMTRDTSTSTVYMELSSLRSEDTAVYYCARDQGWGMDVWGQGTTVTVSS

SEQ ID NO: 122 - protein, 108 aa, synthetic construct
  REGION 1..108: note = CD8_3 VL
DIQMTQSPSSLSASVGDRVTITCRASQSISSYLNWYQQKPGKAPKLLIYAASSLQSGVPSRFSGSGSGTDFTLTISSLQPEDFATYYCQQTYSTPYTFGQGTKLEIKR

SEQ ID NO: 123 - protein, 118 aa, synthetic construct
  REGION 1..118: note = CD8_4 VH
QVQLVQSGAEVKKPGASVKVSCKASGYTFTNHYMHWVRQAPGQGLEWMGWMNPNSGNTGYAQKFQGRVTMTRDTSTSTVYMELSSLRSEDTAVYYCASSESGSDLDYWGQGTLVTVSS

SEQ ID NO: 124 - protein, 108 aa, synthetic construct
  REGION 1..108: note = CD8_4 VL
DIQMTQSPSSLSASVGDRVTITCRASQTIGNYVNWYQQKPGKAPKLLIYGASNLHTGVPSRFSGSGSGTDFTLTISSLQPEDFATYYCQQTYSAPLTFGGGTKVEIKR

SEQ ID NO: 125 - protein, 125 aa, synthetic construct
  REGION 1..125: note = CD8 VHH
QVQLVESGGGLVQAGGSLRLSCAASGRTFSGYVMGWFRQAPGKQRKFVAAISRGGLSTSYADSVKGRFTISRDNAKNTVFLQMNTLKPEDTAVYYCAADRSDLYEITAASNIDSWGQGTLVTVSS

SEQ ID NO: 126 - protein, 5 aa, synthetic construct
GGGGS

SEQ ID NO: 127 - protein, 6 aa, synthetic construct
GGGGGS

SEQ ID NO: 128 - protein, 50 aa, synthetic construct
  VARIANT 6..50: note = may or may not be present
GGGGSGGGGSGGGGSGGGGSGGGGSGGGGSGGGGSGGGGSGGGGSGGGGS

SEQ ID NO: 129 - protein, 36 aa, synthetic construct
  VARIANT 7..36: note = may or may not be present
GGGGGSGGGGGSGGGGGSGGGGGSGGGGGSGGGGGS

SEQ ID NO: 130 - protein, 546 aa, synthetic construct
  REGION 1..546: note = Hendra F
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 131 - protein, 546 aa, synthetic construct
  REGION 1..546: note = Nipah F0
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 132 - protein, 557 aa, synthetic construct
  REGION 1..557: note = CedV F
MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 133 - protein, 545 aa, synthetic construct
  REGION 1..545: note = Mojiang F
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 134 - protein, 662 aa, synthetic construct
  REGION 1..662: note = Bat PV F
MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 135 - protein, 520 aa, synthetic construct
  REGION 1..520: note = Hendra virus F Protein, Without signal sequence
ILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 136 - protein, 520 aa, synthetic construct
  REGION 1..520: note = Nipah virus F Protein, without signal sequence
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 137 - protein, 551 aa, synthetic construct
  REGION 1..551: note = Cedar Virus F Protein, without signal sequence
TVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 138 - protein, 519 aa, synthetic construct
  REGION 1..519: note = Mojiang virus, Tongguan 1 F Protein, without signal sequence
IHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 139 - protein, 640 aa, synthetic construct
  REGION 1..640: note = Bat Paramyxovirus F Protein, without signal sequence
SRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 140 - protein, 524 aa, synthetic construct
  REGION 1..524: note = Truncated NiV fusion glycoprotein (FcDelta22) at cytoplasmic tail (with signal sequence)
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 141 - protein, 498 aa, synthetic construct
  REGION 1..498: note = Truncated mature NiV fusion glycoprotein (FcDelta22) at cytoplasmic tail
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 142 - protein, 7 aa, synthetic construct
ENLYFQS

SEQ ID NO: 143 - protein, 604 aa, synthetic construct
  REGION 1..604: note = gb:AF017149|Organism:Hendra virus|Strain Name:UNKNOWN-AF017149|Protein Name:glycoprotein|Gene Symbol:G
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 144 - protein, 622 aa, synthetic construct
  REGION 1..622: note = gb:JQ001776:8170-10275|Organism:Cedar virus|Strain Name:CG1a|Protein Name:attachment glycoprotein|Gene Symbol:G
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 145 - protein, 632 aa, synthetic construct
  REGION 1..632: note = gb:NC_025256:9117-11015|Organism:Bat Paramyxovirus Eid_hel/GH-M74a/GHA/2009|Strain Name:BatPV/Eid_hel/GH-M74a/GHA/2009|Protein Name:glycoprotein|Gene Symbol:G
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 146 - protein, 625 aa, synthetic construct
  REGION 1..625: note = gb:NC_025352:8716-11257|Organism:Mojiang virus|Strain Name:Tongguan1|Protein Name:attachment glycoprotein|Gene Symbol:G
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 147 - protein, 602 aa, synthetic construct; identical to SEQ ID NO: 13
  REGION 1..602: note = gb:AF212302|Organism:Nipah virus|Strain Name:UNKNOWN-AF212302|Protein Name:attachment glycoprotein|Gene Symbol:G

SEQ ID NO: 148 - protein, 21 aa, synthetic construct
  REGION 1..21: note = CD8Alpha signal peptide
MALPVTALLLPLALLLHAARP

SEQ ID NO: 149 - protein, 20 aa, synthetic construct
  REGION 1..20: note = IgK signal peptide
METDTLLLWVLLLWVPGSTG

SEQ ID NO: 150 - protein, 22 aa, synthetic construct
  REGION 1..22: note = GMCSFR-Alpha (CSF2RA) signal peptide
MLLLVTSLLLCELPHPAFLLIP

SEQ ID NO: 151 - protein, 45 aa, synthetic construct
  REGION 1..45: note = CD8Alpha hinge domain
TTTPAPRPPTPAPTIASQPLSLRPEACRPAAGGAVHTRGLDFACD

SEQ ID NO: 152 - protein, 39 aa, synthetic construct
  REGION 1..39: note = CD28 hinge domain
IEVMYPPPYLDNEKSNGTIIHVKGKHLCPSPLFPGPSKP

SEQ ID NO: 153 - protein, 42 aa, synthetic construct
  REGION 1..42: note = CD28 hinge domain
AAAIEVMYPPPYLDNEKSNGTIIHVKGKHLCPSPLFPGPSKP

SEQ ID NO: 154 - protein, 12 aa, synthetic construct
  REGION 1..12: note = IgG4 hinge domain
ESKYGPPCPPCP

SEQ ID NO: 155 - protein, 12 aa, synthetic construct
  REGION 1..12: note = IgG4 hinge domain
ESKYGPPCPSCP

SEQ ID NO: 156 - protein, 229 aa, synthetic construct
  REGION 1..229: note = IgG4 hinge-CH2-CH3 domain
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 157 - protein, 24 aa, synthetic construct
  REGION 1..24: note = CD8Alpha transmembrane domain
IYIWAPLAGTCGVLLLSLVITLYC

SEQ ID NO: 158 - protein, 27 aa, synthetic construct
  REGION 1..27: note = CD28 transmembrane domain
FWVLVVVGGVLACYSLLVTVAFIIFWV

SEQ ID NO: 159 - protein, 28 aa, synthetic construct
  REGION 1..28: note = CD28 transmembrane domain
MFWVLVVVGGVLACYSLLVTVAFIIFWV

SEQ ID NO: 160 - protein, 42 aa, synthetic construct
  REGION 1..42: note = 4-1BB costimulatory domain
KRGRKKLLYIFKQPFMRPVQTTQEEDGCSCRFPEEEEGGCEL

SEQ ID NO: 161 - protein, 41 aa, synthetic construct
  REGION 1..41: note = CD28 costimulatory domain
RSKRSRLLHSDYMNMTPRRPGPTRKHYQPYAPPRDFAAYRS

SEQ ID NO: 162 - protein, 112 aa, synthetic construct
  REGION 1..112: note = CD3Zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 163 - protein, 112 aa, synthetic construct
  REGION 1..112: note = CD3Zeta signaling domain (with Q to K mutation at position 14)
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 164 - protein, 245 aa, synthetic construct
  REGION 1..245: note = Anti-CD19 FMC63 scFv entire sequence, with Whitlow linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 165 - protein, 107 aa, synthetic construct
  REGION 1..107: note = Anti-CD19 FMC63 scFv light chain variable region
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 166 - protein, 6 aa, synthetic construct
  REGION 1..6: note = Anti-CD19 FMC63 scFv light chain CDR1
QDISKY

SEQ ID NO: 167 - skipped

SEQ ID NO: 168 - protein, 9 aa, synthetic construct
  REGION 1..9: note = Anti-CD19 FMC63 scFv light chain CDR3
QQGNTLPYT

SEQ ID NO: 169 - protein, 18 aa, synthetic construct
  REGION 1..18: note = Whitlow linker
GSTSGSGKPGSGEGSTKG

SEQ ID NO: 170 - protein, 120 aa, synthetic construct
  REGION 1..120: note = Anti-CD19 FMC63 scFv heavy chain variable region
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 171 - protein, 8 aa, synthetic construct
  REGION 1..8: note = Anti-CD19 FMC63 scFv heavy chain CDR1
GVSLPDYG

SEQ ID NO: 172 - protein, 7 aa, synthetic construct
  REGION 1..7: note = Anti-CD19 FMC63 scFv heavy chain CDR2
IWGSETT

SEQ ID NO: 173 - protein, 14 aa, synthetic construct
  REGION 1..14: note = Anti-CD19 FMC63 scFv heavy chain CDR3
AKHYYYGGSYAMDY

SEQ ID NO: 174 - protein, 242 aa, synthetic construct
  REGION 1..242: note = Anti-CD19 FMC63 scFv entire sequence, with 3xG4S linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 175 - protein, 15 aa, synthetic construct
  REGION 1..15: note = 3xG4S linker
GGGGSGGGGSGGGGS

SEQ ID NO: 176 - DNA, 1467 bp, synthetic construct
  misc_feature 1..1467: note = Exemplary CD19 CAR nucleotide sequence
  source 1..1467: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 177 - protein, 489 aa, synthetic construct
  REGION 1..489: note = Exemplary CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 178 - DNA, 1458 bp, synthetic construct
  misc_feature 1..1458: note = Tisagenlecleucel CD19 CAR nucleotide sequence
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 179 - protein, 486 aa, synthetic construct
  REGION 1..486: note = Tisagenlecleucel CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 180 - DNA, 1383 bp, synthetic construct
  misc_feature 1..1383: note = Lisocabtagene maraleucel CD19 CAR nucleotide sequence
  source 1..1383: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 181 - protein, 461 aa, synthetic construct
  REGION 1..461: note = Lisocabtagene maraleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 182 - DNA, 1467 bp, synthetic construct
  misc_feature 1..1467: note = Axicabtagene ciloleucel CD19 CAR nucleotide sequence
  source 1..1467: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 183 - protein, 489 aa, synthetic construct
  REGION 1..489: note = Axicabtagene ciloleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 184 - protein, 246 aa, synthetic construct
  REGION 1..246: note = Anti-CD20 Leu16 scFv entire sequence, with Whitlow linker
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 185 - protein, 106 aa, synthetic construct
  REGION 1..106: note = Anti-CD20 Leu16 scFv light chain variable region
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 186 - protein, 10 aa, synthetic construct
  REGION 1..10: note = Anti-CD20 Leu16 scFv light chain CDR1
RASSSVNYMD

SEQ ID NO: 187 - protein, 7 aa, synthetic construct
  REGION 1..7: note = Anti-CD20 Leu16 scFv light chain CDR2
ATSNLAS

SEQ ID NO: 188 - protein, 9 aa, synthetic construct
  REGION 1..9: note = Anti-CD20 Leu16 scFv light chain CDR3
QQWSFNPPT

SEQ ID NO: 189 - protein, 122 aa, synthetic construct
  REGION 1..122: note = Anti-CD20 Leu16 scFv heavy chain
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 190 - protein, 5 aa, synthetic construct
  REGION 1..5: note = Anti-CD20 Leu16 scFv heavy chain CDR1
SYNMH

SEQ ID NO: 191 - protein, 17 aa, synthetic construct
  REGION 1..17: note = Anti-CD20 Leu16 scFv heavy chain CDR2
AIYPGNGDTSYNQKFKG

SEQ ID NO: 192 - protein, 13 aa, synthetic construct
  REGION 1..13: note = Anti-CD20 Leu16 scFv heavy chain CDR3
SNYYGSSYWFFDV

SEQ ID NO: 193 - protein, 246 aa, synthetic construct
  REGION 1..246: note = Anti-CD22 m971 scFv entire sequence, with 3xG4S linker
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 194 - protein, 124 aa, synthetic construct
  REGION 1..124: note = Anti-CD22 m971 scFv heavy chain variable region
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 195 - protein, 10 aa, synthetic construct
  REGION 1..10: note = Anti-CD22 m971 scFv heavy chain CDR1
GDSVSSNSAA

SEQ ID NO: 196 - protein, 9 aa, synthetic construct
  REGION 1..9: note = Anti-CD22 m971 scFv heavy chain CDR2
TYYRSKWYN

SEQ ID NO: 197 - protein, 14 aa, synthetic construct
  REGION 1..14: note = Anti-CD22 m971 scFv heavy chain CDR3
AREVTGDLEDAFDI

SEQ ID NO: 198 - protein, 107 aa, synthetic construct
  REGION 1..107: note = Anti-CD22 m971 scFv light chain
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 199 - protein, 6 aa, synthetic construct
  REGION 1..6: note = Anti-CD22 m971 scFv light chain CDR1
QTIWSY

SEQ ID NO: 200 - skipped

SEQ ID NO: 201 - protein, 9 aa, synthetic construct
  REGION 1..9: note = Anti-CD22 m971 scFv light chain CDR3
QQSYSIPQT

SEQ ID NO: 202 - protein, 246 aa, synthetic construct
  REGION 1..246: note = Anti-CD22 m971-L7 scFv entire sequence, with 3xG4S linker
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 203 - protein, 124 aa, synthetic construct
  REGION 1..124: note = Anti-CD22 m971-L7 scFv heavy chain variable region
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 204 - protein, 10 aa, synthetic construct
  REGION 1..10: note = Anti-CD22 m971-L7 scFv heavy chain CDR1
GDSVSSNSVA

SEQ ID NO: 205 - protein, 9 aa, synthetic construct
  REGION 1..9: note = Anti-CD22 m971-L7 scFv heavy chain CDR2
TYYRSTWYN

SEQ ID NO: 206 - protein, 14 aa, synthetic construct
  REGION 1..14: note = Anti-CD22 m971-L7 scFv heavy chain CDR3
AREVTGDLEDAFDI

SEQ ID NO: 207 - protein, 107 aa, synthetic construct
  REGION 1..107: note = Anti-CD22 m971-L7 scFv light chain variable region
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 208 - protein, 6 aa, synthetic construct
  REGION 1..6: note = Anti-CD22 m971-L7 scFv light chain CDR1
QTIWSY

SEQ ID NO: 209 - skipped

SEQ ID NO: 210 - protein, 9 aa, synthetic construct
  REGION 1..9: note = Anti-CD22 m971-L7 scFv light chain CDR3
QQSYSIPQT

SEQ ID NO: 211 - protein, 246 aa, synthetic construct
  REGION 1..246: note = Anti-BCMA C11D5.3 scFv entire sequence, with Whitlow linker
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 212 - protein, 111 aa, synthetic construct
  REGION 1..111: note = Anti-BCMA C11D5.3 scFv light chain variable region
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 213 - protein, 15 aa, synthetic construct
  REGION 1..15: note = Anti-BCMA C11D5.3 scFv light chain CDR1
RASESVSVIGAHLIH

SEQ ID NO: 214 - protein, 7 aa, synthetic construct
  REGION 1..7: note = Anti-BCMA C11D5.3 scFv light chain CDR2
LASNLET

SEQ ID NO: 215 - protein, 9 aa, synthetic construct
  REGION 1..9: note = Anti-BCMA C11D5.3 scFv light chain CDR3
LQSRIFPRT

SEQ ID NO: 216 - protein, 117 aa, synthetic construct
  REGION 1..117: note = Anti-BCMA C11D5.3 scFv heavy chain variable region
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 217 - protein, 5 aa, synthetic construct
  REGION 1..5: note = Anti-BCMA C11D5.3 scFv heavy chain CDR1
DYSIN

SEQ ID NO: 218 - protein, 17 aa, synthetic construct
  REGION 1..17: note = Anti-BCMA C11D5.3 scFv heavy chain CDR2
WINTETREPAYAYDFRG

SEQ ID NO: 219 - protein, 8 aa, synthetic construct
  REGION 1..8: note = Anti-BCMA C11D5.3 scFv heavy chain CDR3
DYSYAMDY

SEQ ID NO: 220 - protein, 246 aa, synthetic construct
  REGION 1..246: note = Anti-BCMA C12A3.2 scFv entire sequence, with Whitlow linker
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 221 - protein, 111 aa, synthetic construct
  REGION 1..111: note = Anti-BCMA C12A3.2 scFv light chain variable region
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 222 - protein, 15 aa, synthetic construct
  REGION 1..15: note = Anti-BCMA C12A3.2 scFv light chain CDR1
RASESVTILGSHLIY

SEQ ID NO: 223 - protein, 7 aa, synthetic construct
  REGION 1..7: note = Anti-BCMA C12A3.2 scFv light chain CDR2
LASNVQT

SEQ ID NO: 224 - protein, 9 aa, synthetic construct
  REGION 1..9: note = Anti-BCMA C12A3.2 scFv light chain CDR3
LQSRTIPRT

SEQ ID NO: 225 - protein, 117 aa, synthetic construct
  REGION 1..117: note = Anti-BCMA C12A3.2 scFv heavy chain variable region
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 226 - protein, 5 aa, synthetic construct
  REGION 1..5: note = Anti-BCMA C12A3.2 scFv heavy chain CDR1
HYSMN

SEQ ID NO: 227 - protein, 17 aa, synthetic construct
  REGION 1..17: note = Anti-BCMA C12A3.2 scFv heavy chain CDR2
RINTESGVPIYADDFKG

SEQ ID NO: 228 - protein, 8 aa, synthetic construct
  REGION 1..8: note = Anti-BCMA C12A3.2 scFv heavy chain CDR3
DYLYSLDF

SEQ ID NO: 229 - protein, 121 aa, synthetic construct
  REGION 1..121: note = Anti-BCMA FHVH33 entire sequence
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 230 - protein, 8 aa, synthetic construct
  REGION 1..8: note = Anti-BCMA FHVH33 CDR1
GFTFSSYA

SEQ ID NO: 231 - protein, 8 aa, synthetic construct
  REGION 1..8: note = Anti-BCMA FHVH33 CDR2
ISGSGDYI

SEQ ID NO: 232 - protein, 14 aa, synthetic construct
  REGION 1..14: note = Anti-BCMA FHVH33 CDR3
AKEGTGANSSLADY

SEQ ID NO: 233 - protein, 243 aa, synthetic construct
  REGION 1..243: note = Anti-BCMA CT103A scFv entire sequence, with Whitlow linker
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 234 - protein, 106 aa, synthetic construct
  REGION 1..106: note = Anti-BCMA CT103A scFv light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 235 - protein, 6 aa, synthetic construct
  REGION 1..6: note = Anti-BCMA CT103A scFv light chain CDR1
QSISSY

SEQ ID NO: 236 - skipped

SEQ ID NO: 237 - protein, 8 aa, synthetic construct
  REGION 1..8: note = Anti-BCMA CT103A scFv light chain CDR3
QQKYDLLT

SEQ ID NO: 238 - protein, 119 aa, synthetic construct
  REGION 1..119: note = Anti-BCMA CT103A scFv heavy chain variable region
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 239 - protein, 10 aa, synthetic construct
  REGION 1..10: note = Anti-BCMA CT103A scFv heavy chain CDR1
GGSISSSSYY

SEQ ID NO: 240 - protein, 7 aa, synthetic construct
  REGION 1..7: note = Anti-BCMA CT103A scFv heavy chain CDR2
ISYSGST

SEQ ID NO: 241 - protein, 11 aa, synthetic construct
  REGION 1..11: note = Anti-BCMA CT103A scFv heavy chain CDR3
ARDRGDTILDV

SEQ ID NO: 242 - DNA, 1503 bp, synthetic construct
  misc_feature 1..1503: note = Exemplary BCMA CAR nucleotide sequence
  source 1..1503: mol_type = other DNA
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaaaatacgacctcctcacttttggcggagggaccaaggttgagatcaaaggcagcaccagcggctccggcaagcctggctctggcgagggcagcacaaagggacagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagatcgtggagacaccatactagacgtatggggtcagggtacaatggtcaccgtcagctcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 243 - protein, 501 aa, synthetic construct
  REGION 1..501: note = Exemplary BCMA CAR amino acid sequence
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 244 - protein, 617 aa, synthetic construct
  REGION 1..617: note = Measles virus H protein
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMEGMGVSCTVTREDGTNRR

SEQ ID NO: 245 - protein, 578 aa, synthetic construct
  REGION 1..578: note = Canine distemper virus H protein
MLPYQDKVGAFYKDNARANSTKLSLVTEGHGGRRPPYLLFVLLILLVGILALLAITGVRFHQVSTSNMEFSRLLKEDMEKSEAVHHQVIDVLTPLFKIIGDEIGLRLPQKLNEIKQFILQKTNFFNPNREFDFRDLHWCINPPSTVKVNFTNYCESIGIRKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQF

SEQ ID NO: 246 - protein, 569 aa, synthetic construct
  REGION 1..569: note = Amino acid at positions 39, 126, 273, 345, 384, 448, 496, respectively: N N N N N Q Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 247 - protein, 569 aa, synthetic construct
  REGION 1..569: note = Amino acid at positions 39, 126, 273, 345, 384, 448, 496, respectively: N N N N Q N Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 248 - protein, 569 aa, synthetic construct
  REGION 1..569: note = Amino acid at positions 39, 126, 273, 345, 384, 448, 496, respectively: N N N N Q Q N
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 249 - protein, 569 aa, synthetic construct
  REGION 1..569: note = Amino acid at positions 39, 126, 273, 345, 384, 448, 496, respectively: N N N N Q Q Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 250 - protein, 569 aa, synthetic construct; identical to SEQ ID NO: 11
  REGION 1..569: note = Amino acid at positions 39, 126, 273, 345, 384, 448, 496, respectively: N N N Q N Q N

SEQ ID NO: 251 - protein, 569 aa, synthetic construct
  REGION 1..569: note = Amino acid at positions 39, 126, 273, 345, 384, 448, 496, respectively: N N N Q N Q Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 252 - protein, 569 aa, synthetic construct
  REGION 1..569: note = Amino acid at positions 39, 126, 273, 345, 384, 448, 496, respectively: N N N Q Q N N
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 253 - protein, 569 aa, synthetic construct; identical to SEQ ID NO: 249
  REGION 1..569: note = Amino acid at positions 39, 126, 273, 345, 384, 448, 496, respectively: N N N Q Q N Q

SEQ ID NO: 254 - protein, 569 aa, synthetic construct
  REGION 1..569: note = Amino acid at positions 39, 126, 273, 345, 384, 448, 496, respectively: N N N Q Q Q Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 255 - protein, 569 aa, synthetic construct; identical to SEQ ID NO: 247
  REGION 1..569: note = Amino acid at positions 39, 126, 273, 345, 384, 448, 496, respectively: N N Q N N N Q

SEQ ID NO: 256 - protein, 569 aa, synthetic construct
  REGION 1..569: note = Amino acid at positions 39, 126, 273, 345, 384, 448, 496, respectively: N N Q N N Q N
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 257 - protein, 569 aa, synthetic construct
  REGION 1..569: note = Amino acid at positions 39, 126, 273, 345, 384, 448, 496, respectively: N N Q N N Q Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 258 - protein, 569 aa, synthetic construct
  REGION 1..569: note = Amino acid at positions 39, 126, 273, 345, 384, 448, 496, respectively: N N Q N Q N N
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 259 - protein, 569 aa, synthetic construct; identical to SEQ ID NO: 251
  REGION 1..569: note = Amino acid at positions 39, 126, 273, 345, 384, 448, 496, respectively: N N Q N Q N Q

SEQ ID NO: 260 - protein, 569 aa, synthetic construct; identical to SEQ ID NO: 258
  REGION 1..569: note = Amino acid at positions 39, 126, 273, 345, 384, 448, 496, respectively: N N Q N Q Q N

SEQ ID NO: 261 - protein, 584 aa, synthetic construct
  REGION 1..584: note = Amino acid at positions 39, 126, 273, 345, 384, 448, 496, respectively: N N Q N Q Q Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCTGGGGSGGGGSGGGGS

SEQ ID NO: 262 - protein, 569 aa, synthetic construct
  REGION 1..569: note = Amino acid at positions 39, 126, 273, 345, 384, 448, 496, respectively: N N Q Q N N N
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 263 - protein, 569 aa, synthetic construct; identical to SEQ ID NO: 1
  REGION 1..569: note = Amino acid at positions 39, 126, 273, 345, 384, 448, 496, respectively: N N Q Q N N Q

SEQ ID NO: 264 - protein, 569 aa, synthetic construct; identical to SEQ ID NO: 246
  REGION 1..569: note = Amino acid at positions 39, 126, 273, 345, 384, 448, 496, respectively: N N Q Q N Q N

SEQ ID NO: 265 - protein, 569 aa, synthetic construct; identical to SEQ ID NO: 252
  REGION 1..569: note = Amino acid at positions 39, 126, 273, 345, 384, 448, 496, respectively: N N Q Q N Q Q

SEQ ID NO: 266 - protein, 569 aa, synthetic construct
  REGION 1..569: note = Amino acid at positions 39, 126, 273, 345, 384, 448, 496, respectively: N N Q Q Q N N
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 267 - protein, 569 aa, synthetic construct
  REGION 1..569: note = Amino acid at positions 39, 126, 273, 345, 384, 448, 496, respectively: N N Q Q Q N Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 268 - protein, 569 aa, synthetic construct; identical to SEQ ID NO: 248
  REGION 1..569: note = Amino acid at positions 39, 126, 273, 345, 384, 448, 496, respectively: N N Q Q Q Q N

SEQ ID NO: 269 - protein, 584 aa, synthetic construct
  REGION 1..584: note = Amino acid at positions 39, 126, 273, 345, 384, 448, 496, respectively: N N Q Q Q Q Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCTGGGGSGGGGSGGGGS

SEQ ID NO: 270 - protein, 584 aa, synthetic construct
  REGION 1..584: note = Amino acid at positions 39, 126, 273, 345, 384, 448, 496, respectively: N Q N N N N Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCTGGGGSGGGGSGGGGS

SEQ ID NO: 271 - protein, 569 aa, synthetic construct
  REGION 1..569: note = Amino acid at positions 39, 126, 273, 345, 384, 448, 496, respectively: N Q N N N Q N
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 272 - protein, 584 aa, synthetic construct
  REGION 1..584: note = Amino acid at positions 39, 126, 273, 345, 384, 448, 496, respectively: N Q N N N Q Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCTGGGGSGGGGSGGGGS

SEQ ID NO: 273 - protein, 569 aa, synthetic construct
  REGION 1..569: note = Amino acid at positions 39, 126, 273, 345, 384, 448, 496, respectively: N Q N N Q N N
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 274 - protein, 584 aa, synthetic construct
  REGION 1..584: note = Amino acid at positions 39, 126, 273, 345, 384, 448, 496, respectively: N Q N N Q N Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCTGGGGSGGGGSGGGGS

SEQ ID NO: 275 - protein, 569 aa, synthetic construct
  REGION 1..569: note = Amino acid at positions 39, 126, 273, 345, 384, 448, 496, respectively: N Q N N Q Q N
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 276 - protein, 569 aa, synthetic construct
  REGION 1..569: note = Amino acid at positions 39, 126, 273, 345, 384, 448, 496, respectively: N Q N N Q Q Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 277 - protein, 569 aa, synthetic construct
  REGION 1..569: note = Amino acid at positions 39, 126, 273, 345, 384, 448, 496, respectively: N Q N Q N N N
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 278 - protein, 569 aa, synthetic construct; identical to SEQ ID NO: 275
  REGION 1..569: note = Amino acid at positions 39, 126, 273, 345, 384, 448, 496, respectively: N Q N Q N N Q

SEQ ID NO: 279 - protein, 569 aa, synthetic construct
  REGION 1..569: note = Amino acid at positions 39, 126, 273, 345, 384, 448, 496, respectively: N Q N Q N Q N
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 280 - protein, 569 aa, synthetic construct
  REGION 1..569: note = Amino acid at positions 39, 126, 273, 345, 384, 448, 496, respectively: N Q N Q N Q Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 281 - protein, 569 aa, synthetic construct
  REGION 1..569: note = Amino acid at positions 39, 126, 273, 345, 384, 448, 496, respectively: N Q N Q Q N Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 282 - protein, 569 aa, synthetic construct
  REGION 1..569: note = Amino acid at positions 39, 126, 273, 345, 384, 448, 496, respectively: N Q N Q Q Q N
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 283 - protein, 569 aa, synthetic construct; identical to SEQ ID NO: 282
  REGION 1..569: note = Amino acid at positions 39, 126, 273, 345, 384, 448, 496, respectively: N Q N Q Q Q Q

SEQ ID NO: 284 - protein, 569 aa, synthetic construct
  REGION 1..569: note = Amino acid at positions 39, 126, 273, 345, 384, 448, 496, respectively: N Q Q N N N N
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 285 - protein, 569 aa, synthetic construct; identical to SEQ ID NO: 284
  REGION 1..569: note = Amino acid at positions 39, 126, 273, 345, 384, 448, 496, respectively: N Q Q N N N Q

SEQ ID NO: 286 - protein, 569 aa, synthetic construct; identical to SEQ ID NO: 277
  REGION 1..569: note = Amino acid at positions 39, 126, 273, 345, 384, 448, 496, respectively: N Q Q N N Q N

SEQ ID NO: 287 - protein, 569 aa, synthetic construct
  REGION 1..569: note = Amino acid at positions 39, 126, 273, 345, 384, 448, 496, respectively: N Q Q N N Q Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 288 - protein, 569 aa, synthetic construct; identical to SEQ ID NO: 271
  REGION 1..569: note = Amino acid at positions 39, 126, 273, 345, 384, 448, 496, respectively: N Q Q N Q N N

SEQ ID NO: 289 - protein, 569 aa, synthetic construct
  REGION 1..569: note = Amino acid at positions 39, 126, 273, 345, 384, 448, 496, respectively: N Q Q N Q N Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 290 - protein, 569 aa, synthetic construct; identical to SEQ ID NO: 279
  REGION 1..569: note = Amino acid at positions 39, 126, 273, 345, 384, 448, 496, respectively: N Q Q N Q Q N

SEQ ID NO: 291 - protein, 569 aa, synthetic construct
  REGION 1..569: note = Amino acid at positions 39, 126, 273, 345, 384, 448, 496, respectively: N Q Q N Q Q Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 292 - protein, 569 aa, synthetic construct
  REGION 1..569: note = Amino acid at positions 39, 126, 273, 345, 384, 448, 496, respectively: N Q Q Q N N N
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 293 - protein, 569 aa, synthetic construct
  REGION 1..569: note = Amino acid at positions 39, 126, 273, 345, 384, 448, 496, respectively: N Q Q Q N N Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 294 - protein, 569 aa, synthetic construct; identical to SEQ ID NO: 284
  REGION 1..569: note = Amino acid at positions 39, 126, 273, 345, 384, 448, 496, respectively: N Q Q Q N Q N

SEQ ID NO: 295 - protein, 569 aa, synthetic construct; identical to SEQ ID NO: 280
  REGION 1..569: note = Amino acid at positions 39, 126, 273, 345, 384, 448, 496, respectively: N Q Q Q N Q Q

SEQ ID NO: 296 - protein, 569 aa, synthetic construct
  REGION 1..569: note = Amino acid at positions 39, 126, 273, 345, 384, 448, 496, respectively: N Q Q Q Q N N
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 297 - protein, 569 aa, synthetic construct; identical to SEQ ID NO: 291
  REGION 1..569: note = Amino acid at positions 39, 126, 273, 345, 384, 448, 496, respectively: N Q Q Q Q N Q

SEQ ID NO: 298 - protein, 569 aa, synthetic construct
  REGION 1..569: note = Amino acid at positions 39, 126, 273, 345, 384, 448, 496, respectively: N Q Q Q Q Q N
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 299 - protein, 584 aa, synthetic construct
  REGION 1..584: note = Amino acid at positions 39, 126, 273, 345, 384, 448, 496, respectively: N Q Q Q Q Q Q
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCTGGGGSGGGGSGGGGS

SEQ ID NO: 300 - protein, 569 aa, synthetic construct
  REGION 1..569: note = Amino acid at positions 39, 126, 273, 345, 384, 448, 496, respectively: Q N N N N N Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 301 - protein, 584 aa, synthetic construct
  REGION 1..584: note = Amino acid at positions 39, 126, 273, 345, 384, 448, 496, respectively: Q N N N N Q N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGS

SEQ ID NO: 302 - protein, 569 aa, synthetic construct
  REGION 1..569: note = Amino acid at positions 39, 126, 273, 345, 384, 448, 496, respectively: Q N N N N Q Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 303 - protein, 584 aa, synthetic construct
  REGION 1..584: note = Amino acid at positions 39, 126, 273, 345, 384, 448, 496, respectively: Q N N N Q N N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGS

SEQ ID NO: 304 - protein, 569 aa, synthetic construct
  REGION 1..569: note = Amino acid at positions 39, 126, 273, 345, 384, 448, 496, respectively: Q N N N Q N Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 305 - protein, 569 aa, synthetic construct
  REGION 1..569: note = Amino acid at positions 39, 126, 273, 345, 384, 448, 496, respectively: Q N N N Q Q Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 306 - protein, 569 aa, synthetic construct
  REGION 1..569: note = Amino acid at positions 39, 126, 273, 345, 384, 448, 496, respectively: Q N N Q N N N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 307 - protein, 569 aa, synthetic construct
  REGION 1..569: note = Amino acid at positions 39, 126, 273, 345, 384, 448, 496, respectively: Q N N Q N N Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 308 - protein, 584 aa, synthetic construct
  REGION 1..584: note = Amino acid at positions 39, 126, 273, 345, 384, 448, 496, respectively: Q N N Q N Q Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCTGGGGSGGGGSGGGGS

SEQ ID NO: 309 - protein, 569 aa, synthetic construct
  REGION 1..569: note = Amino acid at positions 39, 126, 273, 345, 384, 448, 496, respectively: Q N N Q Q N N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 310 - protein, 569 aa, synthetic construct
  REGION 1..569: note = Amino acid at positions 39, 126, 273, 345, 384, 448, 496, respectively: Q N N Q Q N Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 311 - protein, 584 aa, synthetic construct
  REGION 1..584: note = Amino acid at positions 39, 126, 273, 345, 384, 448, 496, respectively: Q N N Q Q Q N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGS

SEQ ID NO: 312 - protein, 569 aa, synthetic construct
  REGION 1..569: note = Amino acid at positions 39, 126, 273, 345, 384, 448, 496, respectively: Q N N Q Q Q Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 313 - protein, 569 aa, synthetic construct
  REGION 1..569: note = Amino acid at positions 39, 126, 273, 345, 384, 448, 496, respectively: Q N Q N N N N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 314 - protein, 569 aa, synthetic construct
  REGION 1..569: note = Amino acid at positions 39, 126, 273, 345, 384, 448, 496, respectively: Q N Q N N N Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 315 - protein, 569 aa, synthetic construct; identical to SEQ ID NO: 314
  REGION 1..569: note = Amino acid at positions 39, 126, 273, 345, 384, 448, 496, respectively: Q N Q N N Q N

SEQ ID NO: 316 - protein, 584 aa, synthetic construct
  REGION 1..584: note = Amino acid at positions 39, 126, 273, 345, 384, 448, 496, respectively: Q N Q N N Q Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCTGGGGSGGGGSGGGGS

SEQ ID NO: 317 - protein, 569 aa, synthetic construct
  REGION 1..569: note = Amino acid at positions 39, 126, 273, 345, 384, 448, 496, respectively: Q N Q N Q N N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 318 - protein, 569 aa, synthetic construct; identical to SEQ ID NO: 317
  REGION 1..569: note = Amino acid at positions 39, 126, 273, 345, 384, 448, 496, respectively: Q N Q N Q N Q

SEQ ID NO: 319 - protein, 569 aa, synthetic construct; identical to SEQ ID NO: 304
  REGION 1..569: note = Amino acid at positions 39, 126, 273, 345, 384, 448, 496, respectively: Q N Q N Q Q N

SEQ ID NO: 320 - protein, 569 aa, synthetic construct; identical to SEQ ID NO: 304
  REGION 1..569: note = Amino acid at positions 39, 126, 273, 345, 384, 448, 496, respectively: Q N Q N Q Q Q

SEQ ID NO: 321 - protein, 569 aa, synthetic construct; identical to SEQ ID NO: 310
  REGION 1..569: note = Amino acid at positions 39, 126, 273, 345, 384, 448, 496, respectively: Q N Q Q N N N

SEQ ID NO: 322 - protein, 569 aa, synthetic construct
  REGION 1..569: note = Amino acid at positions 39, 126, 273, 345, 384, 448, 496, respectively: Q N Q Q N N Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 323 - protein, 569 aa, synthetic construct
  REGION 1..569: note = Amino acid at positions 39, 126, 273, 345, 384, 448, 496, respectively: Q N Q Q N Q N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 324 - protein, 569 aa, synthetic construct; identical to SEQ ID NO: 317
  REGION 1..569: note = Amino acid at positions 39, 126, 273, 345, 384, 448, 496, respectively: Q N Q Q N Q Q

SEQ ID NO: 325 - protein, 569 aa, synthetic construct
  REGION 1..569: note = Amino acid at positions 39, 126, 273, 345, 384, 448, 496, respectively: Q N Q Q Q N N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 326 - protein, 569 aa, synthetic construct; identical to SEQ ID NO: 305
  REGION 1..569: note = Amino acid at positions 39, 126, 273, 345, 384, 448, 496, respectively: Q N Q Q Q N Q

SEQ ID NO: 327 - protein, 569 aa, synthetic construct; identical to SEQ ID NO: 310
  REGION 1..569: note = Amino acid at positions 39, 126, 273, 345, 384, 448, 496, respectively: Q N Q Q Q Q N

SEQ ID NO: 328 - protein, 569 aa, synthetic construct
  REGION 1..569: note = Amino acid at positions 39, 126, 273, 345, 384, 448, 496, respectively: Q N Q Q Q Q Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 329 - protein, 569 aa, synthetic construct
  REGION 1..569: note = Amino acid at positions 39, 126, 273, 345, 384, 448, 496, respectively: Q Q N N N N N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 330 - protein, 569 aa, synthetic construct
  REGION 1..569: note = Amino acid at positions 39, 126, 273, 345, 384, 448, 496, respectively: Q Q N N N N Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 331 - protein, 584 aa, synthetic construct
  REGION 1..584: note = Amino acid at positions 39, 126, 273, 345, 384, 448, 496, respectively: Q Q N N N Q N
MKKINEGXLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGS

SEQ ID NO: 332 - protein, 569 aa, synthetic construct
  REGION 1..569: note = Amino acid at positions 39, 126, 273, 345, 384, 448, 496, respectively: Q Q N N N Q Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 333 - protein, 584 aa, synthetic construct
  REGION 1..584: note = Amino acid at positions 39, 126, 273, 345, 384, 448, 496, respectively: Q Q N N Q N Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCTGGGGSGGGGSGGGGS

SEQ ID NO: 334 - protein, 569 aa, synthetic construct; identical to SEQ ID NO: 332
  REGION 1..569: note = Amino acid at positions 39, 126, 273, 345, 384, 448, 496, respectively: Q Q N N Q Q N

SEQ ID NO: 335 - protein, 569 aa, synthetic construct
  REGION 1..569: note = Amino acid at positions 39, 126, 273, 345, 384, 448, 496, respectively: Q Q N N Q Q Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 336 - protein, 569 aa, synthetic construct
  REGION 1..569: note = Amino acid at positions 39, 126, 273, 345, 384, 448, 496, respectively: Q Q N Q N N N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 337 - protein, 569 aa, synthetic construct
  REGION 1..569: note = Amino acid at positions 39, 126, 273, 345, 384, 448, 496, respectively: Q Q N Q N N Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 338 - protein, 569 aa, synthetic construct
  REGION 1..569: note = Amino acid at positions 39, 126, 273, 345, 384, 448, 496, respectively: Q Q N Q N Q N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 339 - protein, 584 aa, synthetic construct
  REGION 1..584: note = Amino acid at positions 39, 126, 273, 345, 384, 448, 496, respectively: Q Q N Q N Q Q
MKKINEGXLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCTGGGGSGGGGSGGGGS

SEQ ID NO: 340 - protein, 569 aa, synthetic construct
  REGION 1..569: note = Amino acid at positions 39, 126, 273, 345, 384, 448, 496, respectively: Q Q N Q Q N N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 341 - protein, 569 aa, synthetic construct
  REGION 1..569: note = Amino acid at positions 39, 126, 273, 345, 384, 448, 496, respectively: Q Q N Q Q N Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 342 - protein, 569 aa, synthetic construct; identical to SEQ ID NO: 329
  REGION 1..569: note = Amino acid at positions 39, 126, 273, 345, 384, 448, 496, respectively: Q Q N Q Q Q N

SEQ ID NO: 343 - protein, 569 aa, synthetic construct
  REGION 1..569: note = Amino acid at positions 39, 126, 273, 345, 384, 448, 496, respectively: Q Q Q N N N N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 344 - protein, 569 aa, synthetic construct
  REGION 1..569: note = Amino acid at positions 39, 126, 273, 345, 384, 448, 496, respectively: Q Q Q N N N Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 345 - protein, 569 aa, synthetic construct
  REGION 1..569: note = Amino acid at positions 39, 126, 273, 345, 384, 448, 496, respectively: Q Q Q N N Q N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 346 - protein, 569 aa, synthetic construct
  REGION 1..569: note = Amino acid at positions 39, 126, 273, 345, 384, 448, 496, respectively: Q Q Q N Q N N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 347 - protein, 569 aa, synthetic construct; identical to SEQ ID NO: 336
  REGION 1..569: note = Amino acid at positions 39, 126, 273, 345, 384, 448, 496, respectively: Q Q Q N Q N Q

SEQ ID NO: 348 - protein, 584 aa, synthetic construct
  REGION 1..584: note = Amino acid at positions 39, 126, 273, 345, 384, 448, 496, respectively: Q Q Q N Q Q Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCTGGGGSGGGGSGGGGS

SEQ ID NO: 349 - protein, 569 aa, synthetic construct
  REGION 1..569: note = Amino acid at positions 39, 126, 273, 345, 384, 448, 496, respectively: Q Q Q Q N N N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 350 - protein, 569 aa, synthetic construct
  REGION 1..569: note = Amino acid at positions 39, 126, 273, 345, 384, 448, 496, respectively: Q Q Q Q N N Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 351 - protein, 569 aa, synthetic construct
  REGION 1..569: note = Amino acid at positions 39, 126, 273, 345, 384, 448, 496, respectively: Q Q Q Q N Q N
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 352 - protein, 584 aa, synthetic construct
  REGION 1..584: note = Amino acid at positions 39, 126, 273, 345, 384, 448, 496, respectively: Q Q Q Q Q N Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCTGGGGSGGGGSGGGGS

SEQ ID NO: 353 - protein, 569 aa, synthetic construct; identical to SEQ ID NO: 310
  REGION 1..569: note = Amino acid at positions 39, 126, 273, 345, 384, 448, 496, respectively: Q Q Q Q Q Q N

SEQ ID NO: 354 - protein, 569 aa, synthetic construct
  REGION 1..569: note = Amino acid at positions 39, 126, 273, 345, 384, 448, 496, respectively: Q Q Q Q Q Q Q
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 355 - protein, 45 aa, synthetic construct
  REGION 1..45: note = NivG CT_45_FL
MPAENKKVRFENTTSDKGKIPSKVIKSYYGTMDIKKINEGLLDSK

SEQ ID NO: 356 - protein, 5 aa, synthetic construct
  REGION 1..5: note = NivG_CT_05 cytoplasmic tail
MLDSK

SEQ ID NO: 357 - protein, 6 aa, synthetic construct
  REGION 1..6: note = NivG_CT_06 cytoplasmic tail
MLLDSK

SEQ ID NO: 358 - protein, 7 aa, synthetic construct
  REGION 1..7: note = NivG_CT_07 cytoplasmic tail
MGLLDSK

SEQ ID NO: 359 - protein, 8 aa, synthetic construct
  REGION 1..8: note = NivG_CT_08 cytoplasmic tail
MEGLLDSK

SEQ ID NO: 360 - protein, 9 aa, synthetic construct
  REGION 1..9: note = NivG_CT_09 cytoplasmic tail
MNEGLLDSK

SEQ ID NO: 361 - protein, 10 aa, synthetic construct
  REGION 1..10: note = NivG_CT_10 cytoplasmic tail
MINEGLLDSK

SEQ ID NO: 362 - protein, 11 aa, synthetic construct
  REGION 1..11: note = NivG_CT_11 cytoplasmic tail
MKINEGLLDSK

SEQ ID NO: 363 - protein, 13 aa, synthetic construct
  REGION 1..13: note = NivG_CT_13 cytoplasmic tail
MIKKINEGLLDSK

SEQ ID NO: 364 - protein, 14 aa, synthetic construct
  REGION 1..14: note = NivG_CT_14 cytoplasmic tail
MDIKKINEGLLDSK

SEQ ID NO: 365 - protein, 15 aa, synthetic construct
  REGION 1..15: note = NivG_CT_15 cytoplasmic tail
MMDIKKINEGLLDSK

SEQ ID NO: 366 - protein, 16 aa, synthetic construct
  REGION 1..16: note = NivG_CT_16 cytoplasmic tail
MTMDIKKINEGLLDSK

SEQ ID NO: 367 - protein, 17 aa, synthetic construct
  REGION 1..17: note = NivG_CT_17 cytoplasmic tail
MGTMDIKKINEGLLDSK

SEQ ID NO: 368 - protein, 18 aa, synthetic construct
  REGION 1..18: note = NivG_CT_18 cytoplasmic tail
MYGTMDIKKINEGLLDSK

SEQ ID NO: 369 - protein, 19 aa, synthetic construct
  REGION 1..19: note = NivG_CT_19 cytoplasmic tail
MYYGTMDIKKINEGLLDSK

SEQ ID NO: 370 - protein, 20 aa, synthetic construct
  REGION 1..20: note = NivG_CT_20 cytoplasmic tail
MSYYGTMDIKKINEGLLDSK

SEQ ID NO: 371 - protein, 21 aa, synthetic construct
  REGION 1..21: note = NivG_CT_21 cytoplasmic tail
MKSYYGTMDIKKINEGLLDSK

SEQ ID NO: 372 - protein, 22 aa, synthetic construct
  REGION 1..22: note = NivG_CT_22 cytoplasmic tail
MIKSYYGTMDIKKINEGLLDSK

SEQ ID NO: 373 - protein, 23 aa, synthetic construct
  REGION 1..23: note = NivG_CT_23 cytoplasmic tail
MVIKSYYGTMDIKKINEGLLDSK

SEQ ID NO: 374 - protein, 24 aa, synthetic construct
  REGION 1..24: note = NivG_CT_24 cytoplasmic tail
MKVIKSYYGTMDIKKINEGLLDSK

SEQ ID NO: 375 - protein, 25 aa, synthetic construct
  REGION 1..25: note = NivG_CT_25 cytoplasmic tail
MSKVIKSYYGTMDIKKINEGLLDSK

SEQ ID NO: 376 - protein, 30 aa, synthetic construct
  REGION 1..30: note = NivG_CT_30 cytoplasmic tail
MKGKNPSKVIKSYYGTMDIKKINEGLLDSK

SEQ ID NO: 377 - protein, 35 aa, synthetic construct
  REGION 1..35: note = NivG_CT_35 cytoplasmic tail
MNTTSDKGKNPSKVIKSYYGTMDIKKINEGLLDSK

SEQ ID NO: 378 - protein, 40 aa, synthetic construct
  REGION 1..40: note = NivG_CT_40 cytoplasmic tail
MKVRFENTTSDKGKNPSKVIKSYYGTMDIKKINEGLLDSK

SEQ ID NO: 379 - protein, 23 aa, synthetic construct
  REGION 1..23: note = NivG_CSUR38_CT_23 cytoplasmic tail
MIIKSYYGTMDIKKINEGLLDSK

SEQ ID NO: 380 - protein, 29 aa, synthetic construct
  REGION 1..29: note = NivG_CSUR38_CT_29 cytoplasmic tail
MGKIPSKIIKSYYGTMDIKKINEGLLDSK

SEQ ID NO: 381 - protein, 35 aa, synthetic construct
  REGION 1..35: note = NivG_CSUR38_CT_35 cytoplasmic tail
MNTTSDKGKIPSKIIKSYYGTMDIKKINEGLLDSK

SEQ ID NO: 382 - protein, 42 aa, synthetic construct
  REGION 1..42: note = NivG_CSUR38_CT_42 cytoplasmic tail
MSKKVRFENTTSDKGKIPSKIIKSYYGTMDIKKINEGLLDSK

SEQ ID NO: 383 - protein, 45 aa, synthetic construct
  REGION 1..45: note = NivG_CSUR38_CT_45_FL cytoplasmic tail
MPAESKKVRFENTTSDKGKIPSKIIKSYYGTMDIKKINEGLLDSK

SEQ ID NO: 384 - protein, 27 aa, synthetic construct
  REGION 1..27: note = NivG_VRI-0626_CT_27 cytoplasmic tail
MNPSKVIKSYYGTMDIKKINEGLLDSK

SEQ ID NO: 385 - protein, 31 aa, synthetic construct
  REGION 1..31: note = NivG_VRI-0626_CT_31 cytoplasmic tail
MDKGKNPSKVIKSYYGTMDIKKINEGLLDSK

SEQ ID NO: 386 - protein, 35 aa, synthetic construct; identical to SEQ ID NO: 377
  REGION 1..35: note = NivG_VRI-0626_CT_35

SEQ ID NO: 387 - protein, 40 aa, synthetic construct; identical to SEQ ID NO: 378
  REGION 1..40: note = NivG_VRI-0626_CT_40 cytoplasmic tail

SEQ ID NO: 388 - protein, 45 aa, synthetic construct
  REGION 1..45: note = NivG_VRI-0626_CT_45_FL cytoplasmic tail
MPAENKKVRFENTTSDKGKNPSKVIKSYYGTMDIKKINEGLLDSK

SEQ ID NO: 389 - protein, 12 aa, synthetic construct
  REGION 1..12: note = NivG_CT_12 cytoplasmic tail
MKKINEGLLDSK

SEQ ID NO: 390 - protein, 8 aa, synthetic construct
  REGION 1..8: note = HevG_CT_08
MDGLLDSK

SEQ ID NO: 391 - protein, 9 aa, synthetic construct
  REGION 1..9: note = HevG_CT_09
MNDGLLDSK

SEQ ID NO: 392 - protein, 10 aa, synthetic construct
  REGION 1..10: note = HevG_CT_10
MINDGLLDSK

SEQ ID NO: 393 - protein, 11 aa, synthetic construct
  REGION 1..11: note = HevG_CT_11
MKINDGLLDSK

SEQ ID NO: 394 - protein, 12 aa, synthetic construct
  REGION 1..12: note = HevG_CT_12
MKKINDGLLDSK

SEQ ID NO: 395 - protein, 13 aa, synthetic construct
  REGION 1..13: note = HevG_CT_13
MIKKINDGLLDSK

SEQ ID NO: 396 - protein, 14 aa, synthetic construct
  REGION 1..14: note = HevG_CT_14
MDIKKINDGLLDSK

SEQ ID NO: 397 - protein, 15 aa, synthetic construct
  REGION 1..15: note = HevG_CT_15
MMDIKKINDGLLDSK

SEQ ID NO: 398 - protein, 16 aa, synthetic construct
  REGION 1..16: note = HevG_CT_16
MTMDIKKINDGLLDSK

SEQ ID NO: 399 - protein, 17 aa, synthetic construct
  REGION 1..17: note = HevG_CT_17
MGTMDIKKINDGLLDSK

SEQ ID NO: 400 - protein, 18 aa, synthetic construct
  REGION 1..18: note = HevG_CT_18
MYGTMDIKKINDGLLDSK

SEQ ID NO: 401 - protein, 19 aa, synthetic construct
  REGION 1..19: note = HevG_CT_19
MYYGTMDIKKINDGLLDSK

SEQ ID NO: 402 - protein, 20 aa, synthetic construct
  REGION 1..20: note = HevG_CT_20
MNYYGTMDIKKINDGLLDSK

SEQ ID NO: 403 - protein, 21 aa, synthetic construct
  REGION 1..21: note = HevG_CT_21
MKNYYGTMDIKKINDGLLDSK

SEQ ID NO: 404 - protein, 22 aa, synthetic construct
  REGION 1..22: note = HevG_CT_22
MIKNYYGTMDIKKINDGLLDSK

SEQ ID NO: 405 - protein, 23 aa, synthetic construct
  REGION 1..23: note = HevG_CT_23
MVIKNYYGTMDIKKINDGLLDSK

SEQ ID NO: 406 - protein, 24 aa, synthetic construct
  REGION 1..24: note = HevG_CT_24
MKVIKNYYGTMDIKKINDGLLDSK

SEQ ID NO: 407 - protein, 45 aa, synthetic construct
  REGION 1..45: note = HevG_CT_45_FL
MMADSKLVSLNNNLSGKIKDQGKVIKNYYGTMDIKKINDGLLDSK

SEQ ID NO: 408 - protein, 6 aa, synthetic construct
  REGION 1..6: note = BatPV_G_CT_6
MQNDHY

SEQ ID NO: 409 - protein, 7 aa, synthetic construct
  REGION 1..7: note = BatPV_G_CT_7
MNQNDHY

SEQ ID NO: 410 - protein, 8 aa, synthetic construct
  REGION 1..8: note = BatPV_G_CT_8
MKNQNDHY

SEQ ID NO: 411 - protein, 9 aa, synthetic construct
  REGION 1..9: note = BatPV_G_CT_9
MQKNQNDHY

SEQ ID NO: 412 - protein, 10 aa, synthetic construct
  REGION 1..10: note = BatPV_G_CT_10
MKQKNQNDHY

SEQ ID NO: 413 - protein, 11 aa, synthetic construct
  REGION 1..11: note = BatPV_G_CT_11
MKKQKNQNDHY

SEQ ID NO: 414 - protein, 12 aa, synthetic construct
  REGION 1..12: note = BatPV_G_CT_12
MWKKQKNQNDHY

SEQ ID NO: 415 - protein, 13 aa, synthetic construct
  REGION 1..13: note = BatPV_G_CT_13
MNWKKQKNQNDHY

SEQ ID NO: 416 - protein, 14 aa, synthetic construct
  REGION 1..14: note = BatPV_G_CT_14
MRNWKKQKNQNDHY

SEQ ID NO: 417 - protein, 15 aa, synthetic construct
  REGION 1..15: note = BatPV_G_CT_15
MERNWKKQKNQNDHY

SEQ ID NO: 418 - protein, 16 aa, synthetic construct
  REGION 1..16: note = BatPV_G_CT_16
MSERNWKKQKNQNDHY

SEQ ID NO: 419 - protein, 17 aa, synthetic construct
  REGION 1..17: note = BatPV_G_CT_17
MHSERNWKKQKNQNDHY

SEQ ID NO: 420 - protein, 18 aa, synthetic construct
  REGION 1..18: note = BatPV_G_CT_18
MSHSERNWKKQKNQNDHY

SEQ ID NO: 421 - protein, 19 aa, synthetic construct
  REGION 1..19: note = BatPV_G_CT_19
MGSHSERNWKKQKNQNDHY

SEQ ID NO: 422 - protein, 20 aa, synthetic construct
  REGION 1..20: note = BatPV_G_CT_20
MLGSHSERNWKKQKNQNDHY

SEQ ID NO: 423 - protein, 21 aa, synthetic construct
  REGION 1..21: note = BatPV_G_CT_21
MGLGSHSERNWKKQKNQNDHY

SEQ ID NO: 424 - protein, 22 aa, synthetic construct
  REGION 1..22: note = BatPV_G_CT_22
MFGLGSHSERNWKKQKNQNDHY

SEQ ID NO: 425 - protein, 23 aa, synthetic construct
  REGION 1..23: note = BatPV_G_CT_23
MYFGLGSHSERNWKKQKNQNDHY

SEQ ID NO: 426 - protein, 24 aa, synthetic construct
  REGION 1..24: note = BatPV_G_CT_24
MGYFGLGSHSERNWKKQKNQNDHY

SEQ ID NO: 427 - protein, 29 aa, synthetic construct
  REGION 1..29: note = BatPV_G_CT_29
MKITKQGYFGLGSHSERNWKKQKNQNDHY

SEQ ID NO: 428 - protein, 59 aa, synthetic construct
  REGION 1..59: note = BatPV_G_CT_59_FL
MPQKTVEFINMNSPLERGVSTLSDKKTLNQSKITKQGYFGLGSHSERNWKKQKNQNDHY

SEQ ID NO: 429 - protein, 6 aa, synthetic construct
  REGION 1..6: note = CedarV_G_CT_6
MLHDIK

SEQ ID NO: 430 - protein, 7 aa, synthetic construct
  REGION 1..7: note = CedarV_G_CT_7
MSLHDIK

SEQ ID NO: 431 - protein, 8 aa, synthetic construct
  REGION 1..8: note = CedarV_G_CT_8
MESLHDIK

SEQ ID NO: 432 - protein, 9 aa, synthetic construct
  REGION 1..9: note = CedarV_G_CT_9
MNESLHDIK

SEQ ID NO: 433 - protein, 10 aa, synthetic construct
  REGION 1..10: note = CedarV_G_CT_10
MLNESLHDIK

SEQ ID NO: 434 - protein, 11 aa, synthetic construct
  REGION 1..11: note = CedarV_G_CT_8
MLLNESLHDIK

SEQ ID NO: 435 - protein, 12 aa, synthetic construct
  REGION 1..12: note = CedarV_G_CT_9
MNLLNESLHDIK

SEQ ID NO: 436 - protein, 13 aa, synthetic construct
  REGION 1..13: note = CedarV_G_CT_10
MSNLLNESLHDIK

SEQ ID NO: 437 - protein, 14 aa, synthetic construct
  REGION 1..14: note = CedarV_G_CT_11
MVSNLLNESLHDIK

SEQ ID NO: 438 - protein, 15 aa, synthetic construct
  REGION 1..15: note = CedarV_G_CT_15
MNVSNLLNESLHDIK

SEQ ID NO: 439 - protein, 16 aa, synthetic construct
  REGION 1..16: note = CedarV_G_CT_16
MYNVSNLLNESLHDIK

SEQ ID NO: 440 - protein, 17 aa, synthetic construct
  REGION 1..17: note = CedarV_G_CT_17
MNYNVSNLLNESLHDIK

SEQ ID NO: 441 - protein, 18 aa, synthetic construct
  REGION 1..18: note = CedarV_G_CT_18
MKNYNVSNLLNESLHDIK

SEQ ID NO: 442 - protein, 19 aa, synthetic construct
  REGION 1..19: note = CedarV_G_CT_19
MNKNYNVSNLLNESLHDIK

SEQ ID NO: 443 - protein, 20 aa, synthetic construct
  REGION 1..20: note = CedarV_G_CT_20
MKNKNYNVSNLLNESLHDIK

SEQ ID NO: 444 - protein, 21 aa, synthetic construct
  REGION 1..21: note = CedarV_G_CT_21
MVKNKNYNVSNLLNESLHDIK

SEQ ID NO: 445 - protein, 22 aa, synthetic construct
  REGION 1..22: note = CedarV_G_CT_22
MYVKNKNYNVSNLLNESLHDIK

SEQ ID NO: 446 - protein, 23 aa, synthetic construct
  REGION 1..23: note = CedarV_G_CT_23
MYYVKNKNYNVSNLLNESLHDIK

SEQ ID NO: 447 - protein, 24 aa, synthetic construct
  REGION 1..24: note = CedarV_G_CT_24
MSYYVKNKNYNVSNLLNESLHDIK

SEQ ID NO: 448 - protein, 30 aa, synthetic construct
  REGION 1..30: note = CedarV_G_CT_30
MQKDLNKSYYVKNKNYNVSNLLNESLHDIK

SEQ ID NO: 449 - protein, 68 aa, synthetic construct
  REGION 1..68: note = CedarV_G_CT_68_FL
MLSQLQKNYLDNSNQQGDKMNNPDKKLSVNFNPLELDKGQKDLNKSYYVKNKNYNVSNLLNESLHDIK

SEQ ID NO: 450 - protein, 6 aa, synthetic construct
  REGION 1..6: note = CDV-H_CT_06
MQGGRR

SEQ ID NO: 451 - protein, 7 aa, synthetic construct
  REGION 1..7: note = CDV-H_CT_07
MEQGGRR

SEQ ID NO: 452 - protein, 8 aa, synthetic construct
  REGION 1..8: note = CDV-H_CT_08
MEEQGGRR

SEQ ID NO: 453 - protein, 9 aa, synthetic construct
  REGION 1..9: note = CDV-H_CT_09
MTEEQGGRR

SEQ ID NO: 454 - protein, 10 aa, synthetic construct
  REGION 1..10: note = CDV-H_CT_10
MVTEEQGGRR

SEQ ID NO: 455 - protein, 11 aa, synthetic construct
  REGION 1..11: note = CDV-H_CT_11
MLVTEEQGGRR

SEQ ID NO: 456 - protein, 12 aa, synthetic construct
  REGION 1..12: note = CDV-H_CT_12
MSLVTEEQGGRR

SEQ ID NO: 457 - protein, 13 aa, synthetic construct
  REGION 1..13: note = CDV-H_CT_13
MLSLVTEEQGGRR

SEQ ID NO: 458 - protein, 14 aa, synthetic construct
  REGION 1..14: note = CDV-H_CT_14
MKLSLVTEEQGGRR

SEQ ID NO: 459 - protein, 15 aa, synthetic construct
  REGION 1..15: note = CDV-H_CT_15
MSKLSLVTEEQGGRR

SEQ ID NO: 460 - protein, 16 aa, synthetic construct
  REGION 1..16: note = CDV-H_CT_16
MSSKLSLVTEEQGGRR

SEQ ID NO: 461 - protein, 17 aa, synthetic construct
  REGION 1..17: note = CDV-H_CT_17
MNSSKLSLVTEEQGGRR

SEQ ID NO: 462 - protein, 18 aa, synthetic construct
  REGION 1..18: note = CDV-H_CT_18
MANSSKLSLVTEEQGGRR

SEQ ID NO: 463 - protein, 19 aa, synthetic construct
  REGION 1..19: note = CDV-H_CT_19
MRANSSKLSLVTEEQGGRR

SEQ ID NO: 464 - protein, 20 aa, synthetic construct
  REGION 1..20: note = CDV-H_CT_20
MARANSSKLSLVTEEQGGRR

SEQ ID NO: 465 - protein, 34 aa, synthetic construct
  REGION 1..34: note = CDV-H_CT_34_FL
MLSYQDKVGAFYKDNARANSSKLSLVTEEQGGRR

SEQ ID NO: 466 - protein, 6 aa, synthetic construct
  REGION 1..6: note = HPIV2-HN_CT_06
MRIIFR

SEQ ID NO: 467 - protein, 12 aa, synthetic construct
  REGION 1..12: note = HPIV2-HN_CT_12
MIPKRTCRIIFR

SEQ ID NO: 468 - protein, 22 aa, synthetic construct
  REGION 1..22: note = HPIV2-HN_CT_22_FL
MEDYSNLSLKSIPKRTCRIIFR

SEQ ID NO: 469 - protein, 6 aa, synthetic construct
  REGION 1..6: note = MvH_CT_06
MLMIDR

SEQ ID NO: 470 - protein, 7 aa, synthetic construct
  REGION 1..7: note = MvH_CT_07
MHLMIDR

SEQ ID NO: 471 - protein, 8 aa, synthetic construct
  REGION 1..8: note = MvH_CT_08
MEHLMIDR

SEQ ID NO: 472 - protein, 9 aa, synthetic construct
  REGION 1..9: note = MvH_CT_09
MREHLMIDR

SEQ ID NO: 473 - protein, 10 aa, synthetic construct
  REGION 1..10: note = MvH_CT_10
MNREHLMIDR

SEQ ID NO: 474 - protein, 11 aa, synthetic construct
  REGION 1..11: note = MvH_CT_11
MINREHLMIDR

SEQ ID NO: 475 - protein, 12 aa, synthetic construct
  REGION 1..12: note = MvH_CT_12
MVINREHLMIDR

SEQ ID NO: 476 - protein, 13 aa, synthetic construct
  REGION 1..13: note = MvH_CT_13
MIVINREHLMIDR

SEQ ID NO: 477 - protein, 14 aa, synthetic construct
  REGION 1..14: note = MvH_CT_14
MRIVINREHLMIDR

SEQ ID NO: 478 - protein, 15 aa, synthetic construct
  REGION 1..15: note = MvH_CT_15
MSRIVINREHLMIDR

SEQ ID NO: 479 - protein, 16 aa, synthetic construct
  REGION 1..16: note = MvH_CT_16
MGSRIVINREHLMIDR

SEQ ID NO: 480 - protein, 17 aa, synthetic construct
  REGION 1..17: note = MvH_CT_17
MKGSRIVINREHLMIDR

SEQ ID NO: 481 - protein, 18 aa, synthetic construct
  REGION 1..18: note = MvH_CT_18
MPKGSRIVINREHLMIDR

SEQ ID NO: 482 - protein, 19 aa, synthetic construct
  REGION 1..19: note = MvH_CT_19
MHPKGSRIVINREHLMIDR

SEQ ID NO: 483 - protein, 20 aa, synthetic construct
  REGION 1..20: note = MvH_CT_20
MPHPKGSRIVINREHLMIDR

SEQ ID NO: 484 - protein, 34 aa, synthetic construct
  REGION 1..34: note = MvH_CT_34_FL
MSPQRDRINAFYKDNPHPKGSRIVINREHLMIDR

SEQ ID NO: 485 - protein, 6 aa, synthetic construct
  REGION 1..6: note = NDV-HN_CT_06
MRLVFR

SEQ ID NO: 486 - protein, 7 aa, synthetic construct
  REGION 1..7: note = NDV-HN_CT_07
MWRLVFR

SEQ ID NO: 487 - protein, 8 aa, synthetic construct
  REGION 1..8: note = NDV-HN_CT_08
MTWRLVFR

SEQ ID NO: 488 - protein, 9 aa, synthetic construct
  REGION 1..9: note = NDV-HN_CT_09
MNTWRLVFR

SEQ ID NO: 489 - protein, 10 aa, synthetic construct
  REGION 1..10: note = NDV-HN_CT_10
MKNTWRLVFR

SEQ ID NO: 490 - protein, 11 aa, synthetic construct
  REGION 1..11: note = NDV-HN_CT_11
MAKNTWRLVFR

SEQ ID NO: 491 - protein, 12 aa, synthetic construct
  REGION 1..12: note = NDV-HN_CT_12
MEAKNTWRLVFR

SEQ ID NO: 492 - protein, 13 aa, synthetic construct
  REGION 1..13: note = NDV-HN_CT_13
MREAKNTWRLVFR

SEQ ID NO: 493 - protein, 14 aa, synthetic construct
  REGION 1..14: note = NDV-HN_CT_14
MEREAKNTWRLVFR

SEQ ID NO: 494 - protein, 15 aa, synthetic construct
  REGION 1..15: note = NDV-HN_CT_15
MDEREAKNTWRLVFR

SEQ ID NO: 495 - protein, 16 aa, synthetic construct
  REGION 1..16: note = NDV-HN_CT_16
MNDEREAKNTWRLVFR

SEQ ID NO: 496 - protein, 17 aa, synthetic construct
  REGION 1..17: note = NDV-HN_CT_17
MENDEREAKNTWRLVFR

SEQ ID NO: 497 - protein, 18 aa, synthetic construct
  REGION 1..18: note = NDV-HN_CT_18
MLENDEREAKNTWRLVFR

SEQ ID NO: 498 - protein, 19 aa, synthetic construct
  REGION 1..19: note = NDV-HN_CT_19
MALENDEREAKNTWRLVFR

SEQ ID NO: 499 - protein, 20 aa, synthetic construct
  REGION 1..20: note = NDV-HN_CT_20
MVALENDEREAKNTWRLVFR

SEQ ID NO: 500 - protein, 26 aa, synthetic construct
  REGION 1..26: note = NDV-HN_CT_26_FL
MERGVSQVALENDEREAKNTWRLVFR

SEQ ID NO: 501 - protein, 6 aa, synthetic construct
  REGION 1..6: note = SeV_CT_06
MERSGK

SEQ ID NO: 502 - protein, 7 aa, synthetic construct
  REGION 1..7: note = SeV_CT_07
MSERSGK

SEQ ID NO: 503 - protein, 8 aa, synthetic construct
  REGION 1..8: note = SeV_CT_08
MDSERSGK

SEQ ID NO: 504 - protein, 9 aa, synthetic construct
  REGION 1..9: note = SeV_CT_09
MSDSERSGK

SEQ ID NO: 505 - protein, 10 aa, synthetic construct
  REGION 1..10: note = SeV_CT_10
MVSDSERSGK

SEQ ID NO: 506 - protein, 11 aa, synthetic construct
  REGION 1..11: note = SeV_CT_11
MLVSDSERSGK

SEQ ID NO: 507 - protein, 12 aa, synthetic construct
  REGION 1..12: note = SeV_CT_12
MKLVSDSERSGK

SEQ ID NO: 508 - protein, 13 aa, synthetic construct
  REGION 1..13: note = SeV_CT_13
MTKLVSDSERSGK

SEQ ID NO: 509 - protein, 14 aa, synthetic construct
  REGION 1..14: note = SeV_CT_14
MTTKLVSDSERSGK

SEQ ID NO: 510 - protein, 15 aa, synthetic construct
  REGION 1..15: note = SeV_CT_15
MSTTKLVSDSERSGK

SEQ ID NO: 511 - protein, 16 aa, synthetic construct
  REGION 1..16: note = SeV_CT_16
MGSTTKLVSDSERSGK

SEQ ID NO: 512 - protein, 17 aa, synthetic construct
  REGION 1..17: note = SeV_CT_17
MGGSTTKLVSDSERSGK

SEQ ID NO: 513 - protein, 18 aa, synthetic construct
  REGION 1..18: note = SeV_CT_18
MPGGSTTKLVSDSERSGK

SEQ ID NO: 514 - protein, 19 aa, synthetic construct
  REGION 1..19: note = SeV_CT_19
MSPGGSTTKLVSDSERSGK

SEQ ID NO: 515 - protein, 20 aa, synthetic construct
  REGION 1..20: note = SeV_CT_20
MTSPGGSTTKLVSDSERSGK

SEQ ID NO: 516 - protein, 32 aa, synthetic construct
  REGION 1..32: note = SeV_CT_32_FL
MDGDRSKRDSYWSTSPGGSTTKLVSDSERSGK

SEQ ID NO: 517 - protein, 35 aa, synthetic construct
  REGION 1..35: note = BaEVwt_CT
MDQAEEDTRLVQYQQTLVMAHIINLKDNIFATLRN

SEQ ID NO: 518 - protein, 28 aa, synthetic construct
  REGION 1..28: note = BaEVRLess_CT
MAHIINLKDNIFATLRNKKINEGLLDSK

SEQ ID NO: 519 - protein, 17 aa, synthetic construct
  REGION 1..17: note = BaEVRLess_CT
MAHIINLKDNIFATLRN

SEQ ID NO: 520 - protein, 24 aa, synthetic construct
  REGION 1..24: note = Cocal_CT
MKRFRSMEIDNYIRKNNSGQYRYR

SEQ ID NO: 521 - protein, 35 aa, synthetic construct
  REGION 1..35: note = Cocal_CT
MKRFRSMEIDNYIRKNNSGQYRYRKKINEGLLDSK

SEQ ID NO: 522 - protein, 6 aa, synthetic construct
  REGION 1..6: note = EboV-GP_CT
MFVFKC

SEQ ID NO: 523 - protein, 17 aa, synthetic construct
  REGION 1..17: note = EboV-GP_CT
MFVFKCKKINEGLLDSK

SEQ ID NO: 524 - protein, 43 aa, synthetic construct
  REGION 1..43: note = GaLV_CT
MLNGENELAQYKQRLVLIKVASIRDNIFQVLKKKINEGLLDSK

SEQ ID NO: 525 - protein, 32 aa, synthetic construct
  REGION 1..32: note = GaLV_CT
MLNGENELAQYKQRLVLIKVASIRDNIFQVLK

SEQ ID NO: 526 - protein, 28 aa, synthetic construct
  REGION 1..28: note = GaLV-RLess_CT
MLIKVASIRDNIFQVLKKKINEGLLDSK

SEQ ID NO: 527 - protein, 17 aa, synthetic construct
  REGION 1..17: note = GaLV-RLess_CT
MLIKVASIRDNIFQVLK

SEQ ID NO: 528 - protein, 40 aa, synthetic construct
  REGION 1..40: note = LCMV_GP_CT
MRRKWVTKVGPVKFAGCSCIGKNTLRHPKPCSGGKIHRHT

SEQ ID NO: 529 - protein, 44 aa, synthetic construct
  REGION 1..44: note = MLV-A_CT
MPEYEIPKLQHYQQTLVLAQVVSIRDKVFQVLRKKINEGLLDSK

SEQ ID NO: 530 - protein, 33 aa, synthetic construct
  REGION 1..33: note = MLV-A_CT
MPEYEIPKLQHYQQTLVLAQVVSIRDKVFQVLR

SEQ ID NO: 531 - protein, 28 aa, synthetic construct
  REGION 1..28: note = MLV-A-RLess_CT
MLAQVVSIRDKVFQVLRKKINEGLLDSK

SEQ ID NO: 532 - protein, 17 aa, synthetic construct
  REGION 1..17: note = MLV-A-RLess_CT
MLAQVVSIRDKVFQVLR

SEQ ID NO: 533 - protein, 30 aa, synthetic construct
  REGION 1..30: note = VSV-G_CT
MKGLRNMEIDTYIQRKKTHKLKICLHIGVR

SEQ ID NO: 534 - protein, 41 aa, synthetic construct
  REGION 1..41: note = VSV-G_CT
MKGLRNMEIDTYIQRKKTHKLKICLHIGVRKKINEGLLDSK

SEQ ID NO: 535 - protein, 152 aa, synthetic construct
  REGION 1..152: note = HIV-1_gp41_MLLIR-LLP1-LLP3-LLP2-gp41-CT-N
MLLIRELGQRIRRPIHRIARYAAQLVEIVRDTGEAVAIATANLLNVASNKLEQSWYQLLNWWYKLAEWGRRGLLEVIRTVILLLDRLRHYSFLCLSRLDDWILALSGNVLRISRDRDREGGEEEIGEPRDPGRPIPLHTQFSLPSYGQRVRN

SEQ ID NO: 536 - protein, 163 aa, synthetic construct
  REGION 1..163: note = HIV-1_gp41_MLLIR-LLP1-LLP3-LLP2-gp41-CT-N
MLLIRELGQRIRRPIHRIARYAAQLVEIVRDTGEAVAIATANLLNVASNKLEQSWYQLLNWWYKLAEWGRRGLLEVIRTVILLLDRLRHYSFLCLSRLDDWILALSGNVLRISRDRDREGGEEEIGEPRDPGRPIPLHTQFSLPSYGQRVRNKKINEGLLDSK

SEQ ID NO: 537 - protein, 119 aa, synthetic construct
  REGION 1..119: note = HIV-1_gp41_LLP3-LLP2-gp41-CT-N
MAVAIATANLLNVASNKLEQSWYQLLNWWYKLAEWGRRGLLEVIRTVILLLDRLRHYSFLCLSRLDDWILALSGNVLRISRDRDREGGEEEIGEPRDPGRPIPLHTQFSLPSYGQRVRN

SEQ ID NO: 538 - protein, 81 aa, synthetic construct
  REGION 1..81: note = HIV-1_gp41_LLP2-gp41-CT-N
MLLEVIRTVILLLDRLRHYSFLCLSRLDDWILALSGNVLRISRDRDREGGEEEIGEPRDPGRPIPLHTQFSLPSYGQRVRN

SEQ ID NO: 539 - protein, 48 aa, synthetic construct
  REGION 1..48: note = HIV-1_gp41_gp41-CT-N
MSGNVLRISRDRDREGGEEEIGEPRDPGRPIPLHTQFSLPSYGQRVRN

SEQ ID NO: 540 - protein, 105 aa, synthetic construct
  REGION 1..105: note = HIV-1_gp41_MLLIR-LLP1-LLP3-LLP2
MLLIRELGQRIRRPIHRIARYAAQLVEIVRDTGEAVAIATANLLNVASNKLEQSWYQLLNWWYKLAEWGRRGLLEVIRTVILLLDRLRHYSFLCLSRLDDWILAL

SEQ ID NO: 541 - protein, 101 aa, synthetic construct
  REGION 1..101: note = HIV-1_gp41_LLP1-LLP3-LLP2
MELGQRIRRPIHRIARYAAQLVEIVRDTGEAVAIATANLLNVASNKLEQSWYQLLNWWYKLAEWGRRGLLEVIRTVILLLDRLRHYSFLCLSRLDDWILAL

SEQ ID NO: 542 - protein, 72 aa, synthetic construct
  REGION 1..72: note = HIV-1_gp41_LLP3-LLP2
MAVAIATANLLNVASNKLEQSWYQLLNWWYKLAEWGRRGLLEVIRTVILLLDRLRHYSFLCLSRLDDWILAL

SEQ ID NO: 543 - protein, 34 aa, synthetic construct
  REGION 1..34: note = HIV-1_gp41_LLP2
MLLEVIRTVILLLDRLRHYSFLCLSRLDDWILAL

SEQ ID NO: 544 - protein, 68 aa, synthetic construct
  REGION 1..68: note = HIV-1_gp41_MLLIR-LLP1-LLP3
MLLIRELGQRIRRPIHRIARYAAQLVEIVRDTGEAVAIATANLLNVASNKLEQSWYQLLNWWYKLAEW

SEQ ID NO: 545 - protein, 64 aa, synthetic construct
  REGION 1..64: note = HIV-1_gp41_LLP1-LLP3
MELGQRIRRPIHRIARYAAQLVEIVRDTGEAVAIATANLLNVASNKLEQSWYQLLNWWYKLAEW

SEQ ID NO: 546 - protein, 35 aa, synthetic construct
  REGION 1..35: note = HIV-1_gp41_LLP3
MAVAIATANLLNVASNKLEQSWYQLLNWWYKLAEW

SEQ ID NO: 547 - protein, 30 aa, synthetic construct
  REGION 1..30: note = HIV-1_gp41_MLLIR-LLP1
MLLIRELGQRIRRPIHRIARYAAQLVEIVR

SEQ ID NO: 548 - protein, 26 aa, synthetic construct
  REGION 1..26: note = HIV-1_gp41_LLP1
MELGQRIRRPIHRIARYAAQLVEIVR

SEQ ID NO: 549 - protein, 5 aa, synthetic construct
  REGION 1..5: note = HIV-1_gp41_MLLIR
MLLIR

SEQ ID NO: 550 - protein, 11 aa, synthetic construct
  REGION 1..11: note = CD63_CT_1to11
MAVEGGMKCVK

SEQ ID NO: 551 - protein, 10 aa, synthetic construct
  REGION 1..10: note = CD63_CT_73to81
MCGACKENYC

SEQ ID NO: 552 - protein, 14 aa, synthetic construct
  REGION 1..14: note = CD63_CT_225to238
MVEYGSRISKVLCC

SEQ ID NO: 553 - protein, 22 aa, synthetic construct
  REGION 1..22: note = CD63_CT_1to11
MAVEGGMKCVKKKINEGLLDSK

SEQ ID NO: 554 - protein, 21 aa, synthetic construct
  REGION 1..21: note = CD63_CT_73to81
MCGACKENYCKKINEGLLDSK

SEQ ID NO: 555 - protein, 25 aa, synthetic construct
  REGION 1..25: note = CD63_CT_225to238
MVEYGSRISKVLCCKKINEGLLDSK

SEQ ID NO: 556 - protein, 48 aa, synthetic construct
  REGION 1..48: note = CD29_CT
MKGEYKPNVVTTVASKYIPNEGTDWKANMKEKEFKAFERRDHIIMLLK

SEQ ID NO: 557 - protein, 59 aa, synthetic construct
  REGION 1..59: note = CD29_CT
MKGEYKPNVVTTVASKYIPNEGTDWKANMKEKEFKAFERRDHIIMLLKKKINEGLLDSK

SEQ ID NO: 558 - protein, 569 aa, synthetic construct
  REGION 1..569: note = MvH_CT_12
MVINREHLMIDRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 559 - protein, 573 aa, synthetic construct
  REGION 1..573: note = MvH_CT_16
MGSRIVINREHLMIDRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 560 - protein, 577 aa, synthetic construct
  REGION 1..577: note = MvH_CT_20
MPHPKGSRIVINREHLMIDRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 561 - protein, 576 aa, synthetic construct
  REGION 1..576: note = NivG_CT_19
MYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 562 - protein, 565 aa, synthetic construct
  REGION 1..565: note = BatPV_G_CT_8
MKNQNDHYILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 563 - protein, 565 aa, synthetic construct
  REGION 1..565: note = SeV_CT_08
MDSERSGKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 564 - protein, 574 aa, synthetic construct
  REGION 1..574: note = MLV-A-RLess_CT
MLAQVVSIRDKVFQVLRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 565 - protein, 562 aa, synthetic construct
  REGION 1..562: note = HIV-1_gp41_MLLIR
MLLIRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 566 - protein, 568 aa, synthetic construct
  REGION 1..568: note = CD63_CT_1to11
MAVEGGMKCVKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 567 - protein, 567 aa, synthetic construct
  REGION 1..567: note = CD63_CT_73to81
MCGACKENYCILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 568 - protein, 569 aa, synthetic construct
  REGION 1..569: note = HevG_CT_12
MKKINDGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 569 - protein, 590 aa, synthetic construct
  REGION 1..590: note = MLV-A_CT
MPEYEIPKLQHYQQTLVLAQVVSIRDKVFQVLRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 570 - protein, 563 aa, synthetic construct
  REGION 1..563: note = NivG_CT_06
MLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 571 - protein, 570 aa, synthetic construct
  REGION 1..570: note = NivG_CT_13
MIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 572 - protein, 569 aa, synthetic construct
  REGION 1..569: note = BatPV_G_CT_12
MWKKQKNQNDHYILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 573 - protein, 563 aa, synthetic construct
  REGION 1..563: note = HPIV2-HN_CT_06
MRIIFRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 574 - protein, 569 aa, synthetic construct
  REGION 1..569: note = HPIV2-HN_CT_12
MIPKRTCRIIFRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 575 - protein, 569 aa, synthetic construct
  REGION 1..569: note = CD63_CT_1to11
MKLVSDSERSGKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 576 - protein, 238 aa, synthetic construct
  REGION 1..238: note = CD133.S.001.02
EVKLVESGPELVKPGETVKISCKASGYTFTDYSMHWVRQAPGKGLEWMGWINTETGEPSYADDFKGRFAFSLDTSASTAYLQINSLRSEDTATYFCATDYGDYFDYWGQGTTVTVSSGGGGSGGGGSGGGGSDIVLSQSPAIMSASPGEKVTISCSASSSVSYMYWYQQKPGSSPKPWIYRTSNLASGVPARFSGSGSGTSYSLTISSMEPEDAATYYCQQYHSYPPTFGAGTKLELK

SEQ ID NO: 577 - protein, 245 aa, synthetic construct
  REGION 1..245: note = CD133.s.004.01
DVQLQESGPGLVKPSQSLSLTCSVTGYSITSGYYWSWIRQFPGNKLEWMGYISYDGSNNYNPSLKNRISITRDTSKNQIFLKLNSVTTEDTATYYCTRVYFGHPYYYGMDYWGQGISVTVGGGGSGGGGSGGGGSQIVLTQSPAIMSASLGERVTMTCTASSSVSSFYLHWYRQKPGSSPKLWIYSTSNLASGVPARFSGSGSGTSYSLTISSMEAEDAATYYCHQYHRSPYMYMFGGGTKLEIK

SEQ ID NO: 578 - protein, 247 aa, synthetic construct
  REGION 1..247: note = CD133.s.006.01
EVQLVESGGGLVQPGGSLRLSCAASGFNIYYYGSMHWVRQAPGKGLEWVASISPYYGSTYYADSVKGRFTISADTSKNTAYLQMNSLRAEDTAVYYCARHASSGYGHYAVYGIDYWGQGTLVTVSSGGGGSGGGGSGGGGSDIQMTQSPSSLSASVGDRVTITCRASQGSSYVAWYQQKPGKAPKLLIYSASYLYSGVPSRFSGSRSGTDFTLTISSLQPEDFATYYCQQGVWSLITFGQGTKVEIK

SEQ ID NO: 579 - protein, 569 aa, synthetic construct; identical to SEQ ID NO: 2
  REGION 1..569: note = NivG.690

SEQ ID NO: 580 - protein, 569 aa, synthetic construct; identical to SEQ ID NO: 3
  REGION 1..569: note = NivG.691

SEQ ID NO: 581 - protein, 569 aa, synthetic construct; identical to SEQ ID NO: 5
  REGION 1..569: note = NivG.693

SEQ ID NO: 582 - protein, 569 aa, synthetic construct; identical to SEQ ID NO: 6
  REGION 1..569: note = NivG.694

SEQ ID NO: 583 - protein, 569 aa, synthetic construct; identical to SEQ ID NO: 7
  REGION 1..569: note = NivG.695

SEQ ID NO: 584 - protein, 569 aa, synthetic construct; identical to SEQ ID NO: 8
  REGION 1..569: note = NivG.696

SEQ ID NO: 585 - protein, 569 aa, synthetic construct; identical to SEQ ID NO: 9
  REGION 1..569: note = NivG.697

SEQ ID NO: 586 - protein, 569 aa, synthetic construct; identical to SEQ ID NO: 10
  REGION 1..569: note = NivG.699

SEQ ID NO: 587 - protein, 569 aa, synthetic construct; identical to SEQ ID NO: 11
  REGION 1..569: note = NivG.700

SEQ ID NO: 588 - protein, 569 aa, synthetic construct; identical to SEQ ID NO: 12
  REGION 1..569: note = NivG.701

SEQ ID NO: 589 - protein, 569 aa, synthetic construct; identical to SEQ ID NO: 354
  REGION 1..569: note = NivG.702

SEQ ID NO: 590 - protein, 569 aa, synthetic construct; identical to SEQ ID NO: 246
  REGION 1..569: note = NivG.740

SEQ ID NO: 591 - protein, 569 aa, synthetic construct; identical to SEQ ID NO: 247
  REGION 1..569: note = NivG.742

SEQ ID NO: 592 - protein, 569 aa, synthetic construct
  REGION 1..569: note = NivG.743
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 593 - protein, 569 aa, synthetic construct
  REGION 1..569: note = NivG.744
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 594 - protein, 569 aa, synthetic construct
  REGION 1..569: note = NivG.748
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 595 - protein, 569 aa, synthetic construct
  REGION 1..569: note = NivG.749
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 596 - protein, 569 aa, synthetic construct; identical to SEQ ID NO: 249
  REGION 1..569: note = NivG.750

SEQ ID NO: 597 - protein, 569 aa, synthetic construct; identical to SEQ ID NO: 254
  REGION 1..569: note = NivG.752

SEQ ID NO: 598 - protein, 569 aa, synthetic construct
  REGION 1..569: note = NivG.753
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 599 - protein, 569 aa, synthetic construct; identical to SEQ ID NO: 256
  REGION 1..569: note = NivG.754

SEQ ID NO: 600 - protein, 569 aa, synthetic construct; identical to SEQ ID NO: 257
  REGION 1..569: note = NivG.755

SEQ ID NO: 601 - protein, 569 aa, synthetic construct; identical to SEQ ID NO: 258
  REGION 1..569: note = NivG.756

SEQ ID NO: 602 - protein, 569 aa, synthetic construct; identical to SEQ ID NO: 251
  REGION 1..569: note = NivG.757

SEQ ID NO: 603 - protein, 569 aa, synthetic construct
  REGION 1..569: note = NivG.758
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 604 - protein, 569 aa, synthetic construct
  REGION 1..569: note = NivG.759
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 605 - protein, 569 aa, synthetic construct; identical to SEQ ID NO: 262
  REGION 1..569: note = NivG.760

SEQ ID NO: 606 - protein, 569 aa, synthetic construct
  REGION 1..569: note = NivG.761
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 607 - protein, 569 aa, synthetic construct
  REGION 1..569: note = NivG.762
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 608 - protein, 569 aa, synthetic construct; identical to SEQ ID NO: 252
  REGION 1..569: note = NivG.763

SEQ ID NO: 609 - protein, 569 aa, synthetic construct; identical to SEQ ID NO: 266
  REGION 1..569: note = NivG.764

SEQ ID NO: 610 - protein, 569 aa, synthetic construct; identical to SEQ ID NO: 267
  REGION 1..569: note = NivG.765

SEQ ID NO: 611 - protein, 569 aa, synthetic construct; identical to SEQ ID NO: 248
  REGION 1..569: note = NivG.766

SEQ ID NO: 612 - protein, 569 aa, synthetic construct
  REGION 1..569: note = NivG.767
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 613 - protein, 569 aa, synthetic construct
  REGION 1..569: note = NivG.768
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 614 - protein, 569 aa, synthetic construct; identical to SEQ ID NO: 271
  REGION 1..569: note = NivG.769

SEQ ID NO: 615 - protein, 569 aa, synthetic construct
  REGION 1..569: note = NivG.770
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 616 - protein, 569 aa, synthetic construct; identical to SEQ ID NO: 273
  REGION 1..569: note = NivG.771

SEQ ID NO: 617 - protein, 569 aa, synthetic construct
  REGION 1..569: note = NivG.772
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 618 - protein, 569 aa, synthetic construct; identical to SEQ ID NO: 275
  REGION 1..569: note = NivG.773

SEQ ID NO: 619 - protein, 569 aa, synthetic construct; identical to SEQ ID NO: 276
  REGION 1..569: note = NivG.774

SEQ ID NO: 620 - protein, 569 aa, synthetic construct
  REGION 1..569: note = NivG.775
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 621 - protein, 569 aa, synthetic construct
  REGION 1..569: note = NivG.776
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 622 - protein, 569 aa, synthetic construct
  REGION 1..569: note = NivG.777
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 623 - protein, 569 aa, synthetic construct
  REGION 1..569: note = NivG.778
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 624 - protein, 569 aa, synthetic construct
  REGION 1..569: note = NivG.779
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 625 - protein, 569 aa, synthetic construct; identical to SEQ ID NO: 281
  REGION 1..569: note = NivG.780

SEQ ID NO: 626 - protein, 569 aa, synthetic construct; identical to SEQ ID NO: 282
  REGION 1..569: note = NivG.781

SEQ ID NO: 627 - protein, 569 aa, synthetic construct
  REGION 1..569: note = NivG.782
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 628 - protein, 569 aa, synthetic construct
  REGION 1..569: note = NivG.783
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 629 - protein, 569 aa, synthetic construct; identical to SEQ ID NO: 284
  REGION 1..569: note = NivG.784

SEQ ID NO: 630 - protein, 569 aa, synthetic construct; identical to SEQ ID NO: 277
  REGION 1..569: note = NivG.785

SEQ ID NO: 631 - protein, 569 aa, synthetic construct; identical to SEQ ID NO: 287
  REGION 1..569: note = NivG.786

SEQ ID NO: 632 - protein, 569 aa, synthetic construct
  REGION 1..569: note = NivG.787
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 633 - protein, 569 aa, synthetic construct; identical to SEQ ID NO: 289
  REGION 1..569: note = NivG.788

SEQ ID NO: 634 - protein, 569 aa, synthetic construct; identical to SEQ ID NO: 279
  REGION 1..569: note = NivG.789

SEQ ID NO: 635 - protein, 569 aa, synthetic construct; identical to SEQ ID NO: 291
  REGION 1..569: note = NivG.790

SEQ ID NO: 636 - protein, 569 aa, synthetic construct; identical to SEQ ID NO: 292
  REGION 1..569: note = NivG.791

SEQ ID NO: 637 - protein, 569 aa, synthetic construct; identical to SEQ ID NO: 293
  REGION 1..569: note = NivG.792

SEQ ID NO: 638 - protein, 569 aa, synthetic construct
  REGION 1..569: note = NivG.793
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 639 - protein, 569 aa, synthetic construct; identical to SEQ ID NO: 280
  REGION 1..569: note = NivG.794

SEQ ID NO: 640 - protein, 569 aa, synthetic construct; identical to SEQ ID NO: 296
  REGION 1..569: note = NivG.795

SEQ ID NO: 641 - protein, 569 aa, synthetic construct
  REGION 1..569: note = NivG.796
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 642 - protein, 569 aa, synthetic construct; identical to SEQ ID NO: 298
  REGION 1..569: note = NivG.797

SEQ ID NO: 643 - protein, 569 aa, synthetic construct
  REGION 1..569: note = NivG.798
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 644 - protein, 569 aa, synthetic construct; identical to SEQ ID NO: 300
  REGION 1..569: note = NivG.799

SEQ ID NO: 645 - protein, 569 aa, synthetic construct
  REGION 1..569: note = NivG.800
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 646 - protein, 569 aa, synthetic construct; identical to SEQ ID NO: 302
  REGION 1..569: note = NivG.801

SEQ ID NO: 647 - protein, 569 aa, synthetic construct
  REGION 1..569: note = NivG.802
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 648 - protein, 569 aa, synthetic construct
  REGION 1..569: note = NivG.803
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 649 - protein, 569 aa, synthetic construct
  REGION 1..569: note = NivG.804
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 650 - protein, 569 aa, synthetic construct
  REGION 1..569: note = NivG.805
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 651 - protein, 569 aa, synthetic construct; identical to SEQ ID NO: 306
  REGION 1..569: note = NivG.806

SEQ ID NO: 652 - protein, 569 aa, synthetic construct; identical to SEQ ID NO: 307
  REGION 1..569: note = NivG.807

SEQ ID NO: 653 - protein, 569 aa, synthetic construct
  REGION 1..569: note = NivG.808
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 654 - protein, 569 aa, synthetic construct
  REGION 1..569: note = NivG.809
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 655 - protein, 569 aa, synthetic construct; identical to SEQ ID NO: 309
  REGION 1..569: note = NivG.810

SEQ ID NO: 656 - protein, 569 aa, synthetic construct; identical to SEQ ID NO: 310
  REGION 1..569: note = NivG.811

SEQ ID NO: 657 - protein, 569 aa, synthetic construct
  REGION 1..569: note = NivG.812
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 658 - protein, 569 aa, synthetic construct; identical to SEQ ID NO: 312
  REGION 1..569: note = NivG.813

SEQ ID NO: 659 - protein, 569 aa, synthetic construct; identical to SEQ ID NO: 313
  REGION 1..569: note = NivG.814

SEQ ID NO: 660 - protein, 569 aa, synthetic construct; identical to SEQ ID NO: 314
  REGION 1..569: note = NivG.815

SEQ ID NO: 661 - protein, 569 aa, synthetic construct
  REGION 1..569: note = NivG.816
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 662 - protein, 569 aa, synthetic construct
  REGION 1..569: note = NivG.817
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 663 - protein, 569 aa, synthetic construct
  REGION 1..569: note = NivG.818
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 664 - protein, 569 aa, synthetic construct
  REGION 1..569: note = NivG.819
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 665 - protein, 569 aa, synthetic construct; identical to SEQ ID NO: 304
  REGION 1..569: note = NivG.820

SEQ ID NO: 666 - protein, 569 aa, synthetic construct
  REGION 1..569: note = NivG.821
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 667 - protein, 569 aa, synthetic construct
  REGION 1..569: note = NivG.822
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 668 - protein, 569 aa, synthetic construct; identical to SEQ ID NO: 322
  REGION 1..569: note = NivG.823

SEQ ID NO: 669 - protein, 569 aa, synthetic construct; identical to SEQ ID NO: 323
  REGION 1..569: note = NivG.824

SEQ ID NO: 670 - protein, 569 aa, synthetic construct; identical to SEQ ID NO: 317
  REGION 1..569: note = NivG.825

SEQ ID NO: 671 - protein, 569 aa, synthetic construct; identical to SEQ ID NO: 325
  REGION 1..569: note = NivG.826

SEQ ID NO: 672 - protein, 569 aa, synthetic construct; identical to SEQ ID NO: 305
  REGION 1..569: note = NivG.827

SEQ ID NO: 673 - protein, 569 aa, synthetic construct
  REGION 1..569: note = NivG.828
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 674 - protein, 569 aa, synthetic construct; identical to SEQ ID NO: 328
  REGION 1..569: note = NivG.829

SEQ ID NO: 675 - protein, 569 aa, synthetic construct; identical to SEQ ID NO: 329
  REGION 1..569: note = NivG.830

SEQ ID NO: 676 - protein, 569 aa, synthetic construct; identical to SEQ ID NO: 330
  REGION 1..569: note = NivG.831

SEQ ID NO: 677 - protein, 569 aa, synthetic construct
  REGION 1..569: note = NivG.832
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 678 - protein, 569 aa, synthetic construct
  REGION 1..569: note = NivG.833
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 679 - protein, 569 aa, synthetic construct
  REGION 1..569: note = NivG.834
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 680 - protein, 569 aa, synthetic construct
  REGION 1..569: note = NivG.835
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 681 - protein, 569 aa, synthetic construct; identical to SEQ ID NO: 332
  REGION 1..569: note = NivG.836

SEQ ID NO: 682 - protein, 569 aa, synthetic construct; identical to SEQ ID NO: 335
  REGION 1..569: note = NivG.837

SEQ ID NO: 683 - protein, 569 aa, synthetic construct; identical to SEQ ID NO: 336
  REGION 1..569: note = NivG.838

SEQ ID NO: 684 - protein, 569 aa, synthetic construct; identical to SEQ ID NO: 337
  REGION 1..569: note = NivG.839

SEQ ID NO: 685 - protein, 569 aa, synthetic construct; identical to SEQ ID NO: 338
  REGION 1..569: note = NivG.840

SEQ ID NO: 686 - protein, 569 aa, synthetic construct
  REGION 1..569: note = NivG.841
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 687 - protein, 569 aa, synthetic construct; identical to SEQ ID NO: 340
  REGION 1..569: note = NivG.842

SEQ ID NO: 688 - protein, 569 aa, synthetic construct; identical to SEQ ID NO: 341
  REGION 1..569: note = NivG.843

SEQ ID NO: 689 - protein, 569 aa, synthetic construct
  REGION 1..569: note = NivG.844
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 690 - protein, 569 aa, synthetic construct
  REGION 1..569: note = NivG.845
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 691 - protein, 569 aa, synthetic construct; identical to SEQ ID NO: 343
  REGION 1..569: note = NivG.846

SEQ ID NO: 692 - protein, 569 aa, synthetic construct; identical to SEQ ID NO: 344
  REGION 1..569: note = NivG.847

SEQ ID NO: 693 - protein, 569 aa, synthetic construct
  REGION 1..569: note = NivG.848
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 694 - protein, 569 aa, synthetic construct; identical to SEQ ID NO: 350
  REGION 1..569: note = NivG.849

SEQ ID NO: 695 - protein, 569 aa, synthetic construct; identical to SEQ ID NO: 346
  REGION 1..569: note = NivG.850

SEQ ID NO: 696 - protein, 569 aa, synthetic construct
  REGION 1..569: note = NivG.851
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 697 - protein, 569 aa, synthetic construct
  REGION 1..569: note = NivG.852
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 698 - protein, 569 aa, synthetic construct
  REGION 1..569: note = NivG.853
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 699 - protein, 569 aa, synthetic construct; identical to SEQ ID NO: 349
  REGION 1..569: note = NivG.854

SEQ ID NO: 700 - protein, 569 aa, synthetic construct
  REGION 1..569: note = NivG.855
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 701 - protein, 569 aa, synthetic construct; identical to SEQ ID NO: 351
  REGION 1..569: note = NivG.856

SEQ ID NO: 702 - protein, 569 aa, synthetic construct; identical to SEQ ID NO: 345
  REGION 1..569: note = NivG.857

SEQ ID NO: 703 - protein, 569 aa, synthetic construct
  REGION 1..569: note = NivG.858
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 704 - protein, 569 aa, synthetic construct
  REGION 1..569: note = NivG.859
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 705 - protein, 569 aa, synthetic construct
  REGION 1..569: note = NivG.860
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 706 - protein, 121 aa, synthetic construct
  REGION 1..121: note = VHH
EVQLVESGGGLVQSGGSLRLSCAASGFTFSGYWMYWVRQAPGKGLEWVSAISPGGGSTYYPDSVKGRFTISRDNAKNTLYLQMNSLEPEDTALYYCASSLTATHTYEYDYWGQGTQVTVSS